(19) United States
(12) Patent Application Publication
(10) Pub. No.: US 2007/0020631 A1
(43) Pub. Date: Jan. 25, 2007

(54) Identification of streptococcus penumoniae serotypes

(75) Inventors: Fanrong Kong, Westmead (AU); Gwendolyn Gilbert, Riverview (AU); Lei Wang, Tianjin (CN); Dan Liu, Tianjin (CN); Jiang Tao, Tianjin (CN)

Correspondence Address: MARSHALL, GERSTEIN & BORUN LLP, 233 S. WACKER DRIVE, SUITE 6300, SEARS TOWER, CHICAGO, IL, 60606, US

(73) Assignees: Sydney West Area Health Service Swahs Office of Commercialisation, Westmead (AU); Tianjin Biochip Corporation, Tianjin (CN)

(21) Appl. No.: 10/552,394

(22) PCT Filed: Apr. 13, 2004
(86) PCT No.: PCT/AU04/00480

§ 371 Date: Jun. 16, 2006

(30) Foreign Application Priority Data
Apr. 10, 2003 (AU) 2003901717

(51) Int. Cl. C12Q 1/68; C12P 19/34

(52) U.S. Cl. 435/6; 435/91.2

(57) ABSTRACT

The present invention relates to molecular methods of serotyping Streptococcus pneunoniae, as well as polynu-cleotides useful in such methods. These methods rely on analysing at least a portion of the nucleotide sequence between the 3′ end of the cpsA gene and the 5′ end of the cpsB gene, and/or analysing at least a portion of the szy and/or wzx gene(s).

### FIELD OF THE INVENTION

The present invention relates to molecular methods of serotyping Streptococcus pneumoniae, as well as polynucleotides useful in such methods.

### BACKGROUND OF THE INVENTION

Streptococcus pneumoniae is a leading cause of morbidity and mortality causing invasive disease such as meningitis and pneumonia as well as more localised disease such as acute otitis media and sinusitis. Polysaccharide and protein-conjugate pneumococcal vaccines have the potential to prevent a significant proportion of cases. Effective protein-conjugate vaccines are particularly important because of the dramatic increase in prevalence and international dissemination of antibiotic resistant S. pneumoniae serotypes that commonly cause invasive disease in children (Hausdorff et el., 2001; Huebner, et al., 2000). However these vaccines protect against only the relatively small minority (Dunne et al., 2001; Hausdorff et el., 2001) of pneumococcal serotypes that most commonly cause disease. There is theoretical and limited empirical evidence that widespread use of these vaccines could lead to substitution of “vaccine” serotypes with other nonvaccine serotypes, against which the vaccines do not provide protection. Continued surveillance will be critical to monitor vaccine efficacy and changes in incidence and distribution of colonising and invasive serotypes (Hausdorff et el., 2001; Rubins et al., 1999). Any increase in disease caused by previously uncommon nonvaccine serotypes could necessitate a change in vaccine composition (Lipsitch, 2001).

S. pneuinoniae comprises at least 90 serotypes, distinguished by capsular polysaccharide antigens. Pneumococcal serotype/group identification is currently performed, using large panels of expensive antisera, by various methods, including capsular swelling (Quellung) reaction—the traditional “gold standard”—latex agglutination and coagglutination (Arai et al., 2001; Lalitha et al., 1999). Cross-reactions between serotypes and discrepancies between methods can occur and some strains are nonserotypable (Henrichsen, 1999).

The capsular polysaccharide synthesis (cps) gene clusters for at least 16 pneumococcal serotypes have been sequenced and serotype-specific genes identified (Jiang et al., 2001; van Selm et al., 2002). The cps gene cluster contains genes responsible for synthesis of the serotype-specific polysaccharide including—except in serotype 3—wzy (polysaccharide polymerase gene) and wzx (polysccharide flippase gene). At the 5′-end of the cps gene cluster are four relatively conserved open reading frames—cpsA (wzg)-cpsB (wzh)-cpsC (wzd)-cpsD (wze). Sequence differences in this region were used to classify 11 S. pneumoniae serotypes into two classes and, in the region between the 3′-end of cpsA and the 5′-end of cpsB, there were sites of heterogeneity between and within serotypes (Jiang et al., 2001; Lawrence et al., 2000). S. pneumoniae is characterised by high frequency recombination within the cps gene cluster, leading to serotype “switching” among isolates within genetic lineages defined by relationships between their more conserved housekeeping genes (Coffey et al., 1998; Jiang et al., 2001).

The relatively low percentage of polymorphisms between strains which is linked to actual serotype, and the large number of different serotypes, has made the development of assays which can be used for typing a significant portion of S. pneumoniae strains difficult. Accordingly, there is a need for further methods which can be used to identify different Streptococcus pneumoniae serotypes.

### SUMMARY OF THE INVENTION

Through the complex analysis of a large number of polymorphisms which exist between at least 132 molecular capsular sequence types of Streptococcus pneumoniae the present inventors have devised methods which can be used to distinguish between a majority of different S. pneumoniae serotypes. In particular, prior art methods of nucleic acid based typing techniques could serotype only about 20 serotypes of S. pneumoniae. In contrast, the methods of the invention can be used to serotype most of the about 90 serotypes of S. pneumoniae. The methods of the invention can also be used to subtype some serotypes.

Thus, in a first aspect, the present invention provides a method of distinguishing between at least 25 different serotypes of Streptococcus pneumoniae in a sample, the method comprising,

i) analysing at least a portion of the nucleotide sequence between the 3′ end of the cpsA gene and the 5′ end of the cpsB gene, and/or

ii) analysing at least a portion of the wzy and/or wzx gene(s).

Preferably, the method can be used to type at least 40, more preferably at least 50, more preferably at least 70, more preferably at least 90, more preferably at least 100, even more preferably at least about 132 different molecular capsular sequence types of S. pneumoniae.

The present inventors are the first to provide suitable nucleic acid based techniques for typing a large number of Streptococcus pneumoniae serotypes. Accordingly, in another aspect the present invention provides a method of determining the serotype of Streptococcus pneumoniae in a sample, the method comprising,

i) analysing at least a portion of the nucleotide sequence between the 3′ end of the cpsA gene and the 5′ end of the cpsB gene, and/or

ii) analysing at least a portion of the wzy and/or wzx gene(s), wherein the serotype is selected from the group consisting of: 2, 7A, 7B, 7C, 9A, 9L, 10F, 10A, 10B, 10C, 11F, 11A, 11B, 11C, 11D, 12F, 12A, 12B, 13, 15F, 15A, 15B, 15C, 16A, 17F, 17A, 18F, 18A, 18B, 21, 22F, 22A, 24F, 24A, 24B, 25F, 25A, 27, 28F, 28A, 31, 32F, 32A, 33F, 33A, 33B, 33C, 33D, 34, 35A, 35B, 35C, 36, 37, 38, 39, 40, 41F, 41A, 42, 43, 44, 45, 46, 47, 47A and 48.

The present inventors have surprisingly found that at least about 102 molecular capsular sequence types of S. pneumoniae can be directly serotyped by analysing the 3′ end of the cpsA gene and the 5′ end of the cpsB gene of the S. pneumoniae genome.

Thus, in another aspect the present invention provides a method of determining the serotype of Streptococcus pneumoniae in a sample, the method comprising analysing at least a portion of the nucleotide sequence between the 3′ end of the cpsA gene and the 5′ end of the cpsB gene.

In a preferred embodiment, the portion of the nucleotide sequence between the 3′ end of the cpsA gene and the 5′ end of the cpsB gene which is analysed is any nucleotide which is polymorphic between at least some of the S. pneumoniae serotypes referred to in FIG. 2.

In a particularly preferred embodiment, the method comprises amplifying at least a portion of the nucleotide sequence between the 3′ end of the cpsA gene and the 5′ end of the cpsB gene, and sequencing the amplification product. More preferably, the entire approximate 800 bp region as provided in FIG. 2 is amplified and sequenced.

In the case of sequencing to identify the serotype, the sequencing primers are selected such that they hybridise specifically to a region within or near to a region within which a polymorphism is present. The primers need not be specific to particular serotypes since it is the actual sequence information obtained during the sequencing process which is used to determine the S. pneumoniae serotype. Thus the primers may hybridise specifically to genomic DNA from all S. pneumoniae serotypes (or at least those serotypes referred to in FIG. 2), or to genomic DNA from some, but not all, S. pneumoniae serotypes.

When a portion of the nucleotide sequence between the 3′ end of the cpsA gene and the 5′ end of the cpsB gene is amplified, it is preferable that the amplification is performed using primer pairs comprising a sequence selected from the group consisting of:

(SEQ ID NO:68)

1) GGCATT(/C)TATGGAGTTGATTCG(/A)TCCATT(/C)CACAC(C/
T)TTAG
and

(SEQ ID NO:73)

GC(/T)TCAATG(/A)TGG(/A)GCAATG(/T)ACTGGA(/C)GTA(/G)
ATTCCCA(/G)ACATC,

(SEQ ID NO:68)

2) GGCATT(/C)TATGGAGTTGATTCG(/A)TCCATT(/C)CACACC
(/T)TTAG
and

(SEQ ID NO:71)

CCATCAC(/T)ATAGAGGTTAC(/A)TG(/A)TCTGGCATT(/C)GC,

(SEQ ID NO:70)

3) GAAAGTGGG(/A/T)GGG(/A/T)A(/G)A(/C)T(/G)TAT(/C)
AAAGTA(/G)AATTCT(/G)CAAGAT(/C)TTA(/G)AAA(/G)G
and

(SEQ ID NO:72)

T(/G)CATG(/A)CTA(/G)AAC(/T)TCT(/A)ATC(/T)AAG(/A)
GCATAACGACTATC(/T),
and

4) primer pairs that amplify the same region, or diagnostic portion thereof, from the genome of a strain of S. pneumoniae as the primers provided in 1) to 3).

In an alternate embodiment, the nucleotide sequence analysis step comprises determining whether a polynucleotide obtained from S. pneumoniae selectively hybridises to a polynucleotide probe comprising one or more polymorphic regions of the nucleotide sequence between the 3′ end of the cpsA gene and the 5′ end of the cpsB gene, wherein such polymorphic regions are shown in FIG. 2. More preferably, the nucleotide sequence analysis step comprises a plurality of said polynucleotide probes. In a particularly preferred embodiment, where hybridisation to a plurality of probes is used as a means of analysis, the plurality of polynucleotide probes are present as a microarray.

It has been noted that the method of analysing at least a portion of the nucleotide sequence between the 3′ end of the cpsA gene and the 5′ end of the cpsB gene does not enable the identification of all known S. pneumoniae serotypes, for example shared sequences were noted in the following cases; 6A and 6B; 10A and 17A, 10A and 23F, 23F and 23A; 15B, 15C, 22F and 22A; 17F, 35B, 35C and 42. Accordingly, in these instances further analysis will need to be performed to determine the correct serotype. To this end, the present inventors have discovered that polymorphisms in the wzy and/or wzx genes can also be useful for S. pneumoniae serotyping.

Accordingly, in a further aspect the present invention provides a method of determining the serotype of Streptococcus pneumoniae in a sample, the method comprising analysing at least a portion of the wzy and/or wzx gene(s).

In a preferred embodiment, the method comprises amplifying at least a portion of the wzy and/or wzx gene(s), and determining the length of the amplification product.

In a particularly preferred embodiment, at least a portion of the wzy and/or wzx gene(s) is amplified using primer pairs comprising a sequence selected from the group consisting of:

(SEQ ID NO:74)

1) GTAGGTGTAGTTTTTTCAGGGACTTTAATTTTATGCAGTG
and

(SEQ ID NO:75)

TCGCTTAACACAATGGCTTTAGAAGGTAGAG,

(SEQ ID NO:76)

2) GTTATTTTATTTTTTTTGTCGGCATTCTATTCTTTATATCG
and

(SEQ ID NO:77)

CAAATTCATCGTTTGTATCCATTTAACTGCATC,

(SEQ ID NO:78)

3) CTTATATCTAATTATGTTCCGTCTATATTTATATGGGTTTGCTTTC
and

(SEQ ID NO:79)

TTTCTCTTCATTTTCCTGATAATTTTGTACTTCTGAATG,

(SEQ ID NO:80)

4) ATGCTTTTAAATTTCTTATTCATATCTATTTTTC
and

(SEQ ID NO:83)

GTAAACAGAGAGCGAGTGATCATTTTAAAACTTTTGG,

(SEQ ID NO:81)

5) G(/A)GATTTT(/G)TTTCAACCT(/C)GCAGTAATTTTAACAA
(/C)TC(/T)G(/A)
and

(SEQ ID NO:82)

CCTGAAAACAA(/G)TACT(/C)ACTTTCTGAATTTCAC(/T)GGA(/G)
TATAAAG,

(SEQ ID NO:84)

6) GTTTTATTGACTTTAAAGATGTTAGTTTCTTCGATTCCAG
and

(SEQ ID NO:85)

TTTTTATTACTCTTCTTAAATCATAATGAATCGTACCAATCAAC,

(SEQ ID NO:86)

7) GGATCAATGGCAACTATATTTACCCTACTCTCCACAG
and

(SEQ ID NO:87)

GAGTCGAAACCAACCGGAAAAAGCAATTGAG,

(SEQ ID NO:88)

8) CCTTTGGTTTATTATCCTACTTCCAAAACAGTTTATGC
and

(SEQ ID NO:89)

CATATATCTCTTTATCCTGTCAATATTGATTGGCATTTTC,

(SEQ ID NO:91)

9) GATATTAGCTATACCAACAATTGTTCTTTTCCTGTACTCAGTC
and

(SEQ ID NO:93)

GCATTTCTAGTACCGAACCATTGAAACTATCATCTG,

(SEQ ID NO:90)

10) GAAATTATAGTCGGAGCTTTCATTTATATTAGTTTACTGGTTCTG
and

(SEQ ID NO:93)

CAGAATAAAGAGAGCTGTAATAGGTGCAACTTCATGC,

(SEQ ID NO:94)

11) CTGTAATGTTTCTAATTAGTTCAGTATTTGCACTGGTTAATTC
and

(SEQ ID NO:95)

CCCGTATATCCATTACTAAGAACAAGGTTGTATATTTCCTTC,

(SEQ ID NO:96)

12) GTTTCTCATTAGTTCTGTATTTGCCCTTATTAATGTGC
and

(SEQ ID NO:97)

CCATGGCTAAGTGCAAGATTATGAATCTCTCTC,

(SEQ ID NO:98)

13) GTTTCTTATGTTTACCCTCAGCTTATATTGGCACAG
and

(SEQ ID NO:99)

GATACCACAAATCTCCGAATTCTCTTAAAATAGATGG,

(SEQ ID NO:100)

14) TTAAGTAGTTCACAAGTGATAGTGAACTTGGGATTGTC
and

(SEQ ID NO:101)

CACTGAGATTATTTATTAGCTTTATCGGTAAGGTGGATAAG,

(SEQ ID NO:103)

15) ATTACTTGTAATACTATGTATTCAACTAGTCA(/C)AGGATT
TGATGG
and

(SEQ ID NO:104)

GAACAAATTTCCGTATCAGATTTGCGATTTC,

(SEQ ID NO:102)

16) CCAATGAAAAGGAAAGTTCAATGTGTTTTGTTTCTGC
and

(SEQ ID NO:105)

GGTGCTTCAGCAAAAATCCCCGTATTTCTTATCAG,

(SEQ ID NO:106)

17) TAGCTGATGTTCCGATAAATTATGGTGGGGTAATAATAG
and

(SEQ ID NO:107)

CTGCGACACTGTATATACCTACATTATAACTACTAGACATTTGC,

(SEQ ID NO:108)

18) GCAACTTTGGTTCTAAAATTTTAGTCTTTTTAATGGTTCC
and

(SEQ ID NO:109)

TGTTAAACCCCAATATAGAAATTGTATTGAGAATAGCAGC,

(SEQ ID NO:110)

19) CGTTAATAGCTTATGTTCAACTGGTGATTGATTTTGG
and

(SEQ ID NO:111)

TGATAGTTTTAGAAATAATATAAGGAATTGCAACTGCATGC,

(SEQ ID NO:113)

20) TTCATGTC(/T)T(/C)TTTTG(/A)TCTAATCTGATTACAATTG
(/C)TC(/T)ACATCG(/A)
and

(SEQ ID NO:114)

T(/C)GCATTTG(/T)GATCTGTCACAA(/G)TCAATAAGTTAAAACC,

(SEQ ID NO:112)

21) GGTAGGTATTTTAATTGGAGGAAGAGAGTCTTGAATGG
and

(SEQ ID NO:115)

ATCTTCCCTTCATAAATTGACATAGGAAAAATAAGAGCC,

(SEQ ID NO:116)

22) CAATTCTAACTATGTCCAGTTTTATTTTTCCACTCATCAG
and

(SEQ ID NO:117)

GACGTGATAATAATAAGCTGCCATTCCTGTCTAAAACG,

(SEQ ID NO:118)

23) CGGCGGTATTAAGTAGAATATTAACACCTGAAGAGTATGGC
and

(SEQ ID NO:119)

GGCAATCAGACTCAATAAGTTCATCCGTTTAAAGTTC,

(SEQ ID NO:120)

24) GGTATTGCCTTTCCTTTGATAACTTCTCCTTATTTATCAC
and

(SEQ ID NO:121)

TGAACTTGTAACTCGACACCCAAAAATATAAATAAATGAG,

(SEQ ID NO:123)

25) GAATCGGACAATAGCACAGGTACGAACAAG
and

(SEQ ID NO:124)

GCCATGTAATCAACTGACCAAGCAGGGTACTC,

(SEQ ID NO:122)

26) CAAAGGAACGTTATCAGCAATTGTGTCAAATTTCAG
and

(SEQ ID NO:125)

AAGATTAGGGCGCACAAAGTTTACTTGTTTTAGC,

(SEQ ID NO:126)

27) GTTATTTCTTCAAATCTGCTCATAGTTTTAACCTCATCACA
and

(SEQ ID NO:127)

TATCTTGCGTTTTCATCCCTTACAGTTATTAGGTTCAAAG,

(SEQ ID NO:128)

28) TTCTTCAAATCTTTTGACAGTCTTGACCTCTTCCTTG
and

(SEQ ID NO:129)

TATCGTGCATTCGAATCTGTTACAGCTAATACATTTAAAC,

(SEQ ID NO:130)

29) GTCCTGACGCTATCAAATATCATTTTCCCATTAATCAC
and

(SEQ ID NO:131)

CCCACATGTGATCAATAGGAGTGAAAATTCTCTATTC,

(SEQ ID NO:133)

30) GCTTTGGCTAACTTTTCATCAAAGATTTTAATTTTTTTGTTAG
and

(SEQ ID NO:134)

CCAGAGATAGCTGTAACACCAATTTTATCAATTCCCTTAG,

(SEQ ID NO:132)

31) CCTTTGGCTAATTTCTTGGACGATAATGAATTTGTATATG
and

(SEQ ID NO:135)

CCACAAACATTAGCAATAAAGAAACCTAACAATCCC,

(SEQ ID NO:137)

32) GATCATACTCCCTATCATTACGACTCCCTATGTAACG
and

(SEQ ID NO:138)

CCAAGAAATATCCAAACCTTTTGACACTAAACTTAATCC,

(SEQ ID NO:136)

33) GTTGTTTTAGCTCAAGGAGGGATAATGTTGGCTTCG
and

(SEQ ID NO:139)

GCTGATTTTACAAATAGGAAAATAGAGATTGCACCAAC,
and

34) a primer comprising a sequence selected from any one of SEQ ID NO's 144 to 333, and

35) a primer that can be used to amplify the same region, or diagnostic portion thereof, from the genome of a strain of S. pneumoniae as a primer provided as any one of SEQ ID NO's 75 to 139 or 144 to 333.

Guidance regarding the serotypes these primer pairs target, and the length of resulting amplification products, is provided in Tables 2, 3 and 7.

It has been noted that some of the above primer pairs formed non-serotype specific amplicons, for example; PCR targeting serotype 6B also amplified 6A; PCR targeting 18C amplified all serotypes in serogroup 18; PCR targeting wzx (but not wzy) of serotype 23F, amplified three serotype 23A strains; PCR targeting wzx and wzy of serotypes 33/37 amplified a 33A isolate and that targeting wzx amplified a serotype 33B isolate. Accordingly, in these instances further analysis will need to be performed to determine the correct serotype. For instance, traditional serological typing can be performed.

Serotype 3 does not contain wzy and wzx genes. Accordingly, upon obtaining results using the method of analysing at least a portion of the nucleotide sequence between the 3′ end of the cpsA gene and the 5′ end of the cpsB gene, the presence of serotype 3 can be confirmed by analysing the orf2 (wze)-cap3A-cap3B region. Preferably, serotype 3 is identified by amplifying a portion of the orf2 (wze)-cap3A-cap3B region using primer pairs selected from the group consisting of:

(SEQ ID NO:140)

GCACAAAAAAAAGTTTGATATTCCCCTTGACAATAG
and

(SEQ ID NO:141)

GCAGGATCTAAGGAGGCTTCAAGATTCAACTC,

(SEQ ID NO:142)

2) CGAACCTACTATTGAGTGTGATACTTTTATGGGATACAGAG
and

(SEQ ID NO:143)

CTGACAGCATGAAAATATATAACCGCCCAACGAATAAG,
and

3) primer pairs that amplify the same region, or diagnostic portion thereof, from the genome of a strain of S. pneumoniae as the primers provided in 1) or 2).

During routine analysis of a sample comprising bacteria it will typically be desirable to ensure that the sample being analysed actually contains Streptococcus pneumoniae. Thus, it is preferred that the methods of the present invention include detecting any serotype of Streptococcus pneumoniae in the sample.

Such methods are known in the art and include, but are not limited to, amplifying portions of the psaA and/or pneumolysin genes followed by detection of the amplification products.

In a preferred embodiment, a portion of the psaA gene is amplified using primers comprising the sequence TACATTACTCGTTCTCTTTCTTTCTGCAATCATTCTTG (SEQ ID NO:64) and TAGTAGCTGTCGCCTTCTTTACCTTGTTCTGC (SEQ ID NO:65), or primer pairs that amplify the same region, or diagnostic portion thereof, from the genome of a strain of S. pneumoniae as SEQ ID NO:64 and SEQ ID NO:65. In another preferred embodiment, a portion of the pneumolysin gene is amplified using primers comprising the sequence AGAATAATCCCACTCTTCTTGCGGTTGA (SEQ ID NO:66) and CATGCTGTGAGCCGTTATTTTTTCATACTG (SEQ ID NO:67) or primer pairs that amplify the same region, or diagnostic portion thereof, from the genome of a strain of S. pneumoniae as SEQ ID NO:66 and SEQ ID NO:67.

The present inventors have observed a strong correlation between the molecular capsular sequence typing techniques of the invention and the actual serotype of a strain as determined by traditional antibody based serological typing. However, the typing methods of the invention may be assisted by further serotyping the S. pneumoniae strain. For instance, to ensure recombination events have not occurred, upon typing with the methods of the invention the serotype can be confirmed by serologically typing for the strain suggested by the methods of the invention. Furthermore, the inventors have noted that a few serotypes are difficult to resolve using the methods of the invention, for example; 6A and 6B; 15B and 15C; 22F and 22A; and 35C and 42. Upon identification of any of these serotypes by the molecular techniques of the invention the serotype can be unequivocally typed using traditional serological methods.

In another aspect, the present invention provides an isolated polynucleotide comprising a sequence of nucleotides selected from those provided as SEQ ID NO's 2 to 63, or a fragment thereof which is at least 10 nucleotides in length, with the proviso that the polynucleotide does not comprise the entire wzy and/or wzx gene(s) of a S. pneumoniae serotype selected from the group consisting of: 1, 2, 4, 6A, 6B, 8, 9V, 14, 18C, 19F, 19A, 19B, 23F, 33F and 37, or the entire wzx gene of S. pneumoniae serotype 19C.

In a further aspect, the present invention provides an isolated polynucleotide comprising a sequence of nucleotides selected from the group consisting of. 1-AF532632, 10A-AF532633, 10A-AF532634, 10B-AY508586, 10F-AF532635, 10F-AF532636, 10F-AY508587, 11A-AF532637, 11A-AF532638, 11B-AF532639, 11C-AY508588, 11C-AY508589, 12A-AY508590, 12A-AY508591, 12F-AF532640, 12F-AF532641, 13-AF532642, 14-AF532643, 14-AF532644, 14-AF532645, 15A-AF532646, 15A-AF532647, 15B-AF532648, 15B-AF532649, 15B-AF532650, 15C-AF532651, 15C-AF532652, 15C-AY330714, 15C-AY330715, 15C-AY508592, 15C-AY508593, 15F-AY508594, 15F-AY508595, 16A-AY508596, 16F-AF532653, 16F-AF532654, 17A-AF532655, 17A-AY508597, 17F-AF532656, 17F-AF532657, 18A-AF532658, 18A-AF532659, 18B-AF532660, 18C-AF532661, 18F-AF532662, 18F-AY330716, 18F-AY508598, 19A-AF532663, 19A-AF532664, 19B-AY508599, 19C-AY508600, 19C-AY508601, 19F-AF532665, 19F-AF532666, 19F-AF532667, 19F-AF532668, 2-AF532669, 20-AF532670, 21-AF532671, 21-AY508602, 22A-AF532672, 22F-AF532673, 23A-AF532674, 23A-AF532675, 23B-AF532676, 23B-AY330717, 23F-AF532677, 23F-AF532678, 23 F-AF532679, 24A-AY508603, 24B-AY508604, 24F-AY508605, 24F-AY508606, 24F-AY508607, 25F-AF532711, 27-AY508608, 28A-AY508609, 28F-AY508610, 28F-AY508611, 29-AF532680, 29-AY330718, 3-AF532681, 3-AF532682, 3-AF532683, 31-AF532684, 32A-AY508612, 32A-AY508613, 32F-AY508614, 33A-AF532685, 33B-AF532686, 33B-AY508615, 33C-AY508616, 33F-AF532687, 33F-AF532688, 33F-AF532689, 34-AF532690, 35A-AY508617, 35B-AF532691, 35C-AY508618, 35F-AF532692, 36-AY508619, 37-AF532713, 38-AF532712, 39-AY508620, 39-AY508621, 4-AF532693, 40-AY508622, 41A-AY508623, 41F-AY508624, 42-AY508625, 43-AY508626, 45-AY508628, 46-AY508629, 47A-AY508630, 47F-AY508631, 48-AY508632, 48-AY508633, 5-AF532696, 5-AF532697, 5-AY508634, 6A-AF532698, 6A-AF532699, 6A-AF532700, 6A-AF532701, 6A-AF532702, 6A-AY508641, 6B-AF532703, 6B-AF532704, 6B-AF532705, 7A-AY508635, 7B-AY508636, 7C-AF532706, 7F-AF532707, 8-AF532708, 9A-AY508637, 9L-AY508638, 9N-AF532709, 9V-AF532710 and 9V-AY508639 as provided in FIG. 2, or a fragment thereof which is at least 10 nucleotides in length, with the proviso the polynucleotide does not comprise the 3′ end of the cpsA gene to the 5′ end of the cpsB gene of a S. pneumoniae serotype selected from the group consisting of: 1, 2, 3, 4, 6A, 6B, 8, 9V, 14, 18C, 19F, 19A, 23F, 33F and 37.

In a preferred embodiment, the polynucleotide of these aspects is at least 15 nucleotides, more preferably at least 20 nucleotides, more preferably at least 25 nucleotides, more preferably at least 30 nucleotides, more preferably at least 50 nucleotides in length, and even more preferably at least 100 nucleotides in length.

In a further aspect, the present invention provides an isolated polynucleotide consisting essentially of 10 to 50 contiguous nucleotides corresponding to a portion of the 3′ end of the cpsA S. pneumoniae gene or the 5′ end of the cpsB S. pneumoniae gene,.

In a further aspect, the present invention provides a polynucleotide consisting essentially of 10 to 50 contiguous nucleotides corresponding to a portion of the S. pneumoniae wzy and/or wzx gene(s).

Preferably, said polynucleotide of 10 to 50 contiguous nucleotides comprises one or more nucleotides which differ between different S. pneumoniae serotypes.

Polynucleotides of 10 to 50 contiguous nucleotides can be used as amplification primers, or as probes, for the identification of different S. pneumoniae serotypes.

Preferably the nucleotides which differ between S. pneumoniae serotypes correspond to one or more of positions as shown in FIG. 2.

Preferably, the polynucleotide is detectably labelled. The label can be any suitable label known in the art including, but not limited to, radionuclides, enzymes, fluorescent, and chemiluminescent labels.

Also provided is a vector comprising a polynucleotide of the invention. Preferably, the vector is an expression vector. Furthermore, provided is a host cell comprising a vector of the invention. Suitable vectors and host cells would be well known to those skilled in the art.

In yet another aspect, the present invention provides a composition comprising a plurality of polynucleotides according to the invention and an acceptable carrier or excipient. Preferably, the carrier or excipient is water or a suitable buffer. The composition may be used in methods of typing different S. pneumoniae serotypes.

In a further aspect the present invention provides a microarray comprising a plurality of polynucleotides according to the invention. The microarray may be used in methods of typing different S. pneumnoniae serotypes.

In another aspect, the present invention provides a kit comprising at least one polynucleotide of the present invention.

Preferably, the polynucleotide is 10 to 50 nucleotides in length. In one embodiment, the kit further comprises reagents necessary for nucleic acid amplification. In another embodiment, the polynucleotide is detectably labelled and the kit further comprises means for detecting the labelled polynucleotide.

As will be apparent, preferred features and characteristics of one aspect of the invention are applicable to many other aspects of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The invention is hereinafter described by way of the following non-limiting examples and with reference to the accompanying figures.

### BRIEF DESCRIPTION OF THE ACCOMPANYING DRAWINGS

FIG. 1. The genomic sequence of cpsA (wzg) and cpsB (wzh) genes of serotype 4 of S. pneumoniae as published by Jiang et al. (2001) and deposited as GenBank Accession Number AF316639. The remaining 3′ sequence of GenBank Accession Number AF316639 has not been provided. Nucleotides 1520 to 2965 encode cpsA whilst nucleotides 2967 to 3698 encode cpsB.

FIG. 2. Multiple sequence alignments for the region between the 3′-end of cpsA (wzg) and the 5′-end of cpsB (wzh) of 132 molecular capsular sequence types of S. pneumoniae. The alignment numbering start point “1” refer to the position “2470” of S. pneumoniae serotype 4 cpsA (wzg) gene (GenBank accession number: AF316639) (FIG. 1).

FIG. 3. Phylogenetic tree inferred from sequences in the region between the 3′-end of cpsA (wzg) and the 5′-end of cpsB (wzh) genes for 132 molecular capsular sequence types of S. pneumoniae. Most of the tree input sequences are from FIG. 2; for GenBank accession numbers see Tables 1 and 8.

FIG. 4. Phylogenetic tree of wzx genes of 83 S. pneumoniae cps serotypes. The tree is generated by the neighbour-joining method based on all nucleotide sites.

FIG. 5. Phylogenetic tree of wzy genes of total 83 S. pneumoniae cps serotypes. The tree is generated by the neighbour-joining method based on all nucleotide sites.

FIG. 6. Schematic representation of the closely related wzx genes. Each block represents wzx genes from one or more S. pneumoniae serotype cps gene cluster. Similar patterns and shading represent regions with DNA sequence identity >75% among different nucleotide sequences.

### KEY TO THE SEQUENCE LISTING

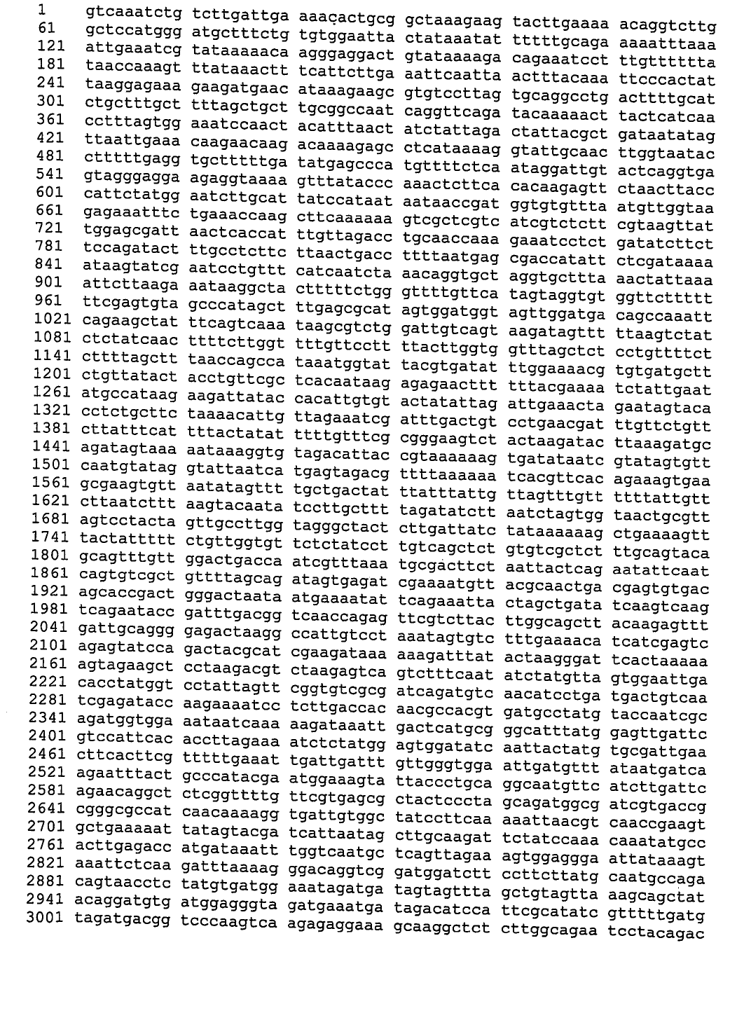

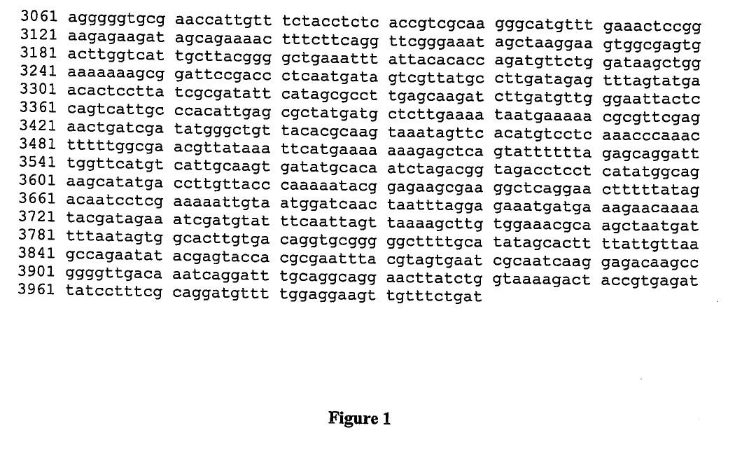

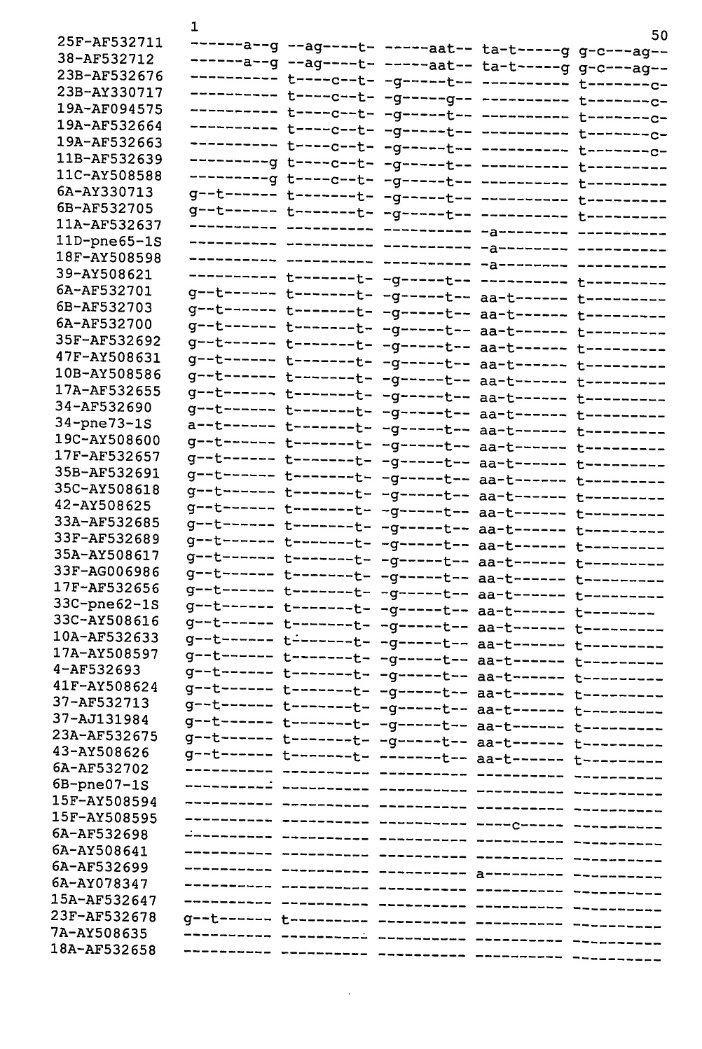

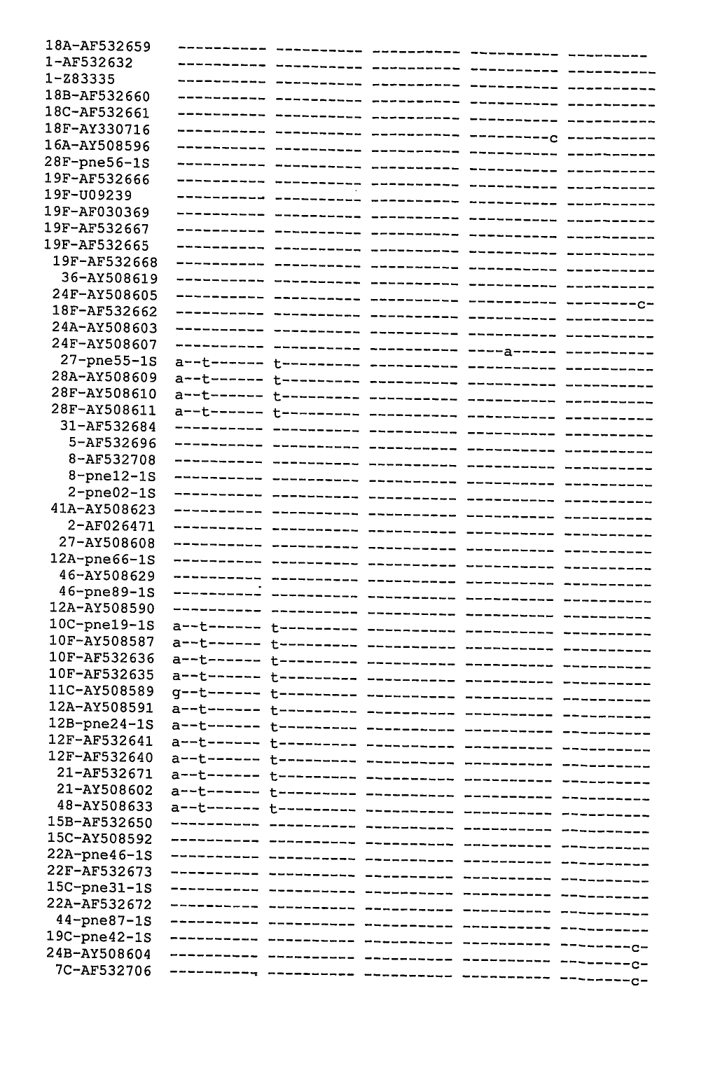

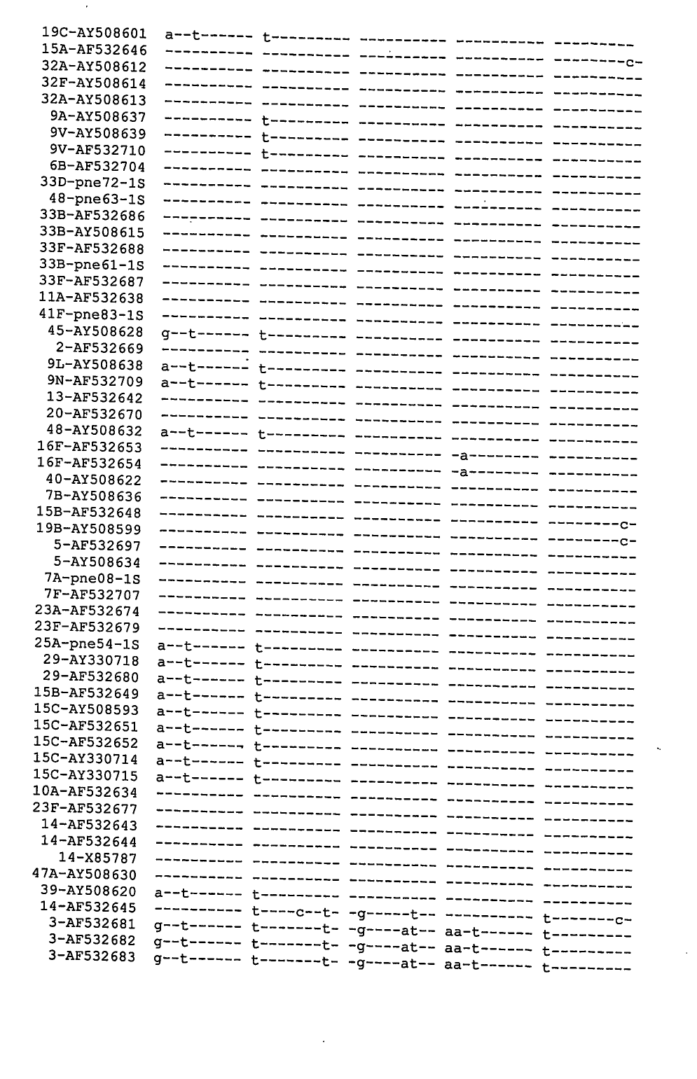

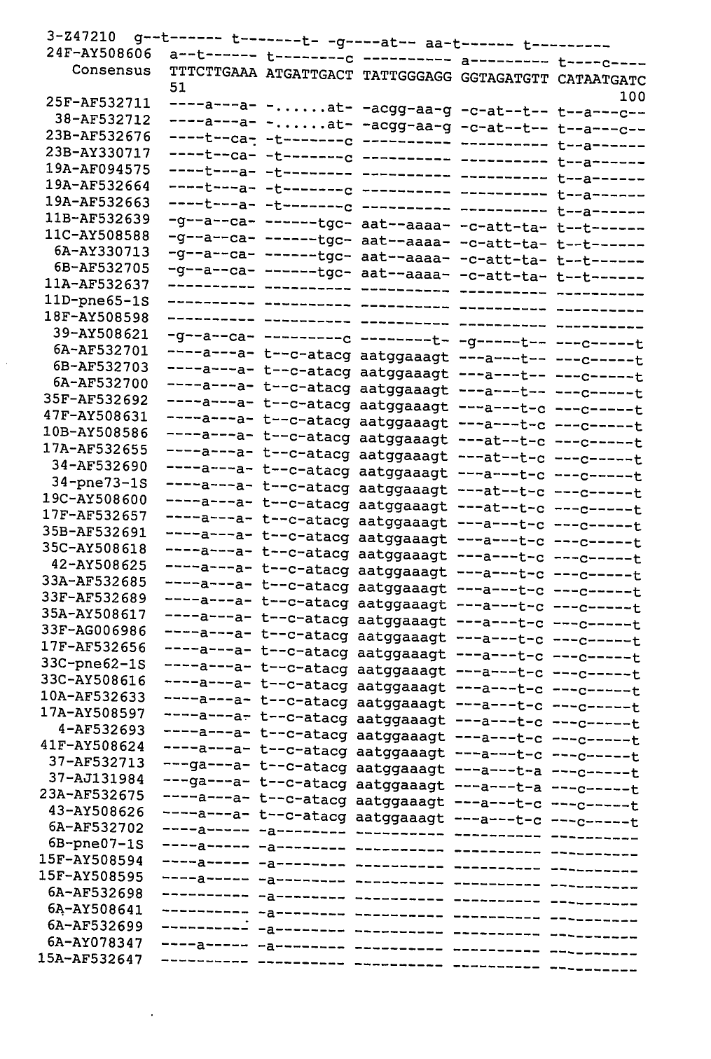

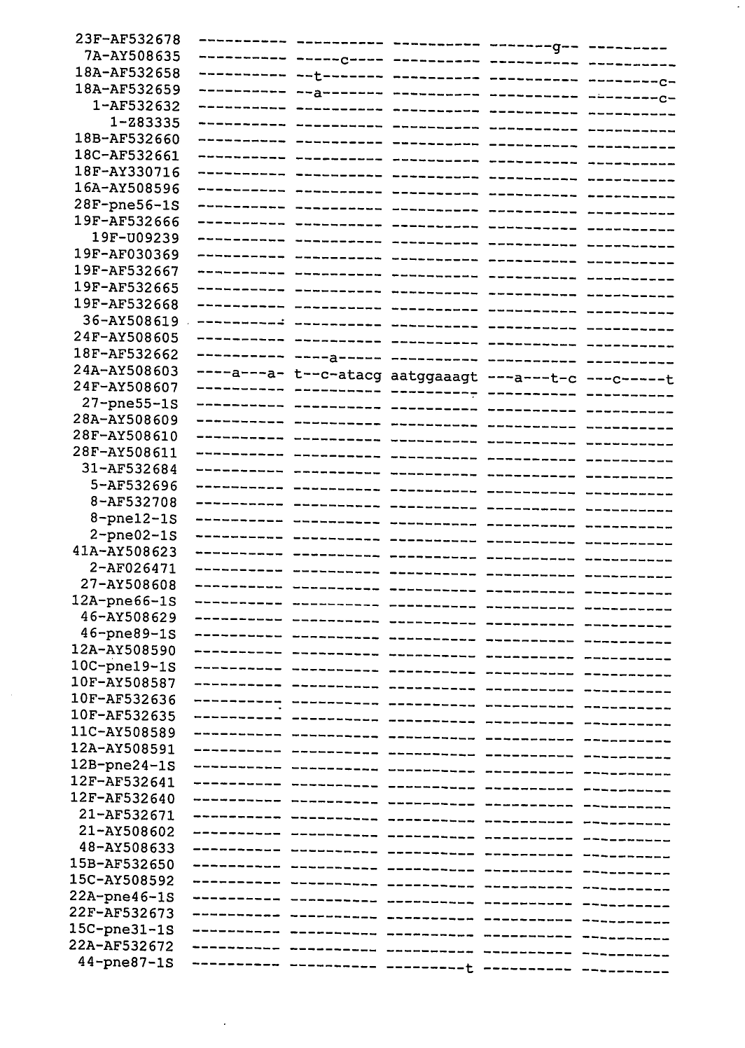

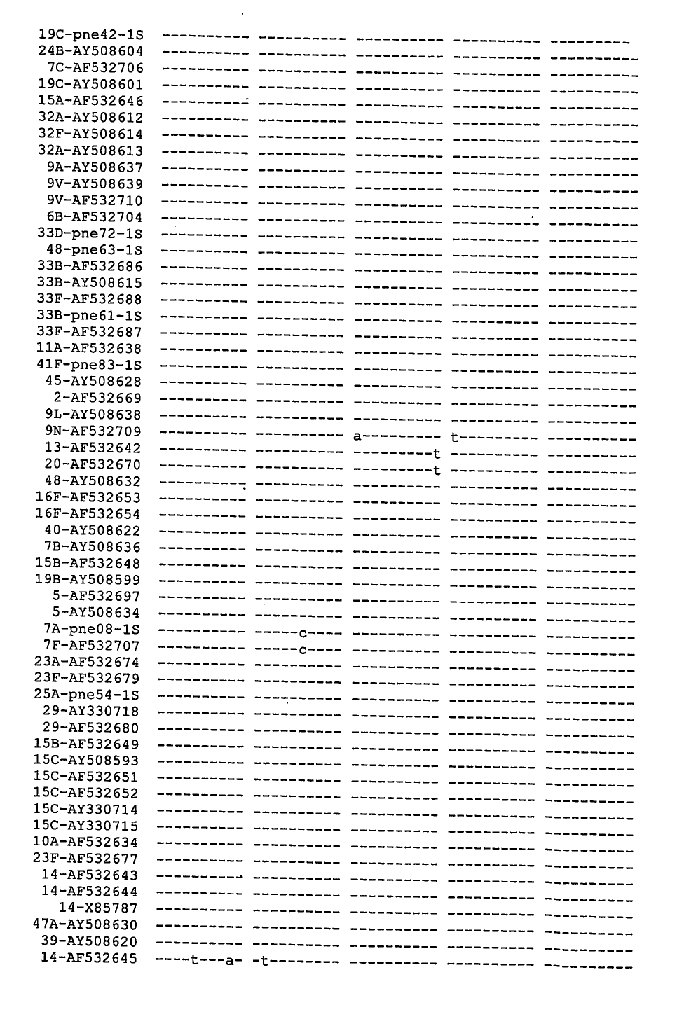

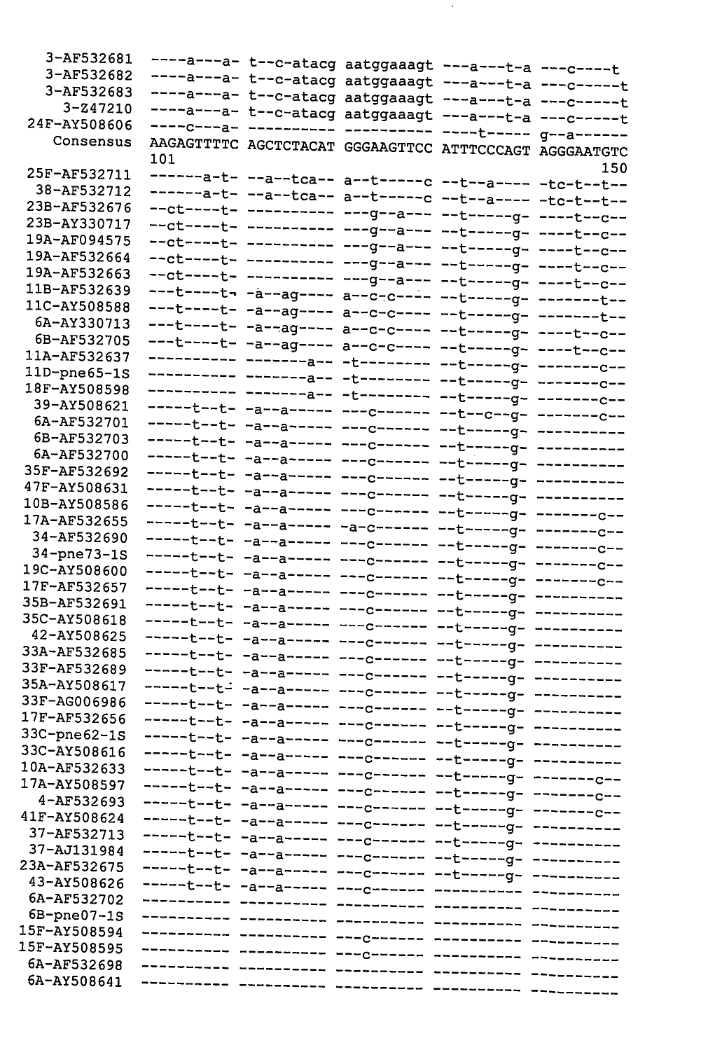

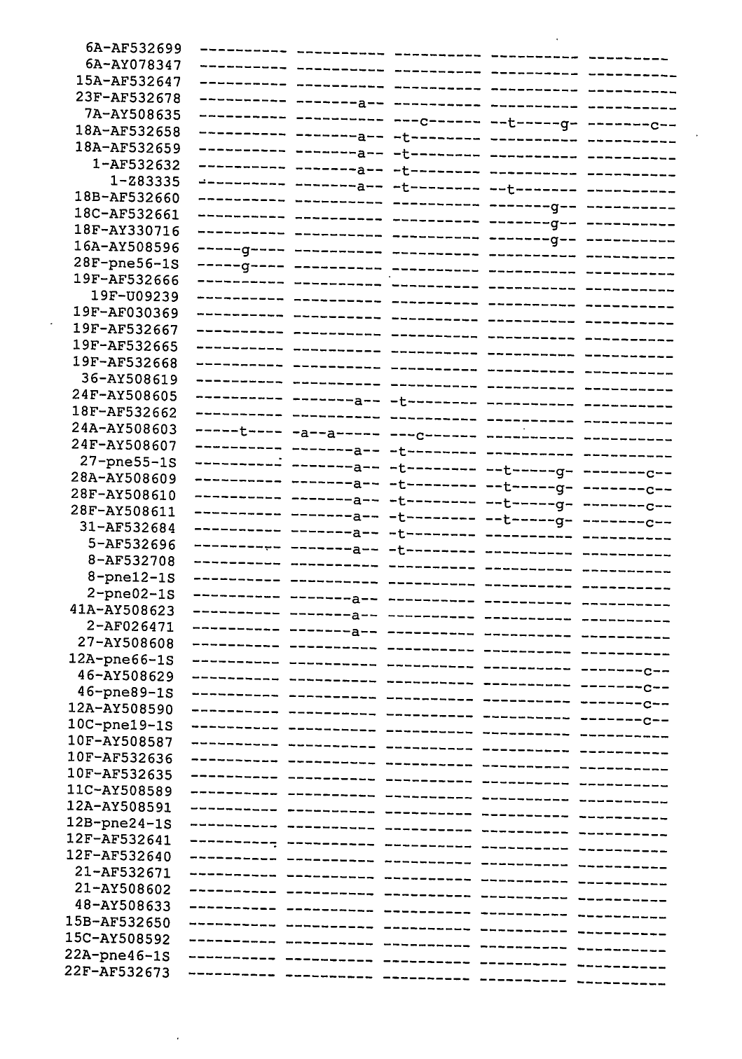

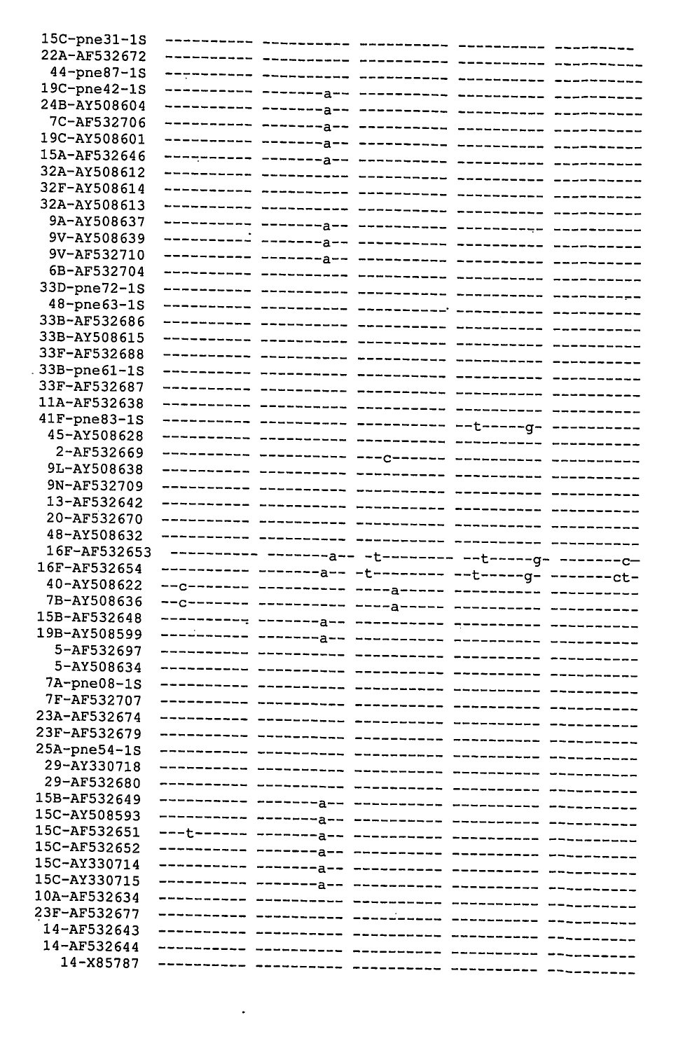

SEQ ID NO:1—Genomic sequence of cpsA (wzg) and cpsB (wzh) genes of serotype 4 of S. pneumoniae (FIG. 1).
SEQ ID NO:2—Partial sequence of strain 00-251-3185 wzx gene.
SEQ ID NO:3—Partial sequence of strain 01-122-0226 wzx gene.
SEQ ID NO:4—Partial sequence of strain 01-192-2471 wzx gene.
SEQ ID NO:5—Partial sequence of strain MA055100 wzx gene.
SEQ ID NO:6—Partial sequence of strain NZSPN01/329 wzx gene.
SEQ ID NO:7—Partial sequence of strain 00-256-1986 wzx gene.
SEQ ID NO:8—Partial sequence of strain NZSPN01/276 wzx gene.
SEQ ID NO:9—Partial sequence of strain 00-201-1422 wzx gene.
SEQ ID NO:10—Partial sequence of strain 00-211-1669 wzx gene.
SEQ ID NO:11—Partial sequence of strain 00S002 wzx gene.
SEQ ID NO:12—Partial sequence of strain 00-251-3185 wzy gene.
SEQ ID NO:13—Partial sequence of strain 01-122-0226 wzy gene.
SEQ ID NO:14—Partial sequence of strain 01-192-2471 wzy gene.
SEQ ID NO:15—Partial sequence of strain MA055100 wzy gene.
SEQ ID NO:16—Partial sequence of strain NZSPN01/329 wzy gene.
SEQ ID NO:17—Partial sequence of strain 00-256-1986 wzy gene.
SEQ ID NO:18—Partial sequence of strain NZSPN01/276 wzy gene.
SEQ ID NO:19—Partial sequence of strain 00-201-1422 wzy gene.
SEQ ID NO:20—Partial sequence of strain 00-211-1669 wzy gene.
SEQ ID NO:21—Partial sequence of strain 00S002 wzy gene.
SEQ ID NO:22—Partial sequence of strain NZSPN01/509 cpsI and wzx genes.
SEQ ID NO:23—Partial sequence of strain MA050408 cpsI and wzx genes.
SEQ ID NO:24—Partial sequence of strain MA052433 cpsI and wzx genes.
SEQ ID NO:25—Partial sequence of strain 00S009 cpsI and wzx genes.
SEQ ID NO:26—Partial sequence of strain 99-325-0373 cpsI and wzx genes.
SEQ ID NO:27—Partial sequence of strain NZSPN00/454 cpsI and wzx genes.
SEQ ID NO:28—Partial sequence of strain NZSPN00/484 cpsI and wzx genes.
SEQ ID NO:29—Partial sequence of strain 00-081-2291 wzy and wzx genes.
SEQ ID NO:30—Partial sequence of strain 00S168 wzy and wzx genes.
SEQ ID NO:31—Partial sequence of strain 00-280-1493 wzy and wzx genes.
SEQ ID NO:32—Partial sequence of strain MA063073 wzy and wzx genes.
SEQ ID NO:33—Partial sequence of strain NZSPN00/410 wzy and wzx genes.
SEQ ID NO:34—Partial sequence of strain NZSPN01/243 wzy and wzx genes.
SEQ ID NO:35—Partial sequence of strain MA063087 wzy and wzx genes.
SEQ ID NO:36—Partial sequence of strain MA063207 wzy and wzx genes.
SEQ ID NO:37—Partial sequence of strain 01S333 wzx gene.
SEQ ID NO:38—Partial sequence of strain MA050663 wciW and wzx genes.
SEQ ID NO:39—Partial sequence of strain 01S319 wciW and wzx genes.
SEQ ID NO:40—Partial sequence of strain NZSPN00/353 wciW and wzx genes.
SEQ ID NO:41—Partial sequence of strain MA062610 wciW and wzx genes.
SEQ ID NO:42—Partial sequence of strain MA053392 wciW and wzx genes.
SEQ ID NO:43—Partial sequence of strain NZSPN00/319 wciW and wzx genes.
SEQ ID NO:44—Partial sequence of strain NZSPN01/278 wciW and wzx genes.
SEQ ID NO:45—Partial sequence of strain 01S009 wciW and wzx genes.
SEQ ID NO:46—Partial sequence of strain MA052628 wciW and wzx genes.
SEQ ID NO:47—Partial sequence of strain 00-081-2291 cpsJ and wzy genes.
SEQ ID NO:48—Partial sequence of strain 00-280-1493 cpsJ and wzy genes.
SEQ ID NO:49—Partial sequence of strain NZSPN00/410 cpsJ and wzy genes.
SEQ ID NO:50—Partial sequence of strain NZSPN01/243 cpsJ and wzy genes.
SEQ ID NO:51—Partial sequence of strain MA063073 cpsJ and wzy genes.
SEQ ID NO:52—Partial sequence of strain 00S168 cpsJ and wzy genes.
SEQ ID NO:53—Partial sequence of strain MA063087 cpsJ and wzy genes.
SEQ ID NO:54—Partial sequence of strain MA063207 cpsJ and wzy genes.
SEQ ID NO:55—Partial sequence of strain 01S319 wzx and wzy genes.
SEQ ID NO:56—Partial sequence of strain NZSPN00/353 wzx and wzy genes.
SEQ ID NO:57—Partial sequence of strain MA062610 wzx and wzy genes.
SEQ ID NO:58—Partial sequence of strain MA053392 wzx and wzy genes.
SEQ ID NO:59—Partial sequence of strain NZSPN00/319 wzx and wzy genes.
SEQ ID NO:60—Partial sequence of strain NZSPN01/278 wzx and wzy genes.
SEQ ID NO:61—Partial sequence of strain MA050663 wzx and wzy genes.
SEQ ID NO:62—Partial sequence of strain MA052628 wzx and wzy genes.
SEQ ID NO:63—Partial sequence of strain 01S009 wzx and wzy genes.
SEQ ID NO's 64 to 143—Oligonucleotide primers provided in Table 2.
SEQ ID NO's 144 to 333—Oligonucleotide primers provided in Table 7.
SEQ ID NO:334*—Sequence of serotype 33C wzx gene.
SEQ ID NO:335*—Sequence of serotype 10B wzx gene.
SEQ ID NO:336*—Sequence of serotype 10C wzx gene.
SEQ ID NO:337*—Sequence of serotype 10F wzx gene.
SEQ ID NO:338*—Sequence of serotype 11A wzx gene.
SEQ ID NO:339*—Sequence of serotype 11D wzx gene.
SEQ ID NO:340*—Sequence of serotype 12A wzx gene.
SEQ ID NO:341*—Sequence of serotype 12B wzx gene.
SEQ ID NO:342*—Sequence of serotype 12F wzx gene.
SEQ ID NO:343*—Sequence of serotype 13 wzx gene.
SEQ ID NO:344*—Sequence of serotype 14 wzx gene.
SEQ ID NO:345*—Sequence of serotype 15A wzx gene.
SEQ ID NO:346*—Sequence of serotype 15B wzx gene.
SEQ ID NO:347*—Sequence of serotype 15C wzx gene.
SEQ ID NO:348*—Sequence of serotype 15F wzx gene.
SEQ ID NO:349*—Sequence of serotype 16A wzx gene.
SEQ ID NO:350*—Sequence of serotype 16F wzx gene.
SEQ ID NO:351*—Sequence of serotype 17A wzx gene.
SEQ ID NO:352*—Sequence of serotype 17F wzx gene.
SEQ ID NO:353*—Sequence of serotype 18A wzx gene.
SEQ ID NO:354*—Sequence of serotype 18B wzx gene.
SEQ ID NO:355*—Sequence of serotype 18F wzx gene.
SEQ ID NO:356*—Sequence of serotype 20 wzx gene.
SEQ ID NO:357*—Sequence of serotype 22A wzx gene.
SEQ ID NO:358*—Sequence of serotype 22F wzx gene.
SEQ ID NO:359*—Sequence of serotype 23A wzx gene.
SEQ ID NO:360*—Sequence of serotype 23B wzx gene.
SEQ ID NO:361*—Sequence of serotype 24B wzx gene.
SEQ ID NO:362*—Sequence of serotype 25A wzx gene.
SEQ ID NO:363*—Sequence of serotype 25F wzx gene.
SEQ ID NO:364*—Sequence of serotype 27 wzx gene.
SEQ ID NO:365*—Sequence of serotype 28A wzx gene.
SEQ ID NO:366*—Sequence of serotype 28F wzx gene.
SEQ ID NO:367*—Sequence of serotype 29 wzx gene.
SEQ ID NO:368*—Sequence of serotype 31 wzx gene.
SEQ ID NO:369*—Sequence of serotype 32A wzx gene.
SEQ ID NO:370*—Sequence of serotype 32F wzx gene.
SEQ ID NO:371*—Sequence of serotype 33A wzx gene.
SEQ ID NO:372*—Sequence of serotype 33B wzx gene.
SEQ ID NO:373*—Sequence of serotype 10A wzx gene.
SEQ ID NO:374*—Sequence of serotype 9N wzx gene.
SEQ ID NO:375*—Sequence of serotype 34 wzx gene.
SEQ ID NO:376*—Sequence of serotype 35A wzx gene.
SEQ ID NO:377*—Sequence of serotype 35B wzx gene.
SEQ ID NO:378*—Sequence of serotype 35C wzx gene.
SEQ ID NO:379*—Sequence of serotype 35F wzx gene.
SEQ ID NO:380*—Sequence of serotype 36 wzx gene.
SEQ ID NO:381*—Sequence of serotype 38 wzx gene.
SEQ ID NO:382*—Sequence of serotype 39 wzx gene.
SEQ ID NO:383*—Sequence of serotype 40 wzx gene.
SEQ ID NO:384*—Sequence of serotype 41A wzx gene.
SEQ ID NO:385*—Sequence of serotype 41F wzx gene.
SEQ ID NO:386*—Sequence of serotype 42 wzx gene.
SEQ ID NO:387*—Sequence of serotype 43 wzx gene.
SEQ ID NO:388*—Sequence of serotype 44 wzx gene.
SEQ ID NO:389*—Sequence of serotype 45 wzx gene.
SEQ ID NO:390*—Sequence of serotype 46 wzx gene.
SEQ ID NO:391*—Sequence of serotype 47A wzx gene.
SEQ ID NO:392*—Sequence of serotype 47F wzx gene.
SEQ ID NO:393*—Sequence of serotype 48 wzx gene.
SEQ ID NO:394*—Sequence of serotype 48(1) wzx gene.
SEQ ID NO:395*—Sequence of serotype 7A wzx gene.
SEQ ID NO:396*—Sequence of serotype 7C wzx gene.
SEQ ID NO:397*—Sequence of serotype 7F wzx gene.
SEQ ID NO:398*—Sequence of serotype 9A wzx gene.
SEQ ID NO:399*—Sequence of serotype 9L wzx gene.
SEQ ID NO:400*—Sequence of serotype 33D wzx gene.
SEQ ID NO:401*—Sequence of serotype 33B wzy gene.
SEQ ID NO:402*—Sequence of serotype 10B wzy gene.
SEQ ID NO:403*—Sequence of serotype 10C wzy gene.
SEQ ID NO:404*—Sequence of serotype 10F wzy gene.
SEQ ID NO:405*—Sequence of serotype 11A wzy gene.
SEQ ID NO:406*—Sequence of serotype 11D wzy gene.
SEQ ID NO:407*—Sequence of serotype 12A wzy gene.
SEQ ID NO:408*—Sequence of serotype 12B wzy gene.
SEQ ID NO:409*—Sequence of serotype 12F wzy gene.
SEQ ID NO:410*—Sequence of serotype 13 wzy gene.
SEQ ID NO:411*—Sequence of serotype 14 wzy gene.
SEQ ID NO:412*—Sequence of serotype 15A wzy gene.
SEQ ID NO:413*—Sequence of serotype 15B wzy gene.
SEQ ID NO:414*—Sequence of serotype 15C wzy gene.
SEQ ID NO:415*—Sequence of serotype 15F wzy gene.
SEQ ID NO:416*—Sequence of serotype 16A wzy gene.
SEQ ID NO:417*—Sequence of serotype 16F wzy gene.
SEQ ID NO:418*—Sequence of serotype 17A wzy gene.
SEQ ID NO:419*—Sequence of serotype 17F wzy gene.
SEQ ID NO:420*—Sequence of serotype 18A wzy gene.
SEQ ID NO:421*—Sequence of serotype 18B wzy gene.
SEQ ID NO:422*—Sequence of serotype 18F wzy gene.
SEQ ID NO:423*—Sequence of serotype 19C wzy gene.
SEQ ID NO:424*—Sequence of serotype 20 wzy gene.
SEQ ID NO:425*—Sequence of serotype 22A wzy gene.
SEQ ID NO:426*—Sequence of serotype 22F wzy gene.
SEQ ID NO:427*—Sequence of serotype 23A wzy gene.
SEQ ID NO:428*—Sequence of serotype 23B wzy gene.
SEQ ID NO:429*—Sequence of serotype 24B wzy gene.
SEQ ID NO:430*—Sequence of serotype 25A wzy gene.
SEQ ID NO:431*—Sequence of serotype 25F wzy gene.
SEQ ID NO:432*—Sequence of serotype 27 wzy gene.
SEQ ID NO:433*—Sequence of serotype 28A wzy gene.
SEQ ID NO:434*—Sequence of seotype 28F wzy gene.
SEQ ID NO:435*—Sequence of serotype 29 wzy gene.
SEQ ID NO:436*—Sequence of serotype 31 wzy gene.
SEQ ID NO:437*—Sequence of serotype 32A wzy gene.
SEQ ID NO:438*—Sequence of serotype 32F wzy gene.
SEQ ID NO:439*—Sequence of serotype 33A wzy gene.
SEQ ID NO:440*—Sequence of serotype 10A wzy gene.
SEQ ID NO:441*—Sequence of serotype 9N wzy gene.
SEQ ID NO:442*—Sequence of serotype 33D wzy gene.
SEQ ID NO:443*—Sequence of serotype 34 wzy gene.
SEQ ID NO:444*—Sequence of serotype 35A wzy gene.
SEQ ID NO:445*—Sequence of serotype 35B wzy gene.
SEQ ID NO:446*—Sequence of serotype 35C wzy gene.
SEQ ID NO:447*—Sequence of serotype 35F wzy gene.
SEQ ID NO:448*—Sequence of serotype 36 wzy gene.
SEQ ID NO:449*—Sequence of serotype 38 wzy gene.
SEQ ID NO:450*—Sequence of serotype 39 wzy gene.
SEQ ID NO:451*—Sequence of serotype 40 wzy gene.
SEQ ID NO:452*—Sequence of serotype 41A wzy gene.
SEQ ID NO:453*—Sequence of serotype 41F wzy gene.
SEQ ID NO:454*—Sequence of serotype 42 wzy gene.
SEQ ID NO:455*—Sequence of serotype 43 wzy gene.
SEQ ID NO:456*—Sequence of serotype 44 wzy gene.
SEQ ID NO:457*—Sequence of serotype 45 wzy gene.
SEQ ID NO:458*—Sequence of serotype 46 wzy gene.
SEQ ID NO:459*—Sequence of serotype 47A wzy gene.
SEQ ID NO:460*—Sequence of serotype 47F wzy gene.
SEQ ID NO:461*—Sequence of serotype 48 wzy gene.
SEQ ID NO:462*—Sequence of serotype 48(1) wzy gene.
SEQ ID NO:463*—Sequence of serotype 7A wzy gene.
SEQ ID NO:464*—Sequence of serotype 7C wzy gene.
SEQ ID NO:465*—Sequence of serotype 7F wzy gene.
SEQ ID NO:466*—Sequence of serotype 9A wzy gene.
SEQ ID NO:467*—Sequence of serotype 9L wzy gene.
SEQ ID NO:468*—Sequence of serotype 33C wzy gene.
SEQ ID NO:469—Sequence of serotype 9V wzx gene (Genbank accesion no. AF402095).
SEQ ID NO:470—Sequence of serotype 19B wzx gene (Genbank accesion no. AF004325).
SEQ ID NO:471—Sequence of serotype 19C wzx gene (Genbank accesion no. AF105116).
SEQ ID NO:472—Sequence of serotype 19F wzx gene (Genbank accesion no. U09239).
SEQ ID NO:473—Sequence of serotype 2 wzx gene (Genbank accesion no. AF026471).
SEQ ID NO:474—Sequence of serotype 23F wzx gene (Genbank accesion no. AF057294).
SEQ ID NO:475—Sequence of serotype 33F wzx gene (Genbank accesion no. AFAJ006986).
SEQ ID NO:476—Sequence of serotype 37 wzx gene (Genbank accesion no. AJ131984).
SEQ ID NO:477—Sequence of serotype 6A wzx gene (Genbank accesion no. AY078347).
SEQ ID NO:478—Sequence of serotype 6B wzx gene (Genbank accesion no. AF316640).
SEQ ID NO:479—Sequence of serotype 8 wzx gene (Genbank accesion no. AF316641).
SEQ ID NO:480—Sequence of serotype 18C wzx gene (Genbank accesion no. AF316642).
SEQ ID NO:481—Sequence of serotype 9V wzy gene (Genbank accesion no. AF402095).
SEQ ID NO:482—Sequence of serotype 19B wzy gene (Genbank accesion no. AF004325).
SEQ ID NO:483—Sequence of serotype 19F wzy gene (Genbank accesion no. U09239).
SEQ ID NO:484—Sequence of serotype 2 wzy gene (Genbank accesion no. AF026471).
SEQ ID NO:485—Sequence of serotype 23F way gene (Genbank accesion no. AF057294).
SEQ ID NO:486—Sequence of serotype 33F wzy gene (Genbank accesion no. AFAJ006986).
SEQ ID NO:487—Sequence of serotype 37 wzy gene (Genbank accesion no. AJ131984).
SEQ ID NO:488—Sequence of serotype 6A wzy gene (Genbank accesion no. AY078347).
SEQ ID NO:489—Sequence of serotype 6B wzy gene (Genbank accesion no. AF316640).
SEQ ID NO:490—Sequence of serotype 8 wzy gene (Genbank accesion no. AF316641).
SEQ ID NO:491—Sequence of serotype 18C wzy gene (Genbank accesion no. AF316642).
SEQ ID NO:492—Consensus sequence for 3′ end of the cpsA gene and the 5′ end of the cpsB gene of S. pneumoniae strains that were analysed.
   * Indicates that these sequences were extracted from unnannotated sequence data from the Sanger Institute website.

### DETAILED DESCRIPTION OF THE INVENTION

Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry).

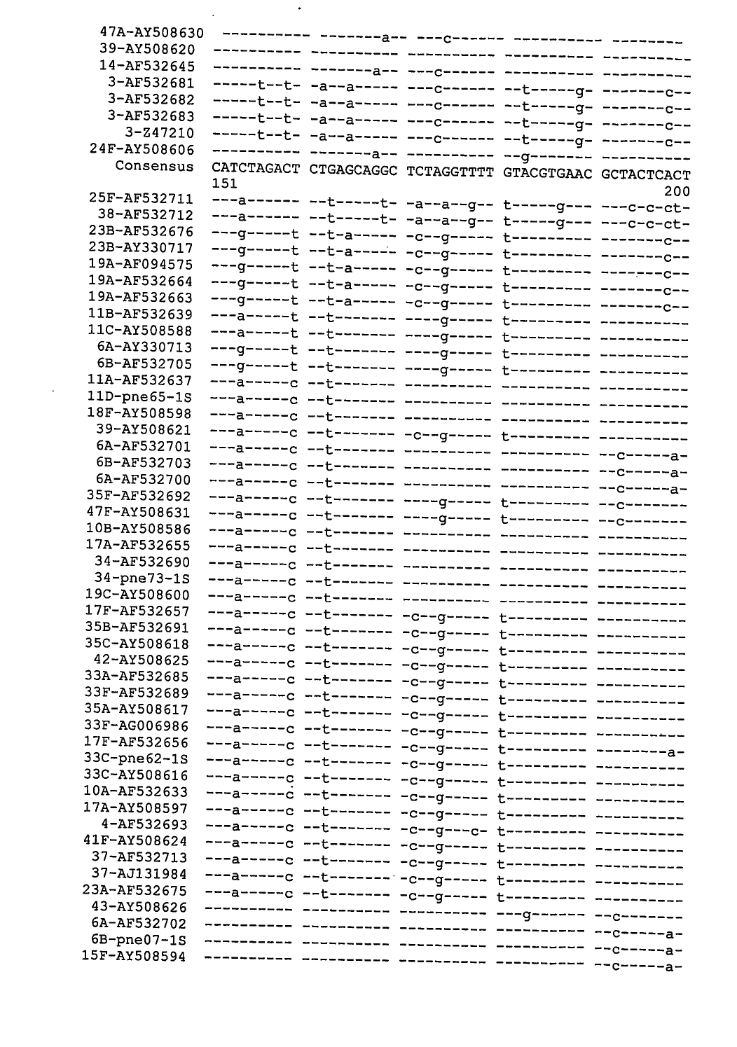

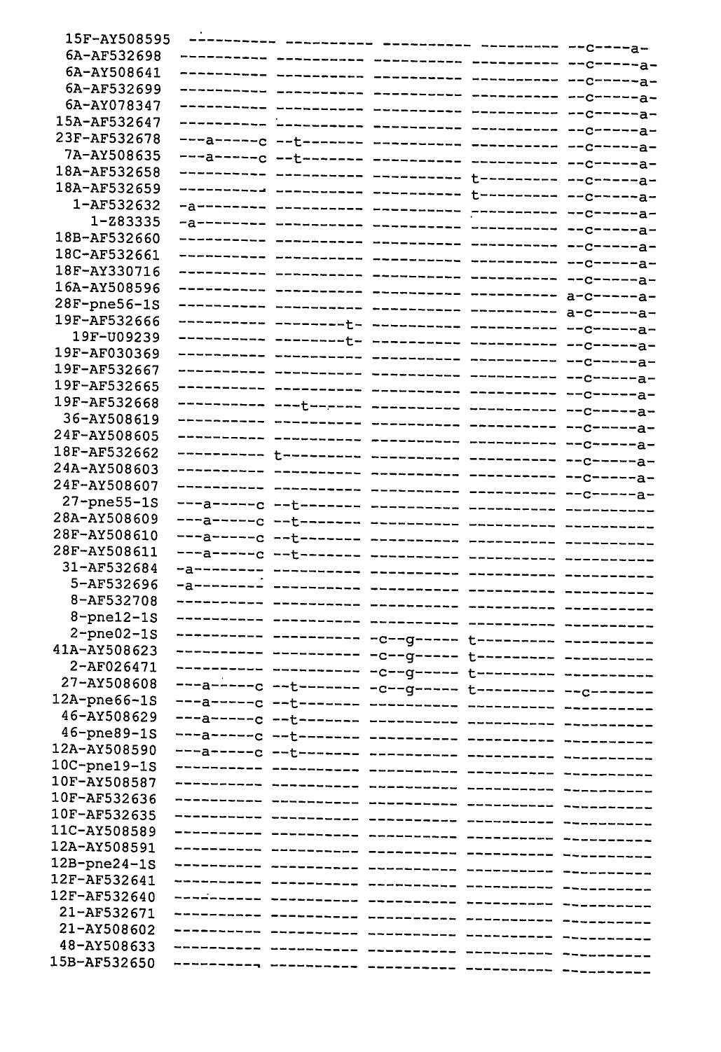

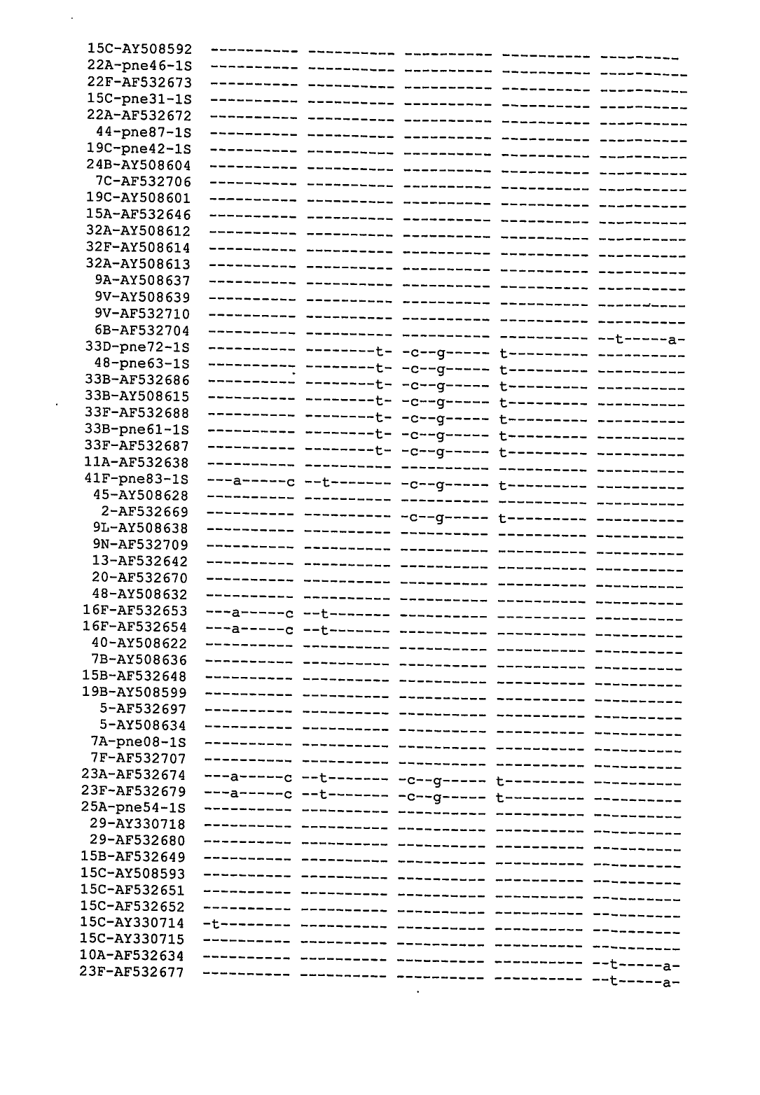

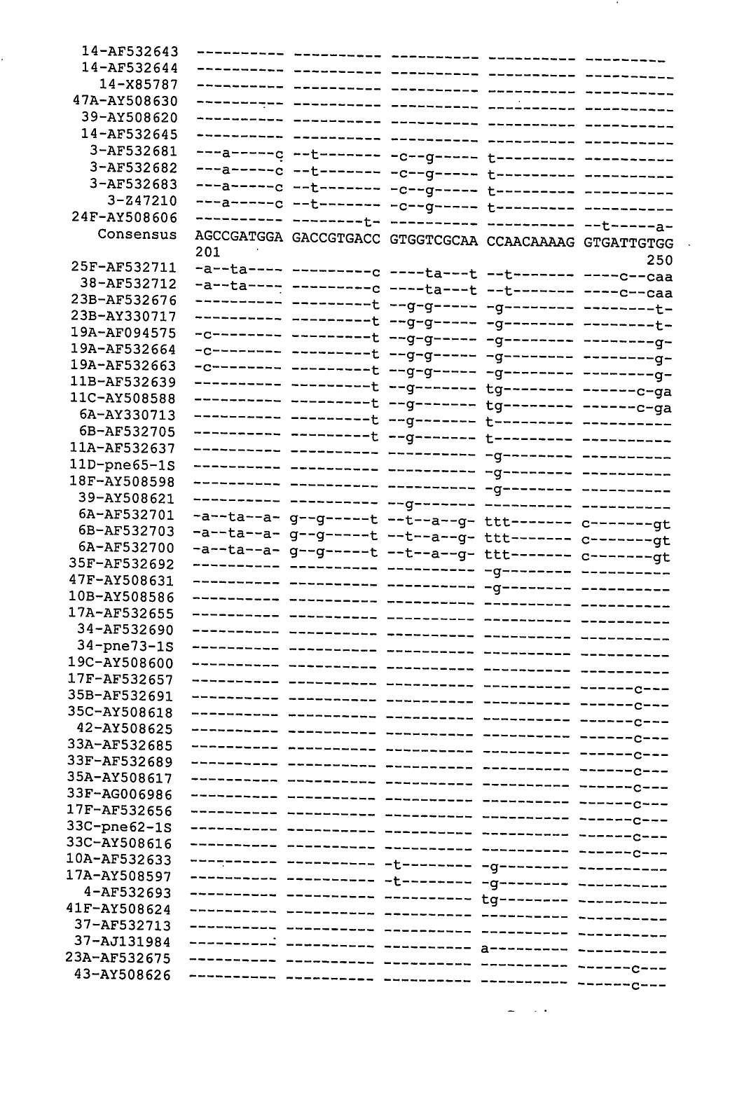

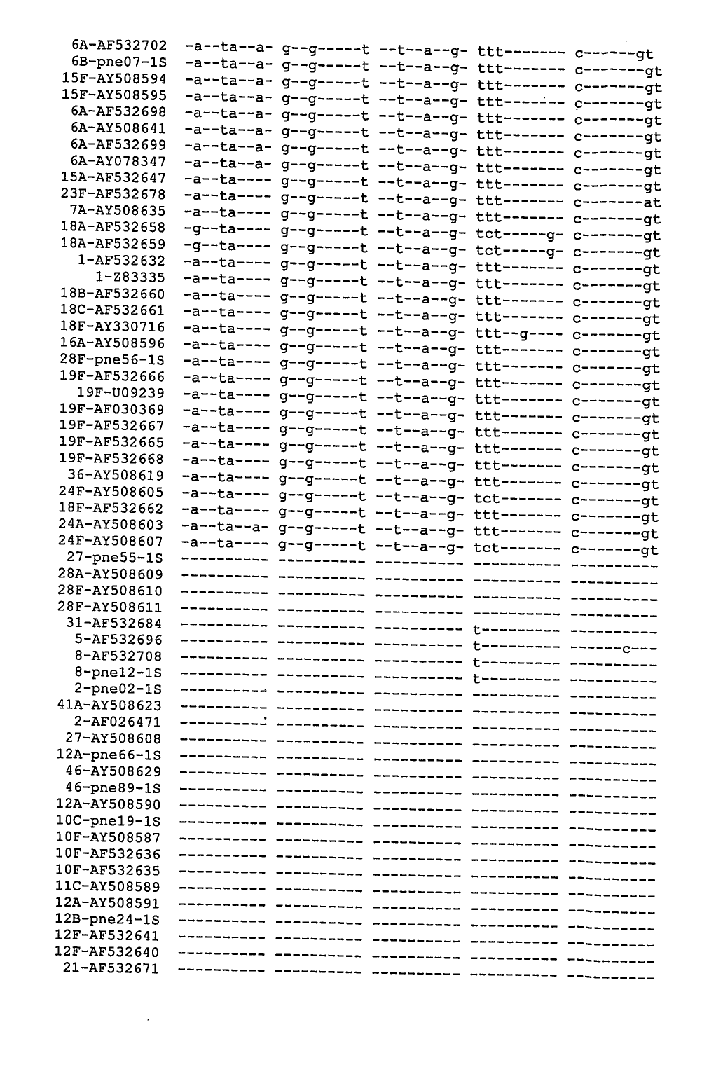

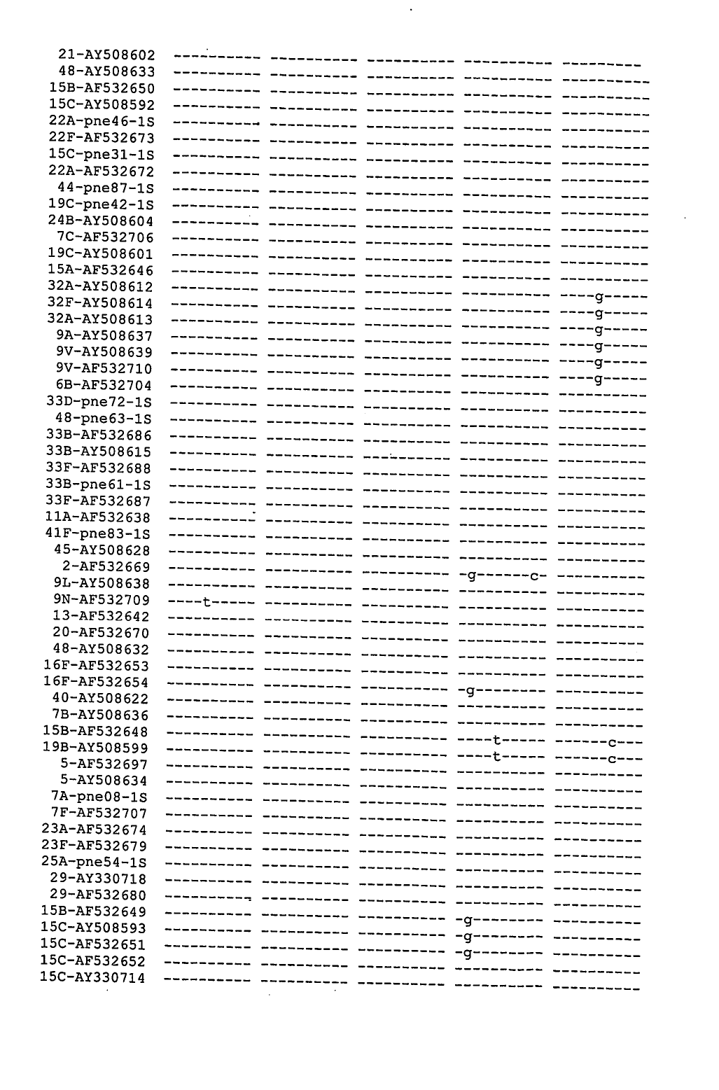

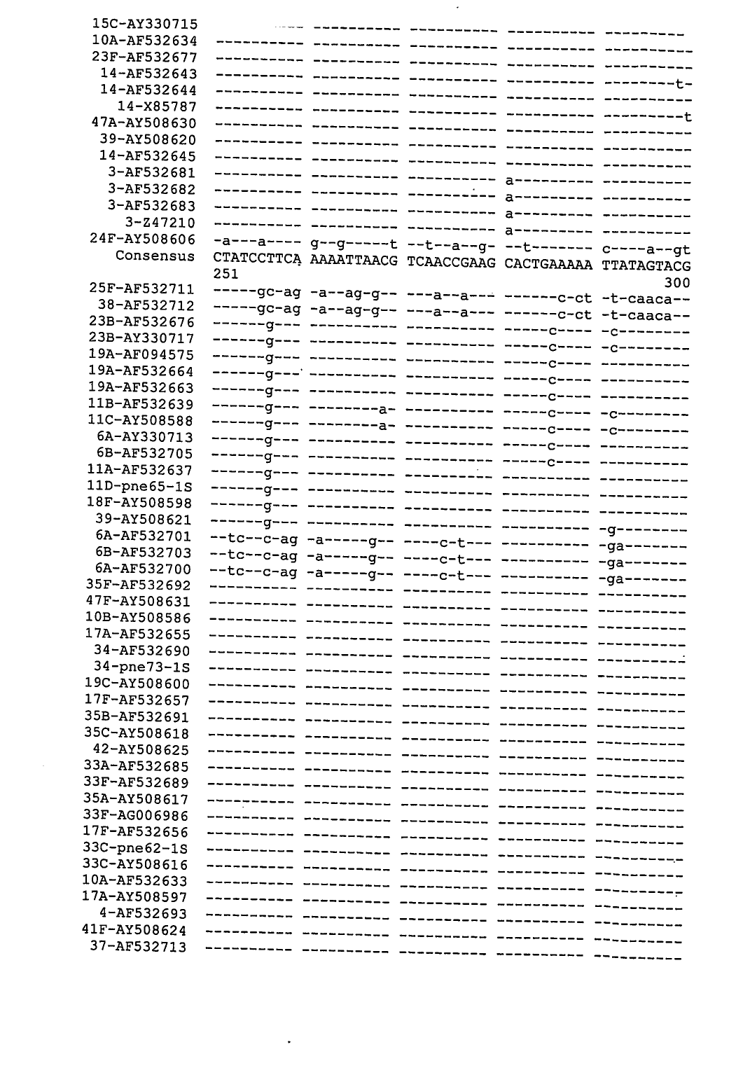

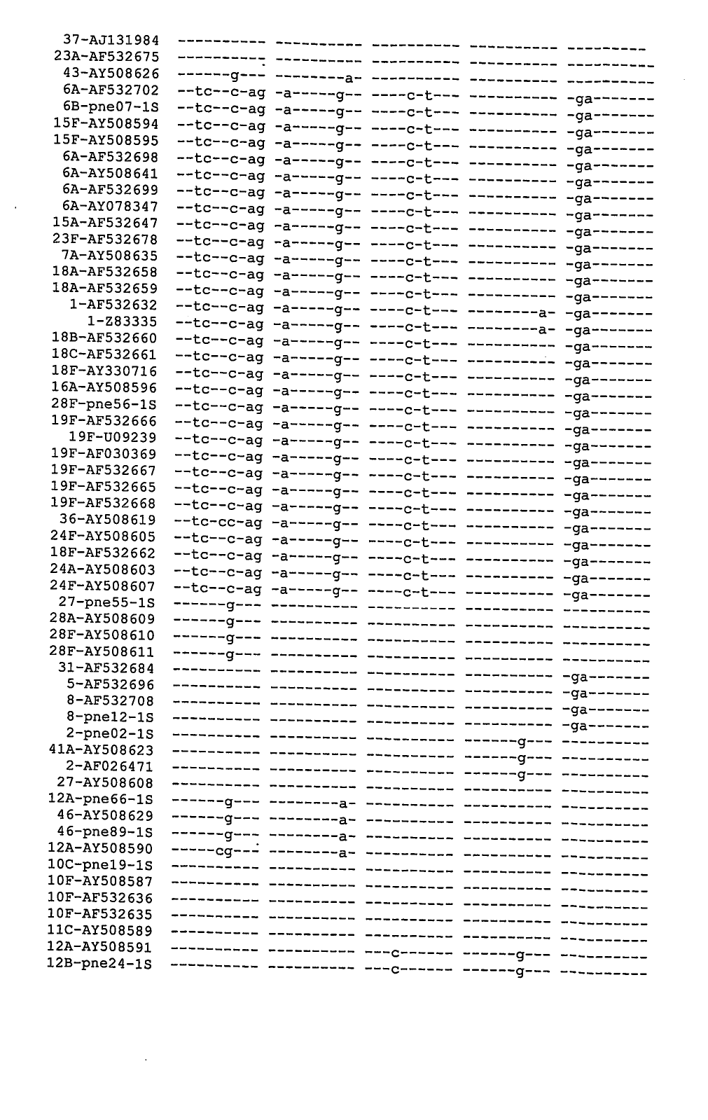

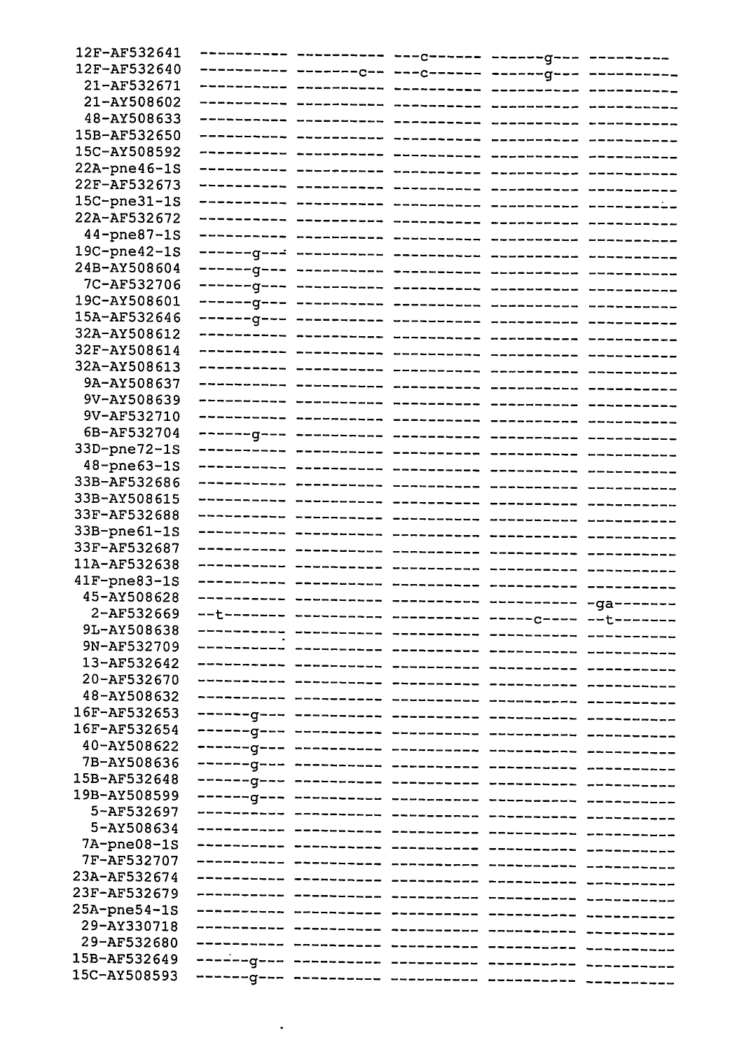

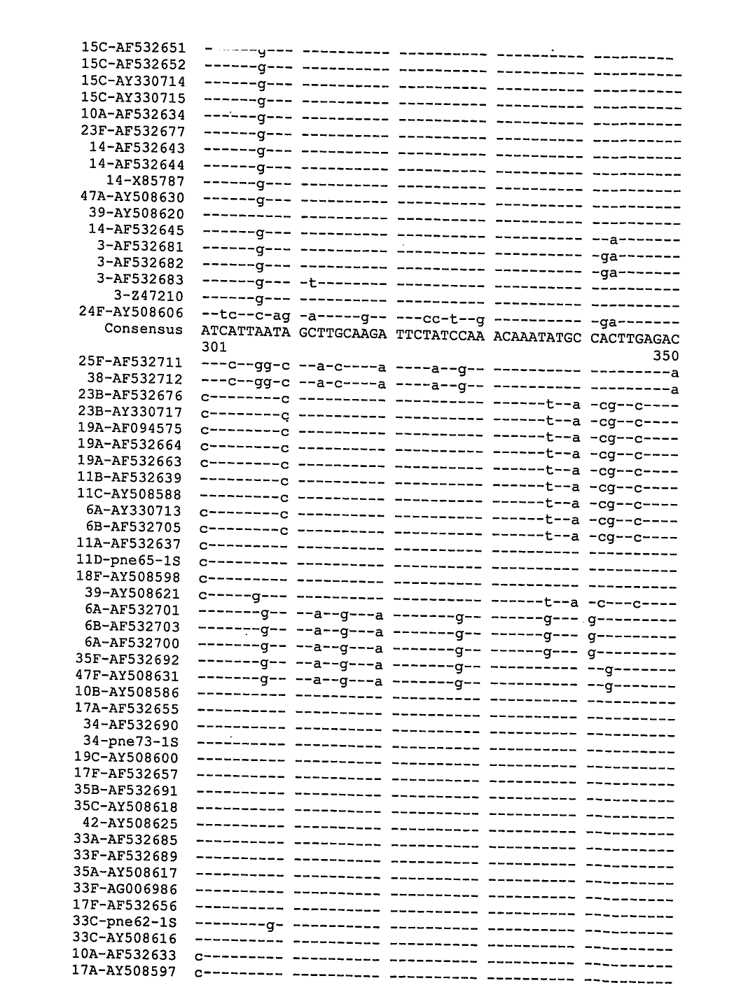

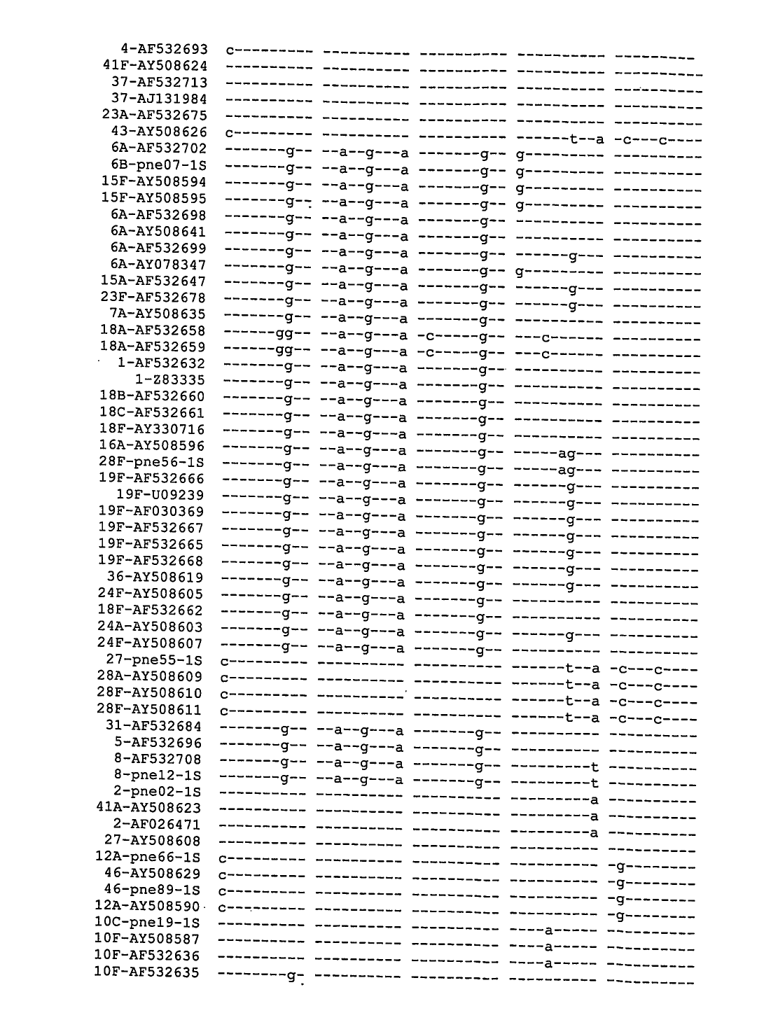

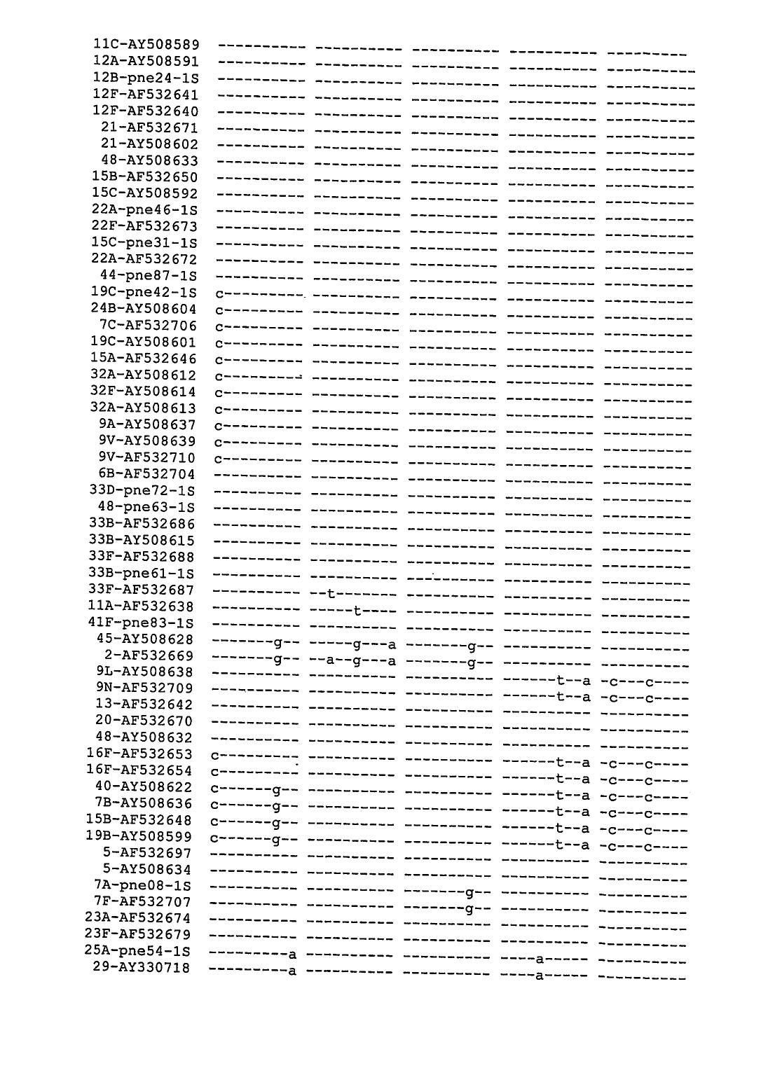

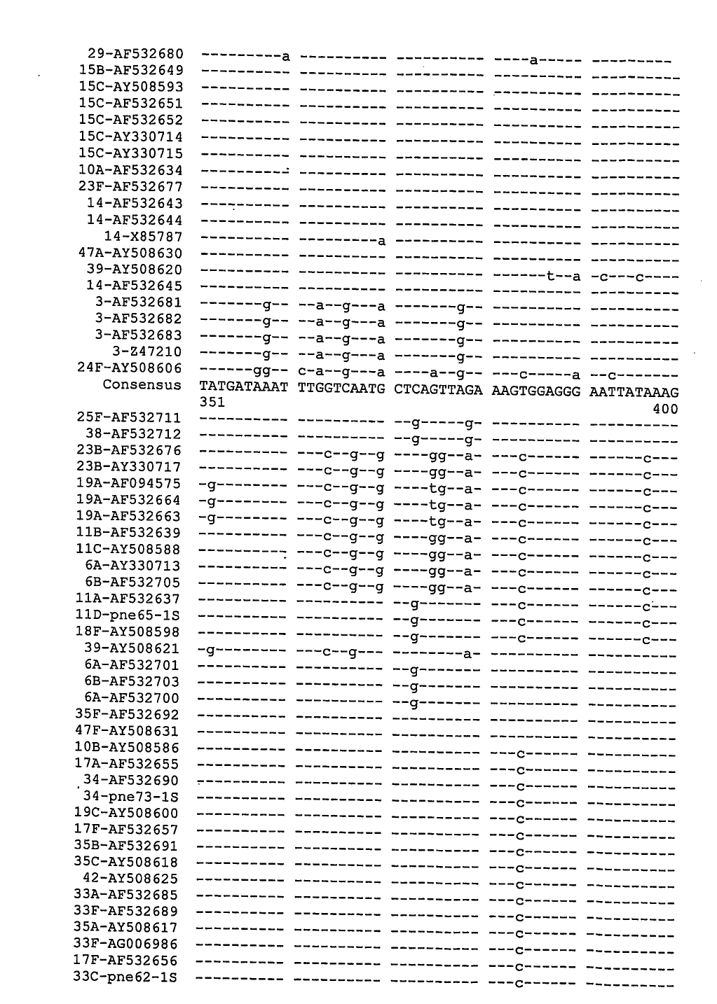

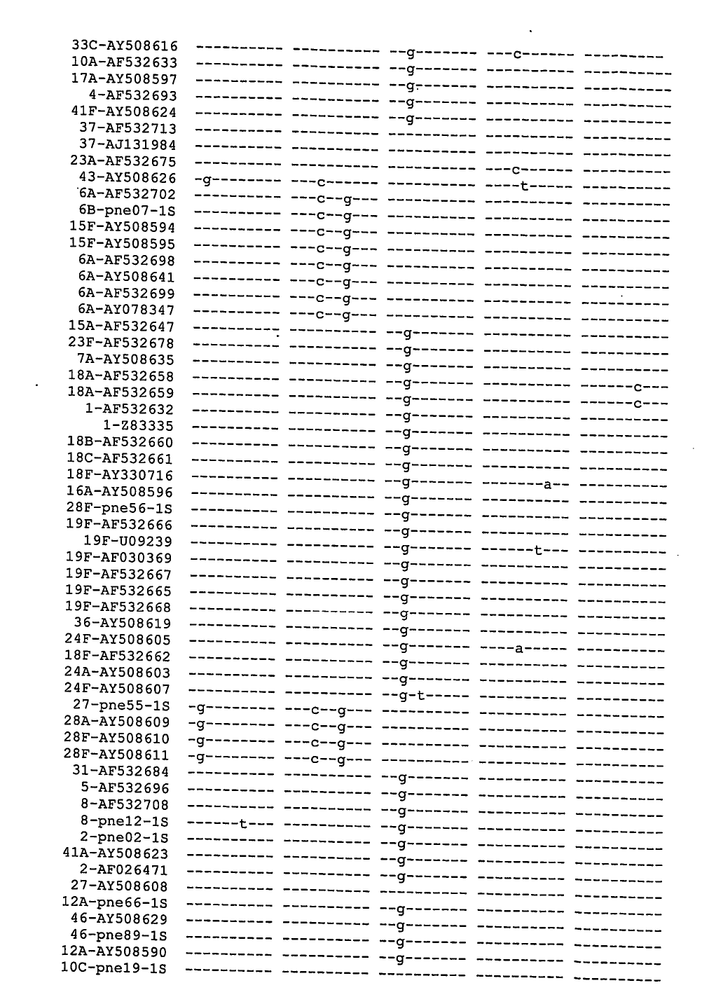

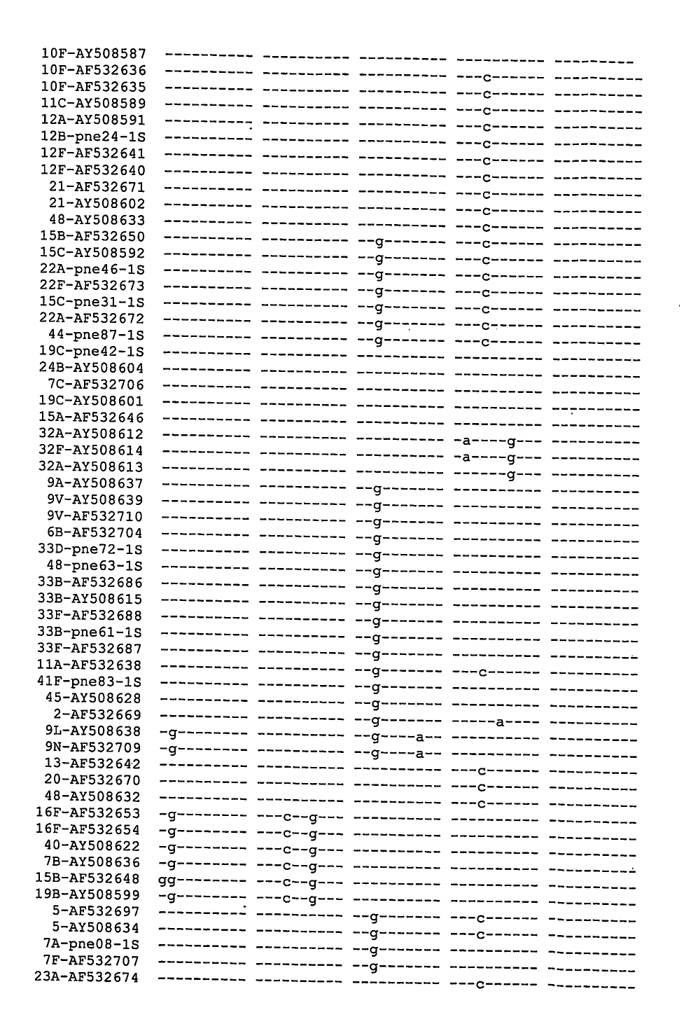

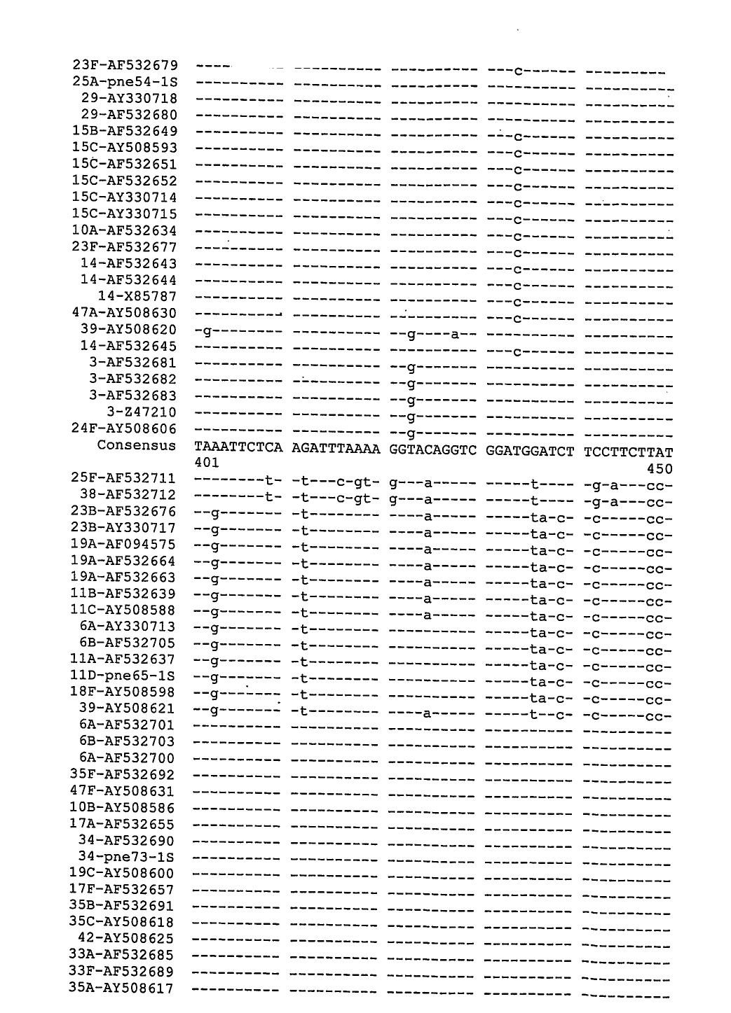

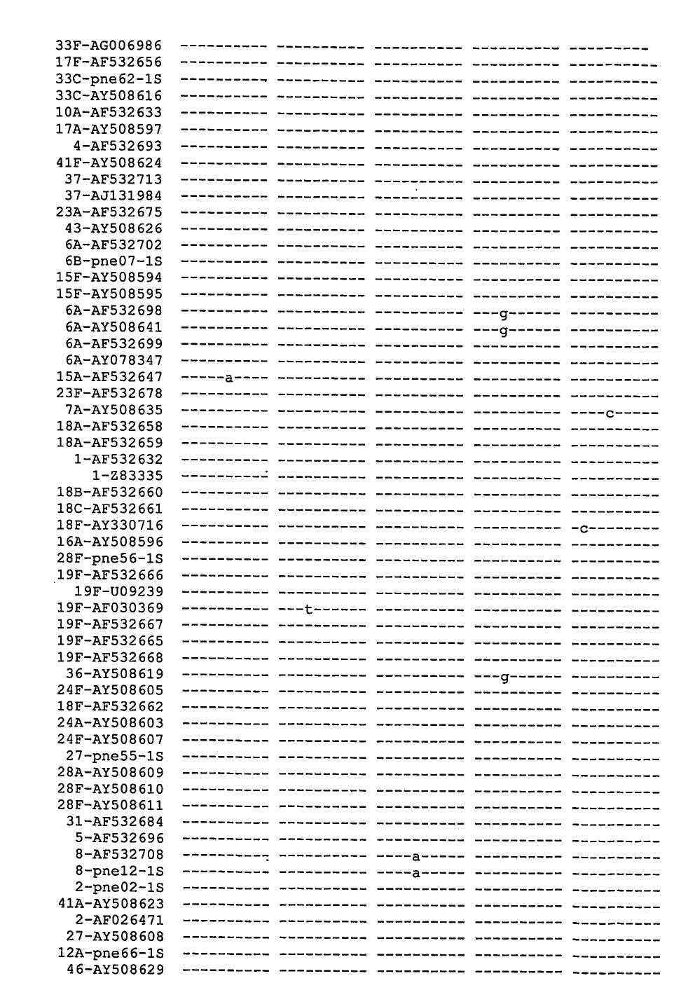

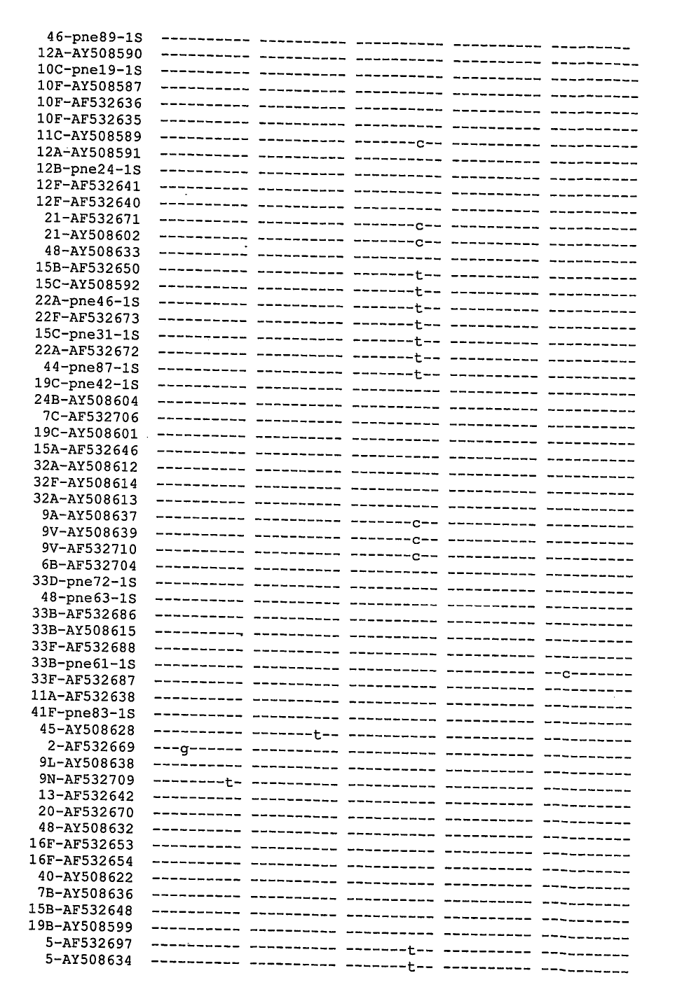

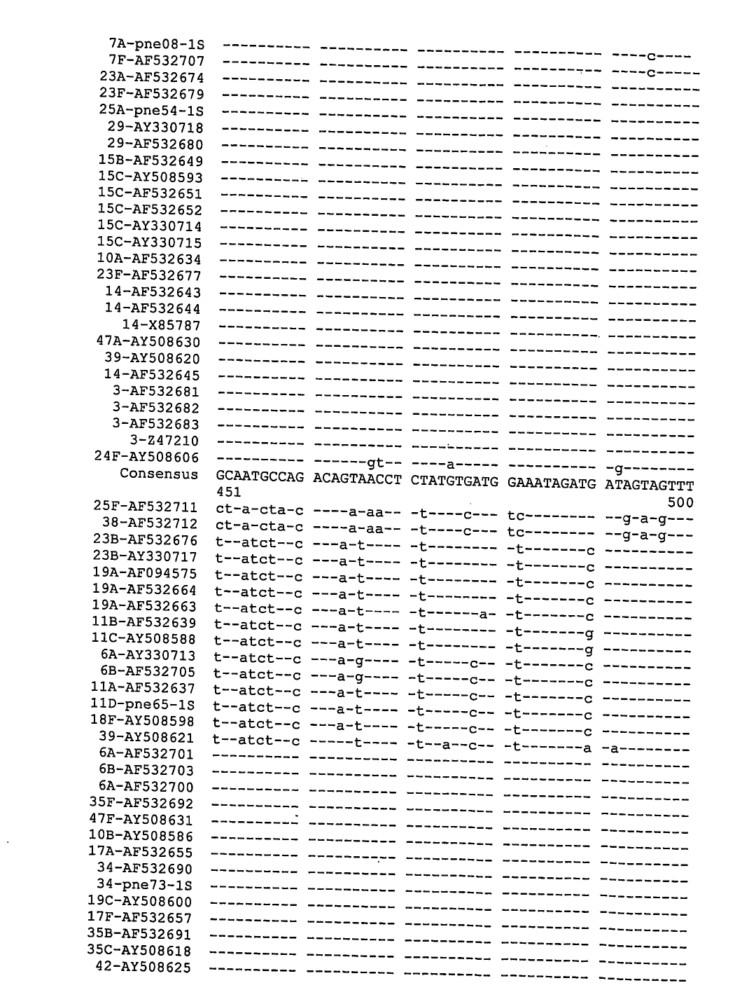

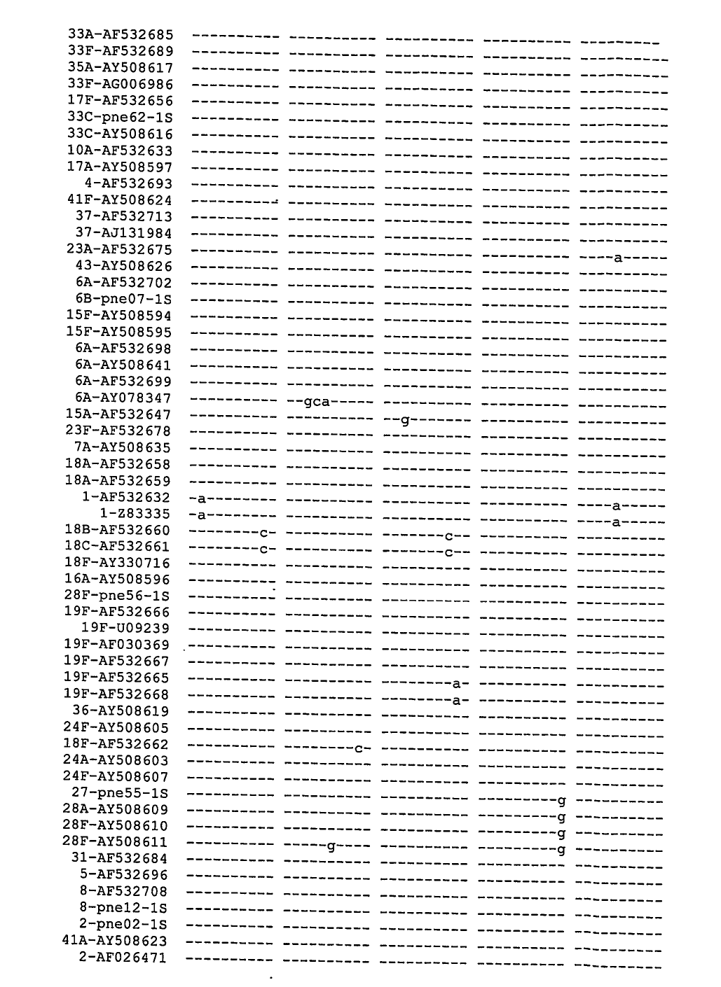

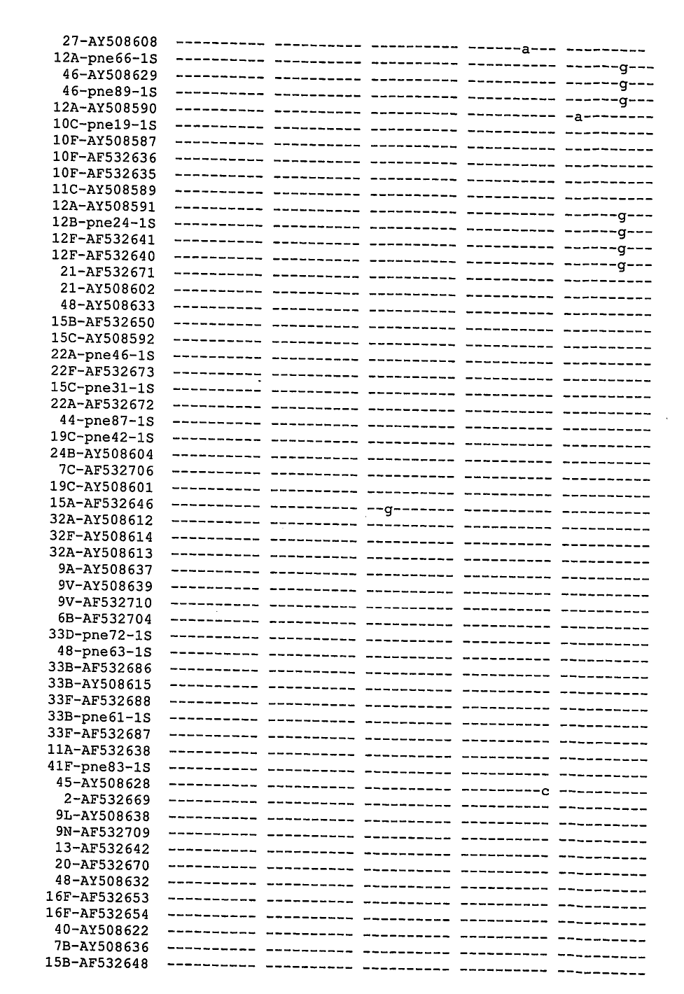

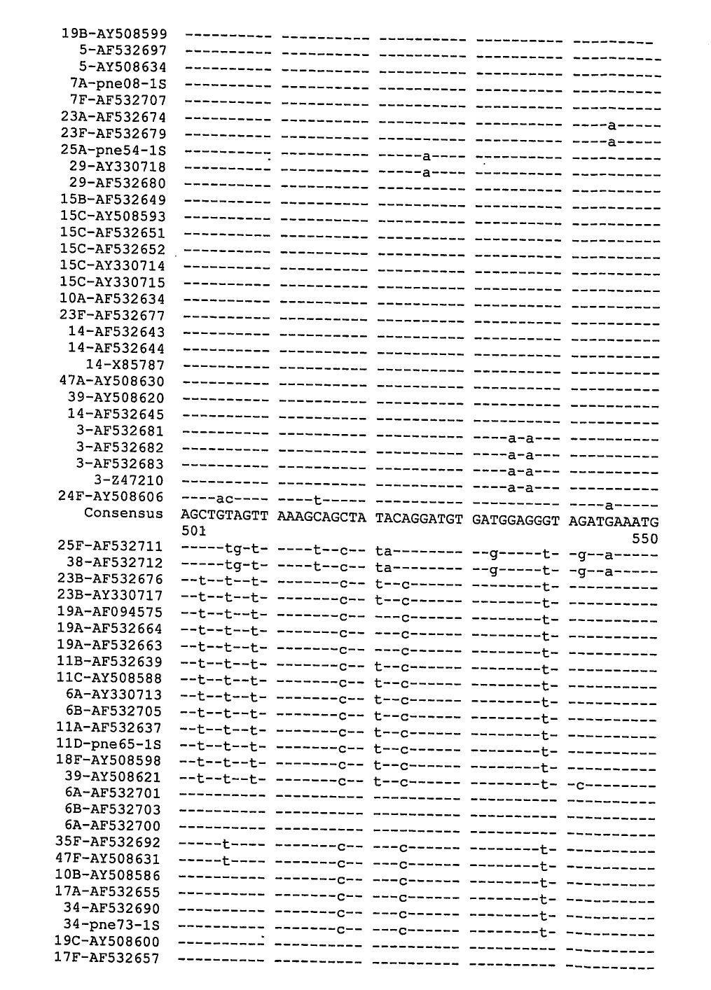

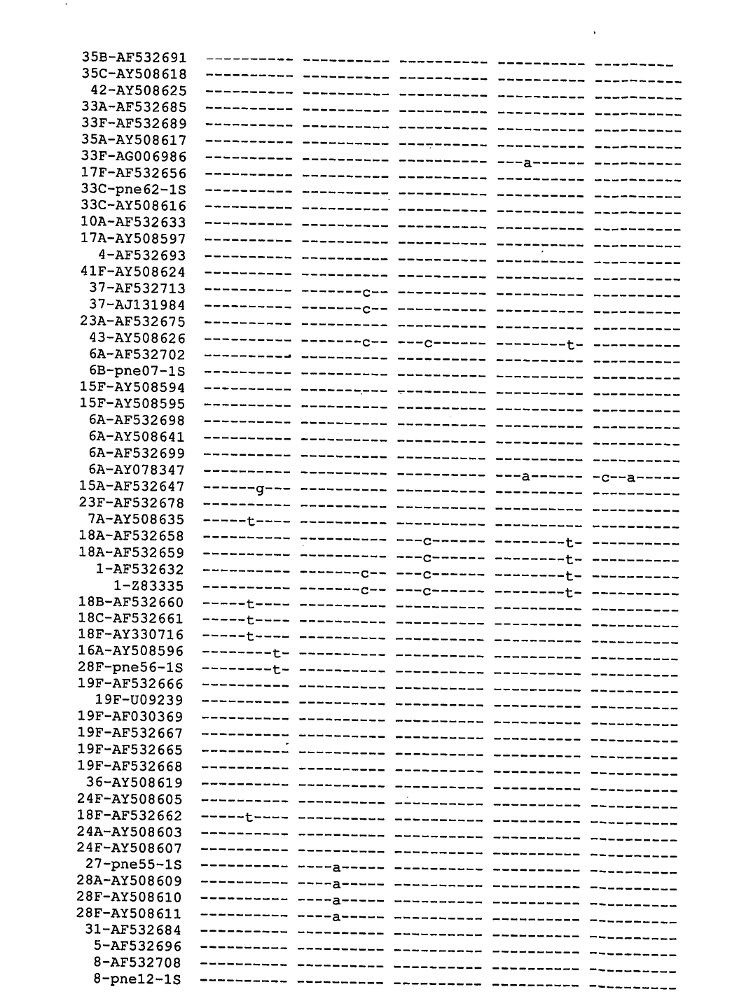

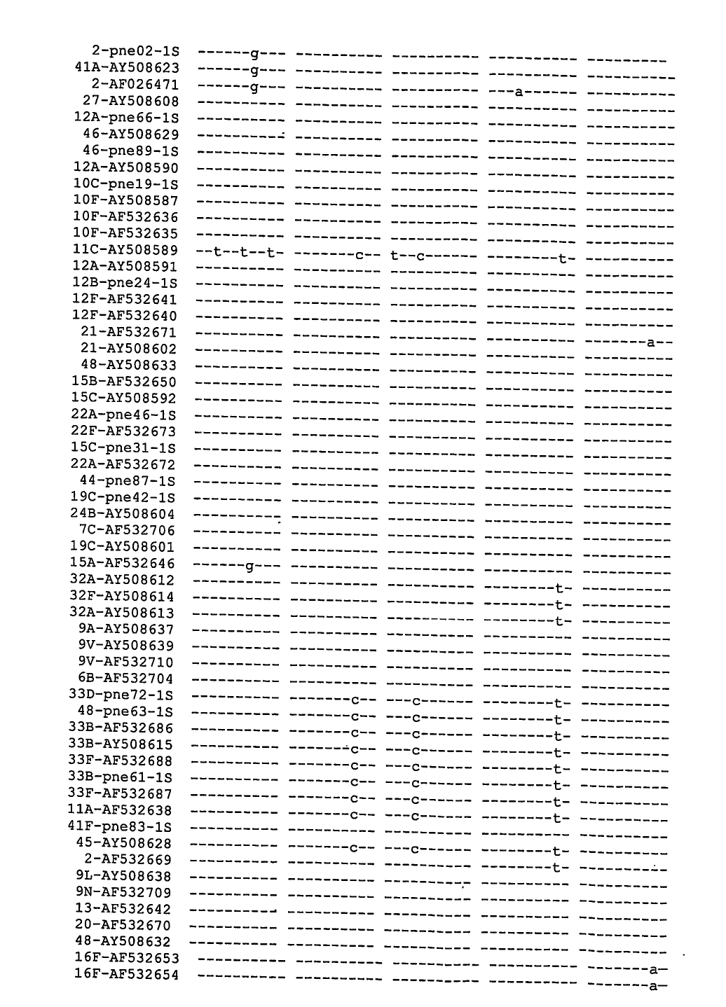

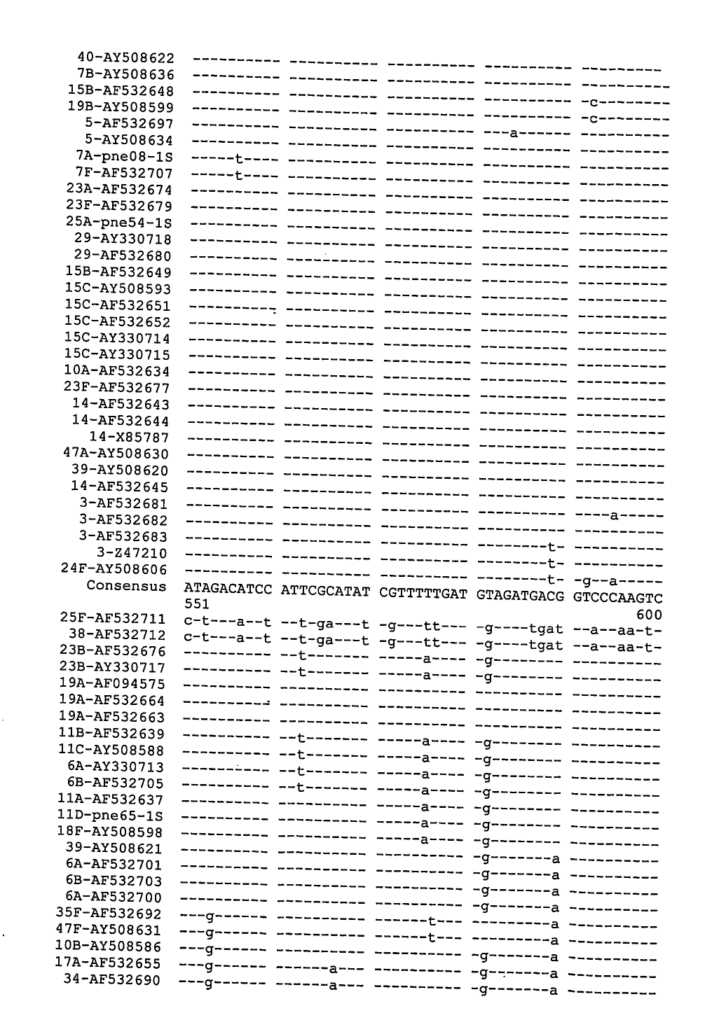

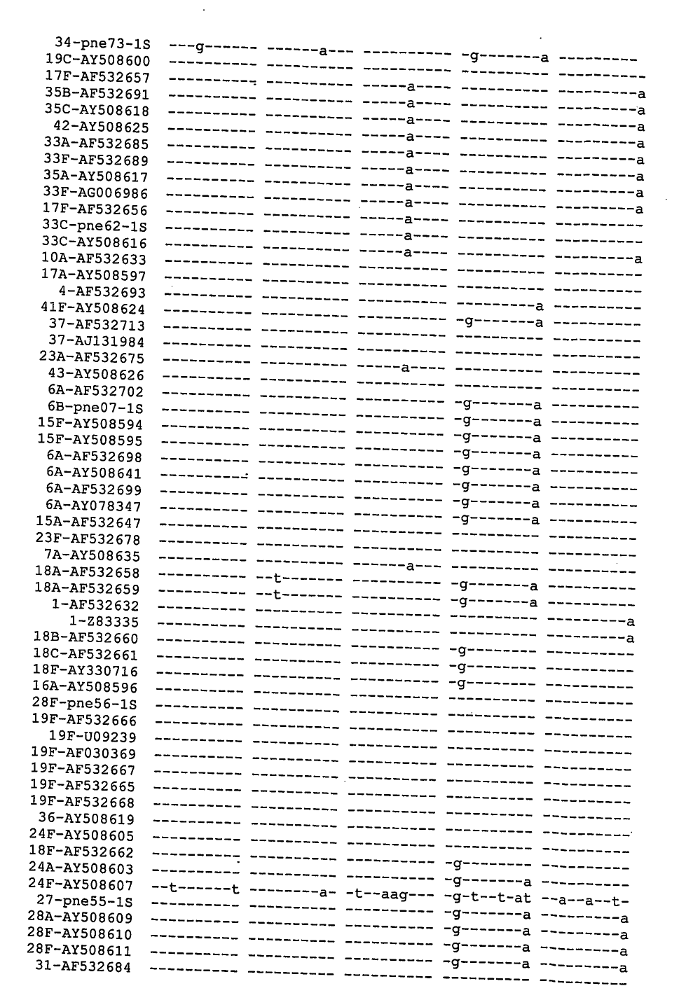

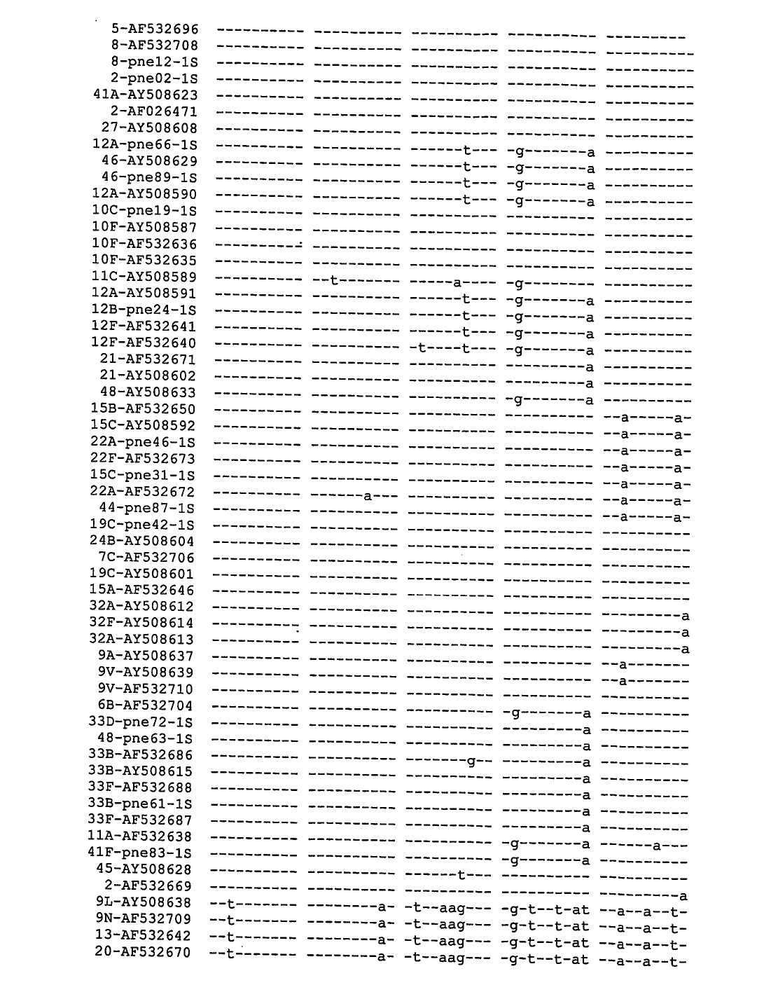

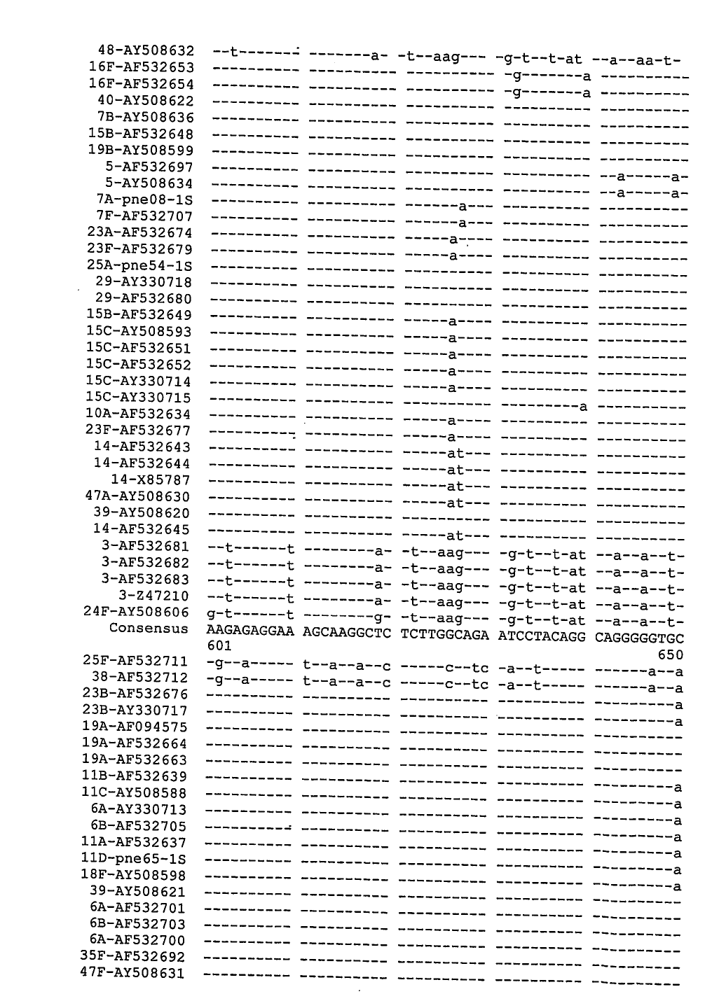

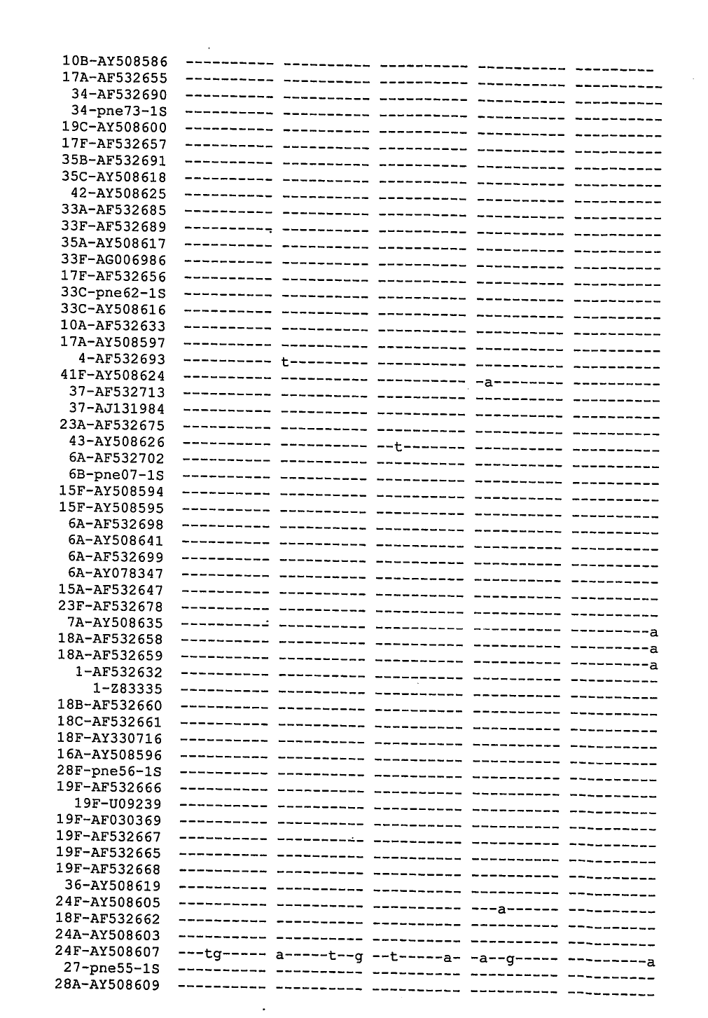

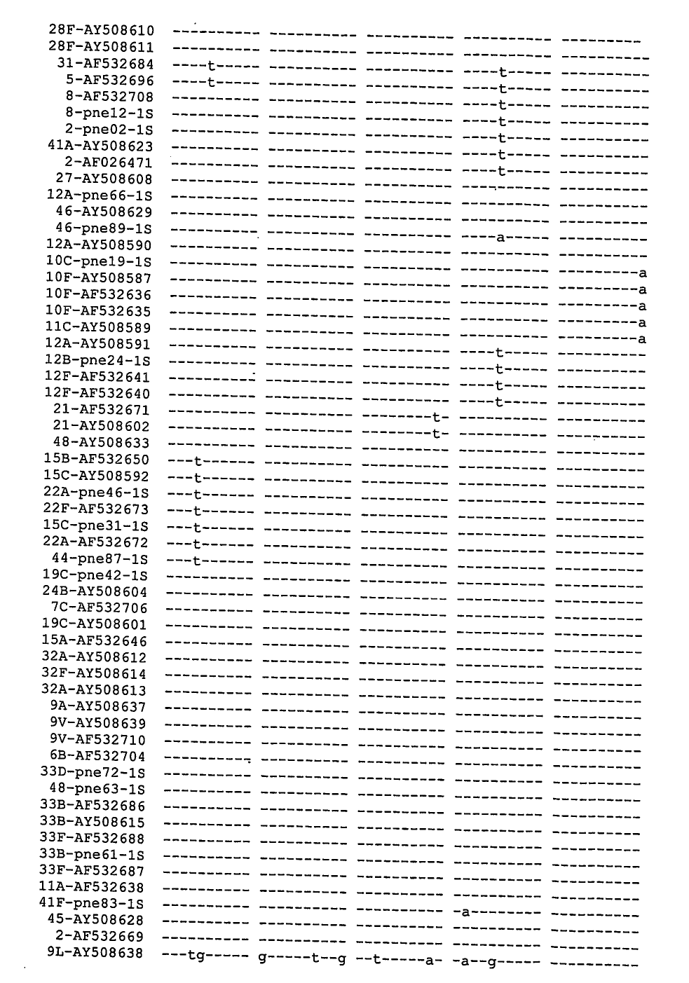

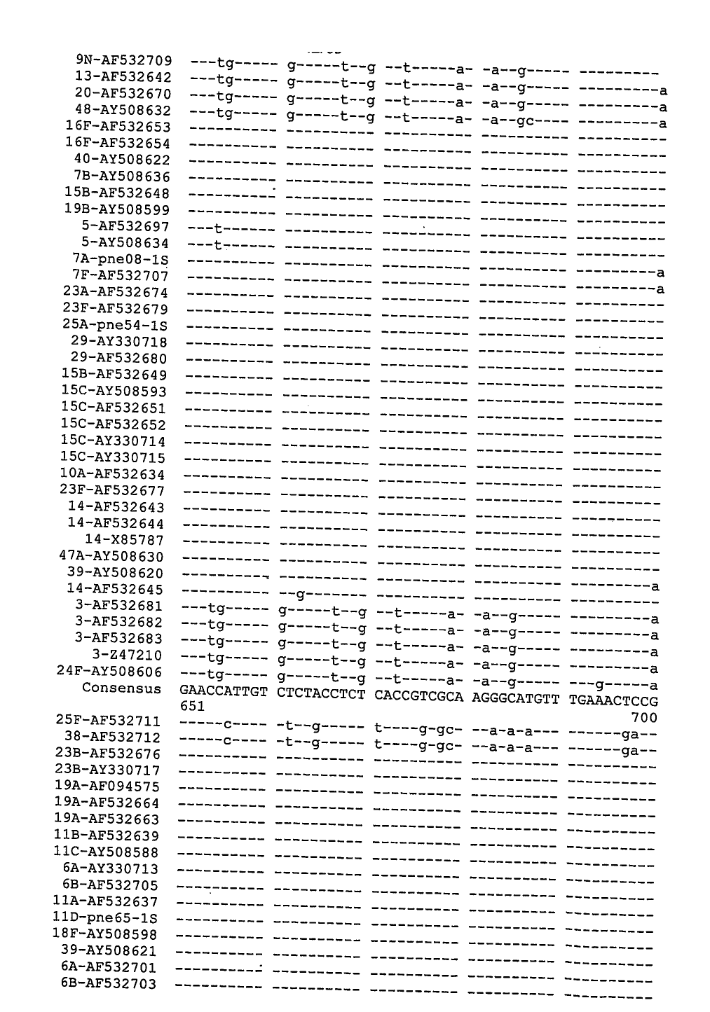

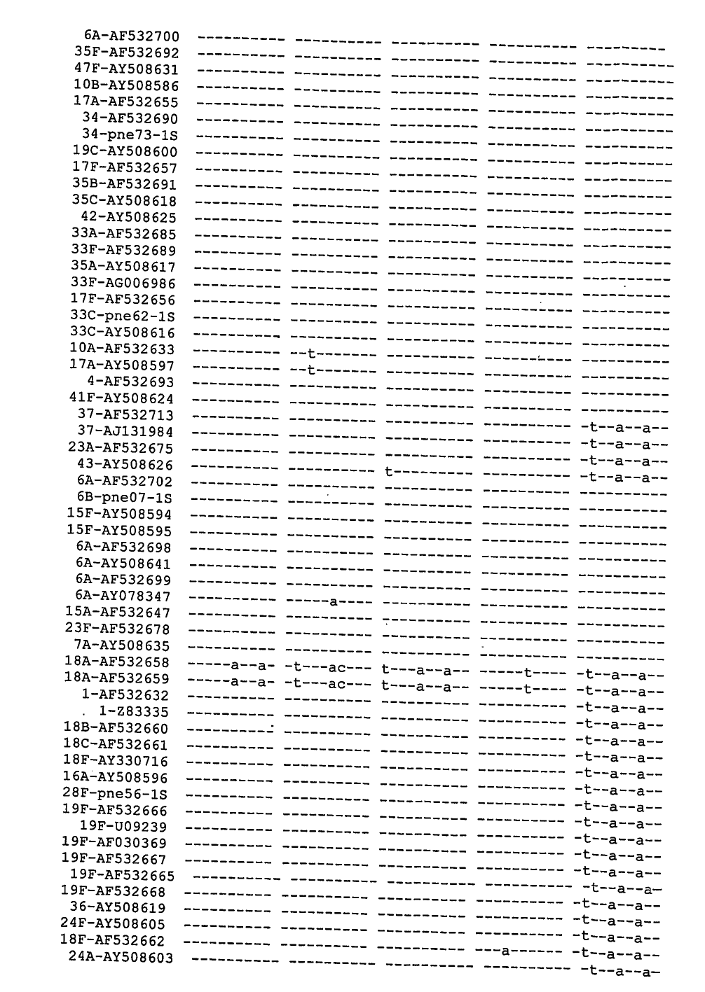

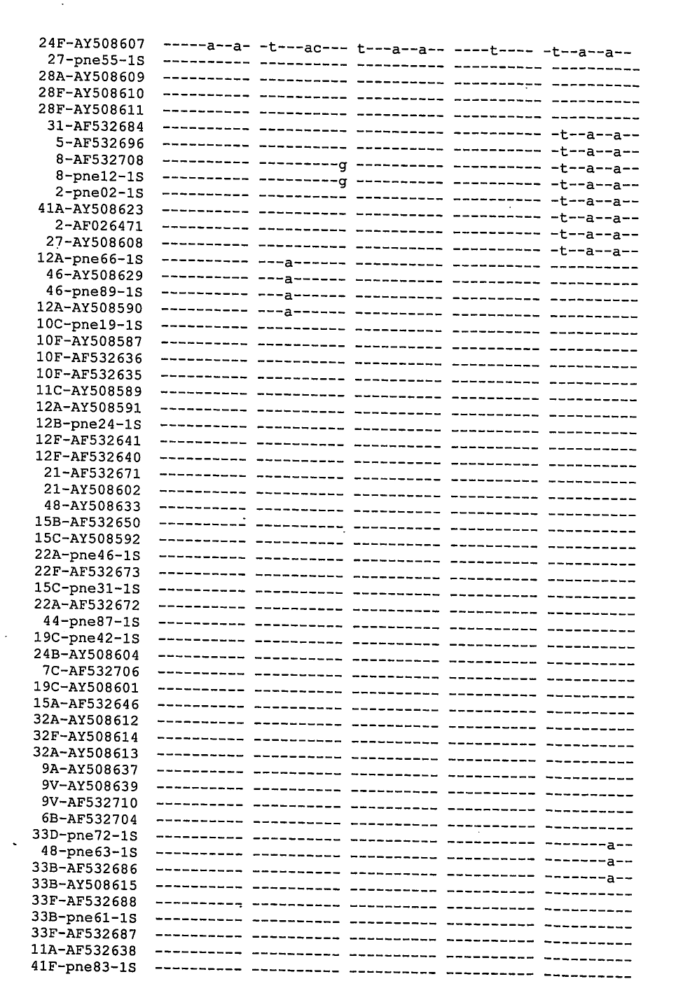

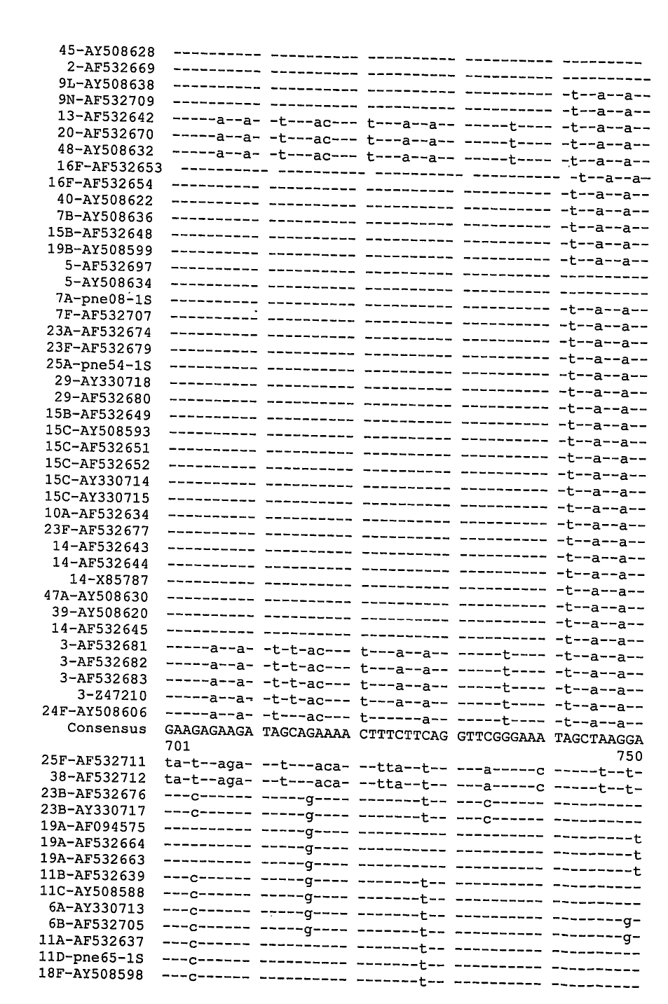

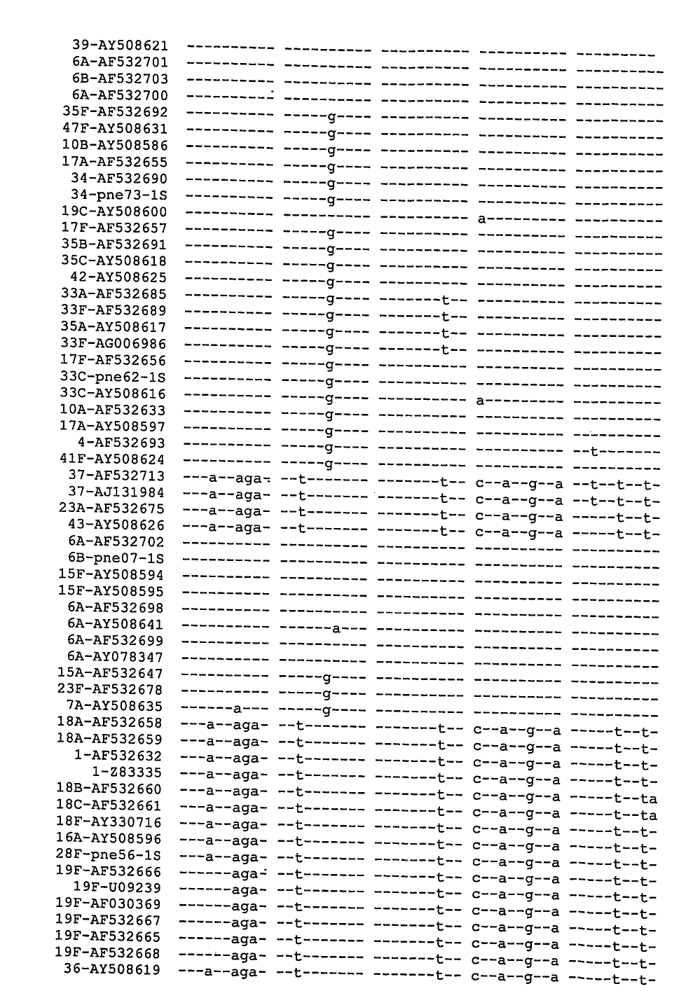

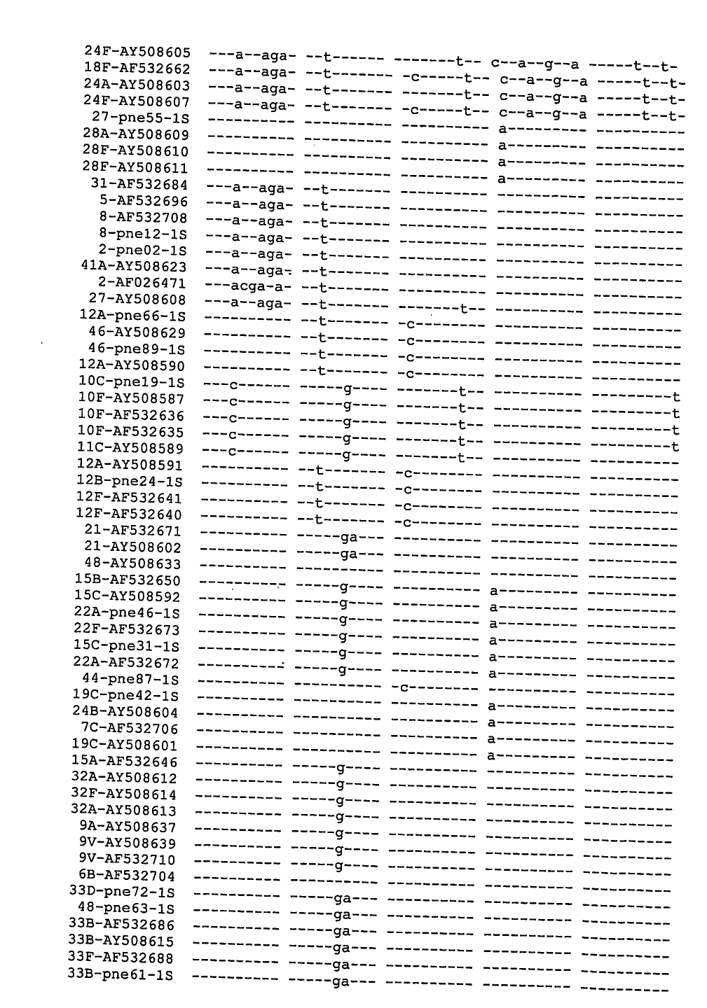

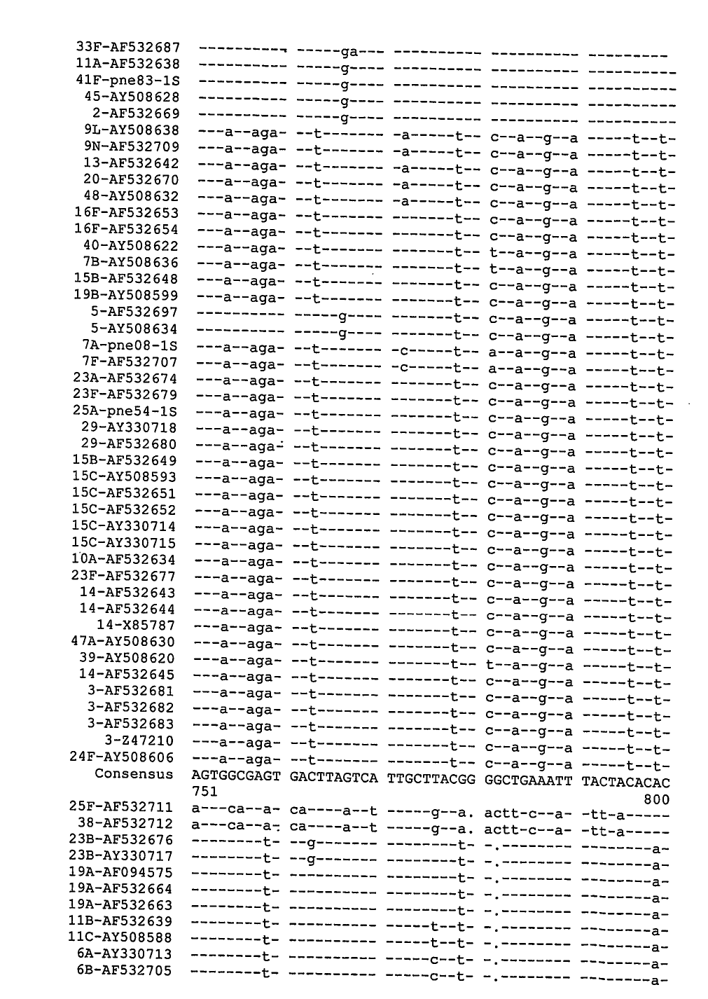

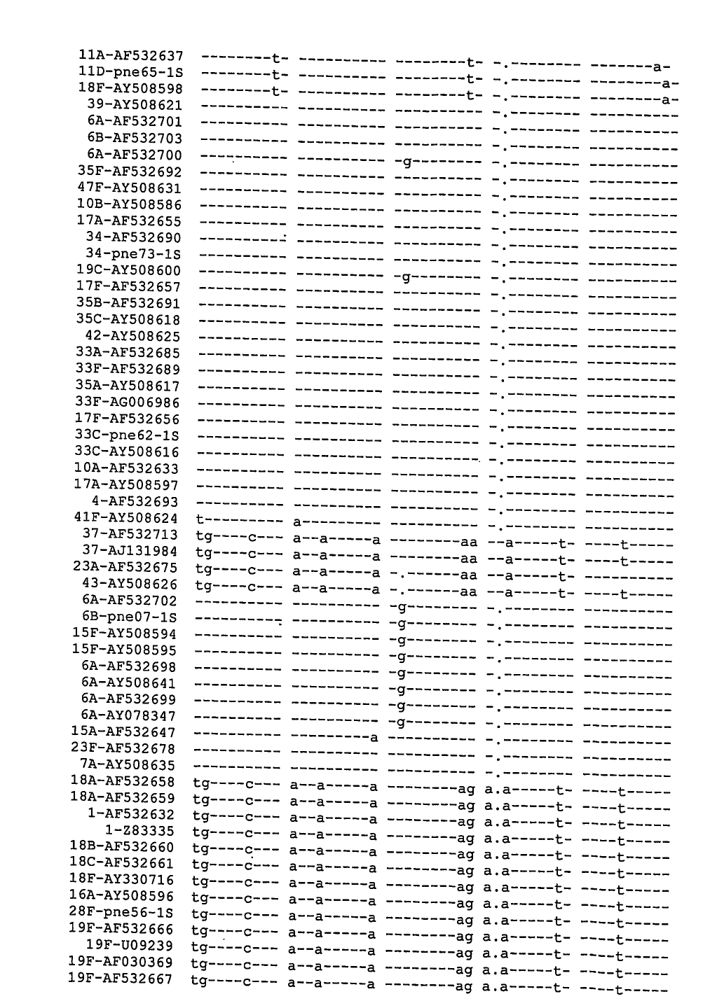

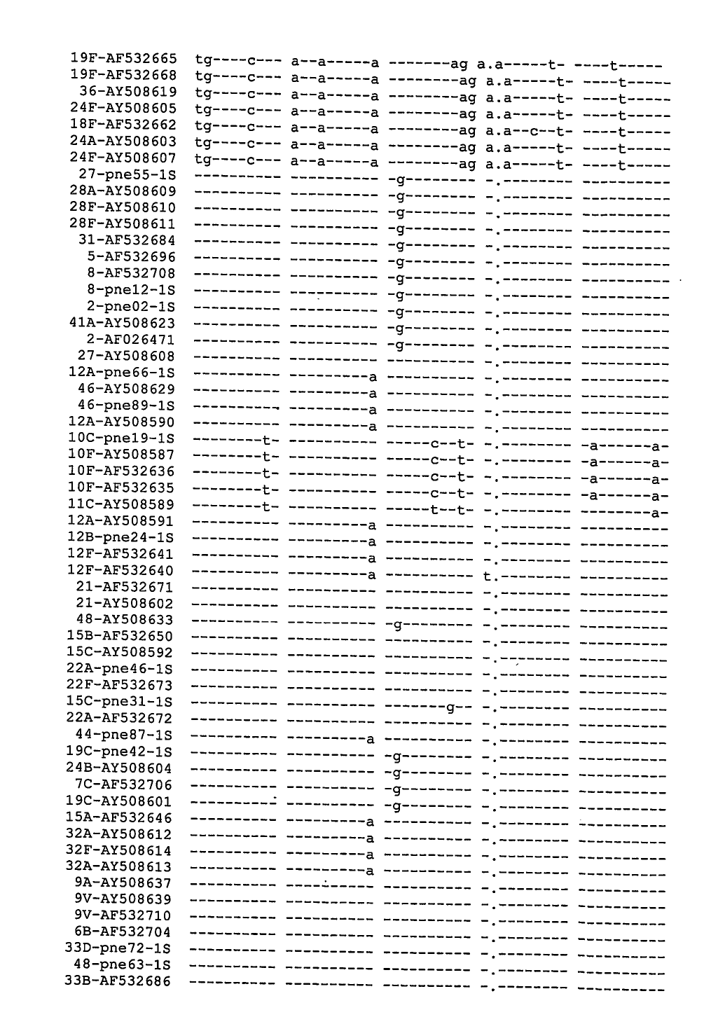

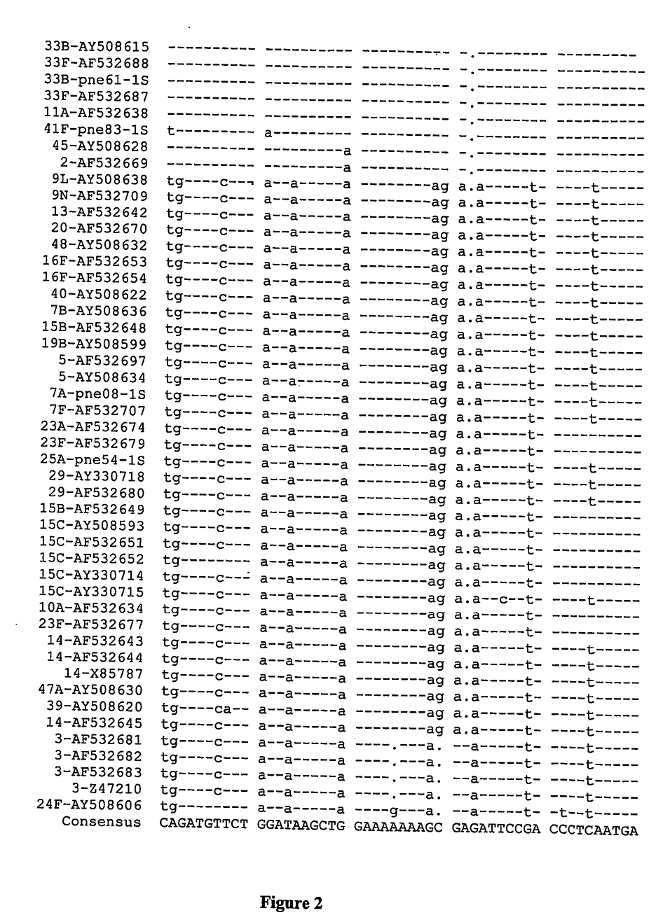

As used herein, the term “nucleotide sequence between the 3′ end of the cpsA gene and the 5′ end of the cpsB gene” at least refers to the region spanning from nucleotide 2470 to nucleotide 3268 of FIG. 1. FIG. 1 provides the genomic sequence of cpsA (wzg) and cpsB (wzh) genes of serotype 4 as published by Jiang et al. (2001) and submitted as GenBank Accession Number AF316639. As the skilled addressee would be aware, the same region from other serotypes of S. pneumoniae can be identified using standard techniques such as DNA cloning, sequencing and nucleotide sequence alignment. Such techniques are described in further detail in the Examples section. In addition, these techniques have been used to determine the nucleotide sequence between the 3′ end of the cpsA gene and the 5′ end of the cpsB gene from many different serotypes of S. pneumoniae, the results of which, including a consensus sequence for this region, are also provided in FIG. 2.

As used herein, the term “primer pairs that amplify the same region, or diagnostic portion thereof, from the genome of a strain of S. pneumoniae”, or variations thereof, refers to the capability of the skilled addressee to determine where the identified primers of the claimed invention hybridize the S. pneumoniae genome of a particular strain(s), and subsequent ability to design alternate primers which can be used for the same purpose as the primers defined herein. Typically, these alternate primers will hybridize the same region of the genome but be larger or smaller in size, or these alternate primers will hybridize to a region of the genome which is in close proximity, for example within 500 basepairs, to where the specifically defined primers hybridize. Naturally, the term “diagnostic portion thereof” refers to the alternate primers being capable of amplifying a portion of the region of the defined primers but still capable of amplifying enough of the region to determine the serotype of a particular S. pneumoniae isolate.

General Techniques

Unless otherwise indicated, the recombinant DNA and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present), and are incorporated herein by reference.

Detection of Polymorphisms

Any technique known in the art can be used to detect a polymorphism described herein. Examples of such techniques include, but are not limited to, sequencing of the DNA at one or more of the relevant positions; differential hybridisation of an oligonucleotide probe designed to hybridise at the relevant positions of a particular S. pneumoniae serotype(s); denaturing gel electrophoresis following digestion with an appropriate restriction enzyme, preferably following amplification of the relevant DNA regions; S1 nuclease sequence analysis; non-denaturing gel electrophoresis, preferably following amplification of the relevant DNA regions; conventional RFLP (restriction fragment length polymorphism) assays; selective DNA amplification using oligonucleotides which are matched for a particular S. pneumoniae serotype(s) unmatched for other S. pneumoniae serotype(s); or the selective introduction of a restriction site using a PCR (or similar) primer matched for a particular S. pneumoniae serotype(s), followed by a restriction digest. As outlined above, it is preferred that the nucleotide sequence between the 3′ end of the cpsA gene and the 5′ end of the cpsB gene is characterized by DNA sequencing, whilst the analysis at least a portion of the wzy and/or wzx gene is performed by procedures involving the detection of amplification products.

In one embodiment, the informative serotyping information provided herein is adapted to produce a molecular capsular sequence typing database as generally described by Robertson et al. (2004).

PCR-based methods of detection may rely upon the use of primer pairs, at least one of which binds specifically to a region of interest in one or more, but not all, serotypes. Unless both primers bind, no PCR product will be obtained. Consequently, the presence or absence of a specific PCR product may be used to determine the presence of a sequence indicative of a specific S. pneumoniae serotype(s). However, as mentioned, only one primer need correspond to a region of heterogeneity in the genes/regions of interest. The other primer may bind to a conserved or heterogenous region within said gene/region or even a region within another part of the S. pneumoniae genome, whether said region is conserved or heterogeneous between serotypes.

Alternatively, primers that bind to conserved regions of the S. pneumoniae genome but which flank a region whose length varies between serotypes may be used. In this case, a PCR product will always be obtained when S. pneumoniae bacteria are present but the size of the PCR product varies between serotypes. Examples of such varying amplification product lengths are disclosed herein in relation to the wzy and wzx genes.

Furthermore, a combination of specific binding of one or both primers and variations in the length of PCR primer may be used as a means of identifying particular molecular serotypes.

In some cases, PCR and other specific hybridisation-based serotyping methods will involve the use of nucleotide primers/probes which bind specifically to a region of the genome of a S. pneumoniae serotype which includes a nucleotide which varies between two or more serotypes. Thus the primers/probes may comprise a sequence which is complementary to one of such regions. Where positions of heterogeneity are close together (for instance within 5 or so nucleotides), it may be desirable to use a primer/probe which hybridises specifically to a region of the S. pneumoniae genome that comprises two or more positions of heterogeneity. Such primers/probes are likely to have improved specificity and reduce the likelihood of false positives.

PCR techniques that utilize fluorescent dyes may be used in the detection methods of the present invention. These include, but are not limited to, the following five techniques.

i) Fluorescent dyes can be used to detect specific PCR amplified double stranded DNA product (e.g. ethidium bromide, or SYBR Green I).

ii) The 5′ nuclease (TaqMan) assay can be used which utilizes a specially constructed primer whose fluorescence is quenched until it is released by the nuclease activity of the Taq DNA polymerase during extension of the PCR product.

iii) Assays based on Molecular Beacon technology can be used which rely on a specially constructed oligonucleotide that when self-hybridized quenches fluorescence (fluorescent dye and quencher molecule are adjacent). Upon hybridization to a specific amplified PCR product, fluorescence is increased due to separation of the quencher from the fluorescent molecule.

iv) Assays based on Amplifluor (Intergen) technology can be used which utilize specially prepared primers, where again fluorescence is quenched due to self-hybridization. In this case, fluorescence is released during PCR amplification by extension through the primer sequence, which results in the separation of fluorescent and quencher molecules.

v) Assays that rely on an increase in fluorescence resonance energy transfer can be used which utilize two specially designed adjacent primers, which have different fluorochromes on their ends. When these primers anneal to a specific PCR amplified product, the two fluorochromes are brought together. The excitation of one fluorochrome results in an increase in fluorescence of the other fluorochrome.

Probes and primers may be fragments of DNA isolated from nature or may be synthetic. In one embodiment, primers/probes have a high melting temperature of >70° C. so that they may be used in rapid cycle PCR. Preferably, the primers/probes comprise at least 10, 15 or 20 nucleotides. Typically, primers/probes consist of fewer than 50 or 30 nucleotides. Primers/probes are generally polynucleotides comprising deoxynucleotides. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Primers/probes may be labelled with any suitable detectable label such as radioactive atoms, fluorescent molecules or biotin.

The primers be synthesized using techniques which are well known in the art. Generally, the primers can be made using synthesizing machines which are commercially available.

If required, in order to facilitate subsequent cloning of amplified sequences, primers may have restriction enzyme sites appended to their 5′ ends. Thus, all nucleotides of the primers are derived from the gene sequence of interest or sequences adjacent to that gene except the few nucleotides necessary to form a restriction enzyme site. Such enzymes and sites are well known in the art.

A sample to be typed for the presence and/or identification of a S. pneumoniae serotype may be from a bacterial culture or a clinical sample from a patient, typically a human patient. Clinical samples may be cultured to produce a bacterial culture. However, it is also possible to test clinical samples directly with a culturing step.

The methods of the present invention can be used in a multi-step serotyping strategy. An example of such a multi-step serotyping strategy (algorithm) is shown in Table 6. However, a variety of other strategies are envisaged and can be designed by the skilled person using the sequence heterogeneity information presented herein. In particular, it is preferred that the serotyping procedure comprise at least one analysis step based on analysing one or regions between the 3′ end of the cpsA gene and the 5′ end of the cpsB gene. This analysis may optionally be combined with an analysis of one or more regions within the wzy and/or wzx genes.

Microarrays

Analysis of S. pneumoniae genomic sequences using the above techniques may take place in solution followed by standard resolution using methods such as gel electrophoresis. However in a preferred aspect of the invention, the primers/probes are immobilised onto a solid substrate to form arrays.

The polynucleotide probes are typically immobilised onto or in discrete regions of a solid substrate. The substrate may be porous to allow inumobilisation within the substrate or substantially non-porous, in which case the probes are typically immobilised on the surface of the substrate. Examples of suitable solid substrates include flat glass (such as borosilicate glass), silicon wafers, mica, ceramics and organic polymers such as plastics, including polystyrene and polymethacrylate. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal.

Preferably, the solid substrate is generally a material having a rigid or semi-rigid surface. In preferred embodiments, at least one surface of the substrate will be substantially flat, although in some embodiments it may be desirable to physically separate synthesis regions for different polymers with, for example, raised regions or etched trenches. It is also preferred that the solid substrate is suitable for the high density application of DNA sequences in discrete areas of typically from 50 to 100 μm, giving a density of 10000 to 40000 cm⁻².

The solid substrate is conveniently divided up into sections. This may be achieved by techniques such as photoetching, or by the application of hydrophobic inks, for example teflon-based inks (Cel-line, USA). Discrete positions, in which each different probes are located may have any convenient shape, e.g., circular, rectangular, elliptical, wedge-shaped, etc.

Attachment of the library sequences to the substrate may be by covalent or non-covalent means. The library sequences may be attached to the substrate via a layer of molecules to which the library sequences bind. For example, the probes may be labelled with biotin and the substrate coated with avidin and/or streptavidin. A convenient feature of using biotinylated probes is that the efficiency of coupling to the solid substrate can be determined easily. Since the polynucleotide probes may bind only poorly to some solid substrates, it is often necessary to provide a chemical interface between the solid substrate (such as in the case of glass) and the probes. Thus, the surface of the substrate may be prepared by, for example, coating with a chemical that increases or decreases the hydrophobicity or coating with a chemical that allows covalent linkage of the polynucleotide probes. Some chemical coatings may both alter the hydrophobicity and allow covalent linkage. Hydrophobicity on a solid substrate may readily be increased by silane treatment or other treatments known in the art. Examples of suitable chemical coatings include polylysine and poly(ethyleneimine). Further details of methods for the attachment of are provided in U.S. Pat. No. 6,248,521.

Techniques for producing immobilised arrays of nucleic acid molecules have been described in the art. A useful review is provided in Schena et al. (1998), which also gives references for the techniques described therein.

Microarray-manufacturing technologies fall into two main categories—synthesis and delivery. In the synthesis approaches, microarrays are prepared in a stepwise fashion by the in situ synthesis of nucleic acids from biochemical building blocks. With each round of synthesis, nucleotides are added to growing chains until the desired length is achieved. A number of prior art methods describe how to synthesise single-stranded nucleic acid molecule libraries in situ, using for example masking techniques (photolithography) to build up various permutations of sequences at the various discrete positions on the solid substrate. U.S. Pat. No. 5,837,832 describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

The delivery technologies, by contrast, use the exogenous deposition of prepared biochemical substances for chip fabrication. For example, DNA may also be printed directly onto the substrate using for example robotic devices equipped with either pins (mechanical microspotting) or piezo electric devices (ink jetting). In mechanical microspotting, a biochemical sample is loaded into a spotting pin by capillary action, and a small volume is transferred to a solid surface by physical contact between the pin and the solid substrate. After the first spotting cycle, the pin is washed and a second sample is loaded and deposited to an adjacent address. Robotic control systems and multiplexed printheads allow automated microarray fabrication. Ink jetting involves loading a biochemical sample, such as a polynucleotide into a miniature nozzle equipped with a piezoelectric fitting and an electrical current is used to expel a precise amount of liquid from the jet onto the substrate. After the first jetting step, the jet is washed and a second sample is loaded and deposited to an adjacent address. A repeated series of cycles with multiple jets enables rapid microarray production.

In one embodiment, the microarray is a high density array, comprising greater than about 50, preferably greater than about 100 or 200 different nucleic acid probes. Such high density probes comprise a probe density of greater than about 50, preferably greater than about 500, more preferably greater than about 1,000, most preferably greater than about 2,000 different nucleic acid probes per cm². The array may further comprise mismatch control probes and/or reference probes (such as positive controls).

Microarrays of the invention will typically comprise a plurality of primers/probes as described above. The primers/probes may be grouped on the array in any order.

Elements in an array may contain only one type of probe/primer or a number of different probes/primers.

Detection of binding of S. pneumoniae DNA to immobilised probes/primers may be performed using a number of techniques. For example, the immobilised probes which are specific for one or a number of serotypes, may function as capture probes. Following binding of the genomic DNA to the array, the array is washed and incubated with one or more labelled detection probes which hybridise specifically to regions of the S. pneumoniae genome which are conserved (for example the S. pneumoniae psaA or pneumolysin probes/primers described herein could be utilized for this purpose). The binding of these detection probes may then be determined by detecting the presence of the label. For example, the label may be a fluorescent label and the array may be placed in an X-Y reader under a charge-coupled device (CCD) camera.

Other techniques include labelling the genomic DNA prior to contact with the array (using nick-translation and labelled dNTPs for example). Binding of the genomic DNA can then be detected directly.

It is also possible to employ a single PCR amplification step using labelled dNTPs. In this embodiment, the genomic DNA fragment binds to a first primer present in the array. The addition of polymerase, dNTPs, including some labelled dNTPs and a second primer results in synthesis of a PCR product incorporating labelled nucleotides. The labelled PCR fragment captured on the plate may then be detected.

A number of available detection techniques do not require labels but instead rely on changes in mass upon ligand binding (e.g. surface plasmon resonance—SPR). The principles of SPR and the types of solid substrates required for use in SPR (e.g. BIACore chips) are described in Ausubel et al., Short Protocols in Molecular Biology (1999) 4^{th} Ed, John Wiley & Sons, Inc.

Examples of the utilization of microarrays in genotyping include the use of microarrays to differentiate between closely related Cryptosporidium parvum isolates and Cryptosporidium species (Straub et al., 2002), the use of microarrays to differentiate between species of Listeria (Volokhov et al., 2002), and the use of microarrays to differentiate within species of Staphylococcus aureus (van Leeuwen et al., 2003). The detection principles applied in these studies can be used with the polymorphisms/primers/probes identified by the present inventors to identify different serotypes of S. pneumoniae in a sample.

In the present instance, according to 800 bp cpsA-cpsB alignment results (FIG. 2) regions, such as the first 20 nucleotides provided in FIG. 2, are scanned to see whether they contains polymorphisms. Where polymorphisms occur, probes can be designed for each “type” (allele)-specific probes (and name them as 1-1, 1-2, etc.), which will cover all the cpsA-cpsB regions for all the known sequence types. The combination of all the above allele-specific probes (about or less than 20 allele×40˜50=800˜1000 probes all together) hybridisation results will define the microarray hybridisation types like MLST (1-0-10 . . . etc), which would be nearly equal to the sequencing results. Bioinformatics software will tell which sequence type the “specimen/strain” is.

Kits

In one embodiment, kits of the present invention include, in an amount sufficient for at least one assay, a polynucleotide probe of the invention which preferentially hybridizes to a target nucleic acid sequence in a test sample under hybridization assay conditions. Kits containing multiple probes are also contemplated by the present invention where the multiple probes are designed to target different nucleic acid sequences from different S. pneumoniae serotypes and may include distinct labels which permit the probes to be differentially detected in a test sample. Kits according to the present invention may further comprise at least one of the following: (i) one or more amplification primers for amplifying a target sequence contained in or derived from the target nucleic acid; (ii) a capture probe for isolating and purifying target nucleic acid present in a test sample; and (iii) if a capture probe is included, a solid support material (e.g., magnetically responsive particles) for immobilizing the capture probe, either directly or indirectly, in a test sample. Kits of the present invention may further include one or more helper probes.

Typically, the kits will also include instructions recorded in a tangible form (e.g., contained on paper or an electronic medium) for using the packaged polynucleotide in a detection assay for determining the presence or amount of a target nucleic acid sequence in a test sample. The assay described in the written instructions may include steps for isolating and purifying the target nucleic acid prior to detection with the polynucleotide probe, and/or amplifying a target sequence contained in the target nucleic acid. The instructions will typically indicate the reagents and/or concentrations of reagents and at least one assay method parameter which might be, for example, the relative amounts of reagents to use per amount of sample. In addition, such specifics as maintenance, time periods, temperature and buffer conditions may also be included.

Uses

As discussed above, S. pneumoniae is a leading cause of morbidity and mortality causing invasive disease such as meningitis and pneumonia as well as more localised disease such as acute otitis media and sinusitis. Continued surveillance is critical to monitor vaccine efficacy and changes in incidence and distribution of colonising and invasive serotypes. Any increase in disease caused by previously uncommon nonvaccine serotypes could necessitate a change in vaccine composition. Thus, the detection methods, probes/primer and microarrays of the invention may be used to monitor the epidemiology of invasive S. pneumoniae infections to assist in disease control and to inform vaccine policy.

The molecular typing methods of the invention may also assist in comprehensive serotype identification that will be useful for epidemiological and other related studies that will be needed to monitor S. pneumoniae before and after introduction of S. pneumoniae vaccines.

### EXAMPLES

### Example 1

Serotying Based on the Polymorphisms of the 3′ End of the cpsA Gene and the 5′ End of the cpsB Gene, Combined in Some Instances with the Analysis of the wzx and/or wzy Genes

Materials and Methods

Pneumococcal Reference Panels (Table 1)

Reference panels 1-4, which consisted of 118 isolates, were kindly provided and serotyped by colleagues in Australia and Canada. All had been serotyped using the standard Quellung method and included all 23 serotypes represented in the polysaccharide vaccine, and 28 additional serotypes; there were multiple isolates of 40 serotypes and five isolates that could not be serotyped with available antisera. Reference panel 5 consisted of 21 invasive isolates from our diagnostic laboratory at the Centre for Infectious Diseases and Microbiology (CIDM), Sydney, for which serotypes were known at the beginning of the study. These five reference panels were used for the development and preliminary evaluation of molecular capsular sequence methods. Panels 2 and 4 were tested by molecular capsular sequence, initially, without knowledge of the conventional serotyping (CS) results.

Clinical Isolates

179 consecutive S. pneumoniae clinical isolates from normally sterile sites, collected during the period January 1999 to June 2001, by the CIDM diagnostic laboratory, were studied; 21 were randomly selected to make up reference panel 5 (see above). Dr Diana Martin, Institute of Environmental Science and Research (ESR), Wellington, New Zealand provided 103 clinical isolates from diagnostic laboratories throughout New Zealand. Clinical isolates were initially tested using the MCT method, without knowledge of their CS results (single-blind study). Isolates were retrieved from storage by subculture on blood agar plates (Columbia II agar base supplemented with 5% horse blood) and incubated overnight at 37° C. CO₂ incubator.

TABLE 1
Conventional serotyping (CS) and molecular capsular typing (MCT)
results of S. pneumoniae strains used in this study.

| Strain numbers and | | | | GenBank² |
|---|---|---|---|---|
| geographic origin | CS¹ | MCT-Seq² | MCT-PCR² | accession numbers |
| Reference panel 1³ | | | | |
| Queensland | | | | |
| 00S001 | 19F | 19F | 19F | AF532666 |
| 00S002 | 6B | 6B-q | 6B | AF532705; |
| | | | | AY163180, AY163190 |
| 00S006 | 19A | 19A | 19A | AF532663 |
| 00S009 | 23F | 23F-g | 23F | AF532677; |
| | | | | AY163214, AY163232 |
| 00S014 | 1 | 1 | 1 | AF532632 |
| 00S016 | 9V | 9V | 9V | AF532710 |
| 00S023 | 5 | 5-q | | AF532697 |
| 00S033 | 17F | 17F-35B | | AF532657 |
| 00S036 | 11A | 11A-q | | AF532637 |
| 00S042 | 18C | 18C/18B | 18C | AF532661 |
| 00S059 | 9N | 9N | | AF532709 |
| 00S063 | 12F | 12F | | AF532640 |
| 00S067 | 8 | 8 | 8 | AF532708 |
| 00S124 | 7F | 7F | | AF532707 |
| 00S154 | 15B | 15B-q | | AF532649 |
| 00S159 | 4 | 4 | 4 | |
| 00S168 | 33F | 33F-q | 33F/37 | AF532687; |
| | | | | AY163199, AY163221 |
| 00S246 | 22F | 22F | | AF532673 |
| 00S259 | 2 | 2-q | 2 | AF532669 |
| 00S300 | 22A | 22A | | AF532672 |
| 01S009 | 18C | 18C/18B | 18C | |
| 01S020 | 7C | 7C | | AF532706 |
| 01S043 | 10A | 10A-q | | AF532633 |
| 01S143 | 3 | 3 | 3 | AF532682 |
| 01S146 | 10F | 10F | | AF532635 |
| 01S305 | 20 | 20/13 | | AF532670 |
| 01S319 | 18A | 18A | 18C | AF532658; |
| | | | | AY163208, AY163224 |
| 01S333 | 33B | 33B | 33F-X; | AF532686 |
| | | | 33F-Y-NEG | |
| 01S358 | 35B | 35B | | AF532691 |
| 01S666 | 14 | 14-g | 14 | AF532643 |
| 01S682 | 16F | 16F | | AF532653 |
| 01S691 | 15C | 15C-q | | AF532651 |
| 01S753 | 4 | 4 | 4 | AF532693 |
| Reference panel 2⁴ | | | | |
| Victoria | | | | |
| 0013856 | 35B | 35B | | |
| 0013976 | 6A | 6A-ca | 6B | |
| 0017666 | 9V | 9V | 9V | |
| 0019532 | 23F | 23F-g | 23F | |
| 0102206 | 8 | 8 | 8 | |
| 0103678 | 19F | 19F | 19F | |
| 0104603 | 6B | 6B-q | 6B | |
| 0104604 | 22F | 22F | | |
| 0104912 | 4 | 4 | 4 | |
| 0105015 | 14 | 14-g | 14 | AF532644 |
| Reference panel 3⁵ | | | | |
| Canada | | | | |
| MA007753 | 31 | 31 | | AF532684 |
| MA007765 | 5 | 5-q | | |
| MA008229 | 10F | 10F | | AF532636 |
| MA008562 | 11A | 11A-q | | |
| MA008622 | 31 | 31 | | |
| MA050408 | 23A | 23A-23F | 23F-X; | AF532674 |
| | | | 23F-Y-NEG | |
| MA050663 | 18F | 18F | 18C | AF532662; |
| | | | | AY163207, AY163230 |
| MA050910 | 2 | 2-q | 2 | |
| MA050947 | 38 | 38/25F | | AF532712 |
| MA051117 | 22A | 22A | | |
| MA051617 | 35F | 35F | | AF532692 |
| MA051950 | 31 (see Example | 31 | | AF532695 |
| | 2) | | | |
| MA052002 | 15A | 15A-cal | | AF532646 |
| MA052150 | 11B | 11B | | AF532639 |
| MA052217 | 7C | 7C | | |
| MA052253 | 17F | 17F-35B | | |
| MA052433 | 23A | 23A-ca | 23F-X; | AF532675 |
| | | | 23F-Y-NEG | |
| MA052434 | 15A | 15A-ca2 | | AF532647 |
| MA052628 | 18C | 18C/18B | 18C | —; AY163215, AY163231 |
| MA052979 | 15C | 15C-ca | | AF532652 |
| MA053096 | 20 | 20/13 | | |
| MA053188 | 15B | 15B-q | | |
| MA053392 | 18B | 18B/18C | 18C | AF532660; |
| | | | | AY163211, AY163227 |
| MA053567 | 12F | 12F | | |
| MA053684 | 38 | 38/25F | | |
| MA053782 | 13 | 13/20 | | AF532642 |
| MA053909 | 35B | 35B | | |
| MA054004 | 13 | 13/20 | | |
| MA054006 | 13 | 13/20 | | |
| MA054242 | 38 | 38/25F | | |
| MA054294 | 16F | 16F | | |
| MA054338 | 35F | 35F | | |
| MA054357 | 1 | 1 | 1 | |
| MA054490 | 34 | 34 | | AF532690 |
| MA054545 | 3 | 3 | 3 | |
| MA054735 | 10A | 10A-q | | |
| MA054832 | 34 | 34 | | |
| MA054883 | 7F | 7F | | |
| MA055006 | 9V | 9V | 9V | |
| MA055054 | 22F | 22F | | |
| MA055100 | 6A | 6A-ca | 6B | AF532702; |
| | | | | AY163174, AY163184 |
| MA056382 | 19A | 19A | 19A | AF532664 |
| MA059287 | 25F | 25F/38 | | AF532711 |
| MA061296 | 41A (see Example | 41A | | AF532694 |
| | 2) | | | |
| MA061378 | 17A | 17A | | AF532655 |
| MA061938 | 21 | 21 | | AF532671 |
| MA062028 | 29 | 29 | | AF532680 |
| MA062610 | 18B | 18B/18C | 18C | —; |
| | | | | AY163210, AY163226 |
| MA063013 | 9N | 9N | | |
| MA063073 | 33F | 33F-g/33A | 33F/37 | AF532689; |
| | | | | AY163201, AY163220 |
| MA063087 | 33A | 33A/33F-g | 33F/37 | AF532685; |
| | | | | AY163204, AY163222 |
| MA063189 | Nonserotypeable | No-amplicon | | |
| MA063207 | 37 | 37 | 33F/37 | AF532713; |
| | | | | AY163205, AY163223 |
| MA063745 | Nonserotypeable | Nonserotypeable-ca | | AF532715 |
| Reference panel 4⁶ | | | | |
| New South Wales | | | | |
| 00-177-0145 | 19A | 19A | 19A | |
| 01-184-0091 | 18C | 18C/18B | 18C | |
| 00-237-0230 | 17F | 17F-35B | | AF532656 |
| 01-273-0175 | 16F | 16F | | |
| 00-201-0306 | 14 | 14-g | 14 | |
| 01-117-0176 | 13 | 13/20 | | |
| 01-239-0283 | 12F | 12F | | |
| 00-206-0233 | 11A | 11A-q | | |
| 00-222-0342 | 10A | 10A-23F | 23F-NEG | AF532634 |
| 01-180-0149 | 1 | 1 | 1 | |
| 01-122-0226 | 6A | 6A-ca | 6B | AF532698; |
| | | | | AY163172, AY163182 |
| 99-308-0385 | 4 | 4 | | |
| 00-234-0199 | 38 | 38/25F | | |
| 00-074-0065 | 35F | 35F | | |
| 00-280-0121 | 3 | 3 | 3 | |
| 99-308-0290 | 23F | 23F-g | 23F | |
| 00-244-0101 | 22F | 22F | | |
| 00-250-0302 | 22A | 22A | | |
| 00-244-0108 | 20 | 20/13 | | |
| 01-009-0101 | 19F | 19F | 19F | AF532668 |
| 01-254-0150 | 7F | 7F | | |
| Reference panel 5⁷ | | | | |
| New South Wales, | | | | |
| (CIDM) | | | | |
| 00-163-0650 | 14 | 14-g | 14 | |
| 00-141-1399 | 19F | 19F | 19F | |
| 00-070-0212 | 23F | 23F-g | 23F | |
| 01-018-1842 | 4 | 4 | 4 | |
| 00-201-1422 | 6B | 6B-g | 6B | AF532703; |
| | | | | AY163178, AY163188 |
| 00-180-2749 | 9V | 9V | 9V | |
| 00-339-3084 | 9N | 9N | | |
| 00-017-0985 | 11A | 11A-q | | |
| 01-072-0391 | 12F | 12F | | AF532641 |
| 00-315-3100 | 15B | 15B-c | | AF532648 |
| 99-259-1456 | 18C | 18C/18B | 18C | |
| 00-273-2862 | 4 | 4 | 4 | |
| 00-081-2291 | 33F | 33F-g/33A | 33F/37 | —; |
| | | | | AY163198, AY163216 |
| 00-118-2067 | 5 | 5-c | | AF532696 |
| 01-175-0822 | 7F | 7F | | |
| 00-324-0978 | 8 | 8 | 8 | |
| 00-152-1664 | 22F | 22F | | |
| 00-211-1414 | 22F | 22F | | |
| 00-200-0078 | 14 | 14-g | 14 | |
| 00-118-0159 | 19F | 19F | 19F | |
| 00-310-1104 | 4 | 4 | 4 | |
| Clinical isolates | | | | |
| New South Wales, | | | | |
| (CIDM)⁸ | | | | |
| 01-192-3558 | 6B | 6B-g | 6B | |
| 01-192-2471 | 6A | 6A-c | 6B | AF532699; |
| | | | | AY163173, AY163183 |
| 01-192-1205 | 6B | 6B-g | 6B | |
| 01-191-1265 | 14 | 14-g | 14 | |
| 01-189-0296 | 19F | 19F | 19F | |
| 01-185-0511 | 15B | 15B-22F | | AF532650 |
| 01-184-0328 | 8 | 8 | 8 | |
| 01-179-2448 | 14 | 14-g | 14 | |
| 01-178-0165 | 14 | 14-g | 14 | |
| 01-176-3302 | 1 | 1 | 1 | |
| 01-173-2782 | 4 | 4 | 4 | |
| 01-170-0873 | 9V | 9V | 9V | |
| 01-159-0505 | 14 | 14-g | 14 | |
| 01-157-3399 | 4 | 4 | 4 | |
| 01-157-3394 | 4 | 4 | 4 | |
| 01-157-2062 | 4 | 4 | 4 | |
| 01-152-3295 | 14 | 14-g | 14 | |
| 01-150-3706 | 14 | 14-g | 14 | |
| 01-144-1862 | 7F | 7F | | |
| 01-143-3353 | 4 | 4 | 4 | |
| 01-124-2300 | 12F | 12F | | |
| 01-117-1910 | 4 | 4 | 4 | |
| 01-096-2050a | 9V | 9V | 9V | |
| 01-096-2050b | 9V | 9V | 9V | |
| 01-096-2027 | 9V | 9V | 9V | |
| 01-077-1533 | 7F | 7F | | |
| 01-075-3257 | 9N | 9N | | |
| 01-058-3662 | 14 | 14-g | 14 | |
| 01-048-1320 | 19A | 19A | 19A | |
| 01-005-0764 | 19F | 19F | 19F | AF532650 |
| 00-361-1217 | 6B | 6B-q | 6B | |
| 00-357-1164 | 14 | 14-g | 14 | |
| 00-339-2918 | 9N | 9N | | |
| 00-324-0977 | 8 | 8 | 8 | |
| 00-315-2993 | 23F | 23F-g = 10A-23F | 23F | |
| 00-315-2254 | 23F | 23F-g = 10A-23F | 23F | |
| 00-310-0630 | 14 | 14-g | 14 | |
| 00-303-0303 | 19F | 19F | 19F | |
| 00-293-1660 | 19F | 19F | 19F | |
| 00-280-1493 | 33F | 33F-q | 33F/37 | —; |
| | | | | AY163200, AY163217 |
| 00-267-0653 | 8 | 8 | 8 | |
| 00-258-1120 | 14 | 14-g | 14 | |
| 00-257-0881 | 9V | 9V | 9V | |
| 00-256-1986 | 6A | 6A-ca | 6B | —; |
| | | | | AY163176, AY163186 |
| 00-251-3185 | 6A | 6A-6B-g = 6B-g | 6B | AF532700; |
| | | | | AY163171, AY163181 |
| 00-245-3950 | 23F | 23F-g = 10A-23F | 23F | |
| 00-243-2229 | 3 | 3 | 3 | |
| 00-242-0394 | 14 | 14-g | 14 | |
| 00-241-2964 | 9V | 9V | 9V | |
| 00-238-3448 | 23F | 23F-g = 10A-23F | 23F | |
| 00-235-3584 | 19F | 19F | 19F | AF532665 |
| 00-228-3777 | 35B | 35B | | |
| 00-225-1482 | 3 | 3 | 3 | |
| 00-225-0333 | 19F | 19F | 19F | |
| 00-217-3003 | 4 | 4 | 4 | |
| 00-211-1669 | 6B | 6B-c | 6B | AF532704; |
| | | | | AY163179, AY163189 |
| 00-211-0475 | 22F | 22F | | |
| 00-211-0469 | 22F | 22F | | |
| 00-209-3409 | 3 | 3 | 3 | |
| 00-208-0179 | 4 | 4 | 4 | |
| 00-200-1013 | 14 | 14-g | 14 | |
| 00-200-1012 | 14 | 14-g | 14 | |
| 00-199-0498 | 4 | 4 | 4 | |
| 00-196-2923 | 9V | 9V | 9V | |
| 00-192-2087 | 19A | 19A | 19A | |
| 00-184-1203 | 6B | 6B-q | 6B | |
| 00-181-1568 | 23F | 23F-g = 10A-23F | 23F | |
| 00-181-1567 | 23F | 23F-g = 10A-23F | 23F | |
| 00-173-3686 | 4 | 4 | 4 | |
| 00-164-1705 | 6B | 6B-q | 6B | |
| 00-163-1533 | 14 | 14-g | 14 | |
| 00-149-1265 | 7F | 7F | | |
| 00-149-1264 | 7F | 7F | | |
| 00-143-1473 | 15B | 15B-22F | | |
| 00-138-3435 | 3 | 3 | 3 | |
| 00-118-2891 | 19F | 19F | 19F | |
| 00-093-1315 | 3 | 3 | 3 | AF532681 |
| 00-078-0883 | 14 | 14-g | 14 | |
| 00-074-3370 | 14 | 14-g | 14 | |
| 00-070-0212 | 23F | 23F-g = 10A-23F | 23F | |
| 00-066-3506 | 4 | 4 | 4 | |
| 00-043-0876 | 19A | 19A | 9A | |
| 00-036-1378 | 19F | 19F | 19F | |
| 00-008-0865 | 8 | 8 | 8 | |
| 99-348-3354 | 6A | 6A-ca | 6B | |
| 99-338-1052 | 19F | 19F | 19F | |
| 99-325-0373 | 23F | 23F-c | 23F | AF532678 |
| 99-324-1010 | 4 | 4 | 4 | |
| 99-404-0191 | 4 | 4 | 4 | |
| 99-310-0070 | 4 | 4 | 4 | |
| 99-302-1894 | 9V | 9V | 9V | |
| 99-293-1704 | 19A | 19A | 19A | |
| 99-287-2376 | 35B | 35B | | |
| 99-287-2320 | 35B | 35B | | |
| 99-287-2298 | 35B | 35B | | |
| 99-284-1034 | 14 | 14-c | 14 | AF532645 |
| 99-276-0568 | 9V | 9V | 9V | |
| 99-242-0442A | 6B | 6B-q | 6B | |
| 99-241-1187A | 4 | 4 | 4 | |
| 99-237-2839 | 9V | 9V | 9V | |
| 99-235-2193 | 4 | 4 | 4 | |
| 99-226-1026B | 7F | 7F | | |
| 99-221-2755 | 9V | 9V | 9V | |
| 99-221-2745A1 | 23F | 23F-g = 10A-23F | 23F | |
| 99-221-0278 | 4 | 4 | 4 | |
| 99-218-2527 | 23F | 23F-g = 10A-23F | 23F | |
| 99-201-1708 | 3 | 3 | 3 | |
| 99-196-2909B | 10A | 10A-23F = 23F-g | 23F-NEG | |
| 99-196-2908B | 10A | 10A-23F = 23F-g | 23F-NEG | |
| 99-196-2882A | 10A | 10A-23F = 23F-g | 23F-NEG | |
| 99-196-2880A | 10A | 10A-23F = 23F-g | 23F-NEG | |
| 99-195-0430 | 14 | 14-g | 14 | |
| 99-193-2919A | 4 | 4 | 4 | |
| 99-193-2918B | 4 | 4 | 4 | |
| 99-193-2747B | 4 | 4 | 4 | |
| 99-193-2491A | 18C | 18C/18B | 18C | |
| 99-192-0047B | 23F | 23F-g = 10A-23F | 23F | |
| 99-188-2369A | 4 | 4 | 4 | |
| 99-186-2831 | 7F | 7F | | |
| 99-186-1038 | 14 | 14-g | 14 | |
| 99-186-0417 | 14 | 14-g | 14 | |
| 99-184-0894 | 14 | 14-g | 14 | |
| 99-182-1919 | 4 | 4 | 4 | |
| 99-180-2653 | 4 | 4 | 4 | |
| 99-178-0901 | 14 | 14-g | 14 | |
| 99-177-1060 | 11A | 11A-q | | |
| 99-176-1983 | 18C | 18C/18B | 18C | |
| 99-173-2956 | 4 | 4 | 4 | |
| 99-169-0432 | 6B | 6B-g | 6B | |
| 99-159-2018 | 7F | 7F | | |
| 99-158-1250 | 14 | 14-g | 14 | |
| 99-157-0650 | 19F | 19F | 19F | |
| 99-146-2324 | 19F | 19F | 19F | |
| 99-144-1497 | 22F | 22F | | |
| 99-134-2273 | 3 | 3 | 3 | |
| 99-132-2724 | 15B | 15B-q | | |
| 99-132-2558 | 15B | 15B-q | | |
| 99-132-2557 | 15B | 15B-q | | |
| 99-130-2037 | 14 | 14-g | 14 | |
| 99-110-2820 | 9N | 9N | | |
| 99-108-0976 | 23F | 23F-g = 10A-23F | 23F | |
| 99-107-0715 | 14 | 14-g | 14 | |
| 99-104-1860 | 4 | 4 | 4 | |
| 99-099-0423 | 19F | 19F | 19F | |
| 99-095-1044 | 20 | 20/13 | | |
| 99-091-2295 | 23B | 23B | 23F-NEG | AF532676 |
| 99-090-2551 | 14 | 14-g | 14 | |
| 99-090-2390 | 3 | 3 | 3 | |
| 99-090-2387 | 3 | 3 | 3 | |
| 99-033-2630 | 23F | 23F-g = 10A-23F | 23F | |
| 99-028-0057 | 7C | 7C | | |
| 99-011-0311A | 4 | 4 | 4 | |
| Clinical isolates | | | | |
| New Zealand | | | | |
| (ESR)⁹ | | | | |
| NZSPN00/9 | 4 | 4 | 4 | |
| NZSPN00/42 | 18C | 18C/18B | 18C | |
| NZSPN00/59 | 5 | 5-q | | |
| NZSPN00/87 | 13 | 13/20 | | |
| NZSPN00/88 | 6B | 6B-g | 6B | |
| NZSPN00/91 | 8 | 8 | 8 | |
| NZSPN00/319 | 18B | 18B/18C | 18C | —; |
| | | | | AY163212, AY163228 |
| NZSPN00/366 | 7F | 7F | | |
| NZSPN00/426 | 3 | 3 | 3 | |
| NZSPN00/454 | 23F | 23F-23A = 23A-23F | 23F | AF532679 |
| NZSPN00/470 | 9V | 9V | 9V | |
| NZSPN00/480 | 6A | 6A-ca | 6B | |
| NZSPN00/484 | 23F | 23F-g = 10A-23F | 23F | |
| NZSPN00/499 | 19F | 19F | 19F | |
| NZSPN01/162 | 2 | 2-q | 2 | |
| NZSPN01/243 | 33F | 33F-q | 33F/37 | —; |
| | | | | AY163203, AY163219 |
| NZSPN01/393 | 35F | 35F | | |
| NZSPN01/468 | 11A | 11A-q | | |
| NZSPN01/481 | 16F | 16F | | |
| NZSPN01/484 | 23F | 23F-g = 10A-23F | 23F | |
| NZSPN01/490 | 22F | 22F | | |
| NZSPN01/493 | 9N | 9N | | |
| NZSPN01/509 | 23A | 23A-ca | 23F-X; | |
| | | | 23F-Y-NEG | |
| NZSPN01/510 | 12F | 12F | | |
| NZSPN01/520 | 9V | 9V | 9V | |
| NZSPN01/531 | 8 | 8 | 8 | |
| NZSPN01/534 | 3 | 3 | 3 | |
| NZSPN01/538 | 38 | 38/25F | | |
| NZSPN01/543 | 10A | 10A-q | | |
| NZSPN01/546 | 4 | 4 | 4 | |
| NZSPN01/547 | 20 | 20/13 | | |
| NZSPN01/548 | 7F | 7F | | |
| NZSPN01/549 | 1 | 1 | 1 | |
| NZSPN01/553 | 17F | 17F-c | | |
| NZSPN01/554 | 19F | 19F | 19F | |
| NZSPN01/555 | 18C | 18C/18B | 18C | |
| NZSPN01/557 | 19A | 19A | 19A | |
| NZSPN01/559 | 6A | 6A-c | 6B | |
| NZSPN01/560 | 14 | 14-g | 14 | |
| NZSPN01/561 | 6B | 6B-q | 6B | |
| NZSPN00/12 | 17F | 17F-c | | |
| NZSPN00/50 | Nonserotypeable | Nonserotypeable-nz | | AF532714 |
| NZSPN00/59 | 5 | 5-q | | |
| NZSPN00/75 | Nonserotypeable | No-amplicon | | |
| NZSPN00/180 | 9V + 14 | 9V | 9V + 14 | |
| NZSPN00/221 | 38 | 38/25F | | |
| NZSPN00/225 | 13 | 13/20 | | |
| NZSPN00/242 | 35F | 35F | | |
| NZSPN00/353 | 18A | 18A | 18C | AF532659; |
| | | | | AY163209, AY163225 |
| NZSPN00/410 | 33F | 33F-q | 33F/37 | AF532688; |
| | | | | AY163202, AY163218 |
| NZSPN01/93 | 16F | 16F | | |
| NZSPN01/122 | 10A | 10A-q | | |
| NZSPN01/146 | 38 | 38/25F | | |
| NZSPN01/166 | 16F | 16F | | AF532654 |
| NZSPN01/204 | 35B | 35B | | |
| NZSPN01/209 | 22A | 22A | | |
| NZSPN01/240 | 12F | 12F | | |
| NZSPN01/254 | 35F | 35F | | |
| NZSPN01/262 | 8 | 8 | 8 | |
| NZSPN01/276 | 6A | 6A-6B-q = 6B-q | 6B | —; |
| | | | | AY163177, AY163187 |
| NZSPN01/278 | 18B | 18B/18C | 18C | —; |
| | | | | AY163213, AY163229 |
| NZSPN01/291 | 6B | 6B-q | 6B | |
| NZSPN01/303 | Nonserotypeable | No-amplicon | | |
| NZSPN01/313 | 18C | 18C/18B | 18C | |
| NZSPN01/329 | 6A | 6A-6B-g = 6B-g | 6B | AF532701; |
| | | | | AY163175, AY163185 |
| NZSPN01/335 | 19A | 19A | 19A | |
| NZSPN01/344 | 18C | 18C/18B | 18C | |
| NZSPN01/361 | 9N | 9N | | |
| NZSPN01/363 | 18C | 18C/18B | 18C | |
| NZSPN01/366 | 6A | 6A-ca | 6B | |
| NZSPN01/369 | 18C | 18C/18B | 18C | |
| NZSPN01/374 | 35B | 35B | | |
| NZSPN01/387 | 22F | 22F | | |
| NZSPN01/388 | 12F | 12F | | |
| NZSPN01/389 | 20 | 20/13 | | |
| NZSPN01/403 | 20 | 20/13 | | |
| NZSPN01/411 | 11A | 11A-nz | | AF532638 |
| NZSPN01/418 | 8 | 8 | 8 | |
| NZSPN01/428 | 3 | 3 | 3 | AF532683 |
| NZSPN01/431 | 1 | 1 | 1 | |
| NZSPN01/437 | 1 | 1 | 1 | |
| NZSPN01/438 | 22F | 22F | | |
| NZSPN01/448 | 11A | 11A-q | | |
| NZSPN01/455 | 19A | 19A | 19A | |
| NZSPN01/463 | 10A | 10A-q | | |
| NZSPN01/465 | 22F | 22F | | |
| NZSPN01/477 | 10A | 10A-23F = 23F-g | 23F-NEG | |
| NZSPN01/478 | 20 | 20/13 | | |
| NZSPN01/483 | 8 | 8 | 8 | |
| NZSPN01/485 | 12F | 12F | | |
| NZSPN01/489 | 3 | 3 | 3 | |
| NZSPN01/497 | 9N | 9N | | |
| NZSPN01/505 | 19A | 19A | 19A | |
| NZSPN01/512 | 7F | 7F | | |
| NZSPN01/515 | 3 | 3 | 3 | |
| NZSPN01/516 | 1 | 1 | 1 | |
| NZSPN01/529 | 1 | 1 | 1 | |
| NZSPN01/532 | 4 | 4 | 4 | |
| NZSPN01/535 | 7F | 7F | | |
| NZSPN01/539 | 19F | 19F | 19F | |
| NZSPN01/545 | 18C | 18C/18B | 18C | |
| NZSPN01/556 | 6B | 6B-q | 6B | |
| NZSPN01/558 | 14 | 14-g | 14 | |

Notes.
¹CS of selected S. pneumoniae isolates from reference panels 1 and 3 was repeated by Gail Stewart and Robert Gange at Department of Microbiology, Children's Hospital at Westmead, New South Wales, Australia.
²MCT was performed and GenBank accession numbers generated by Fanrong Kong at Centre for Infectious Diseases and Microbiology (CIDM), Institute of Clinical Pathology and Medical Research (ICPMR), Westmead Hospital, Westmead, New South Wales, Australia. See text for molecular capsular subtype (mctsp) nomenclature.
³Provided by Denise Murphy, Pneumococcal Reference Laboratory, Public Health Microbiology, Queensland Health Scientific Services, Queensland, Australia.
⁴Provided by Associate Professor Geoff Hogg and Jenny Davis, Microbiological Diagnostic Unit (MDU), Public Health Laboratory, Department of Microbiology and Immunology, University of Melbourne, Victoria, Australia.
⁵Provided by Dr. Louise P. Jette, Institut National de Sante Publique du Quebec-Laboratoire de Sante Publique du Quebec, Sainte-Anne-de-Bellevue, Quebec H9X 3R5, Canada.
⁶Provided by Dr. Michael Watson, Department of Microbiology, Children's Hospital at Westmead, New South Wales, Australia.
⁷Selected 21 S. pneumoniae clinical isolates, of which CS results were known, from the CIDM diagnostic laboratory.
⁸152 Australian S. pneumoniae clinical isolates, of which CS results were known, from the CIDM diagnostic laboratory.
⁹103 New Zealand S. pneumoniae clinical isolates Provided by Dr. Diana Martin, from Streptococcus Reference Laboratory, at Institute of Environmental Science and Research (ESR), Wellington, New Zealand.

Conventional Serotyping (CS)

CS was performed by the Quellung reaction using rabbit polyclonal antisera from the Statens Serum Institute, Copenhagen, Denmark (Sorensen, 1993). Briefly, 2 μL of a suspension of isolate, in 10% formalin saline, and 1 μL of antisera, under a glass coverslip were examined for capsular swelling using a light microscope at 400× magnification. Clinical isolates from CIDM were serotyped at Department of Microbiology, Children's Hospital at Westmead, Sydney, Australia and those from New Zealand by the Streptococcus Reference Laboratory, at ESR, Wellington, New Zealand. Selected New Zealand clinical isolates for which only serogroup results were available and selected isolates from reference panels 1 and 3 were re-tested at Children's Hospital at Westmead.

Molecular Capsular Sequence Typing—Development of Method

Oligonucleotide Primers

The oligonucleotide primers used in this study, their target sites and melting temperatures are shown in Table 2 and the primer pair specificities and expected amplicon lengths in Table 3. Primers were designed with high melting temperatures to be used in rapid cycle PCR (Kong et al., 2000).

Four previously published S. pneumoniae-specific primers, targeting psaA (P1, P2) (Morrison et al., 2000) and pneumolysin (IIa, IIb) (Salo et al., 1995) were modified to give high melting temperatures and used to confirm that isolates were S. pneumoniae. Primers were designed to amplify and sequence portion of the cpsA-cpsB gene region and to amplify serotype/serogroup-specific sequences in the wzy and wzx genes of 16 S. pneumoniae serotypes for which cps gene cluster sequences were available. In order to further explore the sequence heterogeneity, part of the wzx and wzy genes of isolates belonging to serogroups 6, 18, 23 and 33/37 were also sequenced. For serotype 3, which does not contain wzy and wzx genes, serotype-specific PCR targeted the orf2 (wze)-cap3A-cap3B region (Arrecubieta et al., 1996).

TABLE 2
Oligonucleotide primers used in this study.

| | | | | |
|---|---|---|---|---|
| | | | GenBank | |
| | | | accession | |
| Primer | Target gene | Tm ° C.¹ | numbers | Sequence²⁻⁴ |

| | | | | |
|---|---|---|---|---|
| *P1⁵ | psaA | 72.9 | U53509 | 203TAC ATT ACT CGT TCT CTT TCT TTC TGC AAT |
| | | | | CAT TCT TG240 |
| | | | | (SEQ ID NO:64) |
| *P2⁵ | psaA | 72.7 | U53509 | 1066TAG TAG CTG TCG CCT TCT TTA CCT TGT TCT |
| | | | | GC1035 |
| | | | | (SEQ ID NO:65) |
| *IIa⁶ | pneumolysin | 71.9 | M17717 | 457AGA ATA ATC CCA CTC TTC TTG CGG TTG |
| | | | | A484 |
| | | | | (SEQ ID NO:66) |
| *IIb⁶ | pneumolysin | 71.4 | M17717 | 680CAT GCT GTG AGC CGT TAT TTT TTC ATA |
| | | | | CTG651 |
| | | | | (SEQ ID NO:67) |
| cpsS1⁷ | cpsA (wzg) | 75.4 | U09239 | 1030GGC ATT(/C) TAT GGA GTT GAT TCG(/A) TCC |
| | | | | ATT(/C) CAC ACC(/T) TTA G1066 |
| | | | | (SEQ ID NO:68) |
| cpsS2⁷ | cpsA (wzg) | 71.9 | U09239 | 1O57CAC ACC(/T) TTA GAA AAT(/C) CTC TAT GGA |
| | | | | GTG GAT ATC AAT TAC TAT G1099 |
| | | | | (SEQ ID NO:69) |
| cpsS3⁷ | cpsA (wzg) | 68.7 | U09239 | 1447GAA AGT GGG(/A/T) GGG(/A/T) A(/G)A(/C)T(/G) |
| | | | | TAT(/C) AAA GTA(/G) AAT TCT(/G) CAA GAT(/C) |
| | | | | TTA(/G) AAA(/G) G1489 |
| | | | | (SEQ ID NO:70) |
| cpsA1⁷ | cpsA (wzg) | 71.5 | U09239 | 1549CCA TCA C(/T)AT AGA GGT TAC(/A) TG(/A)T |
| | | | | CTG GCA TT(/C)G C1519 |
| | | | | (SEQ ID NO:71) |
| cpsA2⁷ | cpsB (wzh) | 67.0 | U09239 | 1949T(/G)CA TG(/A)C TA(/G)A AC(/T)T CT(/A)A |
| | | | | TC(/T)A AG(/A)G CAT AAC GAC TAT C(/T)1916 |
| | | | | (SEQ ID NO:72) |
| cpsA3⁷ | cpsB (wzh) | 75.6 | U09239 | 2030GC(/T)T CAA TG(/A)T GG(/A)G CAA TG(/T)A |
| | | | | CTG GA(/C)G TA(/G)A TTC CCA(/G) ACA TC1993 |
| | | | | (SEQ ID NO:73) |
| 1YS | cap1H (wzy) | 72.1 | Z83335 | 10289GTA GGT GTA GTT TTT TCA GGG ACT TTA |
| | | | | ATT TTA TGC AGT G10328 |
| | | | | (SEQ ID NO:74) |
| 1YA | cap1H (wzy) | 70.4 | Z83335 | 10584 TCG CTT AAC ACA ATG GCT TTA GAA GGT |
| | | | | AGA G10554 |
| | | | | (SEQ ID NO:75) |
| 2YS | cps2H (wzy) | 70.5 | AF026471 | 9711GTT ATT TTA TTT TTT TTG TCG GCA TTG TAT |
| | | | | TCT TTA TAT CG9751 |
| | | | | (SEQ ID NO:76) |
| 2YA | cps2H (wzy) | 71.3 | AF026471 | 10058CAA ATT CAT CGT TTG TAT CCA TTT AAC |
| | | | | TGC ATC10026 |
| | | | | (SEQ ID NO:77) |
| 4YS | wzy | 70.2 | AF316639 | 9601CTT ATA TCT AAT TAT GTT CCG TCT ATA TTT |
| | | | | ATA TGG GTT TGC TTT C9646 |
| | | | | (SEQ ID NO:78) |
| 4YA | wzy | 71.1 | AF316639 | 9948TTT CTC TTC ATT TTC CTG ATA ATT TTG TAC |
| | | | | TTC TGA ATG9910 |
| | | | | (SEQ ID NO:79) |
| 6A6BYSO⁷ | wzy | 62.6 | AY078347 | 8196/9186ATG CTT TTA AAT TTC TTA TTC ATA TCT |
| | | | & AF316640 | ATT TTT C8229/9219 |
| | | | | (SEQ ID NO:80) |
| 6A6BYS | wzy | 72.0 | AY078347 | 8264/9254G(/A)GA TTT T(/G)TT TCA ACC T(/C)GC |
| | | | & AF316640 | AGT AAT TTT AAC AA(/C)T C(/T)G(/A)8298/9288 |
| | | | | (SEQ ID NO:81) |
| 6A6BYA | wzy | 71.4 | AY078347 | 8578/9568CCT GAA AAC AA(/G)T ACT(/C) ACT TTC |
| | | | & AF316640 | TGA ATT TCA C(/T)GG A(/G)TA TAA AG8538/9528 |
| | | | | (SEQ ID NO:82) |
| 6A6BYA1⁷ | wzy | 72.4 | AY078347 | 8944/9934GTA AAC AGA GAG CGA GTG ATC ATT |
| | | | & AF316640 | TTA AAA CTT TTG G8808/9898 |
| | | | | (SEQ ID NO:83) |
| 8YS | wzy | 70.5 | AF316641 | 10810GTT TTA TTG ACT TTA AAG ATG TTA GTT |
| | | | | TCT TCG ATT CCA G10849 |
| | | | | (SEQ ID NO:84) |
| 8YA | wzy | 70.5 | AF316641 | 11086TTT TTA TTA CTC TTC TTA AAT CAT AAT |
| | | | | GAA TCG TAC CAA TCA AC11043 |
| | | | | (SEQ ID NO:85) |
| 9VYS | cps9vI (wzy) | 73.5 | AF402095 | 8535GGA TCA ATG GCA ACT ATA TTT ACC CTA |
| | | | | CTC TCC ACA G8571 |
| | | | | (SEQ ID NO:86) |
| 9VYA | cps9vI (wzy) | 76.3 | AF402095 | 8872GAG TCG AAA CCA ACC GGA AAA AGC AAT |
| | | | | TGA G8842 |
| | | | | (SEQ ID NO:87) |
| 14YS | cps14H (wzy) | 71.5 | X85787 | 7361CCT TTG GTT TAT TAT CCT ACT TCC AAA |
| | | | | ACA GTT TAT GC7398 |
| | | | | (SEQ ID NO:88) |
| 14YA | cps14H (wzy) | 71.4 | X85787 | 7670CAT ATA TCT CTT TAT CCT GTC AAT ATT GAT |
| | | | | TGG CAT TTT C7631 |
| | | | | (SEQ ID NO:89) |
| 18CYSO⁷ | wzx | 71.3 | AF316642 | 11856GAA ATT ATA GTC GGA GCT TTC ATT TAT |
| | | | | ATT AGT TTA CTG GTT CTG11900 |
| | | | | (SEQ ID NO:90) |
| 18CYS | wzy | 71.5 | AF316642 | 12190GAT ATT AGC TAT ACC AAC AAT TGT TCT |
| | | | | TTT CCT GTA CTC AGT C12232 |
| | | | | (SEQ ID NO:91) |
| 18CYA | wzy | 72.5 | AF316642 | 12491GCA TTT CTA GTA CCG AAC CAT TGA AAC |
| | | | | TAT CAT CTG12456 |
| | | | | (SEQ ID NO:92) |
| 18CYA1⁷ | wzy | 73.3 | AF316642 | 12536CAG AAT AAA GAG AGC TGT AAT AGG TGC |
| | | | | AAC TTC ATG C12490 |
| | | | | (SEQ ID NO:93) |
| 19FYS | cps19fI (wzy) | 70.6 | U09239 | 7673CTG TAA TGT TTC TAA TTA GTT CAG TAT |
| | | | | TTG CAC TGG TTA ATT C7715 |
| | | | | (SEQ ID NO:94) |
| 19FYA | cps19fI (wzy) | 72.0 | U09239 | 7958CCC GTA TAT CCA TTA CTA AGA ACA AGG |
| | | | | TTG TAT ATT TCC TTC7917 |
| | | | | (SEQ ID NO:95) |
| 19AYS | cps19aI (wzy) | 71.2 | AF094575 | 9245GTT TCT CAT TAG TTC TGT ATT TGC CCT TAT |
| | | | | TAA TGT GC9282 |
| | | | | (SEQ ID NO:96) |
| 19AYA | cps19aI (wzy) | 72.2 | AF094575 | 9514CCA TGG CTA AGT GCA AGA TTA TGA ATC |
| | | | | TCT CTC9482 |
| | | | | (SEQ ID NO:97) |
| 19B19CYS | cps19bI (wzy) | 71.6 | AF004325 | 3519GTT TCT TAT GTT TAC CCT CAG CTT ATA TTG |
| | | | | GCA CAG3554 |
| | | | | (SEQ ID NO:98) |
| 19B19CYA | cps19bI (wzy) | 71.5 | AF004325 | 3946GAT ACC ACA AAT CTC CGA ATT CTC TTA |
| | | | | AAA TAG ATG G3910 |
| | | | | (SEQ ID NO:99) |
| 23FYS | cps23fG (wzy) | 71.6 | AF057294 | 8567TTA AGT AGT TCA CAA GTG ATA GTG AAC |
| | | | | TTG GGA TTG TC8604 |
| | | | | (SEQ ID NO:100) |
| 23FYA | cps23fG (wzy) | 70.7 | AF057294 | 8846CAC TGA GAT TAT TTA TTA GCT TTA TCG |
| | | | | GTA AGG TGG ATA AG8806 |
| | | | | (SEQ ID NO:101) |
| 33F37YSO⁷ | cap33fJ | 76.0 | AJ006986 | 11191CCA ATG AAA AGG AAA GTT CAA TGT GTT |
| | | | | TTG TTT CTG C11227 |
| | | | | (SEQ ID NO:102) |
| 33F37YS | cap33fK & | 70.7 | AJ006986 | 11341/11708ATT ACT TGT AAT ACT ATG TAT TCA |
| | cap37K (wzy) | | & AJ131984 | ACT AGT CA(/C)A GGA TTT GAT GG11384/11751 |
| | | | | (SEQ ID NO:103) |
| 33F37YA | cap33fK & | 71.7 | AJ006986 | 11650/12017GAACAAATTTCCGTATCAGATTTGCGA |
| | cap37K (wzy) | | & AJ131984 | TTTC11620/11987 |
| | | | | (SEQ ID NO:104) |
| 33F37YAI⁷ | cap33fK (wzy) | 72.2 | AJ006986 | 11858GGT GCT TCA GCA AAA ATC CCC GTA TTT |
| | | | | CTT ATC AG11824 |
| | | | | (SEQ ID NO:105) |
| 1XS | cap1I (wzx) | 72.6 | Z83335 | 12017TAG CTG ATG TTC CGA TAA ATT ATG GTG |
| | | | | GGG TAA TAA TAG12055 |
| | | | | (SEQ ID NO:106) |
| 1XA | cap1I (wzc) | 70.6 | Z83335 | 12442CTG CGA CAC TGT ATA TAC CTA CAT TAT |
| | | | | AAC TAC TAG ACA TTT GC12399 |
| | | | | (SEQ ID NO:107) |
| 2XS | cps2J (wzx) | 71.8 | AF026471 | 12167GCA ACT TTG GTT CTA AAA TTT TAG TCT |
| | | | | TTT TAA TGG TTC C12206 |
| | | | | (SEQ ID NO:108) |
| 2XA | cps2J (wzx) | 72.1 | AF026471 | 12595TGT TAA ACC CCA ATA TAG AAA TTG TAT |
| | | | | TGA GAA TAG CAG C12556 |
| | | | | (SEQ ID NO:109) |
| 4XS | wzx | 73.2 | AF316639 | 12119CG TTA ATA GCT TAT GTT CAA CTG GTG |
| | | | | ATT GAT TTT GG12155 |
| | | | | (SEQ ID NO:110) |
| 4XA | wzx | 72.0 | AF316639 | 12442TGA TAG TTT TAG AAA TAA TAT AAG GAA |
| | | | | TTG CAA CTG CAT GC12402 |
| | | | | (SEQ ID NO:111) |
| 6A6BXSO⁷ | cpsI-wzx | 72.7 | AY078347 & | 9581/4550GGT AGG TAT TTT AAT TGG AGG AAG |
| | spacer | | AF246898 | AGA GTC TTG AAT GG961814587 |
| | | | | (SEQ ID NO:112) |
| 6A6BXS | wzx | 72.5 | AY078347 | 9695/10685TTC ATG TC(/T)T(/C) TTT TG(/A)T CTA |
| | | | & AF316640 | ATC TGA TTA CAA TTG(/C) TC(/T)A CAT |
| | | | | CG(/A)9735/10725 |
| | | | | (SEQ ID NO:113) |
| 6A6BXA | wzx | 74.1 | AY078347 | 9999/10989T(/C)GC ATT TG(/T)G ATC TGT GAG |
| | | | & AF316640 | AA(/G)T CAATAA GTTAAAACC9964/10954 |
| | | | | (SEQ ID NO:114) |
| 6A6BXAI⁷ | wzx | 72.5 | AY078347 & | 10682/5651ATC TTC CCT TCA TAA ATT GAT ATA |
| | | | AF246898 | GGA AAA ATA AGA GCC10644/5613 |
| | | | | (SEQ ID NO:115) |
| 8XS | wzx | 71.8 | AF316641 | 8602CAA TTC TAA CTA TGT CCA GTT TTA TTT TTC |
| | | | | CAC TCA TCA G8641 |
| | | | | (SEQ ID NO:116) |
| 8XA | wzx | 74.2 | AF316641 | 8926GAC GTG ATA ATA ATA AGC TGC CAT TCC |
| | | | | TGT CTA AAA CG8889 |
| | | | | (SEQ ID NO:117) |
| 9VXS | cps9vK (wzx) | 74.5 | AF402095 | 10543CGG CGG TAT TAA GTA GAA TAT TAA CAC |
| | | | | CTG AAG AGT ATG GC10583 |
| | | | | (SEQ ID NO:118) |
| 9VXA | cps9vK (wzx) | 73.6 | AF402095 | 10910GGC AAT CAG ACT CAA TAA GTT CAT CCG |
| | | | | TTT AAA GTT C10874 |
| | | | | (SEQ ID NO:119) |
| 14XS | cps14L (wzx) | 72.1 | X85787 | 11463GGT ATT GCC TTT CCT TTG ATA ACT TCT |
| | | | | CCT TAT TTA TCA C11502 |
| | | | | (SEQ ID NO:120) |
| 14XA | cps14L (wzx) | 71.6 | X85787 | 11751TGA ACT TGT AAC TCG ACA CCC AAA AAT |
| | | | | ATA AAT AAA TGA G11712 |
| | | | | (SEQ ID NO:121) |
| 18CXSO⁷ | wciW | 75.0 | AF316642 | 10403CAA AGG AAC GTT ATC AGC AAT TGT GTC |
| | | | | AAA TTT CAG10438 |
| | | | | (SEQ ID NO:122) |
| 18CXS | wzx | 72.5 | AF316642 | 10715GAA TCG GAG AAT AGC ACA GGT ACG AAC |
| | | | | AAG10744 |
| | | | | (SEQ ID NO:123) |
| 18CXA | wzx | 75.2 | AF316642 | 11082GCC ATG TAA TCA ACT GAC CAA GCA GGG |
| | | | | TAG TC11051 |
| | | | | (SEQ ID NO:124) |
| 18CXA1⁷ | wzx | 72.2 | AF316642 | 11123AAG ATT AGG GCG CAC AAA GTT TAC TTG |
| | | | | TTT TAG C11090 |
| | | | | (SEQ ID NO:125) |
| 19FXS | cps19fJ (wzx) | 71.3 | U09239 | 8975GTT ATT TGT TCA AAT GTG CTG ATA GTT TTA |
| | | | | ACC TGA TCA C9014 |
| | | | | (SEQ ID NO:126) |
| 19FXA | cps19fJ (wzx) | 73.5 | U09239 | 9279TAT CTT GCG TTT TCA TGG CTT AGA GTT ATT |
| | | | | AGG TTC AAA G9240 |
| | | | | (SEQ ID NO:127) |
| 19AXS | cps19aJ (wzx) | 74.7 | AF094575 | 10547TTG TTG AAA TGT TTT GAG AGT CTT GAC |
| | | | | CTC TTC CTT G10583 |
| | | | | (SEQ ID NO:128) |
| 19AXA | cps19aJ (wzx) | 72.3 | AF094575 | 10846TAT CGT GGA TTC GAA TCT GTT ACA GCT |
| | | | | AAT AGA TTT AAA G10807 |
| | | | | (SEQ ID NO:129) |
| 19B19CXS | cps19bJ (wzx) | 74.3 | AF004325 | 7778/373GTC CTG AGG CTA TCA AAT ATC ATT TTC |
| | | | & AF105116 | CGA TTA ATC AG7815/410 |
| | | | | (SEQ ID NO:130) |
| 19B19CXA | cps19bJ (wzx) | 73.2 | AF004325 | 8104/699CCC ACA TGT GAT CAA TAG GAG TGA |
| | | | & AF105116 | AAA TTC TCT ATT C8068/663 |
| | | | | (SEQ ID NO:131) |
| 23FXSO⁷ | cps23FI | 73.4 | AF057294 | 11714CCT TTG GCT AAT TTC TTG GAC GAT AAT |
| | | | | GAA TTT GTA TAT G11753 |
| | | | | (SEQ ID NO:132) |
| 23FXS | cps23fJ (wzx) | 72.3 | AF057294 | 11961GCT TTG GCT AAC TTT TCA TCA AAG ATT |
| | | | | TTA ATT TTT TTG TTA G12003 |
| | | | | (SEQ ID NO:133) |
| 23FXA | cps23fJ (wzx) | 73.3 | AF057294 | 12361CCA GAG ATA GCT GTA ACA CCA ATT TTA |
| | | | | TCA ATT CCC TTA G12322 |
| | | | | (SEQ ID NO:134) |
| 23FXA1⁷ | cps23fJ (wzx) | 72.5 | AF057294 | 12457CCA CAA ACA TTA GCA ATA AAG AAA CCT |
| | | | | AAC AAT CCC12422 |
| | | | | (SEQ ID NO:135) |
| 33F37XSO⁷ | cap33fK (wzy) | 76.7 | AJ006986 | 12271GTT GTT TTA GCT CAA GGA GGG ATA ATG |
| | | | | TTG GCT TCG12306 |
| | | | | (SEQ ID NO:136) |
| 33F37XS | cap33fl & | 72.2 | AJ006986 | 12591/12958GAT CAT ACT CCC TAT CAT TAC GAC |
| | cap37L (wzx) | | & AJ131984 | TCC CTA TGT AAC G12627/12994 |
| | | | | (SEQ ID NO:137) |
| 33F37XA | cap33fl & | 72.1 | AJ006986 | 12918/13285CCA AGA AAT ATC CAA ACC TTT TGA |
| | cap37L (wzx) | | & AJ131984 | CAC TAA ACT TAA TCC12880/13247 |
| | | | | (SEQ ID NO:138) |
| 33F37XA1⁷ | cap33fL (wzx) | 73.3 | AJ006986 | 13016GCT GAT TTT ACA AAT AGG AAA ATA GAG |
| | | | | ATT GCA CCA AC12979 |
| | | | | (SEQ ID NO:139) |
| 3S1 | orf2 (wze)- | 72.6 | Z47210 | 5793GCA CAA AAA AAA GTT TGA TAT TCC CCT |
| | cap3A spacer | | | TGA CAA TAG5828 |
| | | | | (SEQ ID NO:140) |
| 3A1 | cap3A | 73.3 | Z47210 | 6113GCA GGA TCT AAG GAG GCT TCA AGA TTC |
| | | | | AAC TC6082 |
| | | | | (SEQ ID NO:141) |
| 3S2 | cap3A | 72.4 | Z47210 | 6933CGA ACC TAC TAT TGA GTG TGA TAC TTT |
| | | | | TAT GGG ATA CAG AG6973 |
| | | | | (SEQ ID NO:142) |
| 3A2 | cap3B | 75.7 | Z47210 | 7229CTG ACA GCA TGA AAA TAT ATA ACC GCC |
| | | | | CAA CGA ATA AG7192 |
| | | | | (SEQ ID NO:143) |

Notes
1. Primer Tm values provided by the primer synthesiser (Sigma-Aldrich).
2. Numbers represent the numbered base positions at which primer sequences start and finish (starting at point “1” of the corresponding gene GenBank sequence).
3. Underlined sequences show bases added to modify previously published primers.
4. Letters in parentheses indicate alternative nucleotides in different serotypes.
5. Morrison, et al. 2000.
6. Salo, et al. 1995.
7. For sequencing use only.
*Primers have been previously published. All others primers designed specifically for this study.

TABLE 3
Specificity and expected lengths of amplicons of primer pairs used
in this study.

| | | Length of amplicons |
|---|---|---|
| Primer pairs¹ | Specificity | (base pairs) |
| P1/P2 | S. pneumoniae | 864 |
| IIa/IIb | S. pneumoniae | 224 |
| cpsS1/cpsA3² | S. pneumoniae | 1001 |
| cpsS1/cpsA1² | S. pneumoniae | 520 |
| cpsS3/cpsA2² | S. pneumoniae | 503 |
| 1YS/1YA | serotype 1 | 296 |
| 2YS/2YA | serotype 2 | 348 |
| 4YS/4YA | serotype 4 | 348 |
| 6A6BYS/6A6BYA | serogroup 6 | 315 |
| 6A6BYS0/6A6BYA1² | serogroup 6 | 747 |
| 8YS/8YA | serotype 8 | 277 |
| 9V9AYS/9V9AYA | serotypes 9V and 9A | 338 |
| 14YS/14YA | serotype 14 | 310 |
| 18CYS/18CYA | serogroup 18 | 302 |
| 18CYS0/18CYA1² | serogroup 18 | 671 |
| 19FYS/19FYA | serotype 19F | 286 |
| 19AYS/19AYA | serotype 19A | 270 |
| 19B19CYS/19B19CYA | serotypes 19B and 19C | 428 |
| 23FYS/23FYA | serotype 23F | 280 |
| 33F37YS/33F37YA | serotypes 33F/33A/37 | 310 |
| 33F37YS0/33F37YA1² | serotypes 33F/33A/37 | 668 |
| 1XS/1XA | serotype 1 | 426 |
| 2XS/2XA | serotype 2 | 429 |
| 4XS/4XA | serotype 4 | 324 |
| 6A6BXS/6A6BXA | serogroup 6 | 305 |
| 6A6BXS0/6A6BXA1² | serogroup 6 | 1102 |
| 8XS/8XA | serotype 8 | 325 |
| 9V9AXS/9V9AXA | serotypes 9V and 9A | 368 |
| 14XS/14XA | serotype 14 | 289 |
| 18CXS/18CXA | serogroup 18 | 368 |
| 18CXS0/18CXA1² | serogroup 18 | 721 |
| 19FXS/19FXA | serotype 19F | 305 |
| 19AXS/19AXA | serotype 19A | 300 |
| 19B19CXS/19B19CXA | serotypes 19B and 19C | 327 |
| 23FXS/23FXA | serotypes 23F/23A | 401 |
| 23FXS0/23FXA1² | serotypes 23F/23A | 744 |
| 33F37XS/33F37XA | serogroups 33/37 | 328 |
| 33F37XS0/33F37XA1² | serotypes 33F/33A/37 | 746 |
| 3S1/3A1 | serotype 3 | 321 |
| 3S2/3A2 | serotype 3 | 297 |

Notes.
¹See Table 2 for primer sequences.
²For sequencing use only.

DNA Preparation, PCR and Sequencing

DNA extraction, PCR and sequencing were performed as previously described (Kong et al., 2002).

Sequence Comparison, Multiple Sequence Alignments, and Phylogenetic Analysis

Sequences were compared using Bestfit in Comparison program group. Multiple sequence alignments were performed with Pileup and Pretty in Multiple Sequence Analysis program group. Phylogenetic relationships were studied using Ednadist and Ekitsch in Evolutionary Analysis program group. All programs are provided in WebANGIS, ANGIS (Australian National Genomic Information Service), 3^{rd} version.

Nucleotide Sequence Accession Numbers

The new partial sequence data for cpsA-cpsB, wzy (polymerase) and wzx (flippase) genes for selected reference and clinical isolates reported in this paper have appeared in the GenBank Nucleotide Sequence Databases, with accession numbers AF532632-AF532715, and AF163171-AF163232, respectively (Table 1).

Previously reported sequence data used in this paper, in addition to those listed in Table 2, have appeared in GenBank Nucleotide Sequence Databases with the following accession numbers: U15171, U66846 and U66845 (cps gene cluster for serotype 3); NC_{—}003028 (serotype 4 genome); AJ239004 (cps gene cluster for serotype 8); AF030367-AF030372 (cps gene cluster for serotype 19F); AF105113 (partial cps gene cluster for serotype 19A); AF105114 and AF106137 (partial cps gene clusters for serotype 19B); AF105115 (partial cps gene clusters for serotype 19C); AF030373 and AF030374 (cps gene clusters for serotype 23F).

Results

Both pairs of S. pneumoniae species-specific primers (targeting psaA and pneumolysin genes) produced amplicons of the expected size from all reference and clinical isolates except six of 179 CIDM isolates, which, on retesting, were optochin resistant and therefore excluded from further study as they were not S. pneumoniae.

The sequencing primers, cpsS1/cpsA3, formed amplicons from all but 13 reference and clinical isolates. Of these 13 isolates, 10 (eight belonging to serotypes 38/25F and two that were nonserotypable) formed amplicons with primer pairs cpsS1/cpsA1 and cpsS3/cpsA2. Three nonserotypable isolates did not form amplicons using any of the primer pairs targeting the cpsA-cpsB region, although they had been confirmed to be S. pneumoniae using both species-specific PCR.

Sequence Heterogeneity in the Region between the 3′-end of cpsA and the 5′-end of cpsB

The present inventors sequenced and analyzed 800 bp fragments of the region between the 3′-end of cpsA (starting at base pair 951) and the 5′-end of cpsB (see FIG. 2). Representative sequences were deposited into GenBank (see Table 1 for accession numbers). There were 424 sites that were identical for all 51 serotypes represented among the isolates examined, leaving 376 (47%) heterogeneity sites.

Intra- and Inter-serotype/subtype Heterogeneity

Only single isolates were available for 11 serotypes and the mixed serotype 9V/14 (see below). Among 40 serotypes, for which multiple isolates were available, 14 were divided into molecular capsular sequence types, on the basis of major and/or stable intra-serotype heterogeneity. Molecular capsular sequence types were named according to their conventional serotype (cs) and, generally, the source of the isolate in which the sequence difference was first identified [-g=Genbank sequence; -c (CIDM); -q (Queensland); -ca (Canada); -nz (New Zealand)]. When sequences characteristic of two serotypes were present in the cpsA-cpsB region subtype names included both, with the CS first (e.g 23F-23A when CS was 23F; 23A-23F when CS was 23A). Seventeen serotypes had no intra-serotype heterogeneity and in nine there were minor and/or less stable variations between isolates and/or between sequences disclosed herein with corresponding sequences in GenBank (Table 4, FIG. 2).

TABLE 4
Molecular capsular type (MCT) heterogeneity sites in the region between the 3′-end of cpsA and the 5′-end of cpsB of 51
S. pneumoniae serotypes.

| | Intra-MCT^{b} | | MCT^{b}-specific | |
|---|---|---|---|---|
| MCT | Heterogeneity | Identity between | heterogeneity | Selected heterogeneity sites shared with |
| (n=)^{a} | Site - base | MCT (%) | site - base | other MCT^{b}- base |
| 1 (9 + g) | 133 - T^{g}/A⁹ | | 289 - A, 452 - A | 122 - T, 152 - A, 495 - A, 600 - A |
| 2-g (g) | — | | 705, 706 - CG | 287 - G, 507 - G, 534 - A |
| 2-q (3) | Nil | 95.9% | 239 - C, 293 - T, | 232 - G, 286 - C, 600 - A |
| | | | 386 - A, 404 - G | |
| 3 (17 + g) | 262 - C^{g+16}/T¹, 292 - G¹⁶/ | | 485 - A, 487 - A | 27 - A, 90 - A, 231 - A, 590 - T, 686 - T |
| | A^{g+1}, 293 - A¹⁶/G^{g+1}, 539 - | | | |
| | C¹⁶/T^{g+1}, 545 - C^{g+16}/A¹ | | | |
| 4 (36) | Nil | | 179 - C | 231, 232 - TG, 611 - T, 743 - T |
| 5-q (4) | Nil | | | 428 - T, 599 - A |
| 5-c (1) | — | 94.0% | | 122 - T, 152 - A, 247 - C, 605 - T |
| 6A-g (g) | 463-5 - AGC¹²/GCA^{g}, 534 - | | | 62 - A, 209 - A, 534 - A, 542 - C |
| | A^{g}/G¹², 542 - C^{g}/T¹², 545 - | | | |
| | A^{g}/C¹² | | | |
| 6A-ca (7) | 55 - A⁵/G², 331 - A²/G⁵, 434 - | 6A-ca:6A-g = 99.1% | | 62 - A, 209 - A |
| | A⁵/G² | | | |
| 6A-c (2) | Nil | 6A-c:6A-ca = 99.5% | | 62 - A, 209 - A, 337 - G |
| 6A-6B-g (2) | (see 6B-g) 772 - A^{g+1}/G¹ | | | (see 6B-g) |
| 6A-6B-q (1) | (see 6B-q) | | | (see 6B-q) |
| 6B-g (4 + g) | 31 - A¹/G^{g+3} | | | 209 - A, 337 - G, 341 - G, |
| 6B-q (9) | 383 - A⁸/G¹ | 6B-q:6B-g = 84.7% | 749 - G | 52 - G, 58 - C, 68 - G, 82 - C, 85 - T, 94 - |
| | | | | T, 104 - T, 116 - G, 160 - T, 209 - C, 286 - |
| | | | | C, 343 - G, 375 - G, 478 - C, 490 - C, |
| | | | | 521 - T, 563 - T, 704 - C, 776 - C |
| 6B-c (1) | — | 6B-c:6B-g = 92.1% | | 193 - T, 209 - C |
| 7F (15) | Nil | | 66 - C, 445 - C | 722 - C, 731 - A |
| 7C (3) | Nil | | | 49 - C, 731 - A |
| 8 (12) | Nil | | 340 - T, 670 - G | 425 - A |
| 9N (9) | Nil | | 81 - T, 378 - A | 352 - G, 409 - T, 590 - T, 722 - A |
| 9V (17) | Nil | | 245 - G | 428 - C, 704 - C, 750 - T, 776 - C |
| 10F (2) | 309 - G¹/A¹, 335 - G¹/A¹ | | | 704 - C, 750 - T, 776 - C |
| 10A-q (5) | Nil | | 222 - T, 663 - T | 232 - G |
| 10A-23F (6) | (see 23F-g) | 91.2% | | (see 23F-g) |
| 11A-q (7) | Nil | | | 122 - T, 232 - G, 478 - C, 490 - C, 521 - |
| | | | | T, 704 - C |
| 11A-nz (1) | — | 94.0% | 316 - T | 597 - A |
| 11B (1) | — | | 269 - A, 490 - G, | 10 - G, 52 - G, 58 - C, 68 - G, 82 - C, 85 - T, 94 - |
| | | | 776 - T | T, 104 - T, 116 - G, 148 - T, 160 - T, 231, 232 - |
| | | | | TG, 247 - C, 250 - A, 286 - C, 292 - C, 343 - G, |
| | | | | 375 - G, 425 - A, 521 - T, 563 - T, 704 - C |
| 12F (9) | 268 - A¹/C⁸, 572 - C¹/T⁸, 781 - | | 274 - C | 287 - G, 497 - G, 577 - T, 722 - C |
| | G¹/T⁸ | | | |
| 13 (6)/20 (8) | Nil; Nil | | | 590 - T, 686 - T, 722 - A |
| 14-g (32 + g) | 249 - T²³/C^{g+9}, 250 - G³²/T^{g}, | | | 577 - T |
| | 320 - G³²/A^{g} | | | |
| 14-c (1) | — | 98.1% | 613 - G | 16 - C, 49 - C, 54 - T, 62 - T, 406 - G, 577 - T |
| 15A-ca1 (1) | — | | 473 - G | 49 - C, 337 - G, 507 - G |
| 15A-ca2 (1) | — | 95.1% | 406 - A, 473 - G | 337 - G, 507 - G |
| 15B-q (5) | Nil | | | 232 - G |
| 15B-c (1) | — | 15B-c:15B-q = 97.4% | 235 - T, 351 - G | 49 - C, 247 - C, 352 - G, 428 - T, 542 - C |
| 15B-22F (2) | (see 22F) | 15B-22F:15B-q = | | (see 22F) |
| | | 95.2% | | |
| 15C-q (1) | as for 15B-q plus 104 - T^{C}/C^{B} | | | 232 - G |
| 15C-CA (1) | as for 15B-q plus 232 - | 99.6% | | pattern |
| | A^{C}/G^{B}, 757 - T^{C}/C^{B} | | | |
| 16F (6) | 149 - C⁵/T¹, 232 - A⁵/G¹ | | | 122 - T, 232 - G, 352 - G, 548 - A |
| 17F-c (3) | Nil | | | 199 - A, 247 - C, 600 - C |
| 17F-35B (2) | (see 35B) | 99.8% | 728 - C | (see 35B) |
| 17A (1) | — | | 122 - A | 85 - T, 554 - G, 567 - A |
| 18F (1) | — | | 65 - A, 161 - T, 469 - | 722 - C, 786 - C |
| | | | C, 684 - A | |
| 18A (2) | 63 - T¹/A¹ | | 99 - C, 202 - G, 232 - | 122 - T, 307 - G, 563 - T, 686 - T |
| | | | C, 239 - G, 322 - | |
| | | | C, 334 - C, | |
| 18B (4)/18C (14) | Nil; Nil | | 138 - G, 459 - C, | 478-C |
| | | | 750 - A | |
| 19F (20 + gx7) | 164 - C^{gx7+17}/T³, 169 - C^{gx6+11}/ | | | 169 - T, 337 - G |
| | T^{g+9}, 387 - A^{gx6+20}/T^{g}, | | | |
| | 414 - G^{gx5+20}/T^{gx2}, | | | |
| | 479-G^{gx7+16}/A⁴ | | | |
| 19A (11 + g) | 70 - T^{g}/C¹¹, 479 - A⁸/G^{g+3} | | 202 - C | 49 - C, 54 - T, 62 - T, 94 - A, 103 - C, 104 - |
| | | | | T, 160 - T, 198 - C, 232 - G, 286 - C, |
| | | | | 343 - G, 352 - G, 375 - T, 425 - A, 490 - |
| | | | | C, 750 - T |
| 21 (1) | — | | | 428 - C, 548 - A, 629 - T, 717 - A |
| 22F (13) | Nil | | | 428 - T, 567 - G, 599 - A, 731 - A |
| 22A (4) | Nil | | | 428 - T, 567 - A, 599 - A, 731 - A |
| 23F-g (17 + gx3) | Nil | | | 193 - T |
| 23F-c (1) | — | 23F-c:23F-g = 91.2% | 88 - G | 249 - A, 337 - G |
| 23F-23A (1) | — | 23F-23A:23F-g = | | 495 - A |
| | | 98.7% | | |
| 23A-ca (2) | Nil | | | 247 - C, 495 - A |
| 23A-23F (1) | (as for 23F-23A) | 96.6% | | (as for 23F-23A) |
| 23B (1) | — | | 734 - C, 763 - G | 49 - C, 55 - T, 58 - C, 62 - T, 103 - C, 104 - |
| | | | | T, 160 - T, 198 - C, 223 - G, 232 - G, |
| | | | | 249 - T, 286 - C, 292 - C, 343 - G, 375 - |
| | | | | G, 376 - G, 425 - A, 490 - C, 521 - T, 563 - |
| | | | | T, 704 - C |
| 25F (1)/38 (7) | —; Nil | | Numerous sites | Numerous sites |
| 29 (1) | — | | 310 - A | 335 - A |
| 31 (2)/42 (1) | Nil; — | | | 122 - T, 152 - A, 605 - T |
| 33F-g (2 + g)/ | 534 - A^{g}/G²; - | | | 247 - C, 600 - A, 728 - T |
| 33A (1) | | | | |
| 33F-q (4) | 313 - T¹/G³ | 94.7% | 313 - T | 169 - T, 717 - A |
| 33B (1) | — | | 578 - G | 169 - T, 717 - A |
| 34 (2) | Nil | | | 85 - C, 122 - C, 554 - G, 567 - A |
| 35F (6) | Nil | | | 232 - G, 343 - G, 554 - G, 577 - T |
| 35B (9) | Nil | | | 199 - G, 247 - C, 600 - A, 728 - C |
| 37 (1 + g) | 231 - A^{g}/C¹ | | 54 - G | 90 - A, 231 - A, 743 - T |
| 41F (1) | — | | | 287 - G, 507 - G |

Notes.
^{a}Key to mcst: -g = Genbank sequence; -c (CIDM); -q (Queensland); -ca (Canada); -nz (New Zealand)
^{b}The superscript numbers = number of isolates studied; superscript g = base present in corresponding GenBank sequence

There were 368 heterogeneity sites that allowed differentiation between molecular capsular sequence types, including both specific and shared sites (Table 4, FIG. 2).

Phylogenetic Tree Based on Region of the 3′-end of cpsA-the 5′-end of cpsB Genes

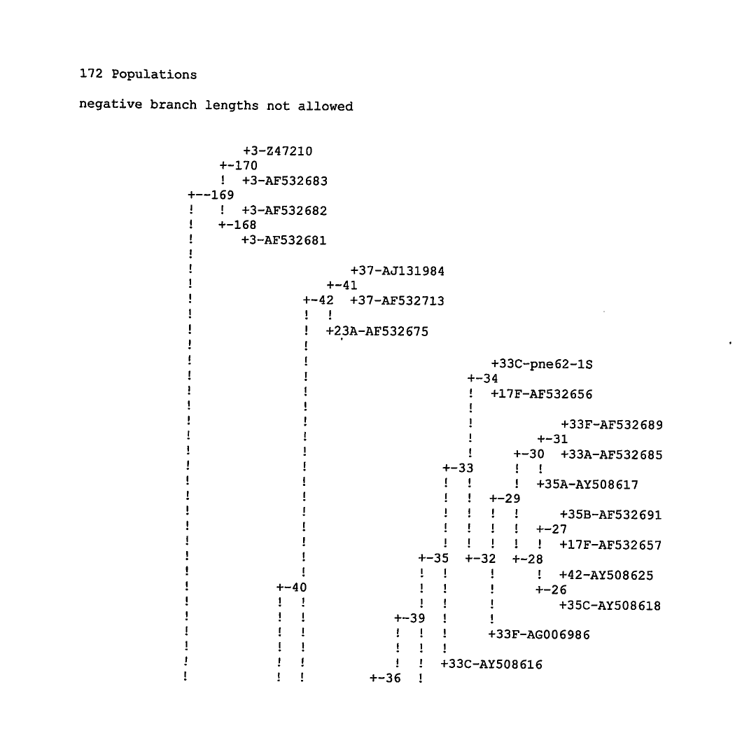

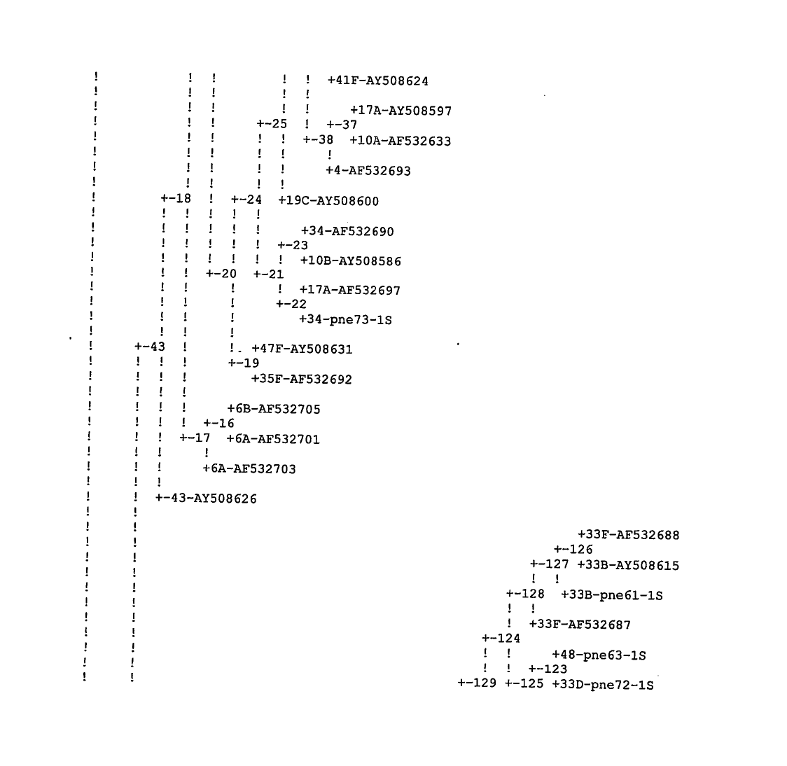

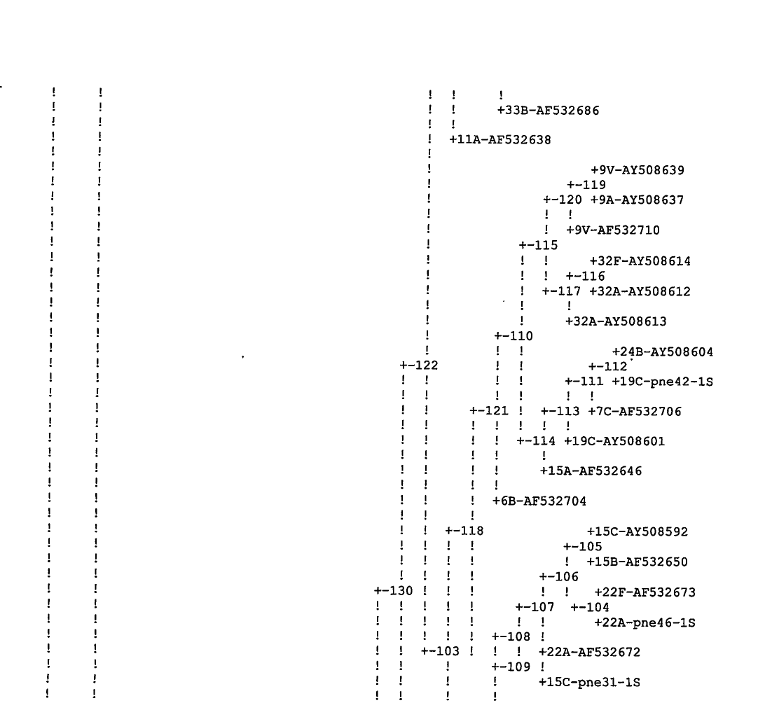

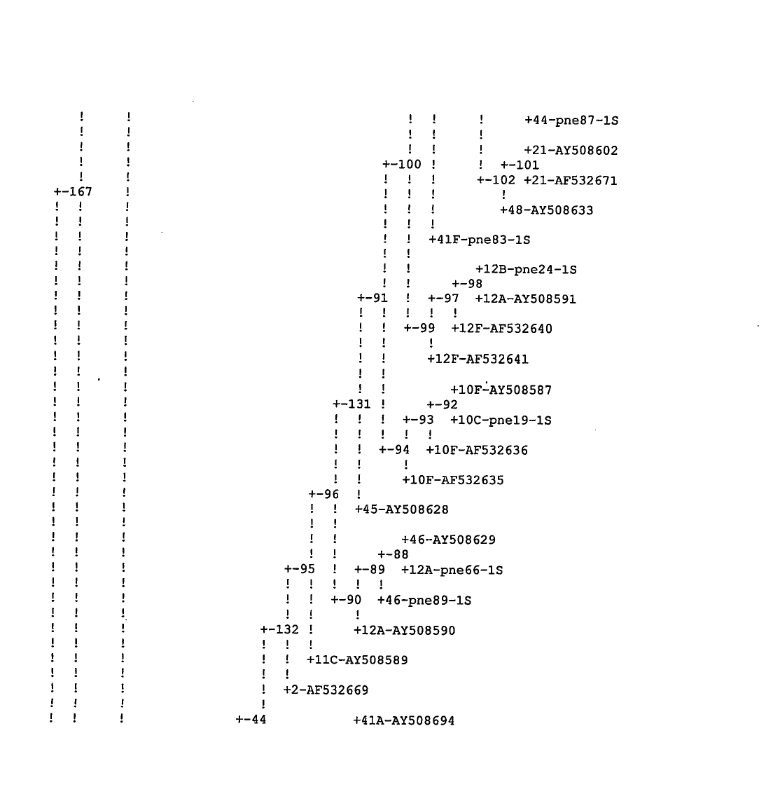

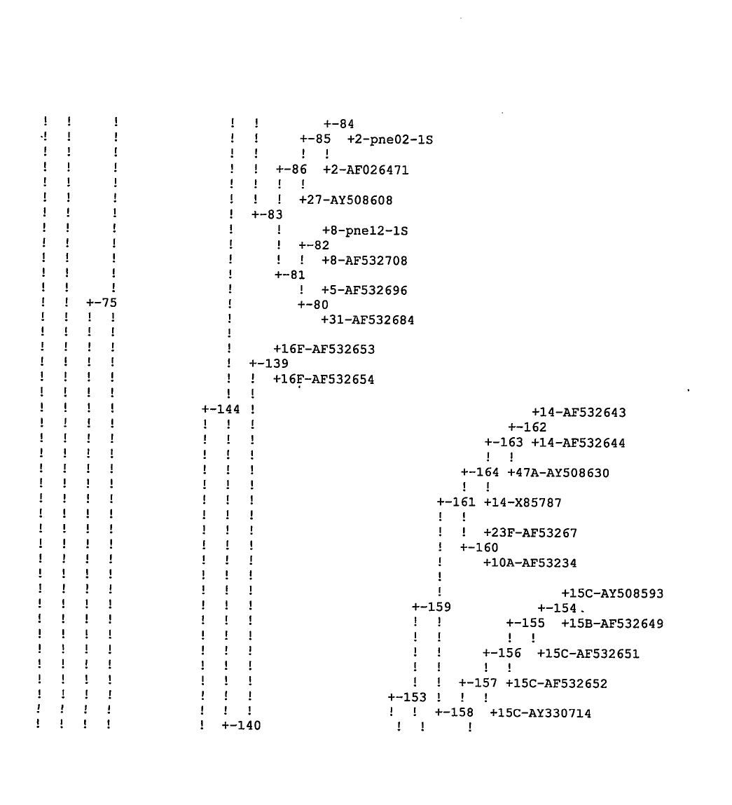

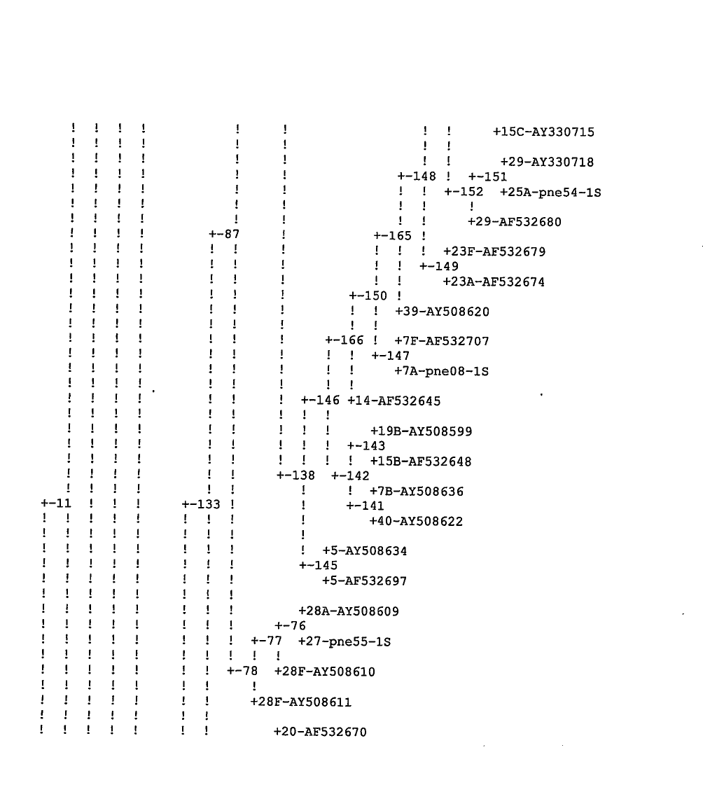

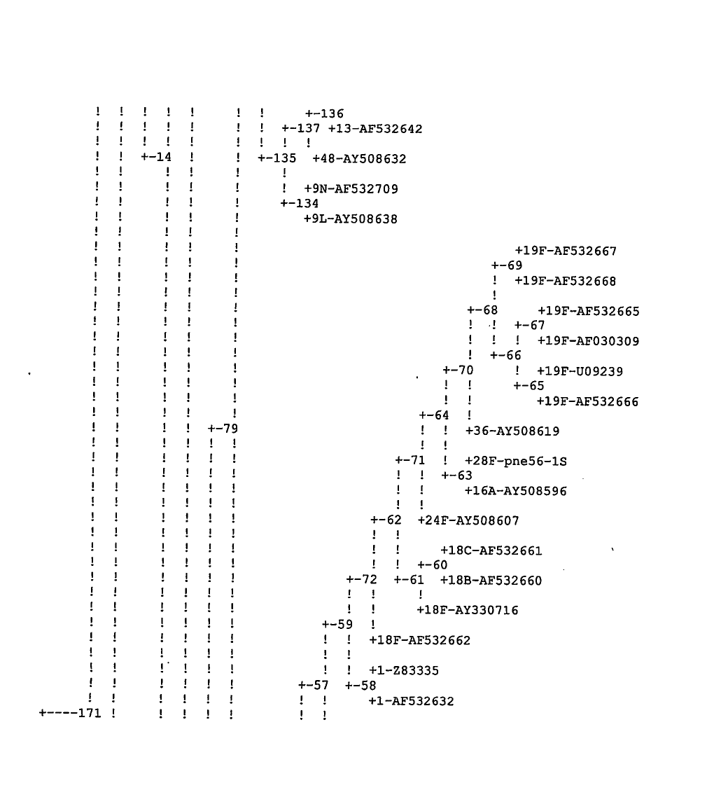

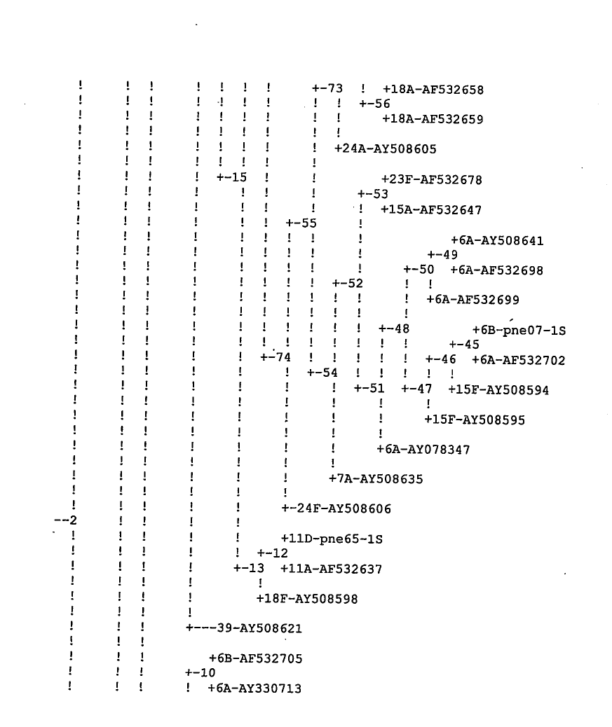

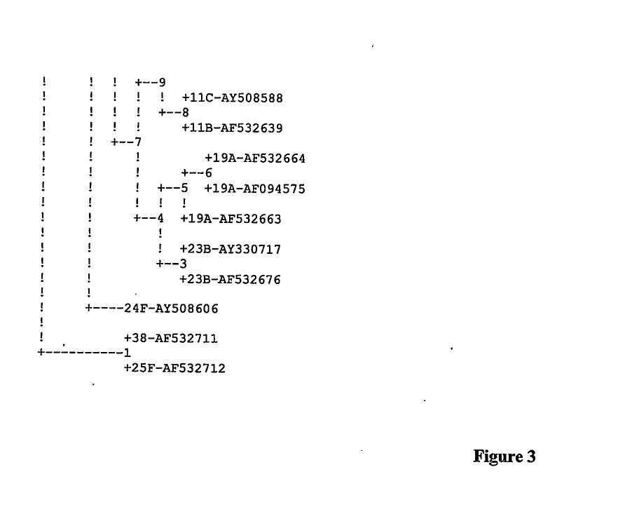

Using these 800 bp sequences, a phylogenetic tree was inferred for the 132 (included the new sequences from Example 2) S. pneumoniae molecular capsular sequence type analysis of the cpsA-cpsB region (FIG. 3—it should be noted that in FIG. 3 the sequence types were renamed based on serotype and their GenBank accession numbers). Typical class I serotypes (e.g. 1, 18C, 19F), a typical class II serotype (e.g 33F, represented by 33F-g) and a nontypical class II serotype (19A) were each in different clusters of the tree (Jiang et al., 2001).

The phylogenetic tree provides evidence for, and suggests possible sources of, recombination between cpsA-cpsB genes of classes I and II. For example, subtype 23F-c (or 23F-AF532678) clustered with 15A-c2 (or 15A-AF532647), but in a separate cluster from other 23F and 15A subtypes, suggesting that they may have arisen by recombination between 23F and 15A, respectively, and other serotypes.

Molecular Capsular Sequence Typing Based on cpsA-cpsB Region Sequences

The molecular capsular sequence type, assigned on the basis of cpsA-cpsB sequence, was the same as the CS for all isolates belonging to 36 of 51 serotypes (or 304 of 394 [77%] isolates), and for the majority of isolates (25 of 39) belonging to another five serotypes (Table 5). The remaining isolates in these serotypes shared sequences with other serotypes, namely 6A with 6B, 10A and 23A with 23F, 15B with 22F and 17F with 35B, presumably as -a result of recombination. There were five serotype pairs, represented by 46 isolates, whose members had identical sequences: namely 20/13, 18C/18B, 38/25F, 31/42 and 33F-g/33A.

TABLE 5
Comparison of molecular capsular typing (MCT) and conventional serotyping (CS) results of 394 S. pneumoniae isolates.

| | | MCT-seq: a) cpsA-cpsB or | | | |
|---|---|---|---|---|---|
| CS | N= | b) wzx, wzy type(s) (n)¹ | MCT-PCR (wzy & wzx) | Final MCT | Comment |
| 1 | 9 | 1 | 1 | 1 | Correlate |
| 2 | 3 | 2 | 2 | 2 | ″ |
| 3 | 17 | 3 | 3 | 3 | ″ |
| 4 | 36 | 4 | 4 | 4 | ″ |
| 5 | 5 | 5 | NA | 5 | ″ |
| 6A | 12 | 6A(9); 6B-g (2); 6B-q (1) | Serogroup 6 | 6A (11) | 1 of 12 results |
| | | 6A (11)²; 6B-q (1) | | 6B (1) | discrepant² |
| 6B | 15 | 6B | Serogroup 6 | 6B | Correlate |
| 7C | 3 | 7C | NA | 7C | ″ |
| 7F | 15 | 7F | NA | 7F | ″ |
| 8 | 12 | 8 | NA | 8 | ″ |
| 9N | 9 | 9N | NA | 9N | ″ |
| 9V | 17 | 9V | 9V | 9V | ″ |
| 9V/14 | 1 | 9V | 9V/14 | 9V/14 | See text |
| 10A | 11 | 10A (5); 23F-g (6)³ | 23F wzy/wzx PCR negative (6)³ | 10A (11)³ | Correlate³ |
| 10F | 2 | 10F | NA | 10F | Correlate |
| 11A | 8 | 11A | NA | 11A | ″ |
| 11B | 1 | 11B | NA | 11B | ″ |
| 12F | 9 | 12F | NA | 12F | ″ |
| 13 | 6 | 13/20 | NA | 13/20 | Consistent |
| 14 | 33 | 14 | 14 | 14 | Correlate |
| 15A | 2 | 15A | NA | 15A | Correlate |
| 15B | 8 | 15B (6); 22F (2) | NA | 15B (6); 22F (2) | 2 of 8 results |
| | | | | | discrepant |
| 15C | 2 | 15C | NA | 15C | Correlate |
| 16F | 6 | 16F | NA | 16F | ″ |
| 17A | 1 | 17A | NA | 17A | ″ |
| 17F | 5 | 17F (3); 35B (2) | NA | 17F (3); 35 (2) | 2 of 5 results |
| | | | | | discrepant |
| 18A | 2 | 18A | Serogroup 18 | 18A | Correlate |
| 18B | 4 | 18C/18B | ″ | 18B/C | Consistent |
| 18C | 14 | C/18B | ″ | 18B/C | ″ |
| 18F | 1 | 18F | ″ | 18F | Correlate |
| 19A | 11 | 19A | 19A | 19A | ″ |
| 19F | 20 | 19F | 19F | 19F | ″ |
| 20 | 8 | 13/20 | NA | 20 | Consistent |
| 21 | 1 | 21 | NA | 21 | Correlate |
| 22A | 4 | 22A | NA | 22A | ″ |
| 22F | 13 | 22F | NA | 22F | ″ |
| 23A | 3 | 23A (2); 23F-g (1) | 23F wzy PCR negative/23F wzx PCR | 23A⁴ | ″⁴ |
| | | 23A (3)⁴ | positive⁴ | | |
| 23B | 1 | 23B | NA | 23B | ″ |
| 23F | 20 | 23F | 23F | 23F | ″ |
| 25F | 1 | 25F/38 | NA | 25F/38 | Consistent |
| 29 | 1 | 29 | NA | 29 | Correlate |
| 31 | 2 | 31/42 | NA | 31/42 | Consistent |
| 33A | 1 | 33A/33F-g⁵ | Serogroup 33/37⁵ | 33A/33F⁵ | ″⁵ |
| 33B | 1 | 33B | Serogroup 33/37 PCR (wzy) negative⁶ | 33B | Correlate⁶ |
| 33F | 6 | 33A/33F-g⁵, 33F-q | Serogroup 33/37⁵ | 33A/33F⁵ | Correlate⁵ |
| 34 | 2 | 34 | NA | 34 | Correlate |
| 35B | 9 | 35B | NA | 35B | ″ |
| 35F | 6 | 35F | NA | 35F | ″ |
| 37 | 1 | 37 | Serogroup 33/37 | 37 | ″ |
| 38 | 7 | 25F/38 | NA | 25F/38 | Consistent |
| 41F | 1 | 41F | NA | 41F | Correlate |
| 42 | 1 | 31/42 | NA | 31/42 | Consistent |
| Nonserotypable | 5 | Non-typable⁷ | NA⁷ | Non-typable⁷ | Correlate⁷ |
| TOTAL | 394 | | | | |

Results:
Correlate = 343
Consistent = 46
Discrepant = 5
Notes.
¹For nomenclature, see Table 4 and text.
²cpsA-cpsB sequence 3 discrepancies; 2 resolved by wzx, wzy gene sequences.
³Six serotype 10A isolates shared cpsA-cpsB sequence with 23F-g, but 23F specific PCR (targeting both wzy and wzx) was negative; 10A-23F was identified by exclusion of 23F in our existing database. However, this relationship needs to be confirmed by examination of alarger collection isolates.
⁴cpsA-cpsB sequence 1 discrepancy; resolved by wzx gene sequence; 23F wzx PCR positive/23F negative wzy PCR negative also support its identification by exclusion.
⁵For one serotype 33A isolate, cpsA-cpsB and wzx and wzy sequences were identical with 33F-g but different from 33F-q; 33F/37 wzx and wzy PCR were both positive.
⁶One serotype 33B strain identified by exclusion: 33F/37 wzx PCR positive/33/37 wzy PCR negative.
⁷All isolates confirmed to be S. pneumoniae. These isolates may belong to rare serotypes not represented among our reference isolates.

Molecular Capsular Sequence Typing Based on PCR Targeting wzy and wzx (orf2 [wze]-cap3A-cap3B for Serotype 3)

There is significant sequence heterogeneity in wzy and wzx (data not shown), which made them suitable PCR targets for serogroup or serotype identification (Tables 2 and 3). With few exceptions, primer pairs targeting these genes formed amplicons only from the corresponding serotypes represented in the five reference panels. Exceptions were: PCR targeting serotype 6B also amplified 6A; PCR targeting 18C amplified all serotypes in serogroup 18; PCR targeting wzx (but not wzy) of serotype 23F, amplified three serotype 23A strains; PCR targeting wzx and wzy of serotypes 33/37 amplified a 33A isolate and that targeting wzx amplified a serotype 33B isolate.

The specificity of serotype 3-specific primers targeting the orf2 (wze)-cap3A-cap3B genes (Arrecubieta et al., 1996) was confirmed by production of an amplicon of the expected size from all 17 serotype 3 isolates. Thus, a serotype or serogroup was assigned by PCR to all 239 isolates belonging to serotypes/serogroups for which specific PCR was developed (Table 5).

Comparison of Molecular Capsular Sequence Typing Based on cpsA-cpsB Sequencing and PCR/sequencing Targeting wzx and wzy

The results of PCR and cpsA-cpsB sequencing were consistent except that PCR could not distinguish between some members of serogroups 6, 18, 23 and 33/37 and further sequencing (of wzx, wzy) was required to identify individual molecular capsular sequence types (see below). The cpsA-cpsB sequences of six 10 A isolates were identical to those of 23F, but the isolates were negative in the 23F-specific PCR targeting wzx and wzy (10A-23F).

Relationships within Serogroups

Sequence analysis of the cpsA-cpsB region and wzy and wzx genes (data not shown) showed variable phylogenetic relationships between members of different serogroups.

Serogroup 6

Serotypes 6A and 6B were divided into five and three subtypes, respectively, based on different sequence patterns in the cpsA-cpsB region. Three 6A isolates had sequences in this region characteristic of serotype 6B (Table 4). Serotypes 6A and 6B could not be distinguished by PCR targeting wzx and wzy. Sequencing of these genes correctly identified all except one 6A isolates, but some 6A and 6B subtypes share identical or very similar sequences. The serotype of the discrepant isolate (serotype 6A, 6B-q) was checked independently by two laboratories (Vakevainen et al., 2001).

Serogroup 18

Serotypes 18C and 18B had identical cspA-cpsB region sequences and were close to 18A and 18F in the class I cluster (FIG. 3). PCR targeting both wzx and wzy genes amplified all four serotypes. Sequences of 18C and 18B were identical to each other, but different from those of serotypes 18A and 18F, which were also distinguishable from each other.

Serogroup 23

Serotypes 23F, 23A (except 23F-23A and 23A-23F) and 23B were separated into different clusters based on cpsA-cpsB sequence differences. Serotype 23A (including 23A-23F) was identified on the basis of a positive result with 23F-specific primers targeting wzx and a negative result with the corresponding wzy PCR Sequencing could differentiate individual serotypes (23A, 23F and 23B) except 23F-23A and 23A-23F. Mcst 23F-c, 23A-23F and 23F-23A have apparently arisen by recombination between 23F, 23A and/or others, producing sequences in the cpsA-cpsB regions that are quite different from their parental types.

Serogroups 33 and 37

Serotypes 33A and 33F-g share identical cpsA-cpsB sequences and that of 33B is similar; 37 and 33F-g cluster together, as do 33B and 33F-q (FIG. 3). The 33F/37-specific wzx PCR amplified 37, 33F, 33A and 33B, indicating similarities at that site, although sequencing showed clear differences between 33B and the others. The 33F/37-specific wzy PCR amplified 37, 33F and 33A but not 33B. Thus, mct 33B was identified on the basis of a positive result with 33F/37-specific primers targeting wzx and a negative result with the corresponding wzy PCR.

Other Serogroups

Despite antigenic similarities that determine their membership of the same serogroup, serotypes 9N and 9V appear to be genetically distant, on the basis of significant differences between their cpsA-cpsB sequences and the fact that 9V-specific PCR did not amplify 9N.

Similarly, mct 19F and 19A had quite different cpsA-cpsB region sequences and separated into different clusters. 19F-specific PCR did not amplify 19A and vice versa. There were differences between mct 19F, 19A, 19B, 19C in wzx and wzy sequences (except wzy sequence of 19C was not available in GenBank), but they formed two groups—19F, 19A and 19B, 19C.

Serotypes 7F and 7C separated into different clusters based on cpsA-cpsB sequences, as did 11A and 11B (FIG. 3). Serotypes 15B and 15C had similar cpsA-cpsB sequences and clustered together, except for 15B-22F. Serotypes 17F (including 17F-c and 17F-35B) and 17A were clustered together. Serotypes 35F and 35B are closely related based on similar cpsA-cpsB sequences.

Mixed Culture

One clinical isolate identified as serotype 9/14 using antisera was positive in 9V- and 14-specific PCR (targeting both wzx and wzy), but was identified as mct 9V by sequencing. The isolate was subcultured and 16 individual colonies were rested. All 16 colonies were positive in both mct 9V-specific and negative in both 14-specific PCR assays and were identified as mct 9V by sequencing. The serotype of the original isolate was rechecked and the results (mixed serotype 9/14) were as before. It was therefore assumed that the original isolate was a mixture, predominantly of serotype 9V with a minor component of serotype 14.

Comparison of Serotype Identification Results between Molecular Capsular Sequence Typing and CS

After CS and molecular capsular sequence typing had been completed, the results were compared. Initial results were discrepant for 29 isolates; repeat serotyping and/or correction of clerical errors resolved all but five discrepancies. Final results correlated between CS and molecular capsular sequence typing methods for all isolates of 38 serotypes (318 isolates), 20 of 25 of another three serotypes and all five nonserotypable isolates (total 343 isolates). In addition, there were 46 isolates belonging to pairs of serotypes whose members could not be distinguished from each other by molecular capsular sequence typing but all were assigned to the pair that included the serotype to which they had been assigned by CS. These results were classified as consistent.

The five discrepant results were: one isolate of serotype 6A was identified as 6B-q, two isolates of serotype 15B were identified as 22F and two isolates of serotype 17F as 35B.

Algorithm for Serotype Assignment of S. pneumoniae by Molecular Capsular Sequence Typing

An algorithm for practical use of the molecular capsular sequence typing method for the identification of S. pneumoniae serotypes is shown in Table 6.

Discussion

Sequences of 16 cps gene clusters showed that all have the same four genes at their 5′ ends-cpsA (wzg)-cpsB (wzh)-cpsC (wzd)-cpsD (wze)-which are the sites for recombination events that generate new forms of capsular polysaccharide. The sequences for different serotypes can be divided into two classes and show evidence of interesting recombination patterns.

The study of 51 serotypes, of which 40 were represented by more than one isolate, showed that the cpsA-cpsB sequences for the same serotypes were generally stable or could be consistently divided into a small number of subtypes. This shows that sequence patterns in this region can be used to identify different serotypes/serosubtypes.

It has been shown previously that PCR-RFLP based on the cpsA-cpsB region can predict S. pneumoniae serotypes (Lawrence et al., 2000). However, the method generates a long amplicon (1.8kbp), requires the use of three restriction enzymes and special equipment and has limited discriminatory ability.

The present inventors identified 376 sequence heterogeneity sites, in the cpsA-cpsB region, among the 51 serotypes studied (Table 4, FIG. 2), which allowed a practical MCT assay based on sequencing to be developed. Several pairs of primers were designed to amplify a 1001 bp segment within the cpsA-cpsB region, based on the following considerations. The primers formed amplicons from virtually all, S. pneumoniae isolates (>99% of those examined); the amplicon is small enough to be amplified using normal PCR protocols; the region of interest (800 bp) can be sequenced using a single reaction and the method is objective. The target included most of the variable sites (bp 951 to 1747), providing maximum discrimination between closely related serotypes (e.g. members of serogroups 33 and 37 that could not be distinguished by serotype/group-specific PCR).

TABLE 6
Algorithm for S. pneumoniae molecular capsular sequence type
identification by sequencing and serotype/group-specific PCR.

| | | |
|---|---|---|
| | PCR | |
| Amplification | product size | |
| primer pairs* | (base pairs) | Interpretation |

S. pneumoniae identification primer pairs

| | | |
|---|---|---|
| P1/P2 | 864 | S. pneumoniae |

S. pneumoniae mct identification by sequencing

| | | |
|---|---|---|
| cpsS1/cpsA3 | 1001 | 1. Purification PCR amplicons |
| (for most MCT) | or | 2. Sequencing PCR amplicons |
| or | 520 + 503 | 3. Using programmes (Pileup & Pretty |
| cpsS1/cpsA1 + | | or Ednadist & Ekitsch etc.) in ANGIS |
| cpsS3/cpsA2 | | to analyse sequences to identify |
| (for MCT 38/25F | | mct/mcst |
| and some | | 4. Refer to FIG. 1/Table 4 to identify/ |
| nontypable | | confirm mct/mcst. |
| isolates) | | |

S. pneumoniae mct identification by serotype/group-specific PCR
See Table 2 for primer sequences* and Table 3 for specificity and amplicon lengths of primer pairs. Only selected molecular capsular sequence types and isolates need to be identified using the full testing algorithm.

Some of the 376 heterogeneity sites in the cpsA-cpsB region were specific for individual molecular capsular sequence type (Table 4, FIG. 2), while others were shared between several. Based on these patterns, plus PCR and selective sequencing of type-specific regions of wzx and wzy, most of the 51 serotypes represented among our 394 isolates could be distinguished and further divide them into a total of 71 molecular capsular sequence types, with the aid of sequence analysis software. The final CS and molecular capsular sequence typing results correlated for 343 isolates of 389 (88%) for which results for both methods were available, including five that were nontypable by either method. For 46 isolates belonging to five serotype pairs, members of which could not be distinguished by sequencing, results were classified as consistent leaving unresolved discrepancies between methods for only five (1.2%) isolates.

Sequence analysis of the cps gene clusters of 16 serotypes showed that wzy (capsular polysccharide polymerase gene) and wzx (capsular polysccharide flippase gene) are highly variable, making them suitable targets for direct serotype identification by PCR. The present inventors designed serotype-specific PCR primers for these serotypes, targeting wzx and wzy and, for serotype 3, which has no wzy and wzx genes, targeting orf2 (wze)-cap3A-cap3B (Arrecubieta et al., 1996). It was found that presumed serotype-specific primers for 6A, 18C, 23F and 33F/37 were not serotype-specific, but amplified other related serotypes. To improve the molecular capsular sequence typing methods, portions of the wzy and wzx genes of serotypes within these groups were sequenced, which allowed molecular capsular sequence types to be distinguished within these serotypes/groups and demonstrate relationships between them.

The present inventors have recognized that the large number of pneumococcal serotypes would make it impractical to use serotype-specific PCR for all of them. Nevertheless, wzy and wzx PCR can be used to resolve discrepancies between CS and cpsA-cpsB region sequencing assays e.g. for molecular capsular sequence types 10A-23F and 23A-23F. Moreover, the use of two target regions in the cps gene cluster helps to clarify the relationships between mcst that have apparently arisen by recombination. Serotype/group-specific primers were evaluated using three reference panels, which had been characterised by CS and used to identify clinical isolates of unknown cs. By PCR alone, 239 (61%) of our 394 clinical isolates were assigned to a serotype or serogroup (Table 5). This method can be extended to other mct, when additional wzx and wzy sequences are available.

In some circumstances, sequencing of the cpsA-cpsB region may be more practical than type-specific PCR. For most serotypes only a single method and fewer primers (cpsS1/cpsA3-for most serotypes/isolates) are needed.

Previous studies have shown that serotypes included in 23-valent polysaccharide and 11-, 9-, 7-valent protein conjugate vaccines are those most frequently isolated from normally sterile sites (CSF, blood) (Colman et al., 1998; Huebner et al., 2000). Among 173 consecutive pneumococcal “sterile site” isolates from adults in the CIDM diagnostic laboratory, over a 2.5-year period, correlation between the mct and cs was good (171/173 CIDM isolates were correctly identified). The exceptions were two serotype 15B isolates that were identified as molecular capsular sequence type 22F. Five serotypes (4, 14, 19F, 23F, 9V—covered by all pneumococcal vaccines) accounted for 57% of isolates.

Five of 394 isolates studied were nontypable by both CS and molecular capsular sequence typing (Barker et al., 1999). Isolates may be nonserotypable because of decreased type-specific-antigen synthesis, nonencapsulated phase variation or insertion or mutation of genes of cps gene clusters. Failure to type them by molecular capsular sequence typing reflects the fact that the sequence database is still incomplete (also the reason for the further research in Example 2), although the target regions of two of the five nonserotypable isolates have been sequenced.

In summary, the present inventors have developed a molecular capsular sequence typing system for S. pneumoniae, which is reproducible, can be performed by any laboratory with access to PCR/sequencing and does not require large panels of expensive serotype-specific antisera. Work on an international collection of isolates in our reference panels demonstrated a strong correlation between the cpsA-cpsB sequence and CS. Heterogeneity in a relatively short sequence (800 bp) in this region, supplemented by serotype/group-specific PCR targeting wzx and wzy, correctly predicted the serotype of most unknown isolates belonging to 51 serotypes. These novel molecular capsular sequence typing methods provide comprehensive strain identification that will be useful for epidemiological studies that will be needed to monitor serotype distribution and detect serotype switching, if any, among S. pneumoniae isolates before and following introduction and widespread use of conjugate vaccines.

### EXAMPLE 2

Identification of S. pneumoniae Serotypes by Analysis of the wzx and/or wzv Genes

Materials and Methods

Pneumococcal Clinical Isolates

This study was based on 92 well-characterized S. pneumoniae isolates, which represented 55 serotypes and including about 31 of 39 serotypes that were not included in Example 1. The sources of these isolates were 72 from China Medical Bacteria Culture Collection Center, Beijing, PR China; 17 from Royal College of Pathologists of Australasia, Quality Assurance Program Pty Limited, New South Wales, Australia; three from Associate Professor Geoff Hogg and Ms Jenny Davis, Microbiological Diagnostic Unit (MDU), Public Health Laboratory, Department of Microbiology and Immunology, University of Melbourne, Victoria. Conventional serotyping (CS) had been performed by donor laboratory and serotypes of the 75 strains were known at time of receipt and 23 selected isolates (including all of serotypes 27, 28F and 16A isolates and two from Example 1—which had been identified as one each of serotype 42 and 41F strains each) were re-tested by the Quellung reaction—as described above—at Department of Microbiology, Children's Hospital at Westmead (Henrichsen, 1999).

Isolates were retrieved from storage by subculture on blood agar plates (Columbia II agar base supplemented with 5% horse blood) and incubated overnight at 37° C. in 5% C0₂.

Annotation and Analysis of wzx and wzy

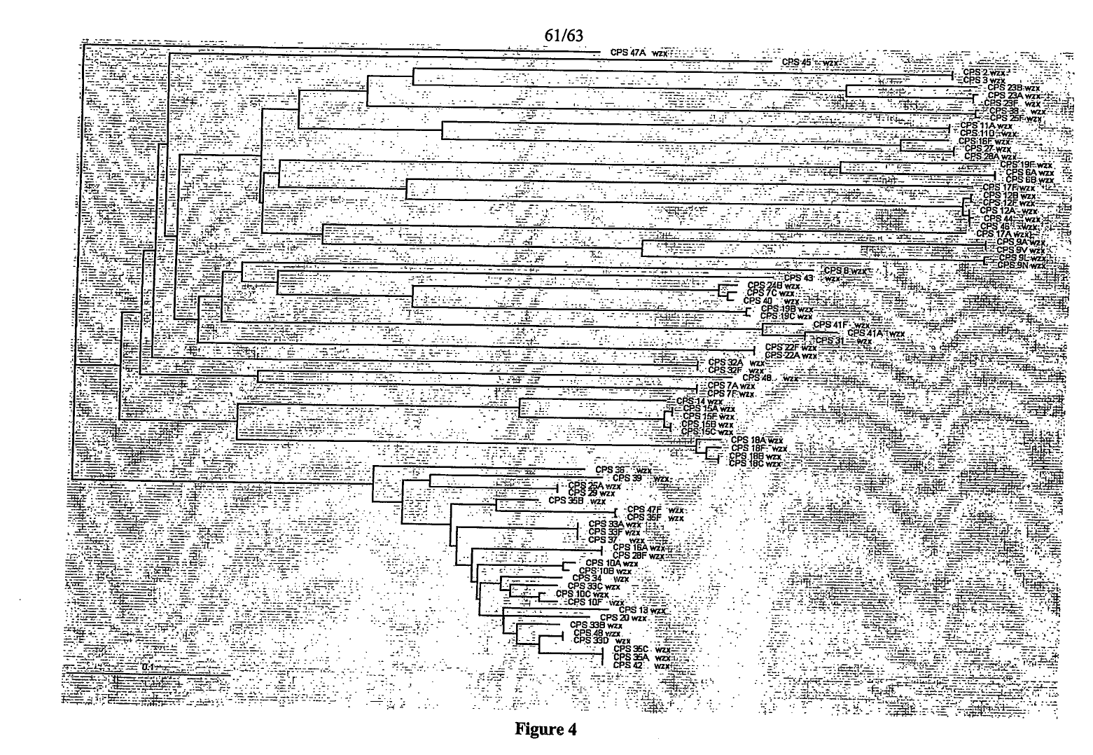

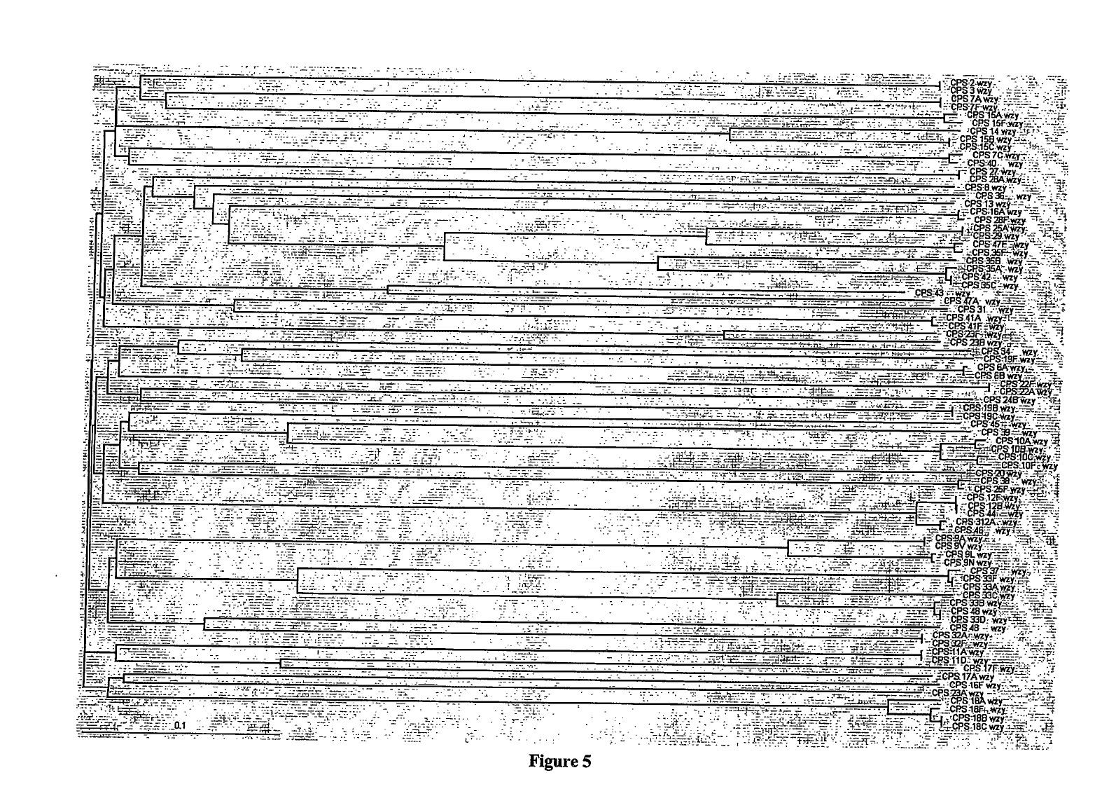

Analysis of homology and protein hydrophobicity was performed to annotate the wzx and wzy genes in S. pneumoniae cps gene cluster. Blast and PSI-blast (Altschul et al., 1997) were used for searching databases including GenBank and Pfam protein motif database (Bateman et al., 2002) for possible gene functions. The TMHMM v2.0 analysis program (Chen et al., 2003) was used to identify potential transmembrane segments from the amino acid sequence. Sequence alignment and comparison were done using the program ClustalW (Thompson et al., 1994). The phylogenetic trees were generated by neighbour-joining method using programme MEGA (Kumar et al. 1994) (FIGS. 4 and 5).

Oligonucleotide Primers

In addition to our previous MCT primers (Example 1) numerous serotype(s)-specific oligonucleotide primers, targeting wzy and wzx (one pair), were designed for this study. The specificity, sequences, numbered base positions and melting temperatures (Tm) are shown in Table 7. Expected amplicon lengths of different primer pairs can be calculated from the 5′-end positions of the corresponding primers.

DNA Preparation, PCR, Sequencing and Sequence Analysis

DNA extraction, PCR, sequencing and sequence analysis were performed as described Example 1. The only exception was that, for the new PCRs, 55-60° C. was used as annealing temperature because of the low Tm values of the new primers.

Nucleotide Sequence Accession Numbers

56 new sequences generated in this study, for partial cpsA (wzg)-cpsB (wzh) genes were deposited in GenBank with accession numbers: AY508586-AY508641. These sequences form part of the present invention.

Results and Discussion

Conventional Serotyping (CS) Results

Conventional serotyping, of 23 strains, was repeated because of apparent sharing of sequence types between two or more serotypes. After careful repetitions by two different persons, a previous serotype 42 isolate was confirmed to be serotype 31 and a previous serotype 41F isolate to be serotype 41A (Example 1); serotypes of three additional isolates were also corrected. The serotypes of the other 15 isolates were confirmed to be as previously defined (including all the serotypes 27, 28F and 16A isolates, one each of serotypes 6A, 38 and 25F isolate). The final results are shown in Table 8.

Partial cpsA-cpsB Sequencing Primers

The sequencing primers cpsS1-cpsA3 produced amplicons from all strains studied in this and our previous study, except for two belonging to rare serotypes, 25F and 38, and five that were non-serotypeable (Example 1). Two additional primer pairs, cpsS1-cpsA1 and cpsS3-cpsA2, formed amplicons from strains belonging to serotypes 25F and 38 and two non-serotypeable isolates.

TABLE 7
Oligonucleotide primers used in this study.

| | | | |
|---|---|---|---|
| | Sequence and orientation of | | |
| Name of rimers | oligo-nucleotides | Positions | Tm |

| | | | |
|---|---|---|---|
| 10A-10B-wzy-sense | 5′-TTGAGCTATTTAAGGACCTGGG-3′ | 395 | 58.4 |
| | (SEQ ID NO:144) | | |
| 10A-10B-wzy-antisense | 3′-AGTTCTTTCACTGCGAACGATT-5′ | 677 | 58.4 |
| | (SEQ ID NO:145) | | |
| 10C-10F-wzy-sense | 5′-GTCAATAAGTTTAAGTGTTATAGGGC-3′ | 51 | 59.0 |
| | (SEQ ID NO:146) | | |
| 10C-10F-wzy-antisense | 3′-CAAGCGTTGTGGGTAGTGATAT-5′ | 337 | 63.5 |
| | (SEQ ID NO:147) | | |
| 13-wzy-sense | 5′-GATGGGAAAATACGATATGCTC-3′ | 427 | 56.1 |
| | (SEQ ID NO:148) | | |
| 13-wzy-antisense | 3′-CGACCTCAAAACAGTACCTCAA-5′ | 736 | 58.5 |
| | (SEQ ID NO:149) | | |
| 20-wzy-sense | 5′-CTTTATCAGGAATACGCCAATC-3′ | 383 | 56.5 |
| | (SEQ ID NO:150) | | |
| 20-wzy-antisense | 3′-GCAACCAAGAGCAATAATATGTCC-5′ | 683 | 58.3 |
| | (SEQ ID NO:151) | | |
| 13-wzx-sense | 5′-CTTTTCTTCGTATGCTTTAGGG-3′ | 93 | 56.3 |
| | (SEQ ID NO:152) | | |
| 13-wzx-antisense | 3′-GACTATCCACATTAGAGATAGAAGG-5′ | 460 | 53.9 |
| | (SEQ ID NO:153) | | |
| 20-wzx-sense | 5′-GTTCTTTGTTTGACCCTTCCTT-3′ | 289 | 57.2 |
| | (SEQ ID NO:154) | | |
| 20-wzx-antisense | 3′-TATCTTATGCGGTCTGTCGTAA-5′ | 604 | 56.4 |
| | (SEQ ID NO:155) | | |
| 16F-wzy-sense | 5′-TTGTTCTTACATTTAGCCGTAGTG-3′ | 434 | 56.9 |
| | (SEQ ID NO:156) | | |
| 16F-wzy-antisense | 3′-GACAGTGAGATAGTGAGTCGTTTA-5′ | 777 | 55.9 |
| | (SEQ ID NO:157) | | |
| 27-wzy-sense | 5′-CAGAGTTTGGTCGAGGTTCCTA-3′ | 455 | 58.7 |
| | (SEQ ID NO:158) | | |
| 27-wzy-antisense | 3′-GAGTTAGTTGCTGCCTTTAGTG-5′ | 782 | 59.7 |
| | (SEQ ID NO:159) | | |
| 28F-16A-wzy-sense | 5′-GATCCGCTCACGGTATGGACTA-3′ | 261 | 61.6 |
| | (SEQ ID NO:160) | | |
| 28F-16A-wzy-antisense | 3′-GAATAACCGACTGTCGTTTTAA-5′ | 581 | 57.1 |
| | (SEQ ID NO:161) | | |
| 16F-wzx-sense | 5′-TTTATGAGGAGAGTACTGTATCAGA-3′ | 1219 | 53.1 |
| | (SEQ ID NO:162) | | |
| 16F-wzx-antisense | 3′-ACTCAAGCTATCGATAGTAATTTGT-5′ | 1433 | 56.6 |
| | (SEQ ID NO:163) | | |
| 27-wzx-sense | 5′-TACATTTTTATGAGAAGAGCATTG-3′ | 1213 | 54.6 |
| | (SEQ ID NO:164) | | |
| 27-wzx-antisense | 3′-GCTATCAGTACTATTTTTTTGTCAC-5′ | 1439 | 56.4 |
| | (SEQ ID NO:165) | | |
| 33A-specific-sense | 5′-TTGTTGTTGGGATTGTCTTGGG-3′ | length | 62.1 |
| | (SEQ ID NO:166) | | |
| 33A-specific-antisense | 3′-GTTTCAAGGCTTTAGGTTTCCG-5′ | 264 bp | 62.9 |
| | (SEQ ID NO:167) | | |
| 9V-specific-sense | 5′-TCTTTGATTTCATCAGGGATTG-3′ | length | 57.0 |
| | (SEQ ID NO:168) | | |
| 9V-specific-antisense | 3′-ATCACCATTGACGCAATCAGGA-5′ | 545 bp | 54.2 |
| | (SEQ ID NO:169) | | |
| 15A-15B-15C-wzx-sense | 5′-ATTGCGACTGTTAAACGAGAAG-3′ | 202 | 57.0 |
| | (SEQ ID NO:170) | | |
| 15A-15B-15C-wzx- | 3′-CCGTGTCTAAATACCTTTATGT-5′ | 514 | 55.0 |
| antisense | (SEQ ID NO:171) | | |
| 15B-15C-wzy-sense | 5′-TAATAAGCGGATGATTGTAGCG-3′ | 693 | 58.1 |
| | (SEQ ID NO:172) | | |
| 15B-15C-wzy-antisense | 3′-GGGTAGACCTTTCAATTAGTCA-5′ | 1041 | 55.5 |
| | (SEQ ID NO:173) | | |
| 15A-wzy-sense | 5′-TATTTCCTTCCTATGGGACAAC-3′ | 840 | 55.6 |
| | (SEQ ID NO:174) | | |
| 15A-wzy-antisense | 3′-CACCACTACTAATCGTAATAACA-5′ | 1100 | 54.2 |
| | (SEQ ID NO:175) | | |
| 22F-22A-wzy-sense | 5′-AGGATGCAGTAGATACCAGTGG-3′ | 398 | 56.1 |
| | (SEQ ID NO:176) | | |
| 22F-22A-wzy-antisense | 3′-CCTGTTGTTGGAGGCAAATATC-5′ | 752 | 56.2 |
| | (SEQ ID NO:177) | | |
| 22F-22A-wzx-sense | 5′-GGTTCTATCAAGGAAAAGAGGAC-3′ | 404 | 56.3 |
| | (SEQ ID NO:178) | | |
| 22F-22A-wzx-antisense | 3′-CAACCCAAGTCACTAACGATAA-5′ | 672 | 56.3 |
| | (SEQ ID NO:179) | | |
| 11A-specific-sense | 5′-CACTTCCATATCCAGCAT-3′ | 727-744 | 47.5 |
| | (SEQ ID NO:180) | | |
| 11A-specific-antisense | 3′-GACAGAGGACTATCAAGAGT-5′ | 970-989 | 46.4 |
| | (SEQ ID NO:181) | | |
| 7A-wzy-specific-sense | 5′-GCAAGTGTTTCAATGGGAGTA-3′ | 76 | 55.3 |
| | (SEQ ID NO:182) | | |
| 7A-wzy-specific-antisense | 3′-GAATAACATACCAGGGAGGCA-5′ | 420 | 56.1 |
| | (SEQ ID NO:183) | | |
| 7A-wzx-specific-sense | 5′-TTTGAGAATGCGGATAAGGTG-3′ | 730 | 58.0 |
| | (SEQ ID NO:184) | | |
| 7A-wzx-specific-antisense | 3′-GAGTAACATTGTCCCGTTTGAA-5′ | 1060 | 56.7 |
| | (SEQ ID NO:185) | | |
| 11A-11D-wzy-specific- | 5′-CGAAATATCGCCATTCATCAG-3′ | 190 | 58.4 |
| sense | (SEQ ID NO:186) | | |
| 11A-11D-wzy-specific- | 3′-TCACCGTGTCAACGACAACTAA-5′ | 570 | 59.8 |
| antisense | (SEQ ID NO:187) | | |
| 11A-11D-wzx-specific- | 5′-CAATCAATAATGCCGCATAC-3′ | 856 | 54.3 |
| sense | (SEQ ID NO:188) | | |
| 11A-11D-wzx-specific- | 3′-CTAAAGCAATCAAAGGTGTCCA-5′ | 1140 | 55.6 |
| antisense | (SEQ ID NO:189) | | |
| 12B-wzy-specific-sense | 5′-TGGAGGAGCAACTGACGTATT-3′ | 518 | 57.3 |
| | (SEQ ID NO:190) | | |
| 12B-wzy-specific- | 3′-GAGAACTTATACCTGCCACCT-5′ | 783 | 57.5 |
| antisense | (SEQ ID NO:191) | | |
| 12B-wzx-specific-sense | 5′-GTATGTTATTCGTTAGACAAACTGG-3′ | 1058 | 55.6 |
| | (SEQ ID NO:192) | | |
| 12B-wzx-specific- | 3′-GACATCCAAATACATAACGCTCAA-5′ | 1363 | 56.0 |
| antisense | (SEQ ID NO:193) | | |
| 17F-wzy-specific-sense | 5′-CTATTTACCTTGTTTCCTGCAAC-3′ | 490 | 56.1 |
| | (SEQ ID NO:194) | | |
| 17F-wzy-specific-antisense | 3′-CTATTGCGATACAGTCGTTAAG-5′ | 838 | 54.9 |
| | (SEQ ID NO:195) | | |
| 17F-wzx-specific-sense | 5′-GGATTACAAGAAATTCCCTCG-3′ | 722 | 56.0 |
| | (SEQ ID NO:196) | | |
| 17F-wzx-specific-antisense | 3′-TCCACTATACGCCTCGGTTAT-5′ | 1094 | 59.8 |
| | (SEQ ID NO:197) | | |
| 47F-wzy-specific-sense | 5′-TTTGGGTCTCCTTTACCTATC-3′ | 725 | 53.2 |
| | (SEQ ID NO:198) | | |
| 47F-wzy-specific-antisense | 3′-CACTACTTCTCAATCCCCTTT-5′ | 1195 | 53.7 |
| | (SEQ ID NO:199) | | |
| 25A-29-wzy-specific-sense | 5′-CCGAAAATTGTTCACAGGATAC-3′ | 112 | 56.8 |
| | (SEQ ID NO:200) | | |
| 25A-29-wzy-specific- | 3′-CTATACGGAACATAGGTAGTTAG-5′ | 474 | 55.9 |
| antisense | (SEQ ID NO:201) | | |
| 47F-wzx-specific-sense | 5′-AGCAGCAATTGTTTCTGTCTTAACA-3′ | 1128 | 60.6 |
| | (SEQ ID NO:202) | | |
| 47F-wzx-specific-antisense | 3′-GAGATTTTCACTATCTACACTATCTT-5′ | 1389 | 52.8 |
| | (SEQ ID NO:203) | | |
| 25A-29-wzx-specific-sense | 5′-CTCCCTATCATTACTACTCCCTATG-3′ | 58 | 56.2 |
| | (SEQ ID NO:204) | | |
| 25A-29-wzx-specific- | 3′-AATCCACGCTGTCAAGAAAGTG-5′ | 274 | 57.4 |
| antisense | (SEQ ID NO:205) | | |
| 10C-10F-wzy-specific- | 5′-GTCAATAAGTTTAAGTGTTATAGGGC-3′ | 51 | 56.2 |
| sense | (SEQ ID NO:206) | | |
| 10C-10F-wzy-specific- | 3′-CAAGCGTTGTGGGTAGTGATAT-5′ | 337 | 57.8 |
| | (SEQ ID NO:207) | | |
| 7C-wzy-sense | 5′-ACTCAAGTATCTGTGC/TCACCTT-3′ | 453 | 55.7 |
| | (SEQ ID NO:208) | | |
| 7C-wzy-antisense | 3′-CCTCGTCCATCTCCTTCACTAA-5′ | 703 | 57.1 |
| | (SEQ ID NO:209) | | |
| 7C-wzx-sense | 5′-TGAGTTTCCGATTAGAGCAG-3′ | 317 | 53.0 |
| | (SEQ ID NO:210) | | |
| 7C-wzx-antisense | 3′-CCTTACTACGCCATCCATA-5′ | 740 | 54.4 |
| | (SEQ ID NO:211) | | |
| 9L-9N-wzy-sense | 5′-TCAATGGCGACTTTATTTGC-3′ | 72 | 55.0 |
| | (SEQ ID NO:212) | | |
| 9L-9N-wzy-antisense | 3′-CGTGGGATGTCCTCTATTATCTGA-5′ | 434 | 56.2 |
| | (SEQ ID NO:213) | | |
| 9L-9N-wzx-sense | 5′-GTACCGCAAGCTATTCTAATGA-3′ | 388 | 54.9 |
| | (SEQ ID NO:214) | | |
| 9L-9N-wzx-antisense | 3′-GTCATTCTATCCGCTTCAAATAG-5′ | 853 | 53.4 |
| | (SEQ ID NO:215) | | |
| 17A-wzy-sense | 5′-TAGACTTCTTAGAGCCTATTGTGG-3′ | 722 | 55.3 |
| | (SEQ ID NO:216) | | |
| 17A-wzy-antisense | 3′-CTGGTTATCGCGTTTGACAATA-5′ | 1040 | 56.9 |
| | (SEQ ID NO:217) | | |
| 17A-wzx-sense | 5′-CAAACCCTTAGTCCAATATGGCTG-3′ | 624 | 62.2 |
| | (SEQ ID NO:218) | | |
| 17A-wzx-antisense | 3′-CCGATGGATAATAAGGGAAGCAAC-5′ | 988 | 61.0 |
| | (SEQ ID NO:219) | | |
| 23A-wzy-sense | 5′-CATTTGGTATGGGAGTAGGGAG-3′ | 1049 | 58.1 |
| | (SEQ ID NO:220) | | |
| 23A-wzy-antisense | 3′-GTGAAAGAGGATTGAGTACGTGG-5′ | 1326 | 58.5 |
| | (SEQ ID NO:221) | | |
| 33B-48-wzy-sense | 5′-TAATCAA/GTGGTCTGGTGGTCA/GA-3′ | 453 | 57.9 |
| | (SEQ ID NO:222) | | |
| 33B-48-wzy-antisense | 3′-GAAAC/TAAT/CGAGGATAACT/CGACT-5′ | 815 | 57.2 |
| | (SEQ ID NO:223) | | |
| 23F-wzy-sense | 5′-TGTCAGGAGAAAATATGACGC-3′ | 402 | 56.4 |
| | (SEQ ID NO:224) | | |
| 23F-wzy-antisense | 3′-CCTTTATGCTGCTTCCCAATAC-5′ | 766 | 58.4 |
| | (SEQ ID NO:225) | | |
| 34-wzy-sense | 5′-TTGTTGTAGTGGCAGTTGCTCC-3′ | 740 | 60.4 |
| | (SEQ ID NO:226) | | |
| 34-wzy-antisense | 3′-CGGATGTCCCTTACAGAAATGTTG-5′ | 1070 | 59.4 |
| | (SEQ ID NO:227) | | |
| 35A-wzy-sense | 5′-TCCTGATTATG/ATTGAGATTTG/CG-3′ | 399 | 54.7 |
| | (SEQ ID NO:228) | | |
| 35A-wzy-antisense | 3′-GACCTAACGCTTCTGAATGAAT-5′ | 747 | 54.8 |
| | (SEQ ID NO:229) | | |
| 36-wzy-sense | 5′-CAATTTCCCCTTATTCTGTAGTTC-3′ | 692 | 56.8 |
| | (SEQ ID NO:230) | | |
| 36-wzy-antisense | 3′-CTCTCTTGTCATATTTGTCCCAGTT-5′ | 1026 | 57.0 |
| | (SEQ ID NO:231) | | |
| 39(1)-wzy-sense | 5′-GATTGGTTTGGGAACTTGATGTC-3′ | 232 | 60.2 |
| | (SEQ ID NO:232) | | |
| 39-wzy-antisease | 3′-CACCATACTCCATAGTAAATCGTCC-5′ | 518 | 59.5 |
| | (SEQ ID NO:233) | | |
| 41A-wzy-sense | 5′-GTAGTTACTGGCCCTTTCTTATTCC-3′ | 511 | 59.7 |
| | (SEQ ID NO:234) | | |
| 41A-wzy-antisense | 3′-GTTCTACGTCTATCAAAGAGCGAT-5′ | 828 | 59.0 |
| | (SEQ ID NO:235) | | |
| 41A-wzx-sense | 5′-CAGCAAATGCAGGTTCTCAAA-3′ | 278 | 59.0 |
| | (SEQ ID NO:236) | | |
| 41A-wzx-antisense | 3′-ACTGTGGAGCAGATCGTATAGTAAT-5′ | 566 | 58.9 |
| | (SEQ ID NO:237) | | |
| 43-wzy-sense | 5′-GATCAAATGGTGGTATTAGGAA-3′ | 251 | 54.0 |
| | (SEQ ID NO:238) | | |
| 43-wzy-antisense | 3′-CGGTCAGTATAAAAGGTTAAGA-5′ | 601 | 55.8 |
| | (SEQ ID NO:239) | | |
| 43-wzx-sense | 5′-TTCTTATCGCTTCCATTGTCAG-3′ | 907 | 57.5 |
| | (SEQ ID NO:240) | | |
| 43-wzx-antisense | 3′-CCACATTCACCTCGTCGTAAA-5′ | 1182 | 57.1 |
| | (SEQ ID NO:241) | | |
| 47A-wzy-sense | 5′-TATTTGCCATAACGGACTCTAGAAC-3′ | 485 | 59.5 |
| | (SEQ ID NO:242) | | |
| 47A-wzy-antisense | 3′-CACCAATACACCCAAATTAAGAAGC-5′ | 830 | 61.5 |
| | (SEQ ID NO:243) | | |
| 47A-wzx-sense | 5′-TTTGGGCTCTTTAGGTAGTGTAT-3′ | 687 | 55.4 |
| | (SEQ ID NO:244) | | |
| 47A-wzx-antisense | 3′-CTGCCTATTACAAGCTATGAAATG-5′ | 1064 | 55.3 |
| | (SEQ ID NO:245) | | |
| 48-wzy-sense | 5′-CATTTGGAGTTATTGCCCTAC-3′ | 602 | 54.5 |
| | (SEQ ID NO:246) | | |
| 48-wzy-antisense | 3′-CCCCAGAATTAAATCTTATACCC-5′ | 909 | 56.6 |
| | (SEQ ID NO:247) | | |
| 48-wzx-sense | 5′-AGGGCTTAACTGTTTCAGTGTT-3′ | 782 | 55.5 |
| | (SEQ ID NO:248) | | |
| 48-wzx-antisense | 3′-CTAAACCATATCGTCCTGACTT-5′ | 1113 | 54.2 |
| | (SEQ ID NO:249) | | |
| 33C-wzy-sense | 5′-CTGTGAAGACTTACAACATG-5′ | 197 | 45.3 |
| | (SEQ ID NO:250) | | |
| 33C-wzy-antisense | 3′-CTGTGAAGACTTACAACATG-5′ | 445 | 43.7 |
| | (SEQ ID NO:251) | | |
| 23B-wzy-sense | 5′-TTGGATCGTTGTTCATAGCGG-3′ | 639 | 61.0 |
| | (SEQ ID NO:252) | | |
| 23B-wzy-antisense | 3′-GACACCTTTACGGCAACGATTC-5′ | 947 | 62.5 |
| | (SEQ ID NO:253) | | |
| 23B-wzx-sense | 5′-AGCGAGCGGTATCATTCTATTTG-3′ | 897 | 60.8 |
| | (SEQ ID NO:254) | | |
| 23B-wzx-antisense | 3′-CTATCACAACTTCTTTAACGAGGTC-5′ | 1219 | 59.6 |
| | (SEQ ID NO:255) | | |
| 24B-wzy-sense | 5′-TCAACACTTATGATGGTGCCTG-3′ | 685 | 58.5 |
| | (SEQ ID NO:256) | | |
| 24B-wzy-antisense | 3′-ATCTTCACCCTAATAGCCCGA-5′ | 1025 | 58.3 |
| | (SEQ ID NO:257) | | |
| 25F-38-wzy-sense | 5′-AATCTGAGGAAACTTGGAGCAA-3′ | 641 | 58.5 |
| | (SEQ ID NO:258) | | |
| 25F-38-wzy-antisense | 3′-GCATAATTGCTAATCTTAACAAGG-5′ | 977 | 55.8 |
| | (SEQ ID NO:259) | | |
| 25F-38-wzx-sense | 5′-GCAATGGTTTATGGATGATAGAGCG-3′ | 702 | 64.3 |
| | (SEQ ID NO:260) | | |
| 25F-38-wzx-antisense | 3′-TGTGCTGCTAACGACCACGAAA-5′ | 1088 | 64.4 |
| | (SEQ ID NO:261) | | |
| 31-wzy-sense | 5′-TGAAAATCCCTTAGTGACATCTG-3′ | 492 | 56.5 |
| | (SEQ ID NO:262) | | |
| 31-wzy-antisense | 3′-GACCAGCATCGTAAAGAGTCTA-5′ | 794 | 56.5 |
| | (SEQ ID NO:263) | | |
| 32A-32F-wzy-sense | 5′-CGGTATGCTTACAATGAGACGC-3′ | 813 | 60.2 |
| | (SEQ ID NO:264) | | |
| 32A-32F-wzy-antisense | 3′-GTAGAATAGGCCCTTGCTTAAG-5′ | 1163 | 60.5 |
| | (SEQ ID NO:265) | | |
| 32A-32F-wzx-sense | 5′-GTAACGATGCCTAGAATGACTT-3′ | 799 | 53.6 |
| | (SEQ ID NO:266) | | |
| 32A-32F-wzx-antisense | 3′-CACACCATTATCCACGACAATAG-5′ | 1107 | 53.9 |
| | (SEQ ID NO:267) | | |
| 35B-wzy-sense | 5′-CTAATTTGGCTATGAAGCTAATCCC-3′ | 626 | 60.6 |
| | (SEQ ID NO:268) | | |
| 35B-wzy-antisense | 3′-CAAATGACTGACGCTGAAATCACTT-5′ | 1019 | 58.2 |
| | (SEQ ID NO:269) | | |
| 45-wzy-sense | 5′-CTATGCAGGAAATATCCGAGAAGG-3′ | 111 | 61.7 |
| | (SEQ ID NO:270) | | |
| 45-wzy-antisense | 3′-GTATCGCAAAGACAAAGTGCCTAG-5′ | 497 | 63.0 |
| | (SEQ ID NO:271) | | |
| 45-wzx-sense | 5′-AATGGCTTGCTCCTATTGCTGT-3′ | 929 | 60.9 |
| | (SEQ ID NO:272) | | |
| 45-wzx-antisense | 3′-CGTTTAGCAAGAACCCTATCATC-5′ | 1306 | 58.1 |
| | (SEQ ID NO:273) | | |
| 41F-wzx-sense | 5′-GTCAAAGACAGGAATGACATCTATG-3′ | 493 | 57.7 |
| | (SEQ ID NO:274) | | |
| 41F-wzx-antisense | 3′-CCCTCCTTCACGAAAATAAAGA-5′ | 972 | 56.9 |
| | (SEQ ID NO:275) | | |
| 18A-18-B-18C-18F-wzx- | 5′-GGAATCGGACAATAGCAC-3′ | 35 | 50.2 |
| sense | (SEQ ID NO:276) | | |
| 18A-18-B-18C-18F-wzx- | 3′-ACCAGAACTTCTCAAAGCAT-5′ | 265 | 50.5 |
| antisense | (SEQ ID NO:277) | | |
| 19B-19C-wzx-sense | 5′-GGCATCAAAGGTTAAGTG-3′ | 744 | 48.0 |
| | (SEQ ID NO:278) | | |
| 19B-19C-wzx-antisense | 3′-GAAGACAGCGTTGAGAAA-5′ | 1171 | 47.5 |
| | (SEQ ID NO:279) | | |
| 19F-wzx-sense | 5′-GCTATCTAACATTGCGAGTA-3′ | 672 | 48.4 |
| | (SEQ ID NO:280) | | |
| 19F-wzx-antisence | 3′-AAACCGAAGGACGAATAT-5′ | 967 | 49.1 |
| | (SEQ ID NO:281) | | |
| 2-wzx-sense | 5′-TAGCGGTGAATGGCATCT-3′ | 644 | 54.1 |
| | (SEQ ID NO:282) | | |
| 2-wzx-antisense | 3′-AGTTGGAATCATCCTCGCT-5′ | 1012 | 50.6 |
| | (SEQ ID NO:283.) | | |
| 23A-23F-wzx-sense | 5′-GGGAAATGGTTTACTATGC-3′ | 623 | 49.7 |
| | (SEQ ID NO:284) | | |
| 23A-23F-wzx-antisense | 3′-GTTCTTCTATTCTCGCC(T)A-5′ | 843 | 47.0 |
| | (SEQ ID NO:285) | | |
| 6A-6B-wzx-sense | 5′-ATTTATGAAGGGAAGATGG-3′ | 1003 | 49.0 |
| | (SEQ ID NO:286) | | |
| 6A-6B-wzx-antisense | 3′-CCGAGCGTCATTATCAAA-5′ | 1324 | 47.6 |
| | (SEQ ID NO:287) | | |
| 8-wzx-sense | 5′-TATGTTTCAAGGGTTCTG-3′ | 88 | 45.2 |
| | (SEQ ID NO :288) | | |
| 8-wzx-antisense | 3′-CCTTACCGTCGAATAATA-5′ | 356 | 47.4 |
| | (SEQ ID NO:289) | | |
| 9A-9V-wzx-sense | 5′-TGATAAGGCTTACCAGTT-3′ | 732 | 44.6 |
| | (SEQ ID NO:290) | | |
| 9A-9V-wzx-antisense | 3′-CTGACCATAACCCTGATT-5′ | 1360 | 44.0 |
| | (SEQ ID NO:291) | | |
| 12F-2B-44-46-wzy-sense | 5′-TGAATATGGACGGTGGAG-3′ | 767 | 51.1 |
| | (SEQ ID NO:292) | | |
| 12F-12B-44-46-wzy- | 3′-GAAAGCCGAAAGAAACGA-5′ | 1008 | 53.1 |
| antisense | (SEQ ID NO:293) | | |
| 14-wzy-sense | 5′-GATTGGCTGTTCAAGTGT-3′ | 230 | 47.3 |
| | (SEQ ID NO:294) | | |
| 14-wzy-antisense | 3′-CCCTGCCTAAATGTAATC-5′ | 463 | 47.2 |
| | (SEQ ID NO:295) | | |
| 16F-wzy-sense | 5′-TTGTTCTTACATTTAGCCGT-3′ | 434 | 50.6 |
| | (SEQ ID NO:296) | | |
| 16F-wzy-antisense | 3′-CCCTGAACCTAAACCATT-5′ | 737 | 49.9 |
| | (SEQ ID NO:297) | | |
| 18A-18-B-18C-18F-wzy- | 5′-CATGAAGTTGCACCTATT-3′ | 409 | 45.2 |
| sense | (SEQ ID NO:298) | | |
| 18A-18-B-18C-18F-wzy- | 3′-CCCTATCCCAAACATTGT-5′ | 840 | 47.2 |
| antisense | (SEQ ID NO:299) | | |
| 19F-wzy-sense | 5′-AAACGGAAAGTTGGATGG-3′ | 667 | 52.8 |
| | (SEQ ID NO:300) | | |
| 19F-wzy-antisense | 3′-CAGAAACGACATCCACGAA-5′ | 1075 | 49.9 |
| | (SEQ ID NO:301) | | |
| 2-3-wzy-sense | 5′-TGTCGGCATTGTATTCTTTA-3′ | 59 | 51.9 |
| | (SEQ ID NO:302) | | |
| 2-3-wzy-antisense | 3′-CCCAGTCCTAAACCACCA-5′ | 855 | 54.4 |
| | (SEQ ID NO:303) | | |
| 37-33F-33A-wzy-sense | 5′-TAGGGAAATGGGCGACTC-3′ | 101 | 55.4 |
| | (SEQ ID NO:304) | | |
| 37-33F-33A-wzy-antisense | 3′-ACCTCAAACCATAACTCGGA-5′ | 596 | 54.7 |
| | (SEQ ID NO:305) | | |
| 6A-6B-wzy-sense | 5′-ATTCCAGCGACTACACTT-3′ | 496 | 46.7 |
| | (SEQ ID NO:306) | | |
| 6A-6B-wzy-antisense | 3′-AATCACCACCATCTAACG-5′ | 634 | 45.2 |
| | (SEQ ID NO:307) | | |
| 8-wzy-sense | 5′-CACGCAGACTAGAACAGC-3′ | 606 | 48.5 |
| | (SEQ ID NO:308) | | |
| 8-wzy-antisense | 3′-GAACCAGATACATACGCCA-5′ | 1055 | 50.5 |
| | (SEQ ID NO:309) | | |
| 9A-9V-wzy-sense | 5′-GTTGGTTTCGACTCTTTG-3′ | 394 | 47.5 |
| | (SEQ ID NO:310) | | |
| 9A-9V-wzy-antisense | 3′-TTTTGCGATGACTGTTAC-5′ | 1017 | 45.7 |
| | (SEQ ID NO:311) | | |
| 19B-19C-wzy-sense | 5′-TTCGGAGATTTGTGGTAT-3′ | 478 | 47.5 |
| | (SEQ ID NO:312) | | |
| 19B-19C-wzy-antisense | 3′-AGCAAATACCTCCACCTA-5′ | 772 | 50.0 |
| | (SEQ ID NO:313) | | |
| 1-wzx-sense | 5′-TGGAGAATTTGCGATTACG-3′ | 744 | 54.5 |
| | (SEQ ID NO:314) | | |
| 1-wzx-antisense | 3′-TAGAGTCCCATTTGTCTCAC-5′ | 886 | 48.6 |
| | (SEQ ID NO:315) | | |
| 4-wzx-sense | 5′-AATGCTTGTACTACTCCCTC-3′ | 88 | 48.5 |
| | (SEQ ID NO:316) | | |
| 4-wzx-antisense | 3′-GATACTAAATGCCTACCG-5′ | 898 | 48.1 |
| | (SEQ ID NO:317) | | |
| 19A-wzx-sense | 5′-TTCCCTATGTCAGTCTATGAA-3′ | 1000 | 49.7 |
| | (SEQ ID NO:318) | | |
| 19A-wzx-antisense | 3′-TCTTCATAGTATCGGCTTAA-5′ | 1214 | 48.8 |
| | (SEQ ID NO:319) | | |
| 1-wzy-sense | 5′-TATTCTATTTCTTACCCGCTAC-3′ | 211 | 51.6 |
| | (SEQ ID NO:320) | | |
| 1-wzy-antisense | 3′-ATTCACCCGTTCAAAGTAGA-5′ | 801 | 52.4 |
| | (SEQ ID NO:321) | | |
| 4-wzy-sense | 5′-GTGCCTAGTAGCATTCCATA-3′ | 1003 | 50.5 |
| | (SEQ ID NO:322) | | |
| 4-wzy-antisense | 3′-GAAACCAATGATACCACCAC-5′ | 1198 | 50.4 |
| | (SEQ ID NO:323) | | |
| 19A-wzy-sease | 5′-TCGCCTAGTCTAAATACCAA-3′ | 235 | 50.7 |
| | (SEQ ID NO:324) | | |
| 19A-wzy-antisense | 3′-AAGTGAATCTTAAAGCCGTC-5′ | 975 | 53.4 |
| | (SEQ ID NO:325) | | |
| 17F-YS2-sense | 5′-AGAGGGATTGTTGAAGGTATTC-3′ | 754 | 59.8 |
| | (SEQ ID NO:326) | | |
| 17F-YA2-antisense | 3′-CCTACTATCTTTACGCTCTGAT-5′ | 1060 | 59.7 |
| | (SEQ ID NO:327) | | |
| 25F-38-YS-sense | 5′-GGCGTTGTCAGTGCTAGTTTAG-3′ | 121 | 62.6 |
| | (SEQ ID NO:328) | | |
| 25F-38-YA-antisense | 3′-CTCATATTACCGACGAAATTGTCC-5′ | 713 | 61.6 |
| | (SEQ ID NO:329) | | |
| 35F-47F-YS-sense | 5′-ATAAAAAGAAAGTCTTTGCCAGAG-3′ | 13 | 60.6 |
| | (SEQ ID NO:330) | | |
| 35F-47F-YA-antisense | 3′-CTACTACTTGTATCAGCGATAAC-5′ | 499 | 60.0 |
| | (SEQ ID NO:331) | | |
| 25A-29-YS-sense | 5′-CCGAAAATTGTTCACAGGATAC-3′ | 112 | 62.0 |
| | (SEQ ID NO:332) | | |
| 25A-29-YA-antisense | 3′-CTATACGGAACATAGGTAGTTAG-5′ | 474 | 60.9 |
| | (SEQ ID NO:333) | | |

Updated Sequence Type Nomenclature (Compared with Example 1)

Sequence types were generally named according to the corresponding serotype, with a suffix representing the source of the isolate for which the sequence type was first identified. When sequences characteristic of two to five serotypes were identified, the sequence type name included all, with the lower number serotype first (e.g 15B-15C-22F-22A etc.) (Henrichsen, 1995). Representative sequences of all sequence types were deposited into GenBank (see Table 8 for sequence type nomenclature and corresponding GenBank accession numbers).

TABLE 8
S. pneumoniae partial cpsA-cpsB sequence (˜800 bp) database and comparison of molecular capsular typing (MCT) and
conventional serotyping (CS) results of 519 S. pneumoniae isolates (and also including 24 GenBank sequences and 92 Sanger
Institute sequences).

| | | GenBank No. | Serotype/group-specific PCR | | |
|---|---|---|---|---|---|
| CS^{a} | Sequence types (n=)^{ab} | (positions)^{c} | (n=)^{a} | Final MCT (n=)^{ab} | Comments^{ad} |
| 1 | 1-g (g) | Z83335 (4545-5343) | 1 (1) | 1 (1) | Correlate |
| | 1-q (9 + 1A) | AF532632 | 1 (9 + 1A) | 1 (9 + 1A) | Correlate |
| 2 | 2-g (g) | AF026471 (2412-3210) | 2 (1) | 2 (1) | Correlate |
| | 2-q (3) | AF532669 | 2 (3) | 2 (3) | Correlate |
| | 2-41A (s) | 20602 (2612-3410) | 2 (1) | 2 (1) | Correlate |
| 3 | 3-g (g) | Z47210 (2413-3210) | 3 (1) | 3 (1) | Correlate |
| | 3-q (15 + qap) | AF532682 | 3 (16) | 3 (16) | Correlate |
| | 3-c (1) | AF532681 | 3 (1) | 3 (1) | Correlate |
| | 3-nz (1) | AF532683 | 3 (1) | 3 (1) | Correlate |
| 4 | 4 (gx2 + 36 + qap) | AF316639 (2470-3268), | 4 (39) | 4 (39) | Correlate |
| | | NC_003028 | | | |
| | | (genome); AF532693 | | | |
| 5 | 5-q (4) | AF532697 | NA | 5 (4) | Correlate |
| | 5-c (1) | AF532696 | NA | 5 (1) | Correlate |
| | 5-qap (qap) | AY508634 | NA | 5 (1) | Correlate |
| 6A | 6A-g (g) | AY078347 (1169-1967) | Serogroup 6 (1) | 6 (1) | Correlate |
| | 6A-c1 (2) | AF532699 | Serogroup 6 (2) | 6A (2) | Correlate |
| | 6A-c2 (1) | AF532700 | Serogroup 6 (1) | 6A (1) | Correlate |
| | 6A-n (2) | AF532698 | Serogroup 6 (2) | 6A (2) | Correlate |
| | 6A-qap (qap) | AY508641 | Serogroup 6 (1) | 6A (1) | Correlate |
| | 6A-6B-g (1) | AF532701 | Serogroup 6 (1) | 6A or 6B (1) | Consistent |
| | 6A-6B-q (1) | AY330713 | Serogroup 6 (1) | 6A or 6B (1) | Consistent |
| | 6A-6B-s (5 + s) | AF532702/ | Serogroup 6 (6) | 6A or 6B (6) | Consistent |
| | | 17611 (2259-3057) | | | |
| 6B | 6B-c (1) | AF532704 | Serogroup 6 (1) | 6B (1) | Correlate |
| | 6A-6B-g (g + 5) | AF316640 (2154-2952); | Serogroup 6 (6) | 6A or 6B (6) | Consistent |
| | | AF532703 | | | |
| | 6A-6B-q (9) | AF532705 | Serogroup 6 (9) | 6A or 6B (9) | Consistent |
| | 6A-6B-s (s) | 17506 (2157-2955) | Serogroup 6 (1) | 6A or 6B (1) | Consistent |
| 7F | 7F-7A (15 + qap + s) | AF532707/ | NA | 7F or 7A (17) | Consistent |
| | | 20024 (2531-3329) | | | |
| 7A | 7A-cn (cn) | AY508635 | NA | 7A (1) | Correlate |
| | 7F-7A (s) | 24019 (2502-3300) | NA | 7F or 7A (1) | Consistent |
| 7B | 7B-40(cnx2) | AY508636, | 7B or 7C or 40 (2) | 7B or 40 (2) | Consistent |
| | | AY508627 | | | |
| 7C | 7C-19C-24B (7 + s) | AF532706/ | 7B or 7C or 40 (8) | 7C (8) | Correlate |
| | | 21759 (2804-3602) | | | |
| 8 | 8-g (gx2 + 12) | AF31664 (2511-3309), | 8 (14) | 8 (14) | Correlate |
| | | AJ239004; | | | |
| | | AF532708 | | | |
| | 8-s (s) | 13844 (2518-3316) | 8 (1) | 8 (1) | Correlate |
| 9A | 9A-9V (cn + s) | AY508637/ | 9A or 9V (2) | 9A or 9V (2) | Consistent |
| | | 20538 (2486-3284) | | | |
| 9L | 9L-cn (cn + s) | AY508638/ | NA | 9L (2) | Correlate |
| | | 17618 (2805-3603) | | | |
| 9N | 9N (9 + s) | AF532709/ | NA | 9N (10) | Correlate |
| | | 17619 (2805-3603) | | | |
| 9V | 9V (g + 17) | AF402095 (1520-2318); | 9A or 9V (18) | 9V (18) | Correlate |
| | | AF532710 | | | |
| | 9A-9V (cn + s) | AY508639/ | 9A or 9V (2) | 9A or 9V (2) | Consistent |
| | | 20856 (2803-3601) | | | |
| 9V and 14 | 9V (1) | AF402095 (1520-2318); | 9V and 14 (1) | 9V and 14 (1) | Correlate |
| | | AF532710 | | | |
| 10F | 10F-q (3) | AF532635 | 10F or 10C (3) | 10F (3) | Correlate |
| | 10F-ca (2) | AF532636 | 10F or 10C (2) | 10F (2) | Correlate |
| | 10F-10C (qap + s) | AY508587/ | 10F or 10C (2) | 10F or 10C (2) | Consistent |
| | | 18532 (2201-2999) | | | |
| 10A | 10A-17A (5 + cn + s) | AF532633/ | 10A or 10B (7) | 10A (7) | Correlate |
| | | 17290 (2451-3249) | | | |
| | 10A-23F (6) | AF532634 | 10A or 10B (6) | 10A (6) | Correlate |
| 10B | 10B-10C (cn + s) | AY508586/ | 10A or 10B (2) | 10B (2) | Correlate |
| | | 16991 (2154-2952) | | | |
| 10C | 10F-10C (s) | 18126 (2095-2893) | 10F or 10C (1) | 10F or 10C (1) | Consistent |
| 11F | | | | | |
| 11A | 11A-nz (1) | AF532638 | 11A or 11D (1) | 11A (1) | Correlate |
| | 11A-11D-18F (7 + s) | AF532637/ | 11A or 11D (8) | 11A or 11D (8) | Consistent |
| | | 17948 (3506-4304) | | | |
| 11B | 11B-11C (1 + cn) | AF532639 | NA | 11B or 11C (2) | Consistent |
| 11C | 11B-11C (cn) | AY508588 | NA | 11B or 11C (1) | Consistent |
| | 11C-cn (cn) | AY508589 | NA | 11C (1) | Correlate |
| 11D | 11A-11D-18F (s) | 17213 (2852-3650) | 11A or 11D (1) | 11A or 11D (1) | Consistent |
| 12F | 12F-q (8 + 1B) | AF532640 | Serogroup 12 or 44 or 46 | 12F (8 + 1B) | Correlate |
| | | | (8 + 1B) | | |
| | 12F-12A-12B (1 + s) | AF532641/ | Serogroup 12 or 44 or 46 (2) | 12F or 12A or 12B (2) | Consistent |
| | | 23778 (2154-2952) | | | |
| 12A | 12A-cn (cn) | AY508590 | Serogroup 12 or 44 or 46 (1) | 12A (1) | Correlate |
| | 12A-46 (s) | 27104 (4224-5022) | Serogroup 12 or 44 or 46 (1) | 12A or 46 (1) | Consistent |
| | 12F-12A-12B (cnx2) | AY508591 | Serogroup 12 or 44 or 46 (2) | 12F or 12A or 12B (2) | Consistent |
| 12B | 12F-12A-12B (s) | 23673 (2153-2951) | Serogroup 12 or 44 or 46 (1) | 12F or 12A or 12B (1) | Consistent |
| 13 | 13-20 (6 + s) | AF532642/ | 13 (7) | 13 (7) | Correlate |
| | | 17717 (2486-3284) | | | |
| 14 | 14-g (g) | X85787 (2369-3167) | 14 (1) | 14 (1) | Correlate |
| | 14-q (23 + 1C) | AF532643 | 14 (23 + 1C) | 14 (23 + 1C) | Correlate |
| | 14-v (9 + s) | AF532644/ | 14 (10) | 14 (10) | Correlate |
| | | 19918 (2150-2948) | | | |
| | 14-c (1) | AF532645 | 14 (1) | 14 (1) | Correlate |
| 15F | 15F-cn1 (cnx2 + s) | AY508594/ | 15F or 15A (3) | 15F (3) | Correlate |
| | | 22405 (2503-3301) | | | |
| | 15F-cn2 (cn) | AY508595 | 15F or 15A (1) | 15F (1) | Correlate |
| 15A | 15A-ca1 (1 + v) | AF532646 | 15F or 15A (2) | 15A (2) | Correlate |
| | 15A-ca2 (3 + s) | AF532647/ | 15F or 15A (4) | 15A (4) | Correlate |
| | | 18517 (2152-2950) | | | |
| 15B | 15B-c (1) | AF532648 | 15B or 15C (1) | 15B (1) | Correlate |
| | 15B-15C (6) | AF532649 | 15B or 15C (6) | 15B or 15C (6) | Consistent |
| | 15B-15C-22F-22A (2 + s) | AF532650/ | 15B or 15C (3) | 15B or 15C (3) | Consistent |
| | | 18624 (2154-2952) | | | |
| 15C | 15C-ca (1) | AF532652 | 15B or 15C (1) | 15C (1) | Correlate |
| | 15C-q1 (1) | AF532651 | 15B or 15C (1) | 15C (1) | Correlate |
| | 15C-q2 (2) | AY330714 | 15B or 15C (2) | 15C (2) | Correlate |
| | 15C-q3 (1) | AY330715 | 15B or 15C (1) | 15C (1) | Correlate |
| | 15C-s (s) | 18262 (2154-2952) | 15B or 15C (1) | 15C (1) | Correlate |
| | 15B-15C (v) | AY508593 | 15B or 15C (1) | 15B or 15C (1) | Consistent |
| | 15B-15C-22F-22A (v) | AY508592 | 15B or 15C (1) | 15B or 15C (1) | Consistent |
| 16F | 16F-q (5 + cn + s) | AF532653/ | NA | 16F (7) | Correlate |
| | | 21481 (2508-3306) | | | |
| | 16F-nz (1) | AF532654 | NA | 16F (1) | Correlate |
| 16A | 16A-28F (cn + s) | AY508596/ | 16A (2) | 16A (2) | Correlate |
| | | 21730 (2147-2945) | | | |
| 17F | 17F-n (3 + s + 1D) | AF532656/ | 17F-n (4 + 1D) | 17F (4 + 1D) | Correlate |
| | | 22896 (2489-3287) | | | |
| | 17F-35B-35C-42 (2) | AF532657 | 17F (2) | 17F (2) | Correlate |
| 17A | 17A-ca (1 + s) | AF532655/ | 17A (2) | 17A (2) | Correlate |
| | | 23198 (1645-2443) | | | |
| | 10A-17A (cn) | AY508597 | 17A (1) | 17A (1) | Correlate |
| 18F | 18F-ca (1) | AF532662 | Serogroup 18 (1) | 18F (1) | Correlate |
| | 18F-w (1) | AY330716 | Serogroup 18 (1) | 18F (1) | Correlate |
| | 11A-11D-18F (cn + s) | AY508598/ | 18F (2)^{e} | 18F (2) | Correlate |
| | | 22849 (2530-3328) | | | |
| 18A | 18A-nz (5 + qap + s) | AF532659/ | Serogroup 18 (7) | 18A-nz (7) | Correlate |
| | | 21887 (2247-3045) | | | |
| | 18A-q (1) | AF532658 | Serogroup 18 (1) | 18A (1) | Correlate |
| 18B | 18B-18C (4 + s) | AF532660/ | Serogroup 18 (5) | 18B or 18C (5) | Consistent |
| | | 21819 (2153-2951) | | | |
| 18C | 18B-18C (g + 14 + s) | AF316642 (2052-2850); | Serogroup 18 (16) | 18B or 18C (16) | Consistent |
| | | AF532661/ | | | |
| | | 21819 (2153-2951) | | | |
| 19F | 19F-g1 (gx4 + 7 + s) | AF030367 (4724-5522), | 19F (12) | 19F (12) | Correlate |
| | | AF030368, | | | |
| | | AF030370, | | | |
| | | AF030371; | | | |
| | | AF532667/ | | | |
| | | 19798 (4425-5223) | | | |
| | 19F-g2 (gx2) | AF030369 (2455-3253), | 19F (2) | 19F (2) | Correlate |
| | | AF030372 | | | |
| | 19F-g3 (g) | U09239 (1119-1917) | 19F (1) | 19F (1) | Correlate |
| | 19F-q (9) | AF532666 | 19F (9) | 19F (9) | Correlate |
| | 19F-n (3) | AF532668 | 19F (3) | 19F (3) | Correlate |
| | 19F-c (1) | AF532665 | 19F (1) | 19F (1) | Correlate |
| 19A | 19A-g (g) | AF094575 (2683-3481) | 19A (1) | 19A (1) | Correlate |
| | 19A-q (8) | AF532663 | 19A (8) | 19A (8) | Correlate |
| | 19A-ca (3) | AF532664 | 19A (3) | 19A (3) | Correlate |
| 19B | 19B-cn (cnx3 + s) | AY508599/ | 19B or 19C (4) | 19B (4) | Correlate |
| | | 21568 (2171-2969) | | | |
| 19C | 19C-cn1 (cn) | AY508600 | 19B or 19C (1) | 19C (1) | Correlate |
| | 19C-cn2 (cnx2) | AY508601 | 19B or 19C (2) | 19C (2) | Correlate |
| | 7C-19C-24B (s) | 25632 (4069-4867) | 19B or 19C (1) | 19C (1) | Correlate |
| 20 | 13-20 (8 + s) | AF532670/ | 20 (9) | 20 (9) | Correlate |
| | | 20453 (2486-3284) | | | |
| 21 | 21-ca (1) | AF532671 | NA | 21 (1) | Correlate |
| | 21-cn (cn) | AY508602 | NA | 21 (1) | Correlate |
| 22F | 15B-15C-22F-22A | AF532673/ | 22F or 22A (15) | 22F or 22A (15) | Consistent |
| | (13 + qap + s) | 22696 (2486-3284) | | | |
| 22A | 22A (4) | AF532672 | 22F or 22A (4) | 22A (4) | Correlate |
| | 15B-15C-22F-22A (s) | 22591 (2486-3284) | 22F or 22A (1) | 22F or 22A (1) | Consistent |
| 23F | 23F-c (1) | AF532678 | 23F (1) | 23F (1) | Correlate |
| | 10A-23F (gx3 + 18 + s) | AF057294 (2991-3789), | 23F (22) | 23F (22) | Correlate |
| | | AF030373, | | | |
| | | AF030374; | | | |
| | | AF532677/ | | | |
| | | 22330 (2852-3650) | | | |
| | 23F-23A (1) | AF532679 | 23F (1) | 23F (1) | Correlate |
| 23A | 23A-ca (3 + s) | AF532675/ | 23A (4) | 23A (4) | Correlate |
| | | 21475 (2154-2952) | | | |
| | 23F-23A (1) | AF532674 | 23A (1) | 23A (1) | Correlate |
| 23B | 23B-c (2 + s) | AF532676/ | NA | 23B (3) | Correlate |
| | | 23047 (3537-4335) | | | |
| | 23B-q (2) | AY330717 | NA | 23B (2) | Correlate |
| 24F | 24F-cn1 (cn) | AY508605 | NA | 24F (1) | Correlate |
| | 24F-cn2 (cn) | AY508606 | NA | 24F (1) | Correlate |
| | 24F-cn3 (cn) | AY508607 | NA | 24F (1) | Correlate |
| 24A | 24A-cn (cn) | AY508603 | NA | 24A (1) | Correlate |
| 24B | 7C-19C-24B (cn + s) | AY508604/ | 24B (2)^{g} | 24B (2) | Correlate |
| | | 23976 (2534-3332) | | | |
| 25F | 25F-38 (1 + cn + s) | AF532711/ | 25F or 38 (3) | 25F or 38 (3) | Consistent |
| | | 28389 (9131-9922) | | | |
| 25A | 25A-29 (s) | 15096 (2153-2951) | NA | 25A or 29 (1) | Consistent |
| 27 | ?27-28F-28A (s) | 22978 (2486-3284) | 27 or 28A (1) | 27 or 28A (1) | Consistent |
| | 27-cn (cnx4) | AY508608 | NA | 27 (4) | Correlate |
| 28F | 16A-28F (s) | 21731 (2147-2945) | 16A or 28F (1) | 16A or 28F (1) | Consistent |
| | ?27-28F-28A (cnx3) | AY508610 | 28F or 28A (3) | 28F or 28A (3) | Consistent |
| | 28F-cn (cn) | AY508611 | 28F or 28A (1) | 28F (1) | Correlate |
| 28A | ?27-28F-28A (cnx3 + s) | AY508609/ | 28F or 28A (4) | 28F or 28A (4) | Consistent |
| | | 22978 (2486-3284) | | | |
| 29 | 29-ca (1) | AF532680 | NA | 29 (1) | Correlate |
| | 25A-29 (3 + s) | AY330718/ | NA | 25A or 29 (4) | Consistent |
| | | 15096 (2153-2951) | | | |
| 31^{h} | 31 (6 + 1^{h} + s + 1E) | AF532684, | 31 (6 + 1^{h} + s + 1E) | 31 (8 + 1E) | Correlate |
| | | AF532695/ | | | |
| | | 22164 (2538-3336) | | | |
| 32F | 32F-32A (cn + s) | AY508614/ | NA | 32F or 32A (2) | Consistent |
| | | 25372 (5428-6226) | | | |
| 32A | 32A-cn (cn) | AY508613 | NA | 32A (1) | Correlate |
| | 32F-32A (cn + s) | AY508612/ | NA | 32A or 32F (2) | Consistent |
| | | 25363 (5327-6125) | | | |
| 33F | 33F-g (g) | AJ006986 (2483-3281) | 33F or 33A or 37 (1) | 33F (1) | Correlate |
| | 33F-q (1) | AF532687 | 33F or 33A or 37 (1) | 33F (1) | Correlate |
| | 33F-33B (3) | AF532688 | 33F or 33A or 37 (3) | 33F (3) | Correlate |
| | 33F-33A-35A (2 + s) | AF532689/ | 33F or 33A or 37 (3) | 33F or 33A (3) | Consistent |
| | | 16989 (2155-2953) | | | |
| 33A | 33F-33A-35A (1 + cn + s) | AF532685/ | 33F or 33A or 37 (3) | 33F or 33A (3) | Consistent |
| | | 18409 (2155-2953) | | | |
| 33B | 33B-q (3 + qap) | AF532686 | 33B or 33C or 33D (4) | 33B (4) | Correlate |
| | 33B-s (s) | 19039 (2508-3306) | 33B or 33C or 33D (1) | 33B (1) | Correlate |
| | 33F-33B (cn) | AY508615 | 33B or 33C or 33D (1) | 33B (1) | Correlate |
| 33C | 33C-s (s) | 15918 (2155-2953) | 33B or 33 C or 33D (1) | 33C (1) | Correlate |
| | 33C-cn (cn) | AY508616 | 33B or 33C or 33D (1) | 33C (1) | Correlate |
| 33D | 33D-48 (s) | 17583 (2508-3306) | 33B or 33C or 33D (1) | 33D or 48 (1) | Consistent |
| 34 | 34-ca (4 + qap) | AF532690 | NA | 34 (5) | Correlate |
| | 34-s (s) | 15938 (2425-3223) | NA | 34 (1) | Correlate |
| 35F | 35F-47F (6 + s) | AF532692/ | 35F or 47F (7) | 35F or 47F (7) | Consistent |
| | | 15137 (2807-3605) | | | |
| 35A | 33F-33A-35A (cn + s) | AY508617/ | 35A or 35C or 42 (2) | 35A (2) | Correlate |
| | | 21463 (2200-2998) | | | |
| 35B | 17F-35B-35C-42 (9 + s) | AF532691/ | 35B (10)^{f} | 35B (10)^{f} | Correlate |
| | | 16658 (2186-2984) | | | |
| 35C | 17F-35B-35C-42 | AY508618, | 35C or 42 (4)^{f} | 35C or 42 (4)^{f} | Consistent |
| | (cnx2 + s + qap) | AY508640/ | | | |
| | | 19741 (2518-3316) | | | |
| 36 | 36-cn (cnx2 + s) | AY508619/ | NA | 36-s (3) | Correlate |
| | | 19113 (2805-3603) | | | |
| 37 | 37-g (g) | AJ131984 (2849-3648) | 33F or 33A or 37 (1) | 37 (1) | Correlate |
| | 37-ca (1 + cnx2 + qap + s) | AF532713/ | 33F or 33A or 37 (5) | 37 (5) | Correlate |
| | | 17777 (2557-3355) | | | |
| 38 | 25F-38 (7 + s) | AF532712/ | 25F or 38 (8) | 25F or 38 (8) | Consistent |
| | | 30298 (10688-11479) | | | |
| 39 | 39-cn (cn + s) | AY508620/ | NA | 39 (2) | Correlate |
| | | 17810 (2202-3000) | | | |
| | 39-cn (cn) | AY508621 | NA | 39 (1) | Correlate |
| 40 | 7B-40(cn + s) | AY508622/ | 7C or 40(2) | 40 (2) | Consistent |
| | | 22089 (2833-3631) | | | |
| 41F | 41F-cn (cn) | AY508624 | 41F-wzx (1) | 41F (1) | Correlate |
| | 41F-s (s) | 22917 (2848-3646) | 41F-wzx (1) | 41F (1) | Correlate |
| 41Aⁱ | 2-41A (1ⁱ + cn + s) | AY508623, | (41F-wzx)41A (3) | 41A (3) | Correlate |
| | | AF532694^{m}/ | | | |
| | | 22520 (2554-3352) | | | |
| 42 | 17F-35B-35C-42 (cn + s) | AY508625/ | 35A or 35C or 42 (2)^{f} | 35B or 35C or 42 (2)^{f} | Consistent |
| | | 19403 (2387-3185) | | | |
| 43 | 43-cn (cnx2 + s) | AY508626/ | NA | 43 (3) | Correlate |
| | | 22097 (2018-2816) | | | |
| 44 | 44-s (s) | 24095 (2181-2979) | Serogroup 12 or 44 or 46 (1) | 44 (1) | Correlate |
| 45 | 45-cn (cn + s) | AY508628/ | NA | 45 (2) | Correlate |
| | | 27591 (2540-3338) | | | |
| 46 | 46-s (s) | 25070 (2186-2984) | Serogroup 12 or 44 or 46 (1) | 46 (1) | Correlate |
| | 12A-46 (cnx2) | AY508629 | Serogroup 12 or 44 or 46 (1) | 12A or 46 (2) | Consistent |
| 47F | 35F-47F (cn + s) | AY508631/ | 35F or 47F (2) | 35F or 47F (2) | Consistent |
| | | 16064 (2538-3336) | | | |
| 47A | 47A-cn (cn + s) | AY508630/ | NA | 47A (2) | Correlate |
| | | 17250 (2535-3333) | | | |
| 48 | 48-cn (cn) | AY508633 | NA | 48 (1) | Correlate |
| | 33D-48 (s) | 17583 (2508-3306) | 33B or 33 C or 33D or 48(1) | 33D or 48 (1) | Consistent |
| 48(1) | 48(1)-cn (cn + s) | AY508632/ | NA | 48(1) (2) | Correlate |
| | | 22062 (2372-3170) | | | |
| NT | NT-nz (1)^{j} | AF532714 | NA | NT (1)^{e} | Correlate |
| | NT-ca (1)^{j} | AF532715 | NA | NT (1)^{e} | Correlate |
| | NT (3)^{j} | NA | NA | NT (3)^{e} | Correlate |

Notes.
^{a}Bold letter/numbers indicate results “consistent” (see below for their definition) between MCT and CS; limited CS is needed to distinguish 2-5 serotypes within sequence types, also see text for further explainations. NT = nonserotypeable or nontypeable. Figures in parentheses indicate number of isolate and strain source for the 87 strains used in the study, the GenBank and Sanger Institute strains were also calculated into the total numbers.
^{b}For explanation of sequence type nomenclature, see text. Key: -g (GenBank sequence); -c (CIDM); -n (New South Wales); -q (Queensland); -w (Western Australia); -v (Victoria); -ca (Canada); -nz (New Zealand); -cn (China); -qap (QAP programme); -s (Sanger). Different serotypes/sequence types that share the same sequences are bolded.
^{c}GenBank sequence accession numbers for corresponding sequence type: Those before “;” are described by the others, one sequence start and stop positions corresponding the ˜800 bp regions were given; those behind “;” are the sequences we studied; the sequence behind “/” were got from Sanger Institute Streptococcus pneumoniae capsular loci sequence project sequence start and stop positions corresponding the ˜800 bp regions are given.
^{d}“Correlate” means that MCT and CS results were identical; “consistent” means that components of MCT results (sequence type or PCR) correlated with more than one (2-5) CS result.
^{e}Serogroup 18 PCR positive and 11A-11D specific PCR negative, which can confirm the strains would be 18F.
^{f}Serotype 17F PCR negative.
^{g}7C and 19B-19C PCR negative, 24B could be identified by exclusion.
^{h}One previous 42 strain (Example 1) was finally proved to be 31 - after twice repeat conventional serotyping and serotype 31-specific PCR positive.
ⁱOne previous 41F strain (Example 1) was finally proved to be 41A - after twice repeat conventional serotyping.
^{j}Some of these isolates may belong to rare sequence types or even serotypes (other than the known 90 serotypes) not represented among our reference isolates.

Are the Shared sequence Types plausible?

In order to explain the many shared sequence types, we studied their antigenic formula (Henrichsen, 1995). Among the 31 shared sequence types (Table 9), six were shared between unrelated serotypes (2-41A, 10A-17A, 10A-23F, 13-20, 25A-29, 33D-48), three were shared between two to three related and at least another unrelated serotype (7B-40, 11A-11D-18F, 27-28F-28A, 17F-35B-35C-42) and 20 were shared between antigenically related serotypes. The remaining shared sequence type involved serotypes 16A and 28F; although they are not directly related, 28F is related to serogroup 16 (Table 9) (Henrichsen, 1995). Thus most shared molecular capsular or sequence types (genotypes) involve closely related serotypes (or phenotypes). The 10 shared sequence types that involve unrelated or more distantly related (such as 16A-28F) serotypes probably can be explained by recombination events between serotypes.

Are wzx and wzy Helpful?

In Example 1 it was shown that wzy and wzx based PCRs increase the accuracy of cpsA-cpsB sequence-based serotype prediction. Thus, in order to extend our serotype-prediction strategy to all 90 serotypes, we examined the wzx and wzy sequences of the 90 serotypes, especially the 31 shared sequence types (Tables 7 and 9). In addition to the sequences we have determined, the unannotated sequences from the cps gene clusters of all 90 serotypes as determined by the Sanger Institute was used to determine the 90 wzx and wzy sequences. The identical of suitable serotype-specific wzx and wzy based primers was far from straightforward. For most of the 90 serotypes, wzy is shorter but more heterogeneous than wzx and therefore a more suitable single target for serotype-specific PCR. The wzy sequencing results showed that it would be helpful for the discrimination of 7C-40, 10F-10C, 12A/46 (identical)-12F/12B/44 (identical), 35A-35C/42 (identical), 35F-47F serotype(s) pairs.

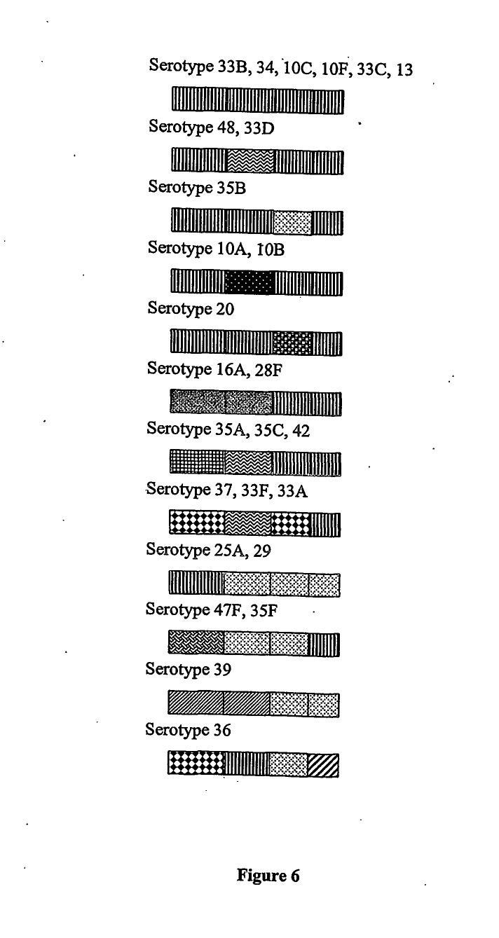

It is shown that wzx genes from 28 different serotypes share high-level homology (72% to 100%). We found three main recombination sites in these 28 wzx (base positions 395, 775 and 1150) using the programme PhylPro 1.0 (Weiller 1998), which generated the diagrammatic representation of polymorphic sites and hypothetical recombination events of the wzx gene shown in FIG. 6.

TABLE 9
The relationship between shared S. pneumoniae partial cpsA-cpsB sequence (798-800 bp) type and conventional serotyping
(CS) antigenic formulas.

| Involved | | | wzx | wzy | Selected | cps gene |
|---|---|---|---|---|---|---|
| CS^{a} | Involved sequence types^{b} | Antigenic formulas^{cd} | identity (%)^{e} | identity (%)^{e} | PCR^{f} | cluster (%)^{g} |
| 2 | 2-41A | 2a (NCR) | — | — | 2 | — |
| 41A | | 41a (NCR) | SD | SD | 41F-41A | — |
| 6A | 6A-6B-g, 6A-6B-q, 6A-6B-s | 6a, 6b | — | — | IP | — |
| 6B | | 6a, 6c | 99.929 | 99.851 | IP | 99.079 |
| 7F | 7F-7A | 7a, 7b | — | — | IP | — |
| 7A | | 7a, 7b, 7c | 100.000 | 100.000 | IP | SD |
| 7B | 7B-40 | 7a, 7d, 7e, 7h | — | — | 7B-7C-40 | — |
| 40 | | 40a, 7g, 7h | | | 7B-7C-40 | — |
| 7C | 7C-19C-24B | 7a, 7d, 7f, 7g, 7h | — | — | 7B-7C-40 | — |
| 19C | | 19a, 19c, 19f, 7h | 67.002 | SD | 19B-19C | — |
| 24B | | 24a, 24b, 24e, 7h | 98.903 | SD | 24B | — |
| 9A | 9A-9V | 9a, 9c, 9d | — | — | IP | — |
| 9V | | 9a, 9c, 9d, 9g | 100.000 | 100.000 | IP | 99.990 |
| | | | | | | (except |
| | | | | | | beginning) |
| 10F | 10F-10C | 10a, 10b | — | — | Seq-wzy | — |
| 10C | | 10a, 10b, 10c, 10f (NCR) | 99.293 | 98.801 | Seq-wzy | 96.970 |
| 10A | 10A-17A | 10a, 10c, 10d (NCR) | — | — | 10A-10B | — |
| 17A | | 17a, 17c (NCR) | SD | SD | 17A | — |
| 10A | 10A-23F | 10a, 10c, 10d (NCR) | — | — | 10A-10B | — |
| 23F | | 23a, 23b, 18b (NCR) | SD | SD (>wzx) | 23F | — |
| 10B | 10B-10C | 10a, 10b, 10c, 10d, 10e | — | — | 10A-10B | — |
| 10C | | 10a, 10b, 10c, 10f | 83.156 | 95.843 | 10A-10B-neg& | — |
| | | | | | Seq-wzy | |
| 11A | 11A-11D-18F | 11a, 11c, 11d, 11e | — | — | 11A-11D | — |
| 11D | | 11a, 11b, 11c, 11e | 100.000 | 100.000 | 11A-11D | 99.393 |
| 18F | | 18a, 18b, 18c, 18f (NCR) | SD | SD | sergroup18 | — |
| 11B | 11B-11C | 11a, 11b, 11f, 11g | — | — | NA | — |
| 11C | | 11a, 11b, 11c, 11d, 11f | — | — | NA | — |
| 12F | 12F-12A-12B | 12a, 12b, 12d | — | — | Seq-wzy | — |
| 12A | | 12a, 12c, 12d | 99.417 | 98.102 | Seq-wzy | SD |
| 12B | | 12a, 12b, 12c, 12e | 99.741 | 100.000 | Seq-wzy | 98.965 |
| | | | | | | (12B:12A = 96.939) |
| 12A | 12A-46 | 12a, 12c, 12d | — | — | Seq-wzy | — |
| 46 | | 46a, 12c, 44b | 100.000 | 99.835 | Seq-wzy | 93.210 |
| 13 | 13-20 | 13a, 13b (NCR) | — | — | 13 | — |
| 20 | | 20a, 20b, 7g (NCR) | 83.828 | SD | 20 | — |
| 15B | 15B-15C | 15a, 15b, 15d, 15e, 15h | — | — | IP | — |
| 15C | | 15a, 15d, 15e | 100.000 | 100.000 | IP | 99.979 |
| 15B | 15B-15C-22F-22A | 15a, 15b, 15d, 15e, 15h | — | — | 15B-15C | — |
| 15C | | 15a, 15d, 15e | 100.000 | 100.000 | 15B-15C | — |
| 22F | | 22a, 22b | SD (55.623) | SD | 22F-22A | — |
| 22A | | 22a, 22c | SD (22A:22F = | SD (22A:22F = | 22F-22A | 22F:22A = 99.996 |
| | | | 100.000) | 100.000) | | |
| 16A | 16A-28F | 16a, 16c (NCR)^{h} | — | — | IP | — |
| 28F | | 28a, 28b, 16b, 23d (NCR)^{h} | 100.000 | 99.900 | IP | 99.991 |
| 17F | 17F-35B-35C-42 | 17a, 17b (NCR) | — | — | 17F | — |
| 35B | | 35a, 35c, 29b | SD | SD | 35B | — |
| 35C | | 35a, 35c, 20b, 42a | SD | SD | Seq-wzy | 35C:35A = 96.135 |
| | | | (35C:35B = | (35C:35B = | | |
| | | | 77.189) | 75.990) | | |
| | | | (35C:35A = | (35C:35B = | | |
| | | | 99.859) | 98.995) | | |
| 42 | | 42a, 20b, 35c | SD | SD | Seq-wzy | 42:35C = 99.830 |
| | | | (42:35C = | (42:35C = | | |
| | | | 100.000) | 99.916) | | |
| 18B | 18B-18C | 18a, 18b, 18e, 18g | — | — | IP | — |
| 18C | | 18a, 18b, 18c, 18e | 100.000 | 100.000 | IP | 99.991 |
| 23F | 23F-23A | 23a, 23b, 18b | — | — | 23F | — |
| 23A | | 23a, 23c, 15a | 99.928 | SD | 23A | — |
| 25F | 25F-38 | 25a, 25b | — | — | Seq-wzy | — |
| 38 | | 38a, 25b | 99.506 | 99.581 | Seq-wzy | 97.378 |
| 25A | 25A-29 | 25a, 25c, 38a (NCR) | — | — | 25A-29 | — |
| 29 | | 29a, 29b, 13b (NCR) | 100.000 | 100.000 | 25A-29 | 100.000 |
| 27 | 27-28F-28A | 27a, 27b (NCR) | — | — | 28F-28A | — |
| 28F | | 28a, 28b, 16b, 23d | SD | SD | 28F-28A | — |
| 28A | | 28a, 28c, 23d | 100.000 | 100.000 | 28F-28A | 99.991 |
| | | | (28A:28F = | (28A:28F = SD) | | |
| | | | SD) | | | |
| 32F | 32F-32A | 32a, 27b | — | — | IP | — |
| 32A | | 32a, 32b, 27b | 100.000 | 100.000 | IP | 99.996 |
| 33F | 33F-33A-35A | 33a, 33b, 33d | — | — | 33F-33A-37 | — |
| 33A | | 33a, 33b, 33d, 20b | 100.000 | 100.000 | 33F-33A-37 | 99.792 |
| 35A | | 35a, 35c, 20b | 77.754 | SD | 35A-35C-42 | — |
| 33F | 33F-33B | 33a, 33b, 33d | — | — | 33F-33A-37 | — |
| 33B | | 33a, 33c, 33d, 33f | 77.951 | SD | 33B-33C-33D-48 | — |
| 33D | 33D-48 | 33a, 33c, 33d, 33f, 6a | — | — | IP | — |
| | | (NCR) | | | | |
| 48 | | 48a (NCR) | 100.000 | 100.000 | IP | 99.989 |
| 35F | 35F-47F | 35a, 35b, 34b | — | — | Seq-wzy | — |
| 47F | | 47a, 35a, 35b | 99.859 | 99.754 | Seq-wzy | 94.749 |
| | | | | | | (except |
| | | | | | | beginning |
| | | | | | | and end) |

Notes.
^{a}Those conventional serotypes (CS) that could share the same sequence types.
^{b}Those sequence types that could be shared by different (2-5) conventional serotypes.
^{c}Bold parts showed that the factor antiserum are shared by all the shared sequence types related serotypes; underline part showed that the factor antiserum are shared by partial (2-4) shared sequence types related serotypes.
^{d}NCR: no cross-reaction of any factor antiserum in the antigenic formulas between serotypes that share sequence types (Henrichsen, 1995).
^{e}Sequence identity was calculated by the comparison of wzx and wzy sequences - the others wzx and wzy compared the first CS in the several sharing ST CS. SD: significant length and sequence differences (heterogeneity) between wzx or wzy.
^{f}Only selected some PCR to show cases and the “serotype-specific” PCR was only evaluated within the related CS that shared sequence types. IP = impossible (or unlikely) to design real serotype-specific PCR primers to differentiate between the share sequence serotypes because the very high wzx and wzy sequence simarility.
^{g}Only those with very high wzx and wzy sequence simarility serotypes cps gene cluster comparison results are shown.

Comprehensive Molecular Capsular Sequence Typing Results

The final molecular capsular sequence typing results for 519 isolates (427 previously studied and 92 new isolates) are shown in Table 9. Our database now includes 90 S. pneumoniae serotypes and 134 sequence types (including two non-serotypeable strains). 83 serotypes are represented by 2 or more strains. 102 sequence types (not including two nonserotypeable strains), including 47 that are represented by two or more isolates, correspond to a single serotype; 23 sequence types are shared by two serotypes, six are shared by three serotypes and two are shared by four serotypes (Table 8).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed above are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Arai, S. et al. (2001) Microbiol. Immunol. 45;159-62.
Astschul, S. F. et al. (1997) Nucl. Acids Res. 25;3389-402.
Arrecubieta, C. et al. (1996) J. Exp. Med. 184;449-55.
Barker, J. H. et al. (1999) J. Clin. Microbiol. 37;4039-41.
Bateman, A.
et al. (2002) Nucl. Acids Res. 30;276-80.
Chen, Y. et al. (2003) Marnm. Genome 14;859-65.
Coffey, T. J. et al. (1998) Mol. Microbiol. 27;73-83.
Colman, G. et al. (1998). J. Med. Microbiol. 47;17-27.
Dunne, W. M., Jr. (2001) J. Clin. Microbiol. 39;1791-1795.
Hausdorff, W. P. et al. (2001) Lancet 357;950-2.
Henrichsen, J. (1995) J. Clin. Microbiol. 33;2759-62.
Henrichsen, J. (1999) Am. J. Med. 107;50S-54S.
Huebner, R. E. et al. (2000) S. Afr. Med. J. 90;1116-21.
Huebner, R. E. et al. (2000) Int. J. Infect. Dis. 4;214-8.
Jiang, S M. et al. (2001) Infect. Immun. 69;1244-55.
Kong, F. et al. (2000) J. Clin. Microbiol. 38;4256-9.
Kong, F. et al. (2002) J. Clin. Microbiol. 40;216-26.
Kumar, S. et al. (1994) Comput. Appl. Biosci. 10:189-91.
Lalitha, M. K. et al. (1999) J. Clin. Microbiol. 37;263-5.
Lawrence, E. R. et al. (2000) J. Clin. Microbiol. 38;1319-23.
Lipsitch, M. (2001) Am. J. Epidemiol. 154;85-92.
Morrison, K. E. et al. (2000) J. Clin. Microbiol. 38;434-7.
Robertson, G. A. et al. (2004) J. Med. Microbiol. 53;35-45.
Rubins, J. B. et al. (1999) Infect. Immun. 67;5979-84.
Salo, P. et al. (1995). J. Infect. Dis. 171;479-82.
Schena. M. et al. (1998) Trends Biotechnol. 16;301-6.
Sorensen, U. B. (1993) J.Clin. Microbiol. 31;2097-2100.
Straub, T. M. et al. (2002) Appl. Environ. Microbiol. 68;1817-26.
Thompson, J. D. et al. (1994) Nucl. Acids Res. 22;4673-80.
Vakevainen, M. et al. (2001) J. Infect. Dis. 184;789-93.
van Leeuwen, W. B. et al. (2003) J. Clin. Microbiol. 41;3323-6.
van Selm, S. et al. (2002) Microbiology 148;1747-55.
Volokhov, D. et al. (2002) J. Clin. Microbiol. 40;4720-8.
Weiller, G. F. (1998) Mol. Biol. Evol. 15;326-35.

1-31. (canceled)

32. A method of distinguishing between at least 25 different serotypes of Streptococcus pneumoniae in a sample, the method comprising,
i) analysing at least a portion of the nucleotide sequence between the 3′ end of the cpsA gene and the 5′ end of the cpsB gene, and/or
ii) analysing at least a portion of the wzy and/or wzx gene(s).

33. The method of claim 32 which distinguishes between at least 70 different serotypes of Streptococcus pneumoniae in a sample.

34. A method of determining the serotype of Streptococcus pneumoniae in a sample, the method comprising,
i) analysing at least a portion of the nucleotide sequence between the 3′ end of the cpsA gene and the 5′ end of the cpsB gene, and/or
ii) analysing at least a portion of the wzy and/or wzx gene(s).

35. The method of claim 34, wherein the serotype is selected from the group consisting of: 2, 7A, 7B, 7C, 9A, 9L, 10F, 10A, 10B, 10C, 11F, 11A, 11B, 11C, 11D, 12F, 12A, 12B, 13, 15F, 15A, 15B, 15C, 16A, 17F, 17A, 18F, 18A, 18B, 21, 22F, 22A, 24F, 24A, 24B, 25F, 25A, 27, 28F, 28A, 31, 32F, 32A, 33F, 33A, 33B, 33C, 33D, 34, 35B, 35C, 36, 37, 38, 39, 40, 41F, 41A, 42, 43, 44, 45, 46, 47, 47A and 48.

36. The method of claim 34, wherein the portion of the nucleotide sequence between the 3′ end of the cpsA gene and the 5′ end of the cpsB gene which is analysed is any nucleotide which is polymorphic between at least some of the S. pneumoniae serotypes referred to in FIG. 2.

37. The method of claim 34, wherein the method comprises amplifying at least a portion of the nucleotide sequence between the 3′ end of the cpsA gene and the 5′ end of the cpsB gene, and sequencing the amplification product.

38. The method of claim 37, wherein the entire approximately 800 bp region as provided in FIG. 2 is amplified and sequenced.

39. The method of claim 38, wherein the amplification is performed using primer pairs comprising a sequence selected from the group consisting of:

| | |
|---|---|
| 1) GGCATT(/C)TATGGAGTTGATTCG(/A)TCCA | (SEQ ID NO:68) |
| TT(/C)CACAC(C/T)TTAG | |
| and | |
| GC(/T)TCAATG(/A)TGG(/A)GCAATG(/T)ACT | (SEQ ID NO:73) |
| GGA(/C)GTA(/G)ATTCCCA(/G)ACATC, | |
| 2) GGCATT(/C)TATGGAGTTGATTCG(/A)TCCA | (SEQ ID NO:68) |
| TT(/C)CACACC(/T)TTAG | |
| and | |
| CCATCAC(/T)ATAGAGGTTAC(/A)TG(/A)TCTG | (SEQ ID NO:71) |
| GCATT(/C)GC, | |
| 3) GAAAGTGGG(/A/T)GGG(/A/T)A(/G)A | (SEQ ID NO:70) |
| (/C)T(/G)TAT(/C)AAAGTA(/G)AATTCT(/G) | |
| CAAGAT(/C)TTA(/G)AAA(/G)G | |
| and | |
| T(/G)CATG(/A)CTA(/G)AAC(/T)TCT(/A)AT | (SEQ ID NO:72) |
| C(/T)AAG(/A)GCATAACGACTATC(/T), | |
| and | |

4) primer pairs that amplify the same region, or diagnostic portion thereof, from the genome of a strain of S. pneumoniae as the primers provided in 1) to 3).

40. The method of claim 34, wherein the nucleotide sequence analysis step comprises determining whether a polynucleotide obtained from S. pneumoniae selectively hybridises to a polynucleotide probe comprising one or more polymorphic regions of the nucleotide sequence between the 3′ end of the cpsA gene and the 5′ end of the cpsB. gene, wherein such polymorphic regions are shown in FIG. 2.

41. The method of claim 40, wherein the nucleotide sequence analysis step comprises a plurality of said polynucleotide probes.

42. The method of claim 40, wherein the polynucleotide probe(s) is present as a microarray.

43. The method of claim 34 which comprises amplifying at least a portion of the wzy and/or wzx gene(s), and determining the length of the amplification product.

44. The method of claim 43, wherein at least a portion of the wzy and/or wzx gene(s) is amplified using a primer comprising a sequence selected from any one of SEQ ID NO's 75 to 139 or 144 to 333, or a primer that can be used to amplify the same region, or diagnostic portion thereof, from the genome of a strain of S. pneumoniae as a primers provided as any one of SEQ ID NO's 75 to 139 or 144 to 333.

45. A method of identifying serotype 3 of Streptococcus pneumoniae in a sample comprising performing a method of claim 34, and analysing the orb2 (wze)-cap3A-cap3B region.

46. The method of claim 45, wherein the or2 (wze)-cap3A-cap3B region is analysed by amplifying a portion of the orb2 (wze)-cap3A-cap3B region using primer pairs selected from the group consisting of:
(SEQ ID NO:140)
1) GCACAAAAAAAAGTTTGATATTCCCCTTGACAATAG
and
(SEQ ID NO:141)
GCAGGATCTAAGGAGGCTTCAAGATTCAACTC,
(SEQ ID NO:142)
2) CGAACCTACTATTGAGTGTGATACTTTTATGGGATACAGAG
(SEQ ID NO:143)
CTGACAGCATGAAAATATATAACCGCCCAACGAATAAG,
and
3) primer pairs that amplify the same region, or diagnostic portion thereof, from the genome of a strain of S. pneumoniae as the primers provided in 1) or 2).

47. The method of claim 32, the method further comprising detecting any serotype of Streptococcus pneumoniae in the sample.

48. The method of claim 47, wherein the psaA and/or pneumolysin genes, or a portion thereof, is amplified.

49. The method of claim 48, wherein a portion of the psaA gene is amplified using primers comprising the sequence TACATTACTCGTTCTCTTTCTTTCTGCAATCATTCTTG (SEQ ID NO:64) and TAGTAGCTGTCGCCTTCTTTACCTTGTTCTGC (SEQ ID NO:65), or primer pairs that amplify the same region, or diagnostic portion thereof, from the genome of a strain of S. pneumoniae as SEQ ID NO:64 and SEQ ID NO:65.

50. The method of claim 48, wherein a portion of the pneumolysin gene is amplified using primers comprising the sequence AGAATAATCCCACTCTTCTTGCGGTTGA (SEQ ID NO:66) and CATGCTGTGAGCCGTTATTTTTTCATACTG (SEQ ID NO:67) or primer pairs that amplify the same region, or diagnostic portion thereof, from the genome of a strain of S. pneumoniae as SEQ ID NO:66 and SEQ ID NO:67.


SEQUENCE LISTING (492 sequences)

SEQ ID NO: 1 - DNA, 4000 bp, Streptococcus pneumoniae
gtcaaatctgtcttgattgaaaacactgcggctaaagaagtacttgaaaaacaggtcttggctccatgggatgctttctgtgtggaattactataaatattttttgcagaaaaatttaaaattgaaatcgtataaaaacaagggaggactgtataaaagacagaaatcctttgttttttataaccaaagtttataaactttcattcttgaaattcaattaactttacaaattcccactattaaggagaaagaagatgaacataaagaagcgtgtccttagtgcaggcctgacttttgcatctgctttgcttttagctgcttgcggccaatcaggttcagatacaaaaacttactcatcaacctttagtggaaatccaactacatttaactatctattagactattacgctgataatatagttaattgaaacaagaacaagacaaaagagcctcataaaaggtattgcaacttggtaatacctttttgaggtgctttttgatatgagcccatgttttctcaataggattgtactcaggtgagtagggaggaagaggtaaaagtttatacccaaactcttcacacaagagttctaacttacccattctatggaatcttgcattatccataataataaccgatggtgtgtttaatgttggtaagagaaatttctgaaaccaagcttcaaaaaagtcgctcgtcatcgtctcttcgtaagttattggagcgattaactcaccatttgttagacctgcaaccaaagaaatcctctgatatcttcttccagatactttgcctcttcttaactgaccttttaatgagcgaccatattctcgataaaaataagtatcgaatcctgtttcatcaatctaaacaggtgctaggtgctttaaactattaaaattcttaagaaataaggctactttttctgggttttgttcatagtaggtgtggttctttttttcgagtgtagcccatagctttgagcgcatagtggatggtagttggatgacagccaaattcagaagctatttcagtcaaataagcgtctggattgtcagtaagatagtttttaagtctatctctatcaacttttcttggttttgttccttttacttggtggtttagctctcctgttttctcttttagctttaaccagccataaatggtattacgtgatatttggaaaacgtgtgatgcttctgttatactacctgttcgctcacaataagagagaacttttttacgaaaatctattgaatatgccataagaagattataccacattgtgtactatattagattgaaactagaatagtacacctctgcttctaaaacattgttagaaatcgatttgactgtcctgaacgatttgttctgttcttatttcattttactatatttttgtttcgcgggaagtctactaagatacttaaagatgcagatagtaaaaataaaggtgtagacattaccgtaaaaaagtgatataatcgtatagtgttcaatgtataggtattaatcatgagtagacgttttaaaaaatcacgttcacagaaagtgaagcgaagtgttaatatagttttgctgactatttatttattgttagtttgttttttattgttcttaatctttaagtacaatatccttgcttttagatatcttaatctagtggtaactgcgttagtcctactagttgccttggtagggctactcttgattatctataaaaaagctgaaaagtttactatttttctgttggtgttctctatccttgtcagctctgtgtcgctctttgcagtacagcagtttgttggactgaccaatcgtttaaatgcgacttctaattactcagaatattcaatcagtgtcgctgttttagcagatagtgagatcgaaaatgttacgcaactgacgagtgtgacagcaccgactgggactaataatgaaaatattcagaaattactagctgatatcaagtcaagtcagaataccgatttgacggtcaaccagagttcgtcttacttggcagcttacaagagtttgattgcaggggagactaaggccattgtcctaaatagtgtctttgaaaacatcatcgagtcagagtatccagactacgcatcgaagataaaaaagatttatactaagggattcactaaaaaagtagaagctcctaagacgtctaagagtcagtctttcaatatctatgttagtggaattgacacctatggtcctattagttcggtgtcgcgatcagatgtcaacatcctgatgactgtcaatcgagataccaagaaaatcctcttgaccacaacgccacgtgatgcctatgtaccaatcgcagatggtggaaataatcaaaaagataaattgactcatgcgggcatttatggagttgattcgtccattcacaccttagaaaatctctatggagtggatatcaattactatgtgcgattgaacttcacttcgtttttgaaattgattgatttgttgggtggaattgatgtttataatgatcaagaatttactgcccatacgaatggaaagtattaccctgcaggcaatgttcatcttgattcagaacaggctctcggttttgttcgtgagcgctactccctagcagatggcgatcgtgaccgcgggcgccatcaacaaaaggtgattgtggctatccttcaaaaattaacgtcaaccgaagtgctgaaaaattatagtacgatcattaatagcttgcaagattctatccaaacaaatatgccacttgagaccatgataaatttggtcaatgctcagttagaaagtggagggaattataaagtaaattctcaagatttaaaagggacaggtcggatggatcttccttcttatgcaatgccagacagtaacctctatgtgatggaaatagatgatagtagtttagctgtagttaaagcagctatacaggatgtgatggagggtagatgaaatgatagacatccattcgcatatcgtttttgatgtagatgacggtcccaagtcaagagaggaaagcaaggctctcttggcagaatcctacagacagggggtgcgaaccattgtttctacctctcaccgtcgcaagggcatgtttgaaactccggaagagaagatagcagaaaactttcttcaggttcgggaaatagctaaggaagtggcgagtgacttggtcattgcttacggggctgaaatttattacacaccagatgttctggataagctggaaaaaaagcggattccgaccctcaatgatagtcgttatgccttgatagagtttagtatgaacactccttatcgcgatattcatagcgccttgagcaagatcttgatgttgggaattactccagtcattgcccacattgagcgctatgatgctcttgaaaataatgaaaaacgcgttcgagaactgatcgatatgggctgttacacgcaagtaaatagttcacatgtcctcaaacccaaactttttggcgaacgttataaattcatgaaaaaaagagctcagtattttttagagcaggatttggttcatgtcattgcaagtgatatgcacaatctagacggtagacctcctcatatggcagaagcatatgaccttgttacccaaaaatacggagaagcgaaggctcaggaactttttatagacaatcctcgaaaaattgtaatggatcaactaatttaggagaaatgatgaaagaacaaaatacgatagaaatcgatgtatttcaattagttaaaagcttgtggaaacgcaagctaatgattttaatagtggcacttgtgacaggtgcgggggcttttgcatatagcacttttattgttaagccagaatatacgagtaccacgcgaatttacgtagtgaatcgcaatcaaggagacaagccggggttgacaaatcaggatttgcaggcaggaacttatctggtaaaagactaccgtgagattatcctttcgcaggatgttttggaggaagttgtttctgat

SEQ ID NO: 2 - DNA, 1019 bp, Streptococcus pneumoniae
atgaaattgaagtttcttataacaaatttatttcatgtctttttgtctaatctgattacaattgtcacatcggttatagttgtactaattttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttatttgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggacttgtaaagatattgtttttcaatccttatccgagttcatattggatctgagagagtcttttgacaatatccgtgttggaatcaacttaatgttatccaatattgcaagtagtatgattattggtattgttcgaatgggaattcaatggaattggaatatcgaaacattcgggaaagtatcactgatgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagat

SEQ ID NO: 3 - DNA, 1019 bp, Streptococcus pneumoniae
atgaaattgaagtttcttataacaaatttgtttcatgtctttttgtctaatctgattacaattgtcacatcggttatagttgtactaatcttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaattttccagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagat

SEQ ID NO: 4 - DNA, 1019 bp, Streptococcus pneumoniae
atgaaattgaagtttcttataacaaatttgtttcatgtctttttgtctaatctgattacaattgtcacatcggttatagttgtactaattttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaattttccagttttttaattttaatttcttttctattatttggttttaacttattgattgtgacagatccaaatgcaaaatatatttataacatgaccattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagat

SEQ ID NO: 5 - DNA, 1019 bp, Streptococcus pneumoniae
atgaaattgaagtttcttataacaaatttgtttcatgtctttttgtctaatctgattacaattgtcacatcggttatagttgtactaatcttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagat

SEQ ID NO: 6 - DNA, 1019 bp, Streptococcus pneumoniae; identical to SEQ ID NO: 2

SEQ ID NO: 7 - DNA, 1019 bp, Streptococcus pneumoniae
atgaaattgaagtttcttataacaaatttgtttcatgtctttttgtctaatctgattacaattgtcacatcggttatagttgtactaatcttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaaacatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagat

SEQ ID NO: 8 - DNA, 1019 bp, Streptococcus pneumoniae
atgaaattgaagtttcttataacaaatttgtttcatgttcttttgtctaatctgattacaattcttacatcagttatagttgtactaattttaccaaaaattatgggagtaactgagtatagttattggcaactatatattttttacctaacatatattggtttttttcatctgggatggattgatggaatttatcttaaatatggcggattagagtaccagaacttagataagaaacagttttattctcaaatacttcaattttccagttttttaattttaatttcttttctattatttggttttaacttattgactgtgacagatcaaaatgcaaaatatatttataacatgactattattagtatgatagttacaaatttaagaatgttattcgtttatattttgcagatgacaaatcgattaaaggatagttccatcattctaatcagtgatcgcgttatatatgttattcttttattcctgtttattatatttaaatggcatgaatacaaggtaatgatttgggcagatgttttgggaaggacattttctctcctactttctttttggatttgtaaagatattgtttttcaatccttatccgagtttatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtttttcctttgttaaaacggacaaaaacggaaaatttatctaaaatttattccaacttaagaaatgttttgatgcttatcatgttcgcgattttgctcatttactatcctttaaaaattgtattagacctctggttgccagcctatcaagatgccttgattttcatggctcttatttttcctatgtcaatttatgaagggaagat

SEQ ID NO: 9 - DNA, 1019 bp, Streptococcus pneumoniae
atgaaattgaagtttcttataacaaatttgtttcatgtctttttatctaatctgattacaattgtcacatcggttatagttgtactaattttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgtcatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagaatcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattactcttttattatcctttaaaaattattctagatctttggttgccagcttatcgggatgcgttgatttttatggctcttatttttcctatgtcaatttatgaagggaagat

SEQ ID NO: 10 - DNA, 1019 bp, Streptococcus pneumoniae
atgaaattgaagtttcttataacaaatttgtttcatgtctttttatctaatctgattacaattgtcacatcggttatagttgtactaattttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcgaaatatatttataacatgactattattagtatgatagttacaaatttaagaatgttattcgtttatattttgcagatgacaaatcgattaaaggatagctctataattctgataagtgatcgcgtcatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagaatcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattactcttttattatcctttaaaaattattctagatctttggttgccagcttatcgggatgcgttgatttttatggctcttatttttcctatgtcaatttatgaagggaagat

SEQ ID NO: 11 - DNA, 1019 bp, Streptococcus pneumoniae; identical to SEQ ID NO: 8

SEQ ID NO: 12 - DNA, 743 bp, Streptococcus pneumoniae
atgcttttaaatttcttattcatatctatttttctattaattatcattacatttatattatttgagggggatttttttcaacctgcagtaattttaacactcacttattttatttcgattgcaagtgctctagttaatagaaatgtttggggaacagaactccatttcaaaacctttggtttgatattgttaggggttgctacatttattatagtttccttgttgacaaaattgtcgtacaggcctaaagtggagggaatttcgtatgaagaattgaaagaaataaatccttcaaagataatctatgtcattcttctgattctaaatcttgttatgctatttctttatacccgtgaaattcagaaagtggtattgttttcaggtagaagtttttctaatattacagatttgataagtaactataggtacctatcttattattcaaatgaagtagaaataagtggaatgattaatcaactatctaaaattattccagcgactacacttatttctttatatatatttataaataattattttataactaaacaaataaagaaaaatttcatttatttgattccaatagctatattctttgtctatgcaatcattagtggtggtagattgccccttataaggttagttgttggagctctgttgatattgtatatatactctgtgtacgggagtcctaaatctcaacttaccaaaagttttaaaatgatcactcgctctctgtttac

SEQ ID NO: 13 - DNA, 749 bp, Streptococcus pneumoniae
atgcttttaaatttcttattcatatctatttttctattaattatcattacatttatattatttgagggggatttttttcaacctgcagtaattttaacaatcgcttattttatttcgattgcaagtgctctagttaatagaaatgtttggggaacagaactccatttcaaaaccttttatttgatattgttaggggttgctacatttgttatagtttccttgttgacaaaattgtcgtacaggcctaaagtggagggaatttcgcatgaagaattgaaagaaataaatccttcaaagataatctatgtcattcttctgactctaaatcttgttatgttatttctttatatccgtgaaattcagaaagtagtattgttttcaggtagaagtttttctaatattacagatttgataagtaactataggtacctatcttattattcaaatgaagtagaaaatcgtgtaagtggaatgattaatcaactatctaaaattattccagcgactacacttatttctttatatatatttatgaataattattttataactaaacaaataaagaaaaatttcatttatttgattccaatagctatattctttgtctatgcaatcattagtggtggtagattgccccttataaggttagttgttggatctctgttgatattgtatatatactctgtgtacgggagtcctaaatctcaacttaccaaaagttttaaaatgatcactcgctctctgtttac

SEQ ID NO: 14 - DNA, 749 bp, Streptococcus pneumoniae
atgcttttaaatttcttattcatatctatttttctattaattatcattacatttatattatttgagggggatttttttcaacctgcagtaattttaacaatcgcttattttatttcgattgcaagtgctctagttaatagaaatgtttggggaacagaactccatttcaaaaccttttatttgatattgttaggggttgctacatttattatagtttccttgttgacaaaattgtcgtacaggcctaaagtggagggaatttcgcatgaagaattgaaagaaataaatccttcaaagataatctatgtcattcttctgattctaaatcttgttatgctatttctttatatccgtgaaattcagaaagtggtattgttttcaggtagaagtttttctaatattacagatttgataagtaactataggtacctatcttattattcaaatgaagtagaaaatcgtgtaagtggaatgattaatcaactatctaaaattattccagcgactacacttatttctttatatatatttataaataattattttataactaaacaaataaagaaaaacttcatttatttgattccaatagctatattctttgtctatgcaatcattagtggtggtagattgccccttataaggttagttgttggagctctgttgatattgtatatatactctgtgtacgggagtcctaaatctcaacttaccaaaagttttaaaatgatcactcgctctctgtttac

SEQ ID NO: 15 - DNA, 749 bp, Streptococcus pneumoniae; identical to SEQ ID NO: 13

SEQ ID NO: 16 - DNA, 743 bp, Streptococcus pneumoniae; identical to SEQ ID NO: 12

SEQ ID NO: 17 - DNA, 749 bp, Streptococcus pneumoniae; identical to SEQ ID NO: 13

SEQ ID NO: 18 - DNA, 749 bp, Streptococcus pneumoniae
atgcttttaaatttcttattcatatctatttttctattaattatcattacatttatattatttgagggagatttgtttcaacccgcagtaattttaacacttgcttattttatttcgattgcaagtgctctagttaatagaaatgtttggggaacagaactccatttcaaaacctttggtttgatattgctaggggttgctacatttattatagtttccttgttgacaaaattgtcgtacaaacctaaagtggagggaatttcgtataaagaattaaaagaaataaatccttcaaagataatatatggcattcttctgattctaaatcttgttatgctatttctttatatccatgaaattcagaaagtggtactgttttcaggtagaggtttttctaatattacagatttgataagtaactataggtacctatcttattattcaaatgaagtagaagatcgtgtaagtggaatgattaatcaactagctaaaattattccagcgactacatttgtttctttatatatatttataaataattattttataacgaagcaaataaagaaaaatttcatttatttgattccaatagctatattctttgtctatgcaatcattagtggtggtagactgccccttataaggttagttattggaactctgttgatattgtatatatactctgtgtacgggagtcataaatctcaacttaccaaaagttttaaaatgatcactcgctctctgtttac

SEQ ID NO: 19 - DNA, 749 bp, Streptococcus pneumoniae
atgcttttaaatttcttattcatatctatttttctattaattattattacatttatattatttgagggggatttttttcaacctgcagtaattttaacaatcgcttattttatttcgattgcaagtgctctagttaatagaaatgtttggggaacagaactccatttcaaaaccttttatttgatattgttaggggttgttacatttgttatagtttccttgttgacaaaattgtcgtacaggcctaaagtggagggaatttcgcatgaagaattgaaagaaataaatccttcaaagataatctatgtcattcttctgactctaaatcttgttatgttatttctttatatccgtgaaattcagaaagtagtattgttttcaggtagaagtttttctaatattacagatttgataagtaactataggtacctatcttattattcaaatgaagtagaaaatcgtgtaagtggaatgattaatcaactatctaaaattattccagcgactacacttatttctttatatatatttatgaataattattttataactaaacaaataaagaaaaatttcatttatttgattccaatagctatattctttgtctatgcaatcattagtggtggtagattgccccttataaggttagttgttggagctctgttgatattgtatatatactctgtgtacgggagtcctaaatctcaacttaccaaaagttttaaaatgatcactcgctctctgtttac

SEQ ID NO: 20 - DNA, 749 bp, Streptococcus pneumoniae; identical to SEQ ID NO: 19

SEQ ID NO: 21 - DNA, 749 bp, Streptococcus pneumoniae; identical to SEQ ID NO: 18

SEQ ID NO: 22 - DNA, 625 bp, Streptococcus pneumoniae
tttgaaatggttgtggagttatagattctttttatttaggttaaatggtattaaagaaggaaatagtgaattcgagaagagttttaagataattaggagatactataaaacaggacgatagaatgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctggggtcataagtacagcagttatgcttgccatgaatgtcattcttctagtagtatttgattg

SEQ ID NO: 23 - DNA, 625 bp, Streptococcus pneumoniae; identical to SEQ ID NO: 22

SEQ ID NO: 24 - DNA, 625 bp, Streptococcus pneumoniae
  misc_feature (87)..(87): n=unknown
  misc_feature (583)..(583): n=unknown
tttgaaatggttgtggagttatagattctttttatttaggttaaatggtattaaagaaggaaatagtgaattcgagaagagttttangataattaggagatactataaaacaggacgatagaatgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctgggatcataagtacagcagntatgcttgccatgaatgtcattcttctagtagtatttgattg

SEQ ID NO: 25 - DNA, 625 bp, Streptococcus pneumoniae; identical to SEQ ID NO: 22

SEQ ID NO: 26 - DNA, 625 bp, Streptococcus pneumoniae; identical to SEQ ID NO: 22

SEQ ID NO: 27 - DNA, 625 bp, Streptococcus pneumoniae
  misc_feature (87)..(87): n=unknown
tttgaaatggttgtggagttatagattctttttatttaggttaaatggtattaaagaaggaaatagtgaattcgagaagagttttangataattaggagatactataaaacaggacgatagaatgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctggggtcataagtacagcagttatgcttgccatgaatgtcattcttctagtagtatttgattg

SEQ ID NO: 28 - DNA, 625 bp, Streptococcus pneumoniae
  misc_feature (613)..(613): n=unknown
tttgaaatggttgtggagttatagattctttttatttaggttaaatggtattaaagaaggaaatagtgaattcgagaagagttttaagataattaggagatactataaaacaggacgatagaatgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctggggtcataagtacagcagttatgcttgccatgaatgtcattcttctagnagtatttgattg

SEQ ID NO: 29 - DNA, 607 bp, Streptococcus pneumoniae
tgcgctattaataattttccttatgataactactatatttatagagacctatatgtttttatttgtcatttctttatactattctcttgattttggggacgatagagattgtcatgagaaacagtacattactaattaataataaaggtgtgaacagaaataagaagaaatgaaaatactaaaaaactatgcctacaatctttcttatcaattgttggtgatcatactccctatcattacgactccctatgtaacgagggtttttagttctgacgatttaggaacgtatggctactttagctccattgttacctattttaccttgcttgcaactcttggtgttgccaactacggtaccaaagagatttcagcacatcgtaaggaaattgggaagaatttctggggaatttattctctccagtttggtgcaacttggctatccattttgctttatcttgccctttgtttcttatttacttcaatgcaaaatccggtagcttatatattgggattaagtttagtgtcaaaaggtttggatatttcttggttatttcaaggtttggaggattttagaaagattacagttcggaacatcactgttaagtta

SEQ ID NO: 30 - DNA, 607 bp, Streptococcus pneumoniae
tgcgctattaataattttccttatgataactactatatttatagagacctatatgtttttatttgtcatttctttatactattctcttgattttggggacgatagagattgtcatgagaaacagtacattactaattaataataaaggtgtgaacagaaataagaagaaatgaaaatactaaaaaattatgcctacaatctttcttatcaattgttggtgatcatactccctatcattacgactccctatgtaacgagggtttttagttctgacgatttaggaacgtatggctactttagctccattgttacctattttaccttgcttgcaactcttggtgttgccaactacggtaccaaagagatttcagcacatcgtaaggaaattgggaagaatttctggggaatttattctctccagtttggtgcaacttggctatccattttgctttatcttgccctttgtttcttatttacttcaatgcaaaatccggtagcttatatattgggattaagtttagtgtcaaaaggtttggatatttcttggttatttcaaggtttggaggattttagaaagattacagttcggaacatcactgttaagtta

SEQ ID NO: 31 - DNA, 607 bp, Streptococcus pneumoniae; identical to SEQ ID NO: 30

SEQ ID NO: 32 - DNA, 607 bp, Streptococcus pneumoniae; identical to SEQ ID NO: 29

SEQ ID NO: 33 - DNA, 607 bp, Streptococcus pneumoniae; identical to SEQ ID NO: 30

SEQ ID NO: 34 - DNA, 607 bp, Streptococcus pneumoniae; identical to SEQ ID NO: 30

SEQ ID NO: 35 - DNA, 607 bp, Streptococcus pneumoniae; identical to SEQ ID NO: 29

SEQ ID NO: 36 - DNA, 607 bp, Streptococcus pneumoniae
tgcgctattaataattttccttatgataactactatatttatagagacctatatgtttttatttgtcatttctttatactattctcttgattttggggacgatagagattgtcatgagaaacagtacattactaattaataataaaggtgtgaacagaaataagaagaaatgaaaatactaaaaaactatgcctacaatctttcttatcaattgttggtgatcatactccctatcattacgactccctatgtaacgagggtttttagttctgacgatttaggaacgtatggctactttagctccattgttacctattttaccttgcttgcaactcttggtgttgccaactacggtaccaaagagatttcagcacatcgtaaggaaattgggaagaatttctggggaatttattctctccagtttggtgcaacttggctatccattttgctttatcttgccctttgtttcttatttacttcaatgcaaaatccggtagcttatatattgggattaagtttagtgtcaaaagggttggatatttcttggttatttcaaggtttggaggattttagaaagattacagttcggaacatcactgttaagtta

SEQ ID NO: 37 - DNA, 389 bp, Streptococcus pneumoniae
tgatcatactccctatcattacgactccctatgtaacgagggtcttttcttcggatgatttagggacgtatggttattttaattccatcgttacttattttatcctcttagcgacgctaggagttgctaactatgggaccaaggtcatttcagggcatcgaaagcaaattcaaaaaaactttttgggaatctattctctgcaattaggtgcaacagttctttctctgtccttgtatgctcttctttgtctaactcttccctttatgcaaaatccggtagcctatattctaggcttgagtttagtttctaaaggtttagacatctcctggctctttcaagggttagaagattttcgtaaaattacggtcagaaatatcacagtgaagctt

SEQ ID NO: 38 - DNA, 607 bp, Streptococcus pneumoniae
gagagtttgtacagtcacttactgaatcagtagaggggagaatcttgcctaatttaaagaaaaacattgtttacaatgtcttatatcagatcttagctgtaatagtaccgtttattacctcaccttacttagcgcgtgtgttaggtgcagagcaaattggagtttattcttttacttattccattgctttttactttatgattctgtccatgttgggaatttctaattatgggaatcggacaatggcacaggtacgaacaagtagagaacatttgaatcaagaattttcgaatatttacgcggttcagttgacgtgttcactagtaatgaccgtctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggagtaccctgcttggtcagttgattacatggccattttt

SEQ ID NO: 39 - DNA, 607 bp, Streptococcus pneumoniae
gagagtttgtacagttatttactgaatcagtagaggggagaatcttgcctagtttaaagaaaaatattgtttacaatgtcttatatcagatcttagctgtaatagtaccatttattacctcaccttacttagcgcgtgtgttaggtgcagagcaaattggagtttattcttttacttattccattgctttttactttatgattctgtccatgttggggatttctaattatgggaatcggacaatagcacgggtacgaacaagtagagaacatttgaatcaggaattttcgaatatttacgcggttcagttgacgtgttcactagtaatgaccatctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggggtaccctgcttggtcagttgattacatggccattttt

SEQ ID NO: 40 - DNA, 607 bp, Streptococcus pneumoniae; identical to SEQ ID NO: 39

SEQ ID NO: 41 - DNA, 607 bp, Streptococcus pneumoniae
gagagtttgtacagtcacttactgaatcagtagaggggagaatcttgcctaatttaaagaaaaacattgtttacaatgtcttatatcagatcttagctgtaatagtaccgtttattacctcaccttacttagcgcgtgtgttaggtgcagagcaaattggagtttattcttttacttattccattgctttttactttatgattctgtccatgttgggaatttctaattatgggaatcggacaatagcacaggtacgaacaagtagagaacatttgaatcaagaattttcgaatatttacgcagttcagttgacgtgttcactagtaatgaccgtctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggagtaccctgcttggtcagttgattacatggcaattttt

SEQ ID NO: 42 - DNA, 607 bp, Streptococcus pneumoniae; identical to SEQ ID NO: 41

SEQ ID NO: 43 - DNA, 607 bp, Streptococcus pneumoniae; identical to SEQ ID NO: 41

SEQ ID NO: 44 - DNA, 607 bp, Streptococcus pneumoniae; identical to SEQ ID NO: 41

SEQ ID NO: 45 - DNA, 607 bp, Streptococcus pneumoniae; identical to SEQ ID NO: 41

SEQ ID NO: 46 - DNA, 607 bp, Streptococcus pneumoniae; identical to SEQ ID NO: 41

SEQ ID NO: 47 - DNA, 547 bp, Streptococcus pneumoniae
caggatatagatgcatgttagattagatggtttgctggactatatatttctatttagtgtgattattacttgtaatactatgtattcaactagtcaaggatttgatggactagggaaatgggcgactctgttacttgtggtatcagtttttctgaaattgcttatctctagaatatctatgaaggcaatcaatgtgattgtgtcgcgttctttaatatttatattaattattctactcatagtaatattaaatggttttaagatttctgagacaagtttcgtctattattttgtattatttccgatttttatgatgattttgcagatgtactatgatgttaatgaaatcgcaaatctgatacggaaatttgttcgtataatatttcttttagcaattggctctctcctattttggcttattggtagtgtatttcatattatatccccaacggtttatgtgttgaattattggaatggtgggggaatagtagaagggtactataatcttcattttgaagcacaaaaaatagagattttgggggcgata

SEQ ID NO: 48 - DNA, 547 bp, Streptococcus pneumoniae
caggatatagatgcatgttagattagatggtttgctggactatatatttctatttagtgtgattattacttgtaatactatgtattcaactagtcaaggatttgatggactagggaaatgggcgactctgttacttgtggtatcagttattctgaaattgcttatctctagaatatctatgaaggcaatcaatgtgattgtgtcgcgttctttaatatttatattaattattctactcatagtaatattaaatggttttaagatttctgagacaagtttcgtctattattttgtattatttccgatttttatgatgattttgcagatgtactatgatgttaatgaaatcgcaaatctgatacggaaatttgttcgtataatatttcttttagcaattggctctctcctattttggcttattggtagtgtatttcatattatatccccaacggtttatgtgttgaattattggaatggtgggggaatagtagaagggtactataatcttcattttgaagcacaaaaaatagagattttgggggcgata

SEQ ID NO: 49 - DNA, 547 bp, Streptococcus pneumoniae; identical to SEQ ID NO: 48

SEQ ID NO: 50 - DNA, 547 bp, Streptococcus pneumoniae; identical to SEQ ID NO: 48

SEQ ID NO: 51 - DNA, 547 bp, Streptococcus pneumoniae; identical to SEQ ID NO: 47

SEQ ID NO: 52 - DNA, 547 bp, Streptococcus pneumoniae; identical to SEQ ID NO: 48

SEQ ID NO: 53 - DNA, 547 bp, Streptococcus pneumoniae; identical to SEQ ID NO: 47

SEQ ID NO: 54 - DNA, 547 bp, Streptococcus pneumoniae; identical to SEQ ID NO: 47

SEQ ID NO: 55 - DNA, 547 bp, Streptococcus pneumoniae
tttttagaacgtactcatttatttaaaaggaagtaatagtgaaatttaaatttaaatttaatccaatcgcgatactgtatatattgctagtatacttagagttagctacagataggcaacatctgtatcctgtaacgtacatgacaaaatattatattggtattttaatcactgtgttgtttgttttgttattagtaggccgtgggaagcttatttttgttaataaaaaattattatatcttgctaagatattagctataccaacaattgttcttttcctgtactcagtcttactagacgtaatgaacccagttgaatttgatggatattttagtaggttatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatgctaacagatgatagtttcaatggttcggtactagaaat

SEQ ID NO: 56 - DNA, 547 bp, Streptococcus pneumoniae; identical to SEQ ID NO: 55

SEQ ID NO: 57 - DNA, 541 bp, Streptococcus pneumoniae
tttttagaacatactcatttatttaaaaggaaataatagtgaaatttaaatttaatccaatcgcgatactgtatatattgctagtatacttagagttggctacagataggcaacatctgtatcctgtaacgtacatgacaaaatattatattggtattttaatcattgtgttgtttgttttattattagtaggccgtgggaagcttatttttgttaataaaaaattattatatcttgctaagatattagctataccaacaattgttcttttcctgtactcagtcttactagacgtaatgaacccagttgaatttaatggatattttagtagattatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatactaacagatgatagtttcaatggttcggtactagaaat

SEQ ID NO: 58 - DNA, 541 bp, Streptococcus pneumoniae; identical to SEQ ID NO: 57

SEQ ID NO: 59 - DNA, 541 bp, Streptococcus pneumoniae; identical to SEQ ID NO: 57

SEQ ID NO: 60 - DNA, 541 bp, Streptococcus pneumoniae; identical to SEQ ID NO: 57

SEQ ID NO: 61 - DNA, 541 bp, Streptococcus pneumoniae
tttttagaacatactcatttatttaaaaggaagtaatagtgaaatttaaatttaatccaatcgcgatactgtatatattgctagtatacttagagttggctacagataggcaacatctgtatcctgtaacgtacatgacaaaatattatattggtattttaatcattgtgttgtttgttttattattagtaggccgtgggaagcttatttttgttaataaaaaattattatatcttgctaagatattagctataccaacaattgttcttttcctgtactcagtcttactagacgtaatgaacccagttgaatttaatggatattttagtaggttatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatactaacagatgatagtttcaatggttcggtactagaaat

SEQ ID NO: 62 - DNA, 541 bp, Streptococcus pneumoniae; identical to SEQ ID NO: 57

SEQ ID NO: 63 - DNA, 541 bp, Streptococcus pneumoniae; identical to SEQ ID NO: 57

SEQ ID NO: 64 - DNA, 38 bp, Artificial Sequence
  Oligonucleotide primer
tacattactcgttctctttctttctgcaatcattcttg

SEQ ID NO: 65 - DNA, 32 bp, Artificial Sequence
  Oligonucleotide primer
tagtagctgtcgccttctttaccttgttctgc

SEQ ID NO: 66 - DNA, 28 bp, Artificial Sequence
  Oligonucleotide primer
agaataatcccactcttcttgcggttga

SEQ ID NO: 67 - DNA, 30 bp, Artificial Sequence
  Oligonucleotide primer
catgctgtgagccgttattttttcatactg

SEQ ID NO: 68 - DNA, 37 bp, Artificial Sequence
  Oligonucleotide primer
ggcatytatggagttgattcrtccatycacacyttag

SEQ ID NO: 69 - DNA, 43 bp, Artificial Sequence
  Oligonucleotide primer
cacacyttagaaaayctctatggagtggatatcaattactatg

SEQ ID NO: 70 - DNA, 43 bp, Artificial Sequence
  Oligonucleotide primer
gaaagtggdggdrmktayaaagtraattckcaagayttraarg

SEQ ID NO: 71 - DNA, 31 bp, Artificial Sequence
  Oligonucleotide primer
ccatcayatagaggttamtrtctggcatygc

SEQ ID NO: 72 - DNA, 34 bp, Artificial Sequence
  Oligonucleotide primer
kcatrctraaytcwatyaargcataacgactaty

SEQ ID NO: 73 - DNA, 38 bp, Artificial Sequence
  Oligonucleotide primer
gytcaatrtgrgcaatkactggmgtrattcccracatc

SEQ ID NO: 74 - DNA, 40 bp, Artificial Sequence
  Oligonucleotide primer
gtaggtgtagttttttcagggactttaattttatgcagtg

SEQ ID NO: 75 - DNA, 31 bp, Artificial Sequence
  Oligonucleotide primer
tcgcttaacacaatggctttagaaggtagag

SEQ ID NO: 76 - DNA, 41 bp, Artificial Sequence
  Oligonucleotide primer
gttattttattttttttgtcggcattgtattctttatatcg

SEQ ID NO: 77 - DNA, 33 bp, Artificial Sequence
  Oligonucleotide primer
caaattcatcgtttgtatccatttaactgcatc

SEQ ID NO: 78 - DNA, 46 bp, Artificial Sequence
  Oligonucleotide primer
cttatatctaattatgttccgtctatatttatatgggtttgctttc

SEQ ID NO: 79 - DNA, 39 bp, Artificial Sequence
  Oligonucleotide primer
tttctcttcattttcctgataattttgtacttctgaatg

SEQ ID NO: 80 - DNA, 34 bp, Artificial Sequence
  Oligonucleotide primer
atgcttttaaatttcttattcatatctatttttc

SEQ ID NO: 81 - DNA, 35 bp, Artificial Sequence
  Oligonucleotide primer
rgatttktttcaaccygcagtaattttaacamtyr

SEQ ID NO: 82 - DNA, 41 bp, Artificial Sequence
  Oligonucleotide primer
cctgaaaacartacyactttctgaatttcayggrtataaag

SEQ ID NO: 83 - DNA, 37 bp, Artificial Sequence
  Oligonucleotide primer
gtaaacagagagcgagtgatcattttaaaacttttgg

SEQ ID NO: 84 - DNA, 40 bp, Artificial Sequence
  Oligonucleotide primer
gttttattgactttaaagatgttagtttcttcgattccag

SEQ ID NO: 85 - DNA, 44 bp, Artificial Sequence
  Oligonucleotide primer
tttttattactcttcttaaatcataatgaatcgtaccaatcaac

SEQ ID NO: 86 - DNA, 37 bp, Artificial Sequence
  Oligonucleotide primer
ggatcaatggcaactatatttaccctactctccacag

SEQ ID NO: 87 - DNA, 31 bp, Artificial Sequence
  Oligonucleotide primer
gagtcgaaaccaaccggaaaaagcaattgag

SEQ ID NO: 88 - DNA, 38 bp, Artificial Sequence
  Oligonucleotide primer
cctttggtttattatcctacttccaaaacagtttatgc

SEQ ID NO: 89 - DNA, 40 bp, Artificial Sequence
  Oligonucleotide primer
catatatctctttatcctgtcaatattgattggcattttc

SEQ ID NO: 90 - DNA, 45 bp, Artificial Sequence
  Oligonucleotide primer
gaaattatagtcggagctttcatttatattagtttactggttctg

SEQ ID NO: 91 - DNA, 43 bp, Artificial Sequence
  Oligonucleotide primer
gatattagctataccaacaattgttcttttcctgtactcagtc

SEQ ID NO: 92 - DNA, 36 bp, Artificial Sequence
  Oligonucleotide primer
gcatttctagtaccgaaccattgaaactatcatctg

SEQ ID NO: 93 - DNA, 37 bp, Artificial Sequence
  Oligonucleotide primer
cagaataaagagagctgtaataggtgcaacttcatgc

SEQ ID NO: 94 - DNA, 43 bp, Artificial Sequence
  Oligonucleotide primer
ctgtaatgtttctaattagttcagtatttgcactggttaattc

SEQ ID NO: 95 - DNA, 42 bp, Artificial Sequence
  Oligonucleotide primer
cccgtatatccattactaagaacaaggttgtatatttccttc

SEQ ID NO: 96 - DNA, 38 bp, Artificial Sequence
  Oligonucleotide primer
gtttctcattagttctgtatttgcccttattaatgtgc

SEQ ID NO: 97 - DNA, 33 bp, Artificial Sequence
  Oligonucleotide primer
ccatggctaagtgcaagattatgaatctctctc

SEQ ID NO: 98 - DNA, 36 bp, Artificial Sequence
  Oligonucleotide primer
gtttcttatgtttaccctcagcttatattggcacag

SEQ ID NO: 99 - DNA, 37 bp, Artificial Sequence
  Oligonucleotide primer
gataccacaaatctccgaattctcttaaaatagatgg

SEQ ID NO: 100 - DNA, 38 bp, Artificial Sequence
  Oligonucleotide primer
ttaagtagttcacaagtgatagtgaacttgggattgtc

SEQ ID NO: 101 - DNA, 41 bp, Artificial Sequence
  Oligonucleotide primer
cactgagattatttattagctttatcggtaaggtggataag

SEQ ID NO: 102 - DNA, 37 bp, Artificial Sequence
  Oligonucleotide primer
ccaatgaaaaggaaagttcaatgtgttttgtttctgc

SEQ ID NO: 103 - DNA, 44 bp, Artificial Sequence
  Oligonucleotide primer
attacttgtaatactatgtattcaactagtcmaggatttgatgg

SEQ ID NO: 104 - DNA, 31 bp, Artificial Sequence
  Oligonucleotide primer
gaacaaatttccgtatcagatttgcgatttc

SEQ ID NO: 105 - DNA, 35 bp, Artificial Sequence
  Oligonucleotide primer
ggtgcttcagcaaaaatccccgtatttcttatcag

SEQ ID NO: 106 - DNA, 39 bp, Artificial Sequence
  Oligonucleotide primer
tagctgatgttccgataaattatggtggggtaataatag

SEQ ID NO: 107 - DNA, 44 bp, Artificial Sequence
  Oligonucleotide primer
ctgcgacactgtatatacctacattataactactagacatttgc

SEQ ID NO: 108 - DNA, 40 bp, Artificial Sequence
  Oligonucleotide primer
gcaactttggttctaaaattttagtctttttaatggttcc

SEQ ID NO: 109 - DNA, 40 bp, Artificial Sequence
  Oligonucleotide primer
tgttaaaccccaatatagaaattgtattgagaatagcagc

SEQ ID NO: 110 - DNA, 37 bp, Artificial Sequence
  Oligonucleotide primer
cgttaatagcttatgttcaactggtgattgattttgg

SEQ ID NO: 111 - DNA, 41 bp, Artificial Sequence
  Oligonucleotide primer
tgatagttttagaaataatataaggaattgcaactgcatgc

SEQ ID NO: 112 - DNA, 38 bp, Artificial Sequence
  Oligonucleotide primer
ggtaggtattttaattggaggaagagagtcttgaatgg

SEQ ID NO: 113 - DNA, 41 bp, Artificial Sequence
  Oligonucleotide primer
ttcatgtyyttttrtctaatctgattacaattstyacatcr

SEQ ID NO: 114 - DNA, 36 bp, Artificial Sequence
  Oligonucleotide primer
ygcatttkgatctgtcacartcaataagttaaaacc

SEQ ID NO: 115 - DNA, 39 bp, Artificial Sequence
  Oligonucleotide primer
atcttcccttcataaattgacataggaaaaataagagcc

SEQ ID NO: 116 - DNA, 40 bp, Artificial Sequence
  Oligonucleotide primer
caattctaactatgtccagttttatttttccactcatcag

SEQ ID NO: 117 - DNA, 38 bp, Artificial Sequence
  Oligonucleotide primer
gacgtgataataataagctgccattcctgtctaaaacg

SEQ ID NO: 118 - DNA, 41 bp, Artificial Sequence
  Oligonucleotide primer
cggcggtattaagtagaatattaacacctgaagagtatggc

SEQ ID NO: 119 - DNA, 37 bp, Artificial Sequence
  Oligonucleotide primer
ggcaatcagactcaataagttcatccgtttaaagttc

SEQ ID NO: 120 - DNA, 40 bp, Artificial Sequence
  Oligonucleotide primer
ggtattgcctttcctttgataacttctccttatttatcac

SEQ ID NO: 121 - DNA, 40 bp, Artificial Sequence
  Oligonucleotide primer
tgaacttgtaactcgacacccaaaaatataaataaatgag

SEQ ID NO: 122 - DNA, 36 bp, Artificial Sequence
  Oligonucleotide primer
caaaggaacgttatcagcaattgtgtcaaatttcag

SEQ ID NO: 123 - DNA, 30 bp, Artificial Sequence
  Oligonucleotide primer
gaatcggacaatagcacaggtacgaacaag

SEQ ID NO: 124 - DNA, 32 bp, Artificial Sequence
  Oligonucleotide primer
gccatgtaatcaactgaccaagcagggtactc

SEQ ID NO: 125 - DNA, 34 bp, Artificial Sequence
  Oligonucleotide primer
aagattagggcgcacaaagtttacttgttttagc

SEQ ID NO: 126 - DNA, 40 bp, Artificial Sequence
  Oligonucleotide primer
gttatttcttcaaatctgctcatagttttaacctcatcac

SEQ ID NO: 127 - DNA, 40 bp, Artificial Sequence
  Oligonucleotide primer
tatcttgcgttttcatcccttacagttattaggttcaaag

SEQ ID NO: 128 - DNA, 37 bp, Artificial Sequence
  Oligonucleotide primer
ttcttcaaatcttttgacagtcttgacctcttccttg

SEQ ID NO: 129 - DNA, 40 bp, Artificial Sequence
  Oligonucleotide primer
tatcgtgcattcgaatctgttacagctaatacatttaaac

SEQ ID NO: 130 - DNA, 38 bp, Artificial Sequence
  Oligonucleotide primer
gtcctgacgctatcaaatatcattttcccattaatcac

SEQ ID NO: 131 - DNA, 37 bp, Artificial Sequence
  Oligonucleotide primer
cccacatgtgatcaataggagtgaaaattctctattc

SEQ ID NO: 132 - DNA, 40 bp, Artificial Sequence
  Oligonucleotide primer
cctttggctaatttcttggacgataatgaatttgtatatg

SEQ ID NO: 133 - DNA, 43 bp, Artificial Sequence
  Oligonucleotide primer
gctttggctaacttttcatcaaagattttaatttttttgttag

SEQ ID NO: 134 - DNA, 40 bp, Artificial Sequence
  Oligonucleotide primer
ccagagatagctgtaacaccaattttatcaattcccttag

SEQ ID NO: 135 - DNA, 36 bp, Artificial Sequence
  Oligonucleotide primer
ccacaaacattagcaataaagaaacctaacaatccc

SEQ ID NO: 136 - DNA, 36 bp, Artificial Sequence
  Oligonucleotide primer
gttgttttagctcaaggagggataatgttggcttcg

SEQ ID NO: 137 - DNA, 37 bp, Artificial Sequence
  Oligonucleotide primer
gatcatactccctatcattacgactccctatgtaacg

SEQ ID NO: 138 - DNA, 39 bp, Artificial Sequence
  Oligonucleotide primer
ccaagaaatatccaaaccttttgacactaaacttaatcc

SEQ ID NO: 139 - DNA, 38 bp, Artificial Sequence
  Oligonucleotide primer
gctgattttacaaataggaaaatagagattgcaccaac

SEQ ID NO: 140 - DNA, 36 bp, Artificial Sequence
  Oligonucleotide primer
gcacaaaaaaaagtttgatattccccttgacaatag

SEQ ID NO: 141 - DNA, 32 bp, Artificial Sequence
  Oligonucleotide primer
gcaggatctaaggaggcttcaagattcaactc

SEQ ID NO: 142 - DNA, 41 bp, Artificial Sequence
  Oligonucleotide primer
cgaacctactattgagtgtgatacttttatgggatacagag

SEQ ID NO: 143 - DNA, 38 bp, Artificial Sequence
  Oligonucleotide primer
ctgacagcatgaaaatatataaccgcccaacgaataag

SEQ ID NO: 144 - DNA, 22 bp, Artificial Sequence
  Oligonucleotide primer
ttgagctatttaaggacctggg

SEQ ID NO: 145 - DNA, 22 bp, Artificial Sequence
  Oligonucleotide primer
  misc_feature: Sequence provided in 3′-5′ orientation
agttctttcactgcgaacgatt

SEQ ID NO: 146 - DNA, 26 bp, Artificial Sequence
  Oligonucleotide primer
gtcaataagtttaagtgttatagggc

SEQ ID NO: 147 - DNA, 22 bp, Artificial Sequence
  Oligonucleotide primer
  misc_feature: Sequence provided in 3′-5′ orientation
caagcgttgtgggtagtgatat

SEQ ID NO: 148 - DNA, 22 bp, Artificial Sequence
  Oligonucleotide primer
gatgggaaaatacgatatgctc

SEQ ID NO: 149 - DNA, 22 bp, Artificial Sequence
  Oligonucleotide primer
  misc_feature: Sequence provided in 3′-5′ orientation
cgacctcaaaacagtacctcaa

SEQ ID NO: 150 - DNA, 22 bp, Artificial Sequence
  Oligonucleotide primer
ctttatcaggaatacgccaatc

SEQ ID NO: 151 - DNA, 24 bp, Artificial Sequence
  Oligonucleotide primer
  misc_feature: Sequence provided in 3′-5′ orientation
gcaaccaagagcaataatatgtcc

SEQ ID NO: 152 - DNA, 22 bp, Artificial Sequence
  Oligonucleotide primer
cttttcttcgtatgctttaggg

SEQ ID NO: 153 - DNA, 25 bp, Artificial Sequence
  Oligonucleotide primer
  misc_feature: Sequence provided in 3′-5′ orientation
gactatccacattagagatagaagg

SEQ ID NO: 154 - DNA, 22 bp, Artificial Sequence
  Oligonucleotide primer
gttctttgtttgacccttcctt

SEQ ID NO: 155 - DNA, 22 bp, Artificial Sequence
  Oligonucleotide primer
  misc_feature: Sequence provided in 3′-5′ orientation
tatcttatgcggtctgtcgtaa

SEQ ID NO: 156 - DNA, 24 bp, Artificial Sequence
  Oligonucleotide primer
ttgttcttacatttagccgtagtg

SEQ ID NO: 157 - DNA, 24 bp, Artificial Sequence
  Oligonucleotide primer
  misc_feature: Sequence provided in 3′-5′ orientation
gacagtgagatagtgagtcgttta

SEQ ID NO: 158 - DNA, 22 bp, Artificial Sequence
  Oligonucleotide primer
cagagtttggtcgaggttccta

SEQ ID NO: 159 - DNA, 22 bp, Artificial Sequence
  Oligonucleotide primer
  misc_feature: Sequence provided in 3′-5′ orientation
gagttagttgctgcctttagtg

SEQ ID NO: 160 - DNA, 22 bp, Artificial Sequence
  Oligonucleotide primer
gatccgctcacggtatggacta

SEQ ID NO: 161 - DNA, 22 bp, Artificial Sequence
  Oligonucleotide primer
  misc_feature: Sequence provided in 3′-5′ orientation
gaataaccgactgtcgttttaa

SEQ ID NO: 162 - DNA, 25 bp, Artificial Sequence
  Oligonucleotide primer
tttatgaggagagtactgtatcaga

SEQ ID NO: 163 - DNA, 25 bp, Artificial Sequence
  Oligonucleotide primer
  misc_feature: Sequence provided in 3′-5′ orientation
actcaagctatcgatagtaatttgt

SEQ ID NO: 164 - DNA, 24 bp, Artificial Sequence
  Oligonucleotide primer
tacatttttatgagaagagcattg

SEQ ID NO: 165 - DNA, 25 bp, Artificial Sequence
  Oligonucleotide primer
  misc_feature: Sequence provided in 3′-5′ orientation
gctatcagtactatttttttgtcac

SEQ ID NO: 166 - DNA, 22 bp, Artificial Sequence
  Oligonucleotide primer
ttgttgttgggattgtcttggg

SEQ ID NO: 167 - DNA, 22 bp, Artificial Sequence
  Oligonucleotide primer
  misc_feature: Sequence provided in 3′-5′ orientation
gtttcaaggctttaggtttccg

SEQ ID NO: 168 - DNA, 22 bp, Artificial Sequence
  Oligonucleotide primer
tctttgatttcatcagggattg

SEQ ID NO: 169 - DNA, 22 bp, Artificial Sequence
  Oligonucleotide primer
  misc_feature: Sequence provided in 3′-5′ orientation
atcaccattgacgcaatcagga

SEQ ID NO: 170 - DNA, 22 bp, Artificial Sequence
  Oligonucleotide primer
attgcgactgttaaacgagaag

SEQ ID NO: 171 - DNA, 22 bp, Artificial Sequence
  Oligonucleotide primer
  misc_feature: Sequence provided in 3′-5′ orientation
ccgtgtctaaatacctttatgt

SEQ ID NO: 172 - DNA, 22 bp, Artificial Sequence
  Oligonucleotide primer
taataagcggatgattgtagcg

SEQ ID NO: 173 - DNA, 22 bp, Artificial Sequence
  Oligonucleotide primer
  misc_feature: Sequence provided in 3′-5′ orientation
gggtagacctttcaattagtca

SEQ ID NO: 174 - DNA, 22 bp, Artificial Sequence
  Oligonucleotide primer
tatttccttcctatgggacaac

SEQ ID NO: 175 - DNA, 23 bp, Artificial Sequence
  Oligonucleotide primer
  misc_feature: Sequence provided in 3′-5′ orientation
caccactactaatcgtaataaca

SEQ ID NO: 176 - DNA, 22 bp, Artificial Sequence
  Oligonucleotide primer
aggatgcagtagataccagtgg

SEQ ID NO: 177 - DNA, 22 bp, Artificial Sequence
  Oligonucleotide primer
  misc_feature: Sequence provided in 3′-5′ orientation
cctgttgttggaggcaaatatc

SEQ ID NO: 178 - DNA, 23 bp, Artificial Sequence
  Oligonucleotide primer
ggttctatcaaggaaaagaggac

SEQ ID NO: 179 - DNA, 22 bp, Artificial Sequence
  Oligonucleotide primer
  misc_feature: Sequence provided in 3′-5′ orientation
caacccaagtcactaacgataa

SEQ ID NO: 180 - DNA, 18 bp, Artificial Sequence
  Oligonucleotide primer
cacttccatatccagcat

SEQ ID NO: 181 - DNA, 20 bp, Artificial Sequence
  Oligonucleotide primer
  misc_feature: Sequence provided in 3′-5′ orientation
gacagaggactatcaagagt

SEQ ID NO: 182 - DNA, 21 bp, Artificial Sequence
  Oligonucleotide primer
gcaagtgtttcaatgggagta

SEQ ID NO: 183 - DNA, 21 bp, Artificial Sequence
  Oligonucleotide primer
  misc_feature: Sequence provided in 3′-5′ orientation
gaataacataccagggaggca

SEQ ID NO: 184 - DNA, 21 bp, Artificial Sequence
  Oligonucleotide primer
tttgagaatgcggataaggtg

SEQ ID NO: 185 - DNA, 22 bp, Artificial Sequence
  Oligonucleotide primer
  misc_feature: Sequence provided in 3′-5′ orientation
gagtaacattgtcccgtttgaa

SEQ ID NO: 186 - DNA, 21 bp, Artificial Sequence
  Oligonucleotide primer
cgaaatatcgccattcatcag

SEQ ID NO: 187 - DNA, 22 bp, Artificial Sequence
  Oligonucleotide primer
  misc_feature: Sequence provided in 3′-5′ orientation
tcaccgtgtcaacgacaactaa

SEQ ID NO: 188 - DNA, 20 bp, Artificial Sequence
  Oligonucleotide primer
caatcaataatgccgcatac

SEQ ID NO: 189 - DNA, 22 bp, Artificial Sequence
  Oligonucleotide primer
  misc_feature: Sequence provided in 3′-5′ orientation
ctaaagcaatcaaaggtgtcca

SEQ ID NO: 190 - DNA, 21 bp, Artificial Sequence
  Oligonucleotide primer
tggaggagcaactgacgtatt

SEQ ID NO: 191 - DNA, 21 bp, Artificial Sequence
  Oligonucleotide primer
  misc_feature: Sequence provided in 3′-5′ orientation
gagaacttatacctgccacct

SEQ ID NO: 192 - DNA, 25 bp, Artificial Sequence
  Oligonucleotide primer
gtatgttattcgttagacaaactgg

SEQ ID NO: 193 - DNA, 24 bp, Artificial Sequence
  Oligonucleotide primer
  misc_feature: Sequence provided in 3′-5′ orientation
gacatccaaatacataacgctcaa

SEQ ID NO: 194 - DNA, 23 bp, Artificial Sequence
  Oligonucleotide primer
ctatttaccttgtttcctgcaac

SEQ ID NO: 195 - DNA, 22 bp, Artificial Sequence
  Oligonucleotide primer
  misc_feature: Sequence provided in 3′-5′ orientation
ctattgcgatacagtcgttaag

SEQ ID NO: 196 - DNA, 21 bp, Artificial Sequence
  Oligonucleotide primer
ggattacaagaaattccctcg

SEQ ID NO: 197 - DNA, 21 bp, Artificial Sequence
  Oligonucleotide primer
  misc_feature: Sequence provided in 3′-5′ orientation
tccactatacgcctcggttat

SEQ ID NO: 198 - DNA, 21 bp, Artificial Sequence
  Oligonucleotide primer
tttgggtctcctttacctatc

SEQ ID NO: 199 - DNA, 21 bp, Artificial Sequence
  Oligonucleotide primer
  misc_feature: Sequence provided in 3′-5′ orientation
cactacttctcaatccccttt

SEQ ID NO: 200 - DNA, 22 bp, Artificial Sequence
  Oligonucleotide primer
ccgaaaattgttcacaggatac

SEQ ID NO: 201 - DNA, 23 bp, Artificial Sequence
  Oligonucleotide primer
  misc_feature: Sequence provided in 3′-5′ orientation
ctatacggaacataggtagttag

SEQ ID NO: 202 - DNA, 25 bp, Artificial Sequence
  Oligonucleotide primer
agcagcaattgtttctgtcttaaca

SEQ ID NO: 203 - DNA, 26 bp, Artificial Sequence
  Oligonucleotide primer
  misc_feature: Sequence provided in 3′-5′ orientation
gagattttcactatctacactatctt

SEQ ID NO: 204 - DNA, 25 bp, Artificial Sequence
  Oligonucleotide primer
ctccctatcattactactccctatg

SEQ ID NO: 205 - DNA, 22 bp, Artificial Sequence
  Oligonucleotide primer
  misc_feature: Sequence provided in 3′-5′ orientation
aatccacgctgtcaagaaagtg

SEQ ID NO: 206 - DNA, 26 bp, Artificial Sequence
  Oligonucleotide primer
gtcaataagtttaagtgttatagggc

SEQ ID NO: 207 - DNA, 22 bp, Artificial Sequence
  Oligonucleotide primer
  misc_feature: Sequence provided in 3′-5′ orientation
caagcgttgtgggtagtgatat

SEQ ID NO: 208 - DNA, 22 bp, Artificial Sequence
  Oligonucleotide primer
actcaagtatctgtgycacctt

SEQ ID NO: 209 - DNA, 22 bp, Artificial Sequence
  Oligonucleotide primer
  misc_feature: Sequence provided in 3′-5′ orientation
cctcgtccatctccttcactaa

SEQ ID NO: 210 - DNA, 20 bp, Artificial Sequence
  Oligonucleotide primer
tgagtttccgattagagcag

SEQ ID NO: 211 - DNA, 20 bp, Artificial Sequence
  Oligonucleotide primer
  misc_feature: Sequence provided in 3′-5′ orientation
cctttactacgccatccata

SEQ ID NO: 212 - DNA, 20 bp, Artificial Sequence
  Oligonucleotide primer
tcaatggcgactttatttgc

SEQ ID NO: 213 - DNA, 24 bp, Artificial Sequence
  Oligonucleotide primer
  misc_feature: Sequence provided in 3′-5′ orientation
cgtgggatgtcctctattatctga

SEQ ID NO: 214 - DNA, 22 bp, Artificial Sequence
  Oligonucleotide primer
gtaccgcaagctattctaatga

SEQ ID NO: 215 - DNA, 23 bp, Artificial Sequence
  Oligonucleotide primer
  misc_feature: Sequence provided in 3′-5′ orientation
gtcattctatccgcttcaaatag

SEQ ID NO: 216 - DNA, 24 bp, Artificial Sequence
  Oligonucleotide primer
tagacttcttagagcctattgtgg

SEQ ID NO: 217 - DNA, 22 bp, Artificial Sequence
  Oligonucleotide primer
  misc_feature: Sequence provided in 3′-5′ orientation
ctggttatcgcgtttgacaata

SEQ ID NO: 218 - DNA, 24 bp, Artificial Sequence
  Oligonucleotide primer
caaacccttagtccaatatggctg

SEQ ID NO: 219 - DNA, 24 bp, Artificial Sequence
  Oligonucleotide primer
  misc_feature: Sequence provided in 3′-5′ orientation
ccgatggataataagggaagcaac

SEQ ID NO: 220 - DNA, 22 bp, Artificial Sequence
  Oligonucleotide primer
catttggtatgggagtagggag

SEQ ID NO: 221 - DNA, 23 bp, Artificial Sequence
  Oligonucleotide primer
  misc_feature: Sequence provided in 3′-5′ orientation
gtgaaagaggattgagtacgtgg

SEQ ID NO: 222 - DNA, 22 bp, Artificial Sequence
  Oligonucleotide primer
taatcartggtctggtggtcra

SEQ ID NO: 223 - DNA, 22 bp, Artificial Sequence
  Oligonucleotide primer
  misc_feature: Sequence provided in 3′-5′ orientation
gaaayaaygaggataacygact

SEQ ID NO: 224 - DNA, 21 bp, Artificial Sequence
  Oligonucleotide primer
tgtcagcagaaaatatgacgc

SEQ ID NO: 225 - DNA, 22 bp, Artificial Sequence
  Oligonucleotide primer
  misc_feature: Sequence provided in 3′-5′ orientation
cctttatgctgcttcccaatac

SEQ ID NO: 226 - DNA, 22 bp, Artificial Sequence
  Oligonucleotide primer
ttgttgtagtggcagttgctcc

SEQ ID NO: 227 - DNA, 24 bp, Artificial Sequence
  Oligonucleotide primer
  misc_feature: Sequence provided in 3′-5′ orientation
cggatgtcccttacagaaatgttg

SEQ ID NO: 228 - DNA, 22 bp, Artificial Sequence
  Oligonucleotide primer
tcctgattatrttgagatttsg

SEQ ID NO: 229 - DNA, 22 bp, Artificial Sequence
  Oligonucleotide primer
  misc_feature: Sequence provided in 3′-5′ orientation
gacctaacgcttctgaatgaat

SEQ ID NO: 230 - DNA, 24 bp, Artificial Sequence
  Oligonucleotide primer
caatttccccttattctgtagttc

SEQ ID NO: 231 - DNA, 25 bp, Artificial Sequence
  Oligonucleotide primer
  misc_feature: Sequence provided in 3′-5′ orientation
ctctcttgtcatatttgtcccagtt

SEQ ID NO: 232 - DNA, 23 bp, Artificial Sequence
  Oligonucleotide primer
gattggtttgggaacttgatgtc

SEQ ID NO: 233 - DNA, 25 bp, Artificial Sequence
  Oligonucleotide primer
  misc_feature: Sequence provided in 3′-5′ orientation
caccatactccatagtaaatcgtcc

SEQ ID NO: 234 - DNA, 25 bp, Artificial Sequence
  Oligonucleotide primer
gtagttactggccctttcttattcc

SEQ ID NO: 235 - DNA, 24 bp, Artificial Sequence
  Oligonucleotide primer
  misc_feature: Sequence provided in 3′-5′ orientation
gttctacgtctatcaaagagcgat

SEQ ID NO: 236 - DNA, 21 bp, Artificial Sequence
  Oligonucleotide primer
cagcaaatgcaggttctcaaa

SEQ ID NO: 237 - DNA, 25 bp, Artificial Sequence
  Oligonucleotide primer
  misc_feature: Sequence provided in 3′-5′ orientation
actgtggagcagatcgtatagtaat

SEQ ID NO: 238 - DNA, 22 bp, Artificial Sequence
  Oligonucleotide primer
gatcaaatggtggtattaggaa

SEQ ID NO: 239 - DNA, 22 bp, Artificial Sequence
  Oligonucleotide primer
  misc_feature: Sequence provided in 3′-5′ orientation
cggtcagtataaaaggttaaga

SEQ ID NO: 240 - DNA, 22 bp, Artificial Sequence
  Oligonucleotide primer
ttcttatcgcttccattgtcag

SEQ ID NO: 241 - DNA, 21 bp, Artificial Sequence
  Oligonucleotide primer
  misc_feature: Sequence provided in 3′-5′ orientation
ccacattcacctcgtcgtaaa

SEQ ID NO: 242 - DNA, 25 bp, Artificial Sequence
  Oligonucleotide primer
tatttgccataacggactctagaac

SEQ ID NO: 243 - DNA, 25 bp, Artificial Sequence
  Oligonucleotide primer
  misc_feature: Sequence provided in 3′-5′ orientation
caccaatacacccaaattaagaagc

SEQ ID NO: 244 - DNA, 23 bp, Artificial Sequence
  Oligonucleotide primer
tttgggctctttaggtagtgtat

SEQ ID NO: 245 - DNA, 24 bp, Artificial Sequence
  Oligonucleotide primer
  misc_feature: Sequence provided in 3′-5′ orientation
ctgcctattacaagctatgaaatg

SEQ ID NO: 246 - DNA, 21 bp, Artificial Sequence
  Oligonucleotide primer
catttggagttattgccctac

SEQ ID NO: 247 - DNA, 23 bp, Artificial Sequence
  Oligonucleotide primer
  misc_feature: Sequence provided in 3′-5′ orientation
ccccagaattaaatcttataccc

SEQ ID NO: 248 - DNA, 22 bp, Artificial Sequence
  Oligonucleotide primer
agggcttaactgtttcagtgtt

SEQ ID NO: 249 - DNA, 22 bp, Artificial Sequence
  Oligonucleotide primer
  misc_feature: Sequence provided in 3′-5′ orientation
ctaaaccatatcgtcctgactt

SEQ ID NO: 250 - DNA, 20 bp, Artificial Sequence
  Oligonucleotide primer
ttatctatatgttagggctg

SEQ ID NO: 251 - DNA, 20 bp, Artificial Sequence
  Oligonucleotide primer
  misc_feature: Sequence provided in 3′-5′ orientation
ctgtgaagacttacaacatg

SEQ ID NO: 252 - DNA, 21 bp, Artificial Sequence
  Oligonucleotide primer
ttggatcgttgttcatagcgg

SEQ ID NO: 253 - DNA, 22 bp, Artificial Sequence
  Oligonucleotide primer
  misc_feature: Sequence provided in 3′-5′ orientation
gacacctttacggcaacgattc

SEQ ID NO: 254 - DNA, 23 bp, Artificial Sequence
  Oligonucleotide primer
agcgagcggtatcattctatttg

SEQ ID NO: 255 - DNA, 25 bp, Artificial Sequence
  Oligonucleotide primer
  misc_feature: Sequence provided in 3′-5′ orientation
ctatcacaacttctttaacgaggtc

SEQ ID NO: 256 - DNA, 22 bp, Artificial Sequence
  Oligonucleotide primer
tcaacacttatgatggtgcctg

SEQ ID NO: 257 - DNA, 21 bp, Artificial Sequence
  Oligonucleotide primer
  misc_feature: Sequence provided in 3′-5′ orientation
atcttcaccctaatagcccga

SEQ ID NO: 258 - DNA, 22 bp, Artificial Sequence
  Oligonucleotide primer
aatctgaggaaacttggagcaa

SEQ ID NO: 259 - DNA, 24 bp, Artificial Sequence
  Oligonucleotide primer
  misc_feature: Sequence provided in 3′-5′ orientation
gcataattgctaatcttaacaagg

SEQ ID NO: 260 - DNA, 25 bp, Artificial Sequence
  Oligonucleotide primer
gcaatggtttatggatgatagagcg

SEQ ID NO: 261 - DNA, 22 bp, Artificial Sequence
  Oligonucleotide primer
  misc_feature: Sequence provided in 3′-5′ orientation
tgtgctgctaacgaccacgaaa

SEQ ID NO: 262 - DNA, 23 bp, Artificial Sequence
  Oligonucleotide primer
tgaaaatcccttagtgacatctg

SEQ ID NO: 263 - DNA, 22 bp, Artificial Sequence
  Oligonucleotide primer
  misc_feature: Sequence provided in 3′-5′ orientation
gaccagcatcgtaaagagtcta

SEQ ID NO: 264 - DNA, 22 bp, Artificial Sequence
  Oligonucleotide primer
cggtatgcttacaatgagacgc

SEQ ID NO: 265 - DNA, 22 bp, Artificial Sequence
  Oligonucleotide primer
  misc_feature: Sequence provided in 3′-5′ orientation
gtagaataggcccttgcttaag

SEQ ID NO: 266 - DNA, 22 bp, Artificial Sequence
  Oligonucleotide primer
gtaacgatgcctagaatgactt

SEQ ID NO: 267 - DNA, 23 bp, Artificial Sequence
  Oligonucleotide primer
  misc_feature: Sequence provided in 3′-5′ orientation
cacaccattatccacgacaatag

SEQ ID NO: 268 - DNA, 25 bp, Artificial Sequence
  Oligonucleotide primer
ctaatttggctatgaagctaatccc

SEQ ID NO: 269 - DNA, 25 bp, Artificial Sequence
  Oligonucleotide primer
  misc_feature: Sequence provided in 3′-5′ orientation
caaatgactgacgctgaaatcactt

SEQ ID NO: 270 - DNA, 24 bp, Artificial Sequence
  Oligonucleotide primer
ctatgcaggaaatatccgagaagg

SEQ ID NO: 271 - DNA, 24 bp, Artificial Sequence
  Oligonucleotide primer
  misc_feature: Sequence provided in 3′-5′ orientation
gtatcgcaaagacaaagtgcctag

SEQ ID NO: 272 - DNA, 22 bp, Artificial Sequence
  Oligonucleotide primer
aatggcttgctcctattgctgt

SEQ ID NO: 273 - DNA, 23 bp, Artificial Sequence
  Oligonucleotide primer
  misc_feature: Sequence provided in 3′-5′ orientation
cgtttagcaagaaccctatcatc

SEQ ID NO: 274 - DNA, 25 bp, Artificial Sequence
  Oligonucleotide primer
gtcaaagacaggaatgacatctatg

SEQ ID NO: 275 - DNA, 22 bp, Artificial Sequence
  Oligonucleotide primer
  misc_feature: Sequence provided in 3′-5′ orientation
ccctccttcacgaaaataaaga

SEQ ID NO: 276 - DNA, 18 bp, Artificial Sequence
  Oligonucleotide primer
ggaatcggacaatagcac

SEQ ID NO: 277 - DNA, 20 bp, Artificial Sequence
  Oligonucleotide primer
  misc_feature: Sequence provided in 3′-5′ orientation
accagaacttctcaaagcat

SEQ ID NO: 278 - DNA, 18 bp, Artificial Sequence
  Oligonucleotide primer
ggcatcaaaggttaagtg

SEQ ID NO: 279 - DNA, 18 bp, Artificial Sequence
  Oligonucleotide primer
  misc_feature: Sequence provided in 3′-5′ orientation
gaagacagcgttgagaaa

SEQ ID NO: 280 - DNA, 20 bp, Artificial Sequence
  Oligonucleotide primer
gctatctaacattgcgagta

SEQ ID NO: 281 - DNA, 18 bp, Artificial Sequence
  Oligonucleotide primer
  misc_feature: Sequence provided in 3′-5′ orientation
aaaccgaaggacgaatat

SEQ ID NO: 282 - DNA, 18 bp, Artificial Sequence
  Oligonucleotide primer
tagcggtgaatggcatct

SEQ ID NO: 283 - DNA, 19 bp, Artificial Sequence
  Oligonucleotide primer
  misc_feature: Sequence provided in 3′-5′ orientation
agttggaatcatcctcgct

SEQ ID NO: 284 - DNA, 19 bp, Artificial Sequence
  Oligonucleotide primer
gggaaatggtttactatgc

SEQ ID NO: 285 - DNA, 19 bp, Artificial Sequence
  Oligonucleotide primer
  misc_feature: Sequence provided in 3′-5′ orientation
gttcttctattctcgccta

SEQ ID NO: 286 - DNA, 19 bp, Artificial Sequence
  Oligonucleotide primer
atttatgaagggaagatgg

SEQ ID NO: 287 - DNA, 18 bp, Artificial Sequence
  Oligonucleotide primer
  misc_feature: Sequence provided in 3′-5′ orientation
ccgagcgtcattatcaaa

SEQ ID NO: 288 - DNA, 18 bp, Artificial Sequence
  Oligonucleotide primer
tatgtttcaagggttctg

SEQ ID NO: 289 - DNA, 18 bp, Artificial Sequence
  Oligonucleotide primer
  misc_feature: Sequence provided in 3′-5′ orientation
ccttaccgtcgaataata

SEQ ID NO: 290 - DNA, 18 bp, Artificial Sequence
  Oligonucleotide primer
tgataaggcttaccagtt

SEQ ID NO: 291 - DNA, 18 bp, Artificial Sequence
  Oligonucleotide primer
  misc_feature: Sequence provided in 3′-5′ orientation
ctgaccataaccctgatt

SEQ ID NO: 292 - DNA, 18 bp, Artificial Sequence
  Oligonucleotide primer
tgaatatggacggtggag

SEQ ID NO: 293 - DNA, 18 bp, Artificial Sequence
  Oligonucleotide primer
  misc_feature: Sequence provided in 3′-5′ orientation
gaaagccgaaagaaacga

SEQ ID NO: 294 - DNA, 18 bp, Artificial Sequence
  Oligonucleotide primer
gattggctgttcaagtgt

SEQ ID NO: 295 - DNA, 18 bp, Artificial Sequence
  Oligonucleotide primer
  misc_feature: Sequence provided in 3′-5′ orientation
ccctgcctaaatgtaatc

SEQ ID NO: 296 - DNA, 20 bp, Artificial Sequence
  Oligonucleotide primer
ttgttcttacatttagccgt

SEQ ID NO: 297 - DNA, 18 bp, Artificial Sequence
  Oligonucleotide primer
  misc_feature: Sequence provided in 3′-5′ orientation
ccctgaacctaaaccatt

SEQ ID NO: 298 - DNA, 18 bp, Artificial Sequence
  Oligonucleotide primer
catgaagttgcacctatt

SEQ ID NO: 299 - DNA, 18 bp, Artificial Sequence
  Oligonucleotide primer
  misc_feature: Sequence provided in 3′-5′ orientation
ccctatcccaaacattgt

SEQ ID NO: 300 - DNA, 18 bp, Artificial Sequence
  Oligonucleotide primer
aaacggaaagttggatgg

SEQ ID NO: 301 - DNA, 19 bp, Artificial Sequence
  Oligonucleotide primer
  misc_feature: Sequence provided in 3′-5′ orientation
cagaaacgacatccacgaa

SEQ ID NO: 302 - DNA, 20 bp, Artificial Sequence
  Oligonucleotide primer
tgtcggcattgtattcttta

SEQ ID NO: 303 - DNA, 18 bp, Artificial Sequence
  Oligonucleotide primer
  misc_feature: Sequence provided in 3′-5′ orientation
cccagtcctaaaccacca

SEQ ID NO: 304 - DNA, 18 bp, Artificial Sequence
  Oligonucleotide primer
tagggaaatgggcgactc

SEQ ID NO: 305 - DNA, 20 bp, Artificial Sequence
  Oligonucleotide primer
  misc_feature: Sequence provided in 3′-5′ orientation
acctcaaaccataactcgga

SEQ ID NO: 306 - DNA, 18 bp, Artificial Sequence
  Oligonucleotide primer
attccagcgactacactt

SEQ ID NO: 307 - DNA, 18 bp, Artificial Sequence
  Oligonucleotide primer
  misc_feature: Sequence provided in 3′-5′ orientation
aatcaccaccatctaacg

SEQ ID NO: 308 - DNA, 18 bp, Artificial Sequence
  Oligonucleotide primer
cacgcagactagaacagc

SEQ ID NO: 309 - DNA, 19 bp, Artificial Sequence
  Oligonucleotide primer
  misc_feature: Sequence provided in 3′-5′ orientation
gaaccagatacatacgcca

SEQ ID NO: 310 - DNA, 18 bp, Artificial Sequence
  Oligonucleotide primer
gttggtttcgactctttg

SEQ ID NO: 311 - DNA, 18 bp, Artificial Sequence
  Oligonucleotide primer
  misc_feature: Sequence provided in 3′-5′ orientation
ttttgcgatgactgttac

SEQ ID NO: 312 - DNA, 18 bp, Artificial Sequence
  Oligonucleotide primer
ttcggagatttgtggtat

SEQ ID NO: 313 - DNA, 18 bp, Artificial Sequence
  Oligonucleotide primer
  misc_feature: Sequence provided in 3′-5′ orientation
agcaaatacctccaccta

SEQ ID NO: 314 - DNA, 19 bp, Artificial Sequence
  Oligonucleotide primer
tggagaatttgcgattacg

SEQ ID NO: 315 - DNA, 20 bp, Artificial Sequence
  Oligonucleotide primer
  misc_feature: Sequence provided in 3′-5′ orientation
tagagtcccatttgtctcac

SEQ ID NO: 316 - DNA, 20 bp, Artificial Sequence
  Oligonucleotide primer
aatgcttgtactactccctc

SEQ ID NO: 317 - DNA, 18 bp, Artificial Sequence
  Oligonucleotide primer
  misc_feature: Sequence provided in 3′-5′ orientation
gatactaaatgcctaccg

SEQ ID NO: 318 - DNA, 21 bp, Artificial Sequence
  Oligonucleotide primer
ttccctatgtcagtctatgaa

SEQ ID NO: 319 - DNA, 20 bp, Artificial Sequence
  Oligonucleotide primer
  misc_feature: Sequence provided in 3′-5′ orientation
tcttcatagtatcggcttaa

SEQ ID NO: 320 - DNA, 22 bp, Artificial Sequence
  Oligonucleotide primer
tattctatttcttacccgctac

SEQ ID NO: 321 - DNA, 20 bp, Artificial Sequence
  Oligonucleotide primer
  misc_feature: Sequence provided in 3′-5′ orientation
attcacccgttcaaagtaga

SEQ ID NO: 322 - DNA, 20 bp, Artificial Sequence
  Oligonucleotide primer
gtgcctagtagcattccata

SEQ ID NO: 323 - DNA, 20 bp, Artificial Sequence
  Oligonucleotide primer
  misc_feature: Sequence provided in 3′-5′ orientation
gaaaccaatgataccaccac

SEQ ID NO: 324 - DNA, 20 bp, Artificial Sequence
  Oligonucleotide primer
tcgcctagtctaaataccaa

SEQ ID NO: 325 - DNA, 20 bp, Artificial Sequence
  Oligonucleotide primer
  misc_feature: Sequence provided in 3′-5′ orientation
aagtgaatcttaaagccgtc

SEQ ID NO: 326 - DNA, 22 bp, Artificial Sequence
  Oligonucleotide primer
agagggattgttgaaggtattc

SEQ ID NO: 327 - DNA, 22 bp, Artificial Sequence
  Oligonucleotide primer
  misc_feature: Sequence provided in 3′-5′ orientation
cctactatctttacgctctgat

SEQ ID NO: 328 - DNA, 22 bp, Artificial Sequence
  Oligonucleotide primer
ggcgttgtcagtgctagtttag

SEQ ID NO: 329 - DNA, 24 bp, Artificial Sequence
  Oligonucleotide primer
  misc_feature: Sequence provided in 3′-5′ orientation
ctcatattaccgacgaaattgtcc

SEQ ID NO: 330 - DNA, 24 bp, Artificial Sequence
  Oligonucleotide primer
ataaaaagaaagtctttgccagag

SEQ ID NO: 331 - DNA, 23 bp, Artificial Sequence
  Oligonucleotide primer
  misc_feature: Sequence provided in 3′-5′ orientation
ctactacttgtatcagcgataac

SEQ ID NO: 332 - DNA, 22 bp, Artificial Sequence
  Oligonucleotide primer
ccgaaaattgttcacaggatac

SEQ ID NO: 333 - DNA, 23 bp, Artificial Sequence
  Oligonucleotide primer
  misc_feature: Sequence provided in 3′-5′ orientation
ctatacggaacataggtagttag

SEQ ID NO: 334 - DNA, 1413 bp, Streptococcus pneumoniae
atgaaagtactaaaaaactacgcttataatctttcttatcaattgttggtgattatactccctatcattacgactccctatgtgacacgggtcttttcttcggatgacctagggacatatggttattttaactccatcgtgacttattttatcctcttagcaacgctaggagttgccaactatgggaccagggtcatttcaggatataggaaagaaattgaaaaaaatttttggggaatttattctttgcaactaggtgcaaccgttctttctctatccttgtatagtgttttttgcctaactcttccctttatgcaaaatcctgtagcctacattttaggcttgagtttggtttctaaaggattagatatatcatggctttttcaagggttggaggattttcgtaagattacggttcgaaatattacagtaaaattagttggagtgttctccatcttcctctttgtcaaatctgcaaatgacctctatctctatgtctttttacttactatatttgaactcttggggcagctaagtacgtggttgcctgctcgtgagtttattggtagacctcattttgatttagaatatgctaagcatcatttgaaacccgttatattattattccttcctcaagtagctatctctttgtatgttactctagatcgtaccatgcttggagccttagcttctacaaaagatgtaggaatttatgaccaggccctaaaattagtaaatatccttctgaccttggtaacttccttgggaagcgttatgttgcctcgtgtcgcgcatttgttagcgacaggtaatcacaaagcggttaacaaaatgcatgagatgtccttcctcatttataatctagtgattttcccaatgatggcaggaattttaatcgtcaatgatgattttgttagctttttccttgggcagaattttcaggatgcacgttatgcaattgccattatgatcttccgtatgttctttatcggttggaccaatatcatgggaattcagatactgatacctcacaatcaaaataaagaattcatgatttcaacaacagttcccgcaattatcagtgtaggtttgaacttactattccttcctaaacttggatatatcggagcagccattgtttctgttttgacagaggcgttggtatgggcgatacaattatactttacccgtagatacctaaaagaagttccgattatcggatttttgacaaaaattattctagcttcagctatcatgtatggtatcttgctaggtttaaaaacagttatacattttttaccaatcataaatgttatagtatttgcagtgcttggtggaatcatttatctttttgcaattctatctttgaaagtggtagatgtaaaagaattaaaacaaatcattaagaaataa

SEQ ID NO: 335 - DNA, 1413 bp, Streptococcus pneumoniae
atgaaagtactaaaaaattacgcctataacctctcctatcaactattactcattatactacctatcattacaactccctatgtgacacgggtcttttctttgaatgatctagggacttatggttattttaactccatcgttacttattttattcttttagcaactttaggagttgctaactatgggaccaaggtcatttcagggcatcgcaaggaaattgaaaaaaatttttgggggatttactccctgcaattgggtgcgacagttctttctatgttcttgtaccttgttctttgtctaactcttccttttatgcaaaatccagtcgcctatattctaggcttgagtttggtttctaaaggcttagatatttcctggctctttcaaggattagaggattttcgtaagattacagcaagaaatatcattgtgaaactcgtaggtgtaagttcgatttttttatttataaaatcagctagtgacctttacctttatgtatttcttttgacaatatttgagttgttgggacagttaagcatgtggttacccgctcgggaatttattgggaaaccacattttgatttaagctatgcaaagcagcatctgaagccaattgttttattattccttcctcaggtagcgatctccttatatgttactctaaatcgtacgatgcttggtgctttatcttctacaaaagatgtaggaatttatgatcaggctcttaaattggtaaatattttattaacattggtaacatcgcttgggagtgttatgttacctcgagttgctaatttattagcaacaggtgattacaaagctgttaataagatgcatgaaatgtccttcctcatttataatttagtgatttttccaattatggctggaattttgattgtgaatgatgattttgttcagtttttccttggtcaagattttcaggatgcacgttatgcaatcgccattatgatcttccgtatgttcttcatcggttggaccaatataatgggaattcagattttaattccccataataaaaataaagaattcatgatttcaacaacagtttctgcaattatcagtgtaggattaaacatgcttttcctaccaaagcttggctatattggagcagccattgtttctgtattaacagaagcacttgtatgggcaattcaattgttctatactcgcagatatctcaaagaagttcctatcatagtatctatgacaaaaattgttctagcatcagttatcatgtatggcattttgctaggttcaaaaatatttatacatttttccccgactataaatgttctagcatttgcagtacttggtggaatcatttacctttttgcaattttatctatgaaagtaatagatgtgaaagaattaaaacaaatcataaaaaaataa

SEQ ID NO: 336 - DNA, 1416 bp, Streptococcus pneumoniae
atgaaagtattaaaaaactacgcctacaatctttcttatcaattgttggtgattatacttccgattattacgactccctatgtaacacgggtcttttcttcggatgatttagggacgtatggatactttaattccattgttacttattttatcctcttggcgacactaggagttgctaactatgggaccaaggtcatttcagggcatcgcaaggaaattgaaaaaaacttttggggtatctactctctacaattaggggcaacagttctttctctgtctttatatgctcttctttgtctgacccttccctttatgcaaaatccggtagcctacattctaggcttgagtttagtttcaaaaggtttagacatttcctggctctttcaagggctagaagattttcgaaagattactgttcgaaatatcacagttaaactcgttggagtcatttcaatctttatctttgtcaaatctgcaaacgacctatatctctatgtttttttgctaaccatatttgaacttttagggcaactaagtatgtggttacctgctcgggagtttatcggtagttctcattttagcatagaatatgctaggcatcatttaaagccggtcatattattattccttcctcaagtagctatttctttgtacattacgctggatcgtaccatgcttggagcactggcgtctacaaaagatgtagggatctatgatcaggcacttaagttggtaaatatccttctgattttggtaacttccttgggaagcgttatgttgcctcgtgtcgcgaatttgttagcgacaggtgatcacaaagcggttaacaaaatgcatgagatgtccttcctcatttataatttagtgattttcccaatgatggcaggaattttgattgtgaatgatgattttgttagctttttccttgggcaggattttcaggatgcacgttatgcaattgccattatgatcttccgtatgttctttatcggttggaccaatatcatgggaattcagattttaattccccataataaaaataaagaattcatgatttcaacaacagctcccgcaattatcagtgtaggtttgaacttactattccttcctaaacttggatatatcggagcagccattgtttctgttttgacagaggcgttggtatgggcgatacaattatactttacccgtagatacctaaaagaagttccgattatcggatctttgacaaaaattattctagcttcagctatcatgtatggtatcttgctaggtttaaaaacagttatacatttttcaccaatcataaatgttatagtatttgcagtgcttggtggaatcatttatctttttgcaattctatctttgaaagtggtagatgtaaaagaattaaaacaaattattaggaaaaaataa

SEQ ID NO: 337 - DNA, 1416 bp, Streptococcus pneumoniae
atgaaagtattaaaaaactacgcctacaatctttcttatcaattgttggtgattatacttccgattattacgactccctatgtaacacgggtcttttcttcggatgatttagggacgtatggatactttaattccattgttacttattttatcctcttggcgacactaggagttgctaactatgggaccaaggtcatttcagggcatcgcaaggaaattaaaaaaaacttttggggtatctactctctacaattaggggcaacagttctttctctgtctttatatgctcttctttgtctgacccttccctttatgcaaaatccggtagcctacattctaggcttgagtttagtttcaaaaggtttagacatttcctggctctttcaagggctagaagattttcgaaagattactgttcgaaatatcacagttaaactcgttggagtcatttcaatctttatctttgtcaaatctgcaaatgacctatatctctatgtttttttgctaaccatatttgaacttttggggcaactaagtatgtggttccctgctcgggagtttatcggtagtgctcattttagcatagaatatgctaggcatcatttaaagccggtcatattattattccttcctcaagtagctatttctttgtacattacgctggatcgtaccatgcttggagcactggcgtctacaaaagatgtagggatctatgatcaggcacttaagttggtaaatatccttctgattttggtaacttccttgggaagcgttatgttgcctcgtgttgcgaatttgttagcgacaggtgaacacaaagcggttaacaaaatgcatgagatgtccttcctcatttataatttagtgattttctcaatgatggcaggaattttgattgtgaatgatgattttgttagctttttccttgggcaggattttcaggatgcacgttatgcaattgccattatgatcttccgtatgttctttatcggttggaccaatatcatgggaattcagattttaattccccataataaaaataaagaattcatgacttcaacaacagctcccgcaattatcagtgtaggtttgaacttactattccttcctaaatttggatatatcggagcagccattgtttctgttttgacagaggcgttggtatgggcgatacaattatactttacccgtagatacctaaaagaagttccgattatcggatctttgacaaaaattattctagcttcagctatcatgtatggtatcttgctaggtttaaaaacagttatacatttttcaccaatcataaatgttatagtatttgcagtgcttggtggaatcatttatctttttgcaattctatctttgaaagtggtagatgtaaaagaattaaaacaaattattaggaaaaaatag

SEQ ID NO: 338 - DNA, 1464 bp, Streptococcus pneumoniae
atggagaatcgtgtgaaagattggcacaaattatggaattataaaagccgttacgaagaaatgtctgtcgggatgaaagcggcactctggtatactgttgcaaattttattcaaaagggttttgtatttctttcaactccgattttaaccagaatacttaatgttaatgattacggtacattgatactttaccaatcatggtttgtactatttgctatttttgcgaccttgaatctttctcaaacagcttataataagggattggtagaatttgagaatgataaagataattttacattttctttgttgtttttgtctaagactattactataattgttgcttttttatattttacttttaatatactggttaaagatagtactggattaactcccagtttcatgatatttttattcagtgatatcttatttaatattagtatagaattttatttggcacgtcagagatttgaattcaaatatagaaaagcagttctaatttcaactttttcaagtttttttattgtagctattagtacaactatcttattagcgattaagaatgatttattcattaaaattgtattggatgctattatccgaattatctttggtttatattgtttttatctcttgtttttttcagggagaaggcaacttaagatagaaaaaaaatatattaaatatggtttgagttattcattacctttgataccgcatttcttatcgcactatattttgaatcagtctgaccgtttgatgattaacatgtttgatggtaaggagaaattagctatatatagtctagcttattctgtatcaatgattatgtttttgtttactaatgcaattaatcaatcaataatgccgcatacatttcaagctttgaaaaaaaaggattattgtgggatacatgggagcaccaaatggctttttgtagtagttggggggattacagctctctcaattctatttgcaccagaactaattgttattttaggcggcaataaatataaggaatcaatttggctagtacctccaattgccatctcggtttatttcctttttgtatattctatgtttagtaatatcagtttttattataagatgaataagctgatttcgttagtttccacaggtgcggctctttcaaatattattttaaattatatttttattaatatatttgggtatcaagctgcagcttacactacattattatgctatatattgttggcgttttctcattttttcttatatagatttttattaaaaaaagaggaaattcatgaggaactttataacatgaagatgattttaataatatctttaatattattaataatactatttttgatacttgttatttataacttagccattataagatatgcaattattgcaattattttatttcttttgtttacaaagagaaataaaataattacatcgctaaaaagttga

SEQ ID NO: 339 - DNA, 1464 bp, Streptococcus pneumoniae; identical to SEQ ID NO: 338

SEQ ID NO: 340 - DNA, 1545 bp, Streptococcus pneumoniae
atgagtcgtgtaaaaaatagtttttttaatattttagctggaatagttggtactatcatatcgagtgttttagctttcatagttagaacagtgtttatccgagttttgggagagacttatctaggtttcaatggtctatacaccaatatcttaacagttttatccctaactgaattagggatcggatcatcaattgcttatttgatgtacaaacctttagcagaaaaagatggggacaaattagctcaagtagttcaattctataaaaaaatatatcgagtgattggtatcattattcttattttaggtttgtgtctcgttccttttttaccagtcattgtaaatttaaaaaatgcagaagatttgaactatacagcattgtatttgctttatttagcagatacagtttctacttatctattctttgcatataaaagaggggtattggctgctgatcaaaagatctatgttgcgaatatctttgatataattataacgacaattttatctattttccaaattatcactttgatgatttttaaagacttttatctatatattattttaaaaattgctaaaaatcttactttgaacctagtaattagtttaaaagtagataaaatgtatccaagtattcataaatttaaagggatagctccgcttccaaaaatggaaagacgactagtttggaaaaatgtatatgctacctctgttcgacaaatctttaacgaattaatgaattcaacagatagtatagttatttccattgtcttaggaatagttatggttggtaagtattcgaactatgcttatattttatcaatcgtatatattttctttggaggaatttttaatccgattcaggcttctattggaaacctatctctgtcagcttctatagagaaaaaaaatgaaatcttcaatagattacgttttataaattttttctttttatcattttgctcaagttgtcttctagtattggtaaatccttttatcactatttggattggagaaaattacactttatcatttacaggtgtgattgcaatagttggtatgttattcgttagacaaactggaaattgtacaaccatttttcgtttgggagaaggatattttagagattatcatttttctcctttaatcgcgggcattttaaatttagttgtttcagtaattttagtgaattatataggtattgcaggtgtttttgttggaacaatgttaggatttggtttccaatttatactggtggatacaattgttacttataaaaaagtacttagtcgtccactatcagagttttatttaagatggttgcagacgatcttattaactgtaggtttatgtattgcgagttactatttatctcgattggttagagtgaatagtatatatgatttgatactgttgtttgtagtggttattggatttaatttttttgctttatgttttatttattggcgaaatgatgattttcaatattttattcaattggttaaaaattttatgaaaaatttggaggaaaaaaatcatgattaa

SEQ ID NO: 341 - DNA, 1545 bp, Streptococcus pneumoniae
atgagtcgtgtaaaaaatagtttttttaatattttagctggaatagttggtactatcatatcgagtgttttagctttcatagttagaacagtgtttatccgagttttgggagagacttatctaggtttcaatggtctatacaccaatatcttaacagttttatccctagctgaattagggatcggatcatcaattgcttatttgatgtacaaacctttagcagaaaaagatggggacaaattagctcaagtagttcaattctataaaaaaatatatcgagtaattggtatcattattcttattttaggtttgtgtctcgttccttttttaccagtcattgtaaatttaaaaaatgcagaagatttgaactatacagcattgtatttgctttatttagcagatacagtttctacttatctattctttgcatataaaagaggggtattggttgctgatcaaaaggtctatgttgcgaatatctttgatataattataacgacaattttatctattttccaaattatcactttgatgatttggaaagatttttatctatatattattttaaaaattgctaaaaatcttactttgaacctagtaattagtttaaaagtagataaaatgtatccaagtattcataaatttaaagagatagctccacttccaaaaatggaaagacgactagtttggaaaaatgtatatgctacctctgttcgacaaatctttaacgaattaatgaattcaacagatagtatagttatttccattgttttaggaatagttatggttggtaagtattcgaactatgcttatattttatcaatcgtatatattttctttggaggaatttttaatccgattcaggcttctattggaaacctatctctgttagcttctatagagaaaaaaaatgaaatcttcaatagattacgttttataaattttttctttttatcattttgctcaagttgtcttctagtattggtaaatccttttatcactatttggattggagaaaattacactttatcatttacaggtgtgattgcaatagttggtatgttattcgttagacaaactggaaattgtacaaccatttttcgtttgggagaaggatattttagagattatcatttttctcctttaatcgcgggcattttaaatttagttgtttcagtaattttagtgaattatataggtattgcaggtgtttttgttggaacaatgttaggatttggtttccaatttatactggtggatacaattgttacttataaaaaagtacttagtcgtccactatcagagttttatttaagatggttgcagacgatcttattaactgtaggtttatgtattgcgagttactatttatctcgattggttagagtgaatagtatatatgatttgatactgttgtttgtagtggttattggatttaatttttttgctttatgttttatttattggcgaaatgatgattttcaatattttattcaattggttaaaaattttatgaaaaatttggaggaaaaaaatcatgattaa

SEQ ID NO: 342 - DNA, 1545 bp, Streptococcus pneumoniae
atgagtcgtgtaaaaaatagtttttttaatattttagctggaatagttggtactatcatatcgagtgttttagctttcatagttagaacagtgtttatccgagttttgggagagacttatctaggtttcaatggtctatacaccaatatcttaacagttttatccctagctgaattagggatcggatcatcaattgcttatttgatgtacaaacctttagcagaaaaagatggggacaaattagctcaagtagttcaattctataaaaaaatatatcgagtaattggtataattattcttattttaggtttgtgtctcgttccttttttaccagtcattgtaaatttaaaaaatgcagaagatttgaactatacagcattgtatttgctttatttagcagatacagtttctacttatctattctttgcatataaaagaggggtattggttgctgatcaaaaggtctatgttgcgaatatctttgatataattataacgacaattttatctattttccaaattatcactttgatgatttttaaagatttttatctatatattattttaaaaattgctaaaaatcttactttgaacctagtaattagtttaaaagtagataaaatgtatccaagtattcataaatttaaagagatagctccacttccaaaaatggaaagacgactagtttggaaaaatgtatatgctacctctgttcgacaaatctttaacgaattaatgaattcaacagatagtatagttatttccattgtcttaggaatagttatggttggtaagtattcgaactatgcttatattttatcaatcgtatatattttctttggaggaatttttaatccgattcaggcttctattggaaacctatctctgttagcttctatagagaaaaaaaatgaaatcttcaatagattacgttttataaattttttctttttatcattttgctcaagttgtcttctagtattggtaaatccttttatcactatttggattggagaaaattacactttatcatttacaggtgtgattgcaatagttggtatgttattcgttagacaaactggaaattgtacaaccatttttcgtttgggagaaggatattttagagattatcatttttctcctttaatcgcgggcattttaaatttagttgtttcagtaattttagtgaattatataggtattgcaggtgtttttgttggaacaatgttaggatttggtttccaatttatactggtggatacaattgttacttataaaaaagtacttagtcgtccactatcagagttttatttaagatggttgcagacgatcttattaactgtaggtttatgtattgcgagttactatttatctcgattggttagagtgaatagtatatatgatttgatactgttgtttgtagtggttattggatttaatttttttgctttatgttttatttattggcgaaatgatgattttcaatattttattcaattggttaaaaattttatgaaaaatttggaggaaaaaaatcatgattaa

SEQ ID NO: 343 - DNA, 1416 bp, Streptococcus pneumoniae
atgaaaatcttaaaaaactacgcctacaatctttcttatcaattgttggtgattatactcccaatcattacaactccctatgtgacacgagtcttttcttcgtatgctttagggacttatggttattttaattccatcgttacttattttatcctcttagctactctaggagttgctaactatggtactaaggtaatctctggaaatcgaaaggaaattgaaaaaaacttttggggaatctactccctgcaattgtgtgcggcagttctgtctctgtccttatacgttgttctttgtctaactctttctttgatgcaaaatccagtcgcctatattctaggattgagtgtggtttctaaaggcttagatatttcttggctctttcaagggttagaggattttagaaagataactgttcgaaatatcacggttaaactgataggtgtaatctctatcttcctttttgtcaaatctgaaagtgacctttatctctatatctttttactaaccatatttgaactcttaggacaactaagtatgtggttgcctgctcgagaatttattggtagagatcattttgatatagcatatgttagacatcatttgaaacctgtcatattattattccttcctcaagtagctatttctatgtatattacgttagatcgtaccatgcttggggcattagcttctacaaaagatgtagggatttatgaccaggctctaaagttggtcaatatccttatgaccctggtaacttctttgggaagcgttatgttacctcgtgttgcgcatctgctatccaaaggggaacaaaaagcggttaataaaatgcatgagatatccttcctcatttataatttagtgatttttccagtgatagtagtgattttgattgtgaatgatgattttgttcaatttttccttgggcaagattttcaggatgcgcgttatgcaattgccatcatgatttttcggatgttctttatcggttggactaacatcatgggaattcagatgctgatacctcataatcaaaataaagaattcatgatttcaacaactgctcctgcaattatcagtgtaggtttaaacttgctattcctgccaaagtttggttatatcggggcagccattgtctcggttttaacagaggcatttgtatgggcaatccaattgttctatactcgcagatatttaaaaggagttcctataatcggatcaatggcaaaaattatactagcatctaccattatgtatggacttttactaagttcaaaaacagttatacatttttcaccgactttaaatgttctagcatttgcagcgcttggtggaataatttatctttttgcaattctatctctgaaagtggtagatgtgaaagaattaaaacaaattattaggaaaaattag

SEQ ID NO: 344 - DNA, 1464 bp, Streptococcus pneumoniae
atgagtaataaaatcagtaaaaatttagcctacaatataggatatcaattaattggtattgcctttcctttgataacttctccttatttatcacggatattaggcgctgaaaatttaggtattcattcttttactatctcagtagcactttactttatgatgtttatgctacttggaattgcaaattatggaaataggacaatagcgaccgttaaacgagaagggaaagagattttatcaaaaacgttttggaatatctactatgtccagttgctaatgtcggttctagtaactatcgcatatctcatttatttatatttttgggtgtcgagttacaagttcattgcgattttacaactttttttgttattatcgaatgcagttgatattacttggttattttatggattagaagattttaaacaaattgtttttagaaatgcattagttaaacttcttggtttgtttttaatattttcgtttgtacatgaaagctcagatttatggaagtacaccttaataaacggaggagtgactttagttgggcaacttttgttatggggacaattaaaaggcagattatcttgggtgaagattcaaaaaaaggatttactatctcatatcaaaccaattttagttcttttcattcctgtgttagcaatcagtatctttagtaatatggataaatatatgcttggtttaatggttggtgttaagcaagttggattttatgataatgcaaatcgtattatcgatattcctaaagcattaattgcagcattagaggctgttatgttaccacgaacatcttatttattagcagaaggtcaggaggaaaaaagtaattactatatagaggttacaatcctttatgcgatgatgatttcgagtgtattgatatttggtataatatcggtttctgatattttctcgcttgtattttggggagaagaattccttgaatcaggaagattaattgctgctatggcaccagtttttgttttttctgttccaggaaatataattagaacacagtatttgattcctagagcaaaggataaagattatgtgttgtctctaataataggtgctctagttaatatattattgaactgtttcttaataaaaccttttggagccatgggtgctacgattagtacagttttggcagaatttgtcttgtatggagtgcaattttggacagtcagaagagatttagactttaaaaaatatttaaagaatggttttatattttacctatttggtatgattatgtatttagctattatagcagcaaaagctcatttgcaatataatataataaatcttgttctccttatagtgctgggaggaatagtatatactggcttttgttgtttctacattttaatatcaagaaacgtacattttgagatattacgggaaaaaattaaacgaaagattggttatgaaaatatattataa

SEQ ID NO: 345 - DNA, 1464 bp, Streptococcus pneumoniae
atgagtaataaaattagtaaaaatttagcctacaatatggggtatcagttaattgggattgcagctcccttgataacttctccttatttatcacggatattaggtgctgaaaatttaggtattcattcttttactatgtcggttgcactttactttatgatgtttatgcttcttggaattgcaaattacggaaatagaacaattgcgactgttaaacgagaaggaaaggatattctatcaaaaacgttttggagtatctactctatccagttgataatgtctattctggtaactatagcttatcttgcttatctatatttgggggcagtacattacaaagttattgcaatattacaactctttttattgttatcaaatgctgttgatattacttggtttttctatggattagaaaattttaagcaaatcgtttttaggaatacattagtcaaacttttaggattatttttaatttttttatgtgtgcgtcaaggcacagatttatggaaatacacatttataaacggatcagtaactttagttggccaacttttactatggggacaattaaaaggaatattaacttggaaaaaaattcaattaaaggaattactacctcatatcaagccaattttaattctttttattcctgtactagccattagtatttttactaatatggataaatatatgcttggtttaatggttgatgtaaagcaggttggattttatgacaatgcaggtagaattattgaaattcctaaagctttgattgcagctttaggggccgttatgttaccaagaacttcctatttattagcggaaggacagaaagaaaagagtaaatactacatagaggtaacaattttatacgtgatgattatttctagtgtattgatgtttgggctaatatcggtatctgatattttttccattatattttggggagaagaatttcttgaatcaggtagattgatttctgctatgtctccggcttttgtcttctctgtattaggaaatatcattaggactcagtatttgattcctagagctaaggataaagattatgtagtgtctctaattgcgggggctgttgttaatctagtgctaaactatttttttataaaaccttttggtgctatgggagcaacagttagtacagttttggcagaatttgtattgtctggaatgcagttttggtcagtaagaagagacttagatttaaaaagatatttgaaaaacggtttgatattctacctatttggtctgattatgtaccttgttattataacactcaaaactcagttaccatacaatataatgagtcttattttactaatagttttgggtggaatagtatatgttattttttgttgttgctatattttaatgtcaagaaatttacattttaagttattgaaggaaaagattatacgaaaggttgtacataaaaatacattataa

SEQ ID NO: 346 - DNA, 1464 bp, Streptococcus pneumoniae
atgagtaataaaattagtaaaaatttagcctacaatatggggtatcagttaattggtattgcagctcccttgataacttctccttatttatcacggatattaggtgctgaaaatttaggtattcattcttttactatgtcggttgcactttactttatgatgtttatgcttcttggaattgcaaattacggaaatagaacaattgcgactgttaaacgagaaggaaaggatattctatcaaaaacgttttggagtatctactctatccagttgataatgtctattctggtaactatagcttatcttgcttatctatatttgggggcagtacattacaaagttattgcaatattacaactctttttattgttatcaaatgctgttgatattacttggtttttctatggattagaaaattttaagcaaatcgtttttaggaatacattagtcaaacttttaggattatttttaatttttttatgtgtgcgtcaaggcacagatttatggaaatacacatttataaacggatcagtaactttagttggccaacttttactatggggacaattaaaaggaatattaacttggaaaaaaattcaattaaaggaattactacctcatatcaagccaattttaattctttttattcctgtactagccattagtatttttactaatatggataaatatatgcttggtttaatggttgatgtaaagcaggttggattttatgacaatgcaggtagaattattgaaattcctaaagctttgattgcagctttaggggccgttatgttaccaagaacttcctatttattagcggaaggacagaaagaaaagagtaaatactacatagaggtaacaattttatacgtgatgattatttctagtgtattgatgtttgggctaatatcggtatctgatattttttccattatattttggggagaagaatttcttgaatcaggtagattgatttctgctatgtctccggcttttgtcttctctgtattaggaaatatcattaggactcagtatttgattcctagagctaaggataaagattatgtagtgtctctaattgcgggggctgttgttaatctagtgctaaactatttttttataaaaccttttggtgctatgggagcaacaattagtacagttttggcagaatttgtattgtctggaatgcagttttggtcagtaagaagagacttagatttaaaaagatatttgaaaaacggtttgatattctacctatttggtctgattatgcacctcattattatagcactcaaaactcagttaccatacaatataatgagtcttattttactaatagttttgggtggaatagtatatgttattttttgttgttgctatattttaatgtcaagaaatttacattttaagttattgaaggaaaagattatacgaaaggttgtacataaaaatacattataa

SEQ ID NO: 347 - DNA, 1464 bp, Streptococcus pneumoniae; identical to SEQ ID NO: 346

SEQ ID NO: 348 - DNA, 1464 bp, Streptococcus pneumoniae
atgagtaataaaattagtaaaaatttagcctacaatatggggtatcagttaattgggattgcagctcccttgataacttctccttatttatcacggatattaggtgctgaaaatttaggtattcattcttttactatgtcggttgcactttactttatgatgtttatgcttcttggaattgcaaattacggaaatagaacaattgcgactgttaaacgagaaggaaaggatattctatcaaaaacgttttggagtatctactctatccagttgataatgtctattctggtaactatagcttatcttgcttatctatatttgggggcagtacattacaaagttattgcaatattacaactctttttattgttatcaaatgctgttgatattacttggtttttctatggattagaaaattttaagcaaatcgtttttaggaatacattagtcaaacttttaggattatttttaatttttttatgtgtgcgtcaaggcacagatttatggaaatacacatttataaacggatcagtaactttagttggccaacttttactatggggacaattaaaaggaatattaacttggaaaaaaattcaattaaaggaattactacctcatatcaagccaattttaattctttttattcctgtactagccattagtatttttactaatatggataaatatatgcttggtttaatggttgatgtaaagcaggttggattttatgacaatgcaggtagaattattgaaattcctaaagctttgattgcagctttaggggccgttatgttgccaagaacttcctatttattagcggaaggacagaaagaaaagagtaaatactacatagaggtaacaattttatacgtgatgattatttctagtgtattgatgtttgggctaatatcggtatctgatattttttccattatattttggggagaagaatttcttgaatcaggtagattgatttctgctatgtctccggcttttgtcttctctgtattaggaaatatcattaggactcagtatttgattcctagagctaaggataaagattatgtagtgtctctaattgcgggggctgttgttaatctagtgctaaactatttttttataaaaccttttggtgctatgggagcaacagttagtacagttttggcagaatttgtattgtctggaatgcagttttggtcagtaagaagagacttagatttaaaaagatatttgaaaaacggtttgatattctacctatttggtctgattatgtaccttgttattataacactcaaaactcagttaccatacaatataatgagtcttattttactaatagttttgggtggaatagtatatgttattttttgttgttgctatattttaatgtcaagaaatttacattttaagttattgaaggaaaagattatacgaaaggttgtacataaaaatacattataa

SEQ ID NO: 349 - DNA, 1416 bp, Streptococcus pneumoniae
atgaagattttaaagaattatgtttataatttgttataccagatactagtagttgttctcccaataataactactccctatattactagaatctttagttctcaagagttaggtgattatggatattatagttctatagtaacttactttatattgctcgctactttaggtgtcgccaactatgggactaaggtaatttctggacatcgtaatgagattaataaaaatttttggggtatctactctttacaattaggtgctacaatcctttcaatctccttatattgtattttttgcgttaaccttcctccaatgcaaaacccagtagcctatattttgggattgagtttagtttctaaaggtttagatatttcctggctttttcaaggattagaggatttccgtaaaattactgttcgaaatattacggtaaaactaattggggtaatctctattttcctgtttgtcaaatctactaacgacctttacctttatgtgttcttattaacaatatttgagctattgggacaattaagtatgtggttgccggcccgagggtttattggtaagccccatgttgatttagaatacgctaggtaccatttgaaaccaattattttattatttcttcctcagatagcaatctctttatacgttactttagatcgtacgatgcttggtgctttatcctctacaaaagatgtaggaatttatgatcaggctcttaaattggtaaatattttattaacattggtaacatcgcttggaagtgttatgttgcctcgagtagctaacttattagcaacaggagatcatagagcagtcaataagatgcatgagatggctttcttgatttataatttagttatttttcctattatatccggaatgttaattgtcaatgatgattttgttcaatttttccttggtcaagattttcaggatgcacgttatgcaatcgccattatgattttccgtatgttctttataggttggaccaatattatgggaattcaaattttgattccacacaataaaaataaggaatttatggtttcaacaacagctcccgcaattatcagtgtaggtttgaacctgctattccttcctaaacttggttatatcggagcagctattgtctctgttttaacagaagcgctagtatgggcaattcaattgtactttacccgcaaatacctaaaggaagtttcaattattggatctatgacaaaaattattctagcatcagctatcatgtatggtattttgcttggttcaaaaacatttatacagttttcaccaaccataaatgttctagcatttgctgtgtttggtggactcatttatctttttgcaattctatctctgaaagtggtagatgtaaaagaattaaaacaaatgattaggaaaaaatag

SEQ ID NO: 350 - DNA, 1440 bp, Streptococcus pneumoniae
atgaaaaaaatattaaataagtattattctttatctaatcctgtaaaagcttcaatttggtttactatttgcaatgttcttcaaaaaggaatttcaatgattactgttccagtttttactagagtattgacaacggaacaatatggtgtatactctgtatatcaatcatggtattcaataattggagtttttgcaacactaaacctatattatggagtctttaataatggaatgattaaatacgagaaagataaaaatgtctttacttcctctatgcagggactaacaacaacagttacagctatatttttactgatatatttaatagggattgatttttggaactcgttgttaggcttacctacactactaatcttagtgatgttctttgatttgttttttacccctgcttattctttttgggttgctcgtcagagatttgaatataaatatagaaacttagtatttatcacatttattatagcgattggaagtcctattattggtatttctgcagttgttttatcgacttataaagcggaggctagagttatctcatttgtgttagttcaatcttgtatagggttatatttctatattttgaacctttaccgaggtaaacattttttttgtaaaaaatattggctctatgctttgaattttaatttacctcttattcctcattatttatcgcaatcagttttaaaccaatcggatagaataatgattaattcaatggtaggaatgggagaagcagcaatatatagtgttgcttacagcatatcaattttgatgcttttagtgacaagtgctataaacagctcttttattccttacacatataagtgtattcgagataagaaatatactgaactagggaaaagtgcaaatcttttaattactctggtaggcattggttcgattttaactatttctcttggaccagaaatcattcaattatttgcaccaaaacaatattacgaagcaatatggattattcctcctgttgctctgtcagtatatttcatgttcttatatcccatttttggtaacattgagttttactttgaagcaaatcattttgtaatgtgggcttcaatcggtggagcaattatgaatatttttttgaattttatttttttaaaacactttggatatattgcggcaggctatacaactttattctgttatattttatttgcgttgggacattacatttttatgaggagagtactgtatcagaatctttcgggtatccaaatttataattccagatatattttatttttttcgattcttcttattattctaatgctactaatcgtgattatttatccatttatactgattcgttatcttactatctcactcatcctaggaggatgttttttaaagaggaaaaagataatgagttcgatagctatcattaaacaacagtga

SEQ ID NO: 351 - DNA, 1443 bp, Streptococcus pneumoniae
atgaataacgaaaaaagtttaagctccaagataatatcaggaagtttttggaaattcggagaaagaatgagtgcacaggcggtttctttttttgtttctctgattttaacacgaatcttatccccaagtgattacgggattattgcgattattaatatttttatagctattgctgatgtacttttagctagtggtttaaatactgctttgatacagaaaaaaaattcaacgagactagattataatacgattttttggtgtaatattatactgggttgtgttctgtatctagtattatttttagtagcaccagtccttgcagatttttataatatttctatacttacaccagtcattcgaatttttgcactgcggttacccatttcttcttttcaatcaattcaaaacgcatttgtttctaagcatatgcagtttaagaagtttttttacgccacctttttgggttcgctagtatctgctgttgttggaattacaatggcttatacaggttttggtgtatgggcacttgtagcacaatatttagtaaatacggttgttggaacagctacactatttgctatcattagttggcgtccaaaatttgaattttctttggaaagtgccaaacccttagtccaatatggctggaagataatgtttacagaccttacaggtacgattttcaacaatttaggagactttatcattggcgcatactataattcatcttcacttgctttttattctaaagggcgccaattaccctatctaattagaaataatatttttacttctttgataagtgtactcttcccaggaatgtctcaggttaatgatgatattgaacgtattaaatctttttcacgtaagagtgtctcaatcttatcttttttaatttttccaacaatggttggtttaattgtgactgctgaaccactaactatcttaatgtatactgaaaaatggctacctattattcccttcgttgtgattgtatgttcggaagcaattttatcagttataccaacggtaacaatgcagacaattaaagcattaggacgtagtgatttaacactaaaaatagaatttataaaaaagcccatttatctattaacgatttttgtatctttaaactttggaatagtggctattgcctttacattaattattaatgcgattattgaaatgattattaatggtttaattgtacaaagactaatcaagtactctttgtgggaacaatttaatgatatatttggttcattaatgatttcaattatgatgggcttattatcatatttagtgatttttttaaatttaaacatttatctcacattattgttacaagttgttactggtgtctctagttatctcattttagctattgtatttaaaaacgaatcattcattgagttgaaaaatagatttttgagcactgagtag

SEQ ID NO: 352 - DNA, 1518 bp, Streptococcus pneumoniae
atgagtaatagacttgttaatactagaagaaatattgtttggggaacattaaataagcttattgcgattgctttaccatttttagttcgaacaactttgatttatcaattagggatacaatatgtcggtattagtggactttttgcatctattttacaagttcttagtttagctgatttaggtttcgctaatgcaattgtatatagtatgtataaacctcttgcccatggggataatgacactgtcggtgcacttctaggattttataaaaaaatatatcgagtcgtcggggtggtaatattaattgcgggccttttaattgtgccctttataaattacttagtaaagggttcctatcctgcagatatcaatatacaactaatttacttaatttatctggcaaatacagttgttagttactttctttttgcttataaacgctctttattgttagctagtcaacgaaaagatatatttgacaacattgattcagttgctcgcataattttaagtggattacaaattttattattgtttctttttcctgatttctatgtttttgctttagtaattccctgtatgacaatccttaataatgtatttaacgaaatcgtgactaagaaaaattacaattatatcgttgaaaatagtaacttagataaaaaagttaaacaagaaattattgaaaaaacaaagggattatttgtctacaaagtatgtgggattacaagaaattccctcgataacatctttatttctacttttttagggattacgacagtgggaatttatagtaattattattacattatgatatcagtaaaaggttttttggatgtggtttctacaggaatgagtgctagtgttggacatagtgttgctactgaaagtgttgaaaaaaatcatcgtgacttagaaaatcttacatttggtttctcttggttagctacctggtttatgacatgtttattatgtctgttccaaccatttatgcttttgtgggtaggacaagctaatattttaccattttcagtcgttgttgctctctgtttttacttttatgttcttgctgcaggtgatatgcggagccaatatattgacgcctcaggtttatgggataaagaaaaactacggtcaattgcagaaacggtaggtaatgttgttttgaattatattttggttcaatttttaggtgttttaggaatagtgctagctactgcactaacaattttgtttattggaattccatggagtacaaaaattgtgtttgataattattttaaaaatggttataaaaagtatttgtggaatcaagcagtatatgctatagtaactataattgttgctagtattacttatctaatctgtacactagttggtggcaataacatcgtggttttaattataagaggaataatttgtttatttgtccccaatattttatattacttattctttttaaaaaatgaacagacgcgtctgtttttcaaaataatcaagtcgaaattattaaaataa

SEQ ID NO: 353 - DNA, 1305 bp, Streptococcus pneumoniae
atgattctgtccatgttggggatttctaattatgggaatcggacaatagcacgggtacgaacaagtagagaacatttgaatcaggaattttcgaatatttacgcggttcagttgacgtgttcactagtaatgaccatctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggggtaccctgcttggtcagttgattacatggccatttttgctaaaacaagtaaactttgtgtgccctagtcttggaaaaataaagaaacacatgaaacctattattattttatttttccccgtcttagcggttagtattttttcgtttctagataaaataatgcttgggatgtatagtagtttgaaggaaactgctttttatgagaattcagataagatcattagtattccaaaagctctgattcaagcctttggagctgtaatgttgcctcgaacagtacatttactgagtataggtgatgaacaaaaaagtttagaatatgttgataaaacgatgtgggctgttttagtgattacaatgggctgtgctttcggtttagcaggagtttctgcaacctttgctcctgtttattggggtgaagaatttagagccagtagccaaattattgcaggaatgacaccggctttagtattttcggcttttggaaatgtcatccgaacccagtttttaattccacgaagttttgacaaggaatatacggtatctttactttatggagccgttgttaatattttgattaatattcttttaattccgaagataggagcaatgggagctgttatcggaacaattgttgcagagttagttctatgttgctatcaaacatggattgctagaaattatttacatatcagagaatatctgataaatgctgggatattatttcttatcggttcagtaatgtatatggttttaattcttatttcaagtattttacctacttcgttacttacacttattattgaaattatagtcgcagctttcatttatattagtttactgattctgcatatcttttcttcaaaaaacagggtaattattggtttacgaacaaattttttagaacgtactcatttatttaaaaggaagtaa

SEQ ID NO: 354 - DNA, 1305 bp, Streptococcus pneumoniae
atgattctgtccatgttgggaatttctaattatgggaatcggacaatagcacaggtacgaacaagtagagaacatttgaatcaagaattttcgaatatttacgcagttcagttgacgtgttcactagtaatgaccgtctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggagtaccctgcttggtcagttgattacatggcaatttttgctaaaacaagtaaactttgtgcgccctaatcttggaaaaataaagaaacacatgaaacctattattattttatttttccccgtcttagcggttagtattttttcgtttctagataaaataatgcttgggatgtatagtagtttgaaggaaactgctttttatgagaattcagataagataattagtattccaaaagctctgattcaagcctttggagctgtaatgttgcctcgaacagtacatttactgagtataggtgatgaacaaaaaagtttagaatatgttgataaaacgatgtgggttgttttagtgattacaatgggctgtgctttcggtttagcaggagtttctgcaacctttgctcctgtttattggggtgaagaatttagggccagtagccaaattattgcaggaatgacaccggctttagtattttcggcttttggaaatgtcatccgaacccagtttttaattccacgaagttttgacaaggaatatacggtatctttgctttatggagccgtagttaatatcttgattaatattcttttgattccgaagatgggagcaatgggagctgttatcggaataattgttgcagagttagttctatgttgctatcaaacatggattgctagaaattatttacatatcagagaatatctgataaatgctgggatattatttcttattggttcagtaatgtatatggttttaattcttatttcaagtattttacctacatcgttacttacacttattattgaaattatagtcggagctttcatttatattagtttactggttctgtatatcttttcttcaaaaaacagggtaattattggtttacgaacaaattttttagaacatactcatttatttaaaaggaaataa

SEQ ID NO: 355 - DNA, 1305 bp, Streptococcus pneumoniae
atgattctgtccatgttgggaatttctaattatgggaatcggacaatagcacaggtacgaacaagtagagaacatttgaatcaagaattttcgaatatttacgcggttcaattgacgtgttcactagtaatgaccgtctcatatttgatttatgcaacagtatttgtgaatagttttcagattgtagcctatatccaagtattacatgttttatcgtatgcaacagatgttagttggtttttttatggtcttgaagagtttcgtattacggttgctaggaattcatttgttaagttattaactttaatatctatctttacatttgtaaaaagccctaatgatatctatttatatacctttataatggcagggagtaccctgcttggtcagttgattacatggccatttttgctaaaacaagtaaactttgtgcgccctagtcttggaaaaataaagaaacacatgaaacctattattattttattttttcccgtcttagcgattagtattttttcgtttatagataaaataatgcttgggatgtatagtagtttgagggaaactgctttttatgagaattcagataagatcattagtattccaaaagctctgattcaagcctttggagctgtaatgttgcctcgaacagtacatttactgagtataggtgatgaacaaaaaagtttagaatatgttgataaaacgatgtgggttgttttagtgattacaatgggctgtgctttcggtttagcaggagtttctgcaacctttgctcctgtttattggggtgaagaatttagggccagtagccaaattattgcaggaatgacaccggctttagtattttcggcttttggaaatgtcatccgaacccagtttttaattccacgaagttttgacaaggaatatacggtatctttgctttatggagccgtagttaatatcttgattaatattcttttgattccgaagataggagcaatgggagctgttatcggaataattgttgcagagttagttctatgttgctatcaaacgtggattgctagaaattatttacatatcagagaatatctgataaatgctgggatattgtttcttattggttcagtaatgtatatggttttaattcttatttcaagtattttacctacatcgttacttacacttattattgaaattatagtcggagctttcatttatattagtttactgattctgtatatcttttcttcaaaaaacagggtaattattggtttacgaacaaattttttagaacatactcatttatttaaaaggaagtaa

SEQ ID NO: 356 - DNA, 1416 bp, Streptococcus pneumoniae
atgaaagtaataaaaaactatgcttacaatctttcatatcaattattggtaattatactccctattattacaacaccttatgtaacacgggtttttagttctaatgatttagggacatacggatactttagttcgatagttacttactttattttgctggctagtcttggcgttgctaactatggtactaaggtaatctctggaaatcgcaaggagattgaaaaaaacttttgggggatttactcattgcaaatgggtgcaacgcttctttctctattcttgtaccttgttctttgtttgacccttccttttatgcaaaatctagtagcctacattcttggattgagtttggtttctaaaggtttagatatttcctggctctttcaaggtttagaggattttcgaaagattactgttcgaaatatcacagtcaaactcgttggtgtcatctccatctttctctttgtcaaatctgcaaacgatctttatctctatgtctttttgttaaccatatttgaactctttggacaactcagtatgtggttgccggctcgagactttatcggcaagtttcatgttgatatagaatacgccagacagcatttgaagccaattattttattgttcctaccacaagtcgcgatttctttgtatgttaccttagatcgtaccatgcttggagcgcttgcttctacaaaagacgttggcatctacgatcaagccctaaaattggtaaatattcttctaacctttgtaacttcattgggaagtgttatgctgccacgagtatctcatctgcttgcgacaggtgaacgtaaggcagtaaacaagatgcatgagatgtcttttttgatttataatttgcttatttttccgattatggcggggattctgatcgtgaatgatgatttcgttcagttatttctaggacaagattttcaagatgcacgctatgcaattgcgattatggttctcaggatgttctttattggttggactaatataatgggaactcagatgttgatcccccataaccaaaataaagaatttatgatttcaacaacagctccagccattattagcgttggattgaatctattattcctacccaagttaggttatataggagcagccattgtctctgttttgacagaggcgctagtttgggtaattcaattattctatactcgcagatatttaaaagaagttcctataatcggatcaatgtcaaaaattatactagcatctgccattatgtatggccttttactaagttcaaaaacagttatacatttttcatcgaccttaaatgttctagcatttgcagcgcttggtggaatcatttatatttttgcaattctatctctgaaagtggtagatgtgaaagaattaaaacaaattattaggaaaaactag

SEQ ID NO: 357 - DNA, 1443 bp, Streptococcus pneumoniae
atgtcaaccagaaattcacttattaaaaattctctttataatgttgcttatagaatgttaaatatcatatttccattagtatccgcaacatatactgcacggataattttagctagtggagttggggaggtctcgtttgcacaaaatgttgtttcttactttacaacgatagctgctatgggtattccaaattatggtatacgagaaattgctaaaactagaaataaagaagaggctacgaataaagttttttcagagctatttctgattaatggattgtctacattactgtgttctatagtatatttaattctgattttgacattcaatagcatttttgataatacatcaatgtatcttgctgtgggtttaacattagtgtttaacttccttaatatcgattggttctatcaaggaaaagaggactatgcatacatcacgaaacgtagtttcagtataaaacttatttctctttttagtttatttatttttgttagagatactgacgatgcagttaactatgcattgatttctagcttagcaattggcgggaataatatttttaatcttgttaatttaaggaaatataatattagatttagttttaataatattaagattagaagacacttaaaacctatttttgttttgttgggttcagtgattgctattgaaatctatactatgctggataccactatgattggtgctatggtaggaacgactgaggtaggttattatacaaattctatgaagctagttaaattgctgataactgtgattaccgctattggaggagtgcttttaccacgactaagtgattattattctaagggagaatttgaaaaattaaatcacatcgttgataaagtttttagaatcatgttgtttctttttttaccagcccaagttgcattaattctagttgcaccagtaattatgccgattttatttggtgatagttttataccagcagtgttaacacttcaacttacttctttgcttatatgtacattagggtttagtaatttatttgggactcaaatattactaacatttggagatgagcgaaagcttttgattagcacaattttaggagcgcttagtaatatcgctttgaacctaattctgattccagtaatggctcaaaatggagcagcgttggcatctgtgattagtgaaacaattgttactttaacagcatactactatgcgaaaaaacgtgtgacaattaggtttgacttaggattttatttgccaactgttgtttcaacaataatcatgggtggagctatatatgtagtaatgcaattgtatattggggatatagaaaaaatgttattttcaactttgttaggaggtgttgtattccttttaattaacgtttttatgaagaatcctattatatatgaatttacaggtattataaagagaaagagataa

SEQ ID NO: 358 - DNA, 1443 bp, Streptococcus pneumoniae; identical to SEQ ID NO: 357

SEQ ID NO: 359 - DNA, 1386 bp, Streptococcus pneumoniae
atgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctggggtcataagtacagcagttatgcttgccatgaatgtcattcttctagtagtatttgattggggattgttaggtttctttattgctaatgtttgtggatatgttataccttgtatttatatagtttctagattaagattgtgggaattatttgaaattaagattgataaaaaattacaatgggaaatggtttactatgctttgccattagttttaaatatattaagctggtgggtaaataatacttcagatagatatattgtgactgcaatagtaggtatacaagctagtgcaattatttcagtcgcttataaaattccacagattttgtctacgattagtgcaatttttattcaatcatggcaaatctctgcaattaaaatacaagaagataagagcggtactacatttgtatcaaacatgctcctatactataatgcacttctattgattatagcgagtggtatcatattatttgtcaaacctatttctaatatattgtttggtatttccttctattctgcgtgggagctggtaccatttttaataattagcagtctattcaatgcaatttcaggttgtataggagcaattatgggagctaaaatggatactcataacattgcaaagtcggctttagttgggatgattgcaaatattattttaaatatagtgttaacattcttaatgggtccacagggaattacaatttctaccctgatagctagttttcttattttttatatgcgaaaagatagtgtaaaagaaattaattcagaaacatatagagcaatttatttatcgtggatactattagttgtagaggcctgtctgctaatttatatggattttattattggtgctttaatagcaatggtaataaacttatttttactaaaagatgttatcaaacctttatatttaaaaattttcaaaagaaattag

SEQ ID NO: 360 - DNA, 1389 bp, Streptococcus pneumoniae
atgagtaaatataaggaattagcaaaaaatacaggtacttttgctttagctaatttttcatcaaaaatattaatttttttgttagtacctatatatacaaaagttctaacaacgacagagtatggtttttatgatttggtttatacaacaattcagctccttgtaccaatcctgacattgaatatatctgaagcagttatgagatttttaatgaaagaagatgtttcaaaaaaatcagttttttcgattgcaatacttgatatatttttagggtctattattttttgtttgttactactggtcaatcaaatattttctttatcggagttaatttctcaatatagcatatatatcatggcgatttttgctttttatacacttaataattttttgatacaatactctaaagggattgataaaataggtgtaacggctatctctggagtcataagtgcagcagttatgctttcgatgaatattttattgttagtggtattaaattggggactactaggtttttttatcgcaaatatttgtgggtatgtcattccttgtgtatatataatagtaaaactaaaattatgggatttatttgaacttaaaattgatagatcgttacagtgggaaatgatatattatactttacctttgattttaaatactttaagttggtgggttaataacacttcagataggtatattataacagtaattataggtatacaagctagtgcgattatttcagttgcttataagattccacaaattttttccacaattagcgctatattcattcaatcgtggcaaatatctgcaataaaaattcaggaagaaaaagaaggtaacacctttatatcaaaaatgcttttatattataatgcactcttgctgattatagcgagcggtatcattctatttgtaaaaccaatctctaatatactcttcggagcttcattttattcagcgtggacgcttgtaccgtttttaattattagcagtttgtttaatgcgatttcgggttatataggagcgataatgggcgctaaaatggatactaaaaatattgctaaatctgctttggttggaatgatagctaatgtttttttaaatattgtattaacatttttaatgggactacaggggattacaatctcaactatgatagctagttttcttattttttatatgagaaaagatagtgttgaagaaattgctccagaaacatatagagctatttatctttcgtggtttttgttagtggttgaggctagtcttttggtttatatagattttattattggggctacattagttacattaatcaatttatttttattaaaggatactcttaaaccattatgtttaaaattattaaaaggatttaaatga

SEQ ID NO: 361 - DNA, 1458 bp, Streptococcus pneumoniae
atgagcatgaaagaaaaatcaataagtaaaaatgctattttgaatattattttaacactaacaaatattgtgtttccattaattacttttccttatatttctagaattctgaatccgtcaggaataggcgcaatctcttttttttcatctattggttcttatggtgttttagttgcctcgcttggaatttcaacttatgggattcgggtgattgcaaagaatcgttatcataaagataagataactaaaatattccaagaattgatagttattaatagtgtaatgtctattatagtgacgttcttccttgttttaatgagtttccgattagagcagttaagttcagaaaaaggtttattaataatcacatgtattactattttgtcctctccttttaatttgaattggttttatagtggaatagaagagtattcatatataacaaaacgttcaatattttttaaattagtttcattgatattaacctttttatttgttaaaagtaaagacgattatattatatatgctgtgataatattgttttcaacattggcttccaattttataaatattttggagagtagaaaatacatcaattttaatttgagaaggaatttagaatttagatatcatctaaaaccaatgtggtatttatttgcttcgttacttgcagttaatatttatacaaatttggattctgtgatgttaggaattattaatggaaatgatgcggtaggtatttactcaatcgcatcaaaagtaaaatggattttattatcagtcgttacttcagtgagtttagttttattacccagattatcattttatagcaataagtatgacgaaacgaagtttaataatattttgagaaagtcatctacaataatttttatgatttcaattcctctaacaattttttttatgataaaagcgaaagagagtatattattgttaggtggagagcagtatattcaggcagttttagcaatgcaaatattaatgccgatcttgataatatcaggtttttccaatattataggtaatcagatattgatccctactggtaatgagaaatattttatgagagcagtttctataggggcaatagtaaatttatgtttaaatttgttgcttatgcctattttgggaattatcggaggtgcaatcgcaactctttgtgcagaatcagtccaaatgattattcaattttatttttctcgtaataaattaatgggtaatatttcattaaattcaatcaaaaaagtagcctattcatcaatttttgcgggtatactgttaatagtaatacaaaatattatagagaattttaatagcttcttaaatttagctgcttctagtttcctatattttggagtatattttttcttattggtattgtttaaagaaagtacaattaagaaatttctaaatcaaattttttataaggatatttcatga

SEQ ID NO: 362 - DNA, 1425 bp, Streptococcus pneumoniae
atgaaagtattaaaaaactatgcctacaacctttcttatcagttgttgataattatactccctatcattactactccctatgtgacacgagtcttttcttcggatgatttaggatcatatggttatttcaattcgatagtgacttattttatcttgttagcaacacttggggttgctaactatggtacaaaggtaatttcaggacatcgtaaaaacattgaaaagaacttttggggcatctattcgttacaattaggtgcgacagttctttcactgttattctatgtcattctatgtctctctatatcttttatgaaaaatccaacagcctatattcttggattgagtttaatatcaaaaggcttggatatttcctggctttttcaagggatagaggattttcgaaaaattacagtaagaaatataactgtaaaagtcattggggtaacctcggttttcttatttgtaaaatcagctagcgacctctatctttatgtttttcttttaacgatatttgagttattagggcaacttagtatgtgggtaccagctcgggaatttactgggaaaccgcactttgactggttgtatgctaaacaacatttgaaaccagttatcttattatttttaccacaggtcgctatatcgctttatgtgactttagatagtactatgcttggttctctatcttcaattagagatgtaggaatttatgatcaatctttgaaacttgtcaaaatattattaacattagtaacttcattaggaagtgtcatgttgccacgagtttcaaatcttttatcttcaggggatcataaagcagttaataaaatgcatgagatgtcatttctggtttataacttggttatttttcctattatggcaggaatgctaattgtaaatgatgactttgtaacctttttcctcgggcaagattttcaagaggcacgttatgcgatagcaattatgatttttagaatgttctttattggttggaccaatattatggggattcaaattttgatacctcataataaaaataaagaatttatgctgtcaacaacaattcctgctattgttagtgtaggcttaaatcttctcttgcttccaaagctgggctatataggtgcggcaattgtatctgttttaacagaggtactagtatggctgatacagttattctatactcgttcttatttgagagaggtgcccattctaggttcattgatcaaaattataatctcatctggagttatgtatggtatcttgttatttataaaacaattcctaaatgtatcaccgataatcaatgtgggactatatgctattctgggagcaatgatctatctcgtattaattttgatttttaggattataaatccaaatgagttaaaacaacaattattaaaaaataaaggagcataa

SEQ ID NO: 363 - DNA, 1416 bp, Streptococcus pneumoniae
atggaaacaaaagaaaaacgtttagtgaataatacgattatttttacaattggaagtttaggttcaaaatttatacagtttttgttagtacctttatatacctatactttaacagctgctcagtttgggattaccgaaattttattaaccgctgtgaatcttttaatacctgtattttctatttccattgctgatggattattaagatttggattagataagaccttaagaagagaaaatgtcttgaaatcagcttttatcatttcgatacttgggactattttatccattatttcgattccaattttttcgctctatcctactttgtcagagtggatggtttattttatcatcattttgaacttgagaatgtatcgggatgtttttgcgattcaattgaaagttgaagggaaaaatacattatttgcttgcgatagtatgatttatacatttgttttatctttagcgagtattgtctttctagttcctttttctttaggcatttcaggttattttttcgcttatattgtcagtaatggaatttccattttctttatcttatttttcggaggagtctggaaaagttttactagcggaagatttgaaaaacaattgatgatacaactattgaaatattcagcacccatgattttaaatggtattgcatggtggattacaaatgcctcagatcggtttatgttgcaatggtttatggatgatagagcggtaggtttgtatggtgttgttgcaaagttacccttattaatcggaacgtttactggagtatttaatcaggcatggattatttcagcagtagaagagtttgaagaagaaaatgaagaatggttttatcaaaaagtttttcaccaatattatgcagcactctttttaagtgtttcagtatttttgcttctacttcaaccatttatgaaagtatatgttagtccaagcttttatgaagcatggcaatatgctccgtttttattattgtcaagtgtagtttcaggaatcgccgcatttatgacaggtttttatgttgcgcaaaagaaaaatttgaatattatatacacgacgattgctggtgcttttgcgaatattttgttaaatgcgatgtttattcctatgcttggagtattaggcgcttccattgcgacctttctatcatggtttgttattgcaatctatcgtatgaaagatgttgaaaactttgcttgttttccattagataaaaaagttttttggtatttatttctattatgtattcagaccattacgatgacttttctgccaatccttggcattgttttttcagtcgtattgattccttatttcttttaccaagaacaggaatttctagcagttttatttgataaaggaaggaaaaaagtatgttcattcaaaaaatcaaaaagataa

SEQ ID NO: 364 - DNA, 1440 bp, Streptococcus pneumoniae
atgaaaaaaatattaaataagtattattctttatctaatcctgtaaaagcttcaatttggtttactatttgcaatgttcttcaaaaaggaatttcaatgattactgttccaatttttactagattattgacaacggaacaatatggtgtatactctgtatatcaatcatggtattcaataattggagtttttgcaacactaaacctatattatggagtctttaataatggaatggttaaatacgagaaagataaaaatgcctttacttcctctatgcagggactaacaacaacagttacagctatatttttagtgatatatttaatagggattgatttttggaactcgttgttaggcttacctacgctactaatcttagtgatgttctttgatttgttttttactcctgcttattctttttgggctacgcgtcagagatttgaatatcaatatcgaaacttagtatttatcacacttattatagcgattggaagccctattattggtatttcttcagttgttttatcgacttataaagcggaggctagagttatctcatttgtgttagttcaatcttgtataggattatatttctatgttttgaacctttaccgaggtaaatattttttttgtaaaaaatattggctctatgctttgaattttaatttacctcttattcctcattatttatcgcaatcagttttaaatcaatcggatagaataatgattaattcaatggtaggaatgggagaagcagctatttatagtgttgcttacagcatatcaactttgatggttttagtgacaagtgctataaacagctcttttattccttacacatataagtgtattcgagataagaaatatactgaactagggaaaagtgcaaatcttttaattactctggtaggcattggttcgattttaactatttctcttggaccagaaatcattcaattatttgcaccaaaacaatattacgaagcaatatggattattcctccagttgctctttcagtatatttcatgttcttatatcccatttttggtaacattgagttttactttgaagcaaatcattttgtaatgtgggcttcaataggtggagcaattatgaatatttttttgaattttatttttataaaacactttggctatattgcggcaggctatacaactttattatgttatattttatttgcattgggacattacatttttatgagaagagcattgtatcagaatctttcgggtatccaaatttatgattctcgatatattttctttttttcgtttcttcttctgattctaatgctactaatcgtgactatttatccatttgtgttgattcgttatattgctatctcactcatcctaggaggatgttttttaaagaggagaaagataatgagttcgatagtcatgataaaaaaacagtga

SEQ ID NO: 365 - DNA, 1440 bp, Streptococcus pneumoniae; identical to SEQ ID NO: 364

SEQ ID NO: 366 - DNA, 1416 bp, Streptococcus pneumoniae; identical to SEQ ID NO: 349

SEQ ID NO: 367 - DNA, 1425 bp, Streptococcus pneumoniae; identical to SEQ ID NO: 362

SEQ ID NO: 368 - DNA, 1449 bp, Streptococcus pneumoniae
gtgattgtgaaaaggaaatccattgcccgaaattatttacttaatttgataaaaacgaccttgtctattctatttcccctgatatcatacccatatgtgtcgcgtattttatctgtggatgggctgggtgcaatcaatttttcagcctcgtttgttaattatttcgtactaattgcaatctttggaattaatgtctacgctgttagagaaggtgccaaatatcgagatgataaaaagttacttgggaagtttgtaacggagatgttacttgttagtatttgcacggcaatgatttcattctctttattaactgtttccttactcctacctgctctatcagaatatagaagtcttattctaattttttcaattaccattatatttaatgttgtggggatggagtggttttttcaattgatggaagactatcaatatataacacttcgtgcaattatatttcaaattctatctttgatatttttattcatatttgtcaaagataggaatgatatttatgtgtacgctctaattacagttgcagcaaatgcaggttctcaaatactgaatttatttagacttagaaaagaagttgaaatatttagatataaagactataatattagaaattattttaaaccgatgtttctgatatttttaaccttattgtctatgaatatatatagatatttagatgttacttttctgggcttctttaaaaatgatcgaagtgtgggctactatagcttagctacaaaaataacaagtgctataatttcgatggtatcttctgttacagttattttgacacctcgtctagcatatcattataagaaggaggagtttgataaattctataaaattgcttatagttcctttgattttattttattacttgctataccagttgttataggtgtattgagttttagttctattttagtagattttttgggtgggagtacttttgtttcttctgtgttaacggtggagatattaagtttaacaattctattctctaatttgaatgctttattaattacacctattttaacggttatgaatcgtgagaaagcggttttaaaaatttttattatcgcattgattttcaatgttattacaaacatgttacttattccagtaatggattttaatggttcagctttggttacagttttgacagaaggaattatttgcatattatctttaattagtataaaaagtgtgtttaatgttagaaggctatttaagaatctttttcaatatctggtagctagtatctttataattgttgtcaagattgttatttctcagtacgtttcatcaaattatattatttttatttcaactgctttactaagcgcaattttatatttccttactttgatattattgagaaatgagttagtaccacaattaataatagaagttagaaagaaaatatatagatag

SEQ ID NO: 369 - DNA, 1461 bp, Streptococcus pneumoniae
atggaaagtcaaaaaaagagcgttgaaaagaattatatttataatttgatttatcaaatgctaactgtagttattccaattatcataacaccctatatttcacgtgttctaaacgctgatgggatagggatttttagctacacaacagcagtggtaggttattttgttttattgggtaatttaggaattgcaacttacggacaactacaagttgctaaataccaagaagacagatttaaattatccaaaatattttttgagttattgattttacgaactatattgcttttactagttttactgatatatcttgtatttatccaatttcaagatataatttataaatctatttattacgttttaataattcaaatactttgtagtttgctggatatttcatggtttttgcagggtcttgaagaatttaaaagtattgtgatgcgtaataccctaataaaggtgttaagtgtaatatctatcttgatttttgtaaaaaaagatacagatttgattttatatgctttgattatgaacgcatcaacactgcttggaaatatttctatttttgcatatgtacctaaatatgttcaaagagttaagttggctgagattaatttattaaagcatatttcacactgtttagtttactttataccaacaatagcaactacaatatatttaactttggataagacaatgataggttggttttccttaaatacttttgaaaatggttattatgagcaggctcaaaaaattgagcagatggttgtgacaatagttacatctttaagtgtagtaacgatgcctagaatgacttatttattgaataacaatcaatttcaagaatttaaagtgcgacttgaacaatcgataaggtttatcctttttttatccttacctatgagtttaggattagtagggatagcaaataattttatacctttgtttttaggtgtaggatttgaaaagtctatatcaatattaaagatttttagtctactagttatcattataggtttaaataatgcattagggaagcaaatacttatgccttcagggcagcaaagggcttataatattagtgtggtaataggtgctgttatcaatatagtcttcaatttaatattgataccgcaatttttttcactgggtgctgcgatttcttccgttcttgcagagtttgcaattttaataatatttctctattattctagaaattgtattcctcctaaatgggttattagaacagcagtgaaataccttggatctagtatagtaatgtttcttattattaggacaatagagctactatctcctccttcgtggacagttgttataatacaagtcattgttggagtcgcagtatatattctatcgttatatatattaaaagatcaaattgtaagaaaatactatctaagattgagaaaactaaatgttcttgcaaggaaagtatga

SEQ ID NO: 370 - DNA, 1461 bp, Streptococcus pneumoniae; identical to SEQ ID NO: 369

SEQ ID NO: 371 - DNA, 1416 bp, Streptococcus pneumoniae
atgaaaatactaaaaaactatgcctacaatctttcttatcaattgttggtgatcatactccctatcattacgactccctatgtaacgagggtttttagttctgacgatttaggaacgtatggctactttagctccattgttacctattttaccttgcttgcaactcttggtgttgccaactacggtaccaaagagatttcagcacatcgtaaggaaattgggaagaatttctggggaatttattctctccagtttggtgcaacttggctatccattttgctttatcttgccctttgtttcttatttacttcaatgcaaaatccggtagcttatatattgggattaagtttagtgtcaaaaggtttggatatttcttggttatttcaaggtttggaggattttagaaagattacagttcggaacatcactgttaagttagttggtgcaatctctattttcctatttgtaaaatcagctaatgacttatacttatatgtatttcttcttaccattttcgaattgttagggcaattaagcatgtggttacccgcaagacaatttattggtaaatcatattttgattggaaatacgctaagaagcatttgaaaccagttatcttgttattcttgccacagattgccatctcgctttatgtgactttggaccgtacgatgctgggggtgttggcttcgacaaaggatgttggaatttatgatcaagctttgaagcttgttaatattttactgactttggttacctcactggggagtgttatgttgccacgagtatctagccttttatcgtctggggattacaaagccgttaataagatgcacgagatgtcttttttaatctataacttggtgattttcccaatcatagcaggaatgttgattgtcaacgatgattttgttcgtttcttccttgggcaaaattttcaagatgctcgctatgcgattgctattatgaccttccgtatgttctttattggttggaccaatataatgggaattcaaatcttgattccgcacaataaaaataaggaatttatggtttcaacgacagttcccgctattctgagtgtgggattgaatttgttgcttcttccccaatttggttatataggtgctgcgattgtttcagttttgactgaggcgtttgtatgggggattcagttattctatacacgtttttatttaaaagaagttcctattatcggttcaatgacaaaaactgcacttgcatctgctgttatgtatggtctcttgctaggttcaaaaacatctatacatttttcgccaaccataaatgttttaatattttcggtgctaggcggaattatctatctttttgcaattctatctctgaaagtggtagatgtgaaagaattaaaacaaataatcaggaaaaattag

SEQ ID NO: 372 - DNA, 1416 bp, Streptococcus pneumoniae
atgaaagtactaaaaaattacgcctacaatctttcttaccaattgttggtgattatactccctatcattacgacgccctatgtaacaagggtcttttcttcggatgatttagggacgtatggttattttaattccatcgttacttattttatcctcttagcgacgctaggagttgctaactatgggaccaaggtcatttcagggcatcgaaagcaaattcaaaaaaactttttgggaatctattctctgcaattaggtgcaacagttctttctctgtccttgtatgctcttctttgtctaactcttccctttatgcaaaatccggtagcctatattctaggcttgagtttagtttctaaaggtttagacatctcctggctctttcaagggttggaggattttcgaaagattactgtccgaaatataacagttaaactagttggggtcatttccatcttcctctttgttaaatcatcaaatgacctttacctctatgtctttttgctaaccatttttgaactctttggtcaattcagtatgtggataccagctcgagaatttattggtagacctcattttgacatagaatatgctagacatcatttgaaaccagtcatattattgttccttccgcaagtggcgatttccttgtatgttacgctagatcgtactatgcttggagcgttagcttctacaaaagatgtagggatttatgaccaggctttaaagttggtaaatatccttctgaccttggtaacttccttgggaagcgttatgttacctcgagtcgctcatttgttagcgacagatgatcataaggcagtcaataggatgcatgaaatgtctttcttcatttataatttagtgatttttccaatgatggcaggaatcttgattgtcaatgatgattttgttcagtttttccttggtcaagattttcaagatgcgcgttatgcaatcgccattatgatcttccgtatgttctttatcggttggaccaatatcatgggaattcagatgctgatacctcataatcaaaataaagaattcatgatttcaacaacagctcccgcaattatcagtgtaggtttgaacttactattccttcctaaactgggatatatcggggcagccattgtctctgttttaacagaggcacttgtatgggcaatccaattattctatactcgcagatatttaaaagaagttcctataatcggatcaatgtcaaaaattatactagcatctgccattatgtatggccttttactaagttcaaaaacagttatacatttttcaccgaccttaaatgttctagcatttgcagcgcttggtggaatcatttatctttttgcaattctatctctgaaagtggtagatgtgaaagaattaaaacaaattattaggaaaaactaa

SEQ ID NO: 373 - DNA, 1413 bp, Streptococcus pneumoniae
atgaaagtactaaaaaattacgcctataacctctcctatcaactattactcattatactacctatcattacaactccctatgtgacacgggtcttttctttgaatgatctagggacttatggttattttaactccatcgttacttattttattcttttagcaactttaggagttgctaactatgggaccaaggtcatttcagggcatcgcaaggaaattgaaaaaaatttttgggggatttactccctgcaattgggtgcgacagttctttctatgtttttgtaccttgttctttgtctaattcttccttttatgcaaaatccattcgcctatattctaggcttgagtttggtttctaaaggcttagatatttcctggctctttcaaggattagaggattttcgtaagattacagcaagaaatatcattgtgaaactcgtaggtgtaagttcgatttttttatttataaaatcagctagtgacctttacctttacgtatttcttttgacaatatttgagttgttgggacagttaagcatgtggttacccgctcgggaatttattgggaaaccatattttgatttaagctatgcaaagcagcatctgaagccaattgttttattattccttcctcaggtagcgatctccttatatgttactctagaacgtacgatgcttggtgctttatcttctacaaaagatgtaggaatttatgatcaggctcttaaattggtaaatattttattaacattggtaacatcgcttgggagtgttatgttacctcgagttgctaatttattagcaacaggtgattacaaagctgttaataagatgcatgaaatgtccttcctcatttataatttagtgatttttccaattatggctggaattttgattgtgaatgatgattttgttcagtttttccttggtcaagattttcaggatgcacgttatgcaatcgccattatgatcttccgtatgttcttcatcggttggaccaatataatgggaattcagattttaattccccataataaaaataaagaattcatgatttcaacaacagcttctgcaattatcagtgtaggattaaacatgcttttcctaccaaagcttggctatattggagcagccattgtttctgtattaacagaagcacttgtatgggcaattcaattgttctatactcgcagatatctcaaagaagttcctatcatagtatctatgacaaaaattgttctagcatcagttatcatgtatggcattttgctaggttcaaaaatatttatacatttttccccgactataaatgttctagcatttgcagtacttggtggaatcatttacctttttgcaattttatctatgaaagtaatagatgtgaaagaattaaaacaaatcataaaaaaataa

SEQ ID NO: 374 - DNA, 1425 bp, Streptococcus pneumoniae
atgaatgttgaaaaaaatttgaaacgtggaattttctatactgcaatagggaagtattcgaatgtagttattcaattacttgtaacagcaattcttagccggattctctcacctgtagaatatggtatcgtagcagtagtaaatgtatttcttttattttttcaaatgttagcagattcaggaataggacctgctattgttcaaaataaagaactgaataagtcagatttaatcagcattttctcattaacgatttatagtggaatcgtactttcttgtatttttgctttactaggatatcctatgggggtgttatacggcgaaactatttatgttagtttatttccgcttttaggtctttgtgttttattttataccattacaattgtaccgcaagctattctaatgaaaacgatgaactttaaaatagttaattttctaactattttttctaacattgctagtggcttggtgggcgttattctagcggtatcacattttggtgtctattctctaattttttctaatattgtaaaagcaatggttttatttatcgctttatattctaaagcaaagataaatttctattggaaagtttcaaaaagttctattggtaaaatttttgagttttcaaaattccaatttttgtttaatttttggaattattttgctagaaatttagataatttattaattggacgattgattagcccgtcagatcttggttattatgataaggcttatcaattatctttgtaccctaatcaaattttatcacaggttgtgacaccagcattgcatcctattatgtccaattttcaagataatatcagtaagataggcgaagtttatcttcagatttctagaatattcgtaatagttggcatacctatttcggcttatttatactttaatgctcaatatgttgttacatttatgtttggagataattggtcccaaagcgttccggtgtttcaaatattagcagcaacaatttggttacaaatggctaatagccctactggtgcattttaccaagcaactaatcagacaaagttattgtttagaattggcttattgacatcttttattaatattttagctattgttattggtgtaatgttgcaaagcatccaatgtgttgcgtacatgcttttgatttctttcagtcttagtttagtaatcaatactctttacttaacaaaaaaagtattaaatatttctgctcaaaaatatataaaaccgatattaattaatttgacaattatcagtccctatattgtgtttaatttgtttatctcggattttgtaaacgaccttattttacatttatcagttcagtttgttattttatttttaatatggggaattgggatgtatattagcggagagtatcgacgagtcttcgcggttataagaaaataa

SEQ ID NO: 375 - DNA, 1413 bp, Streptococcus pneumoniae
atgaaagtactaaaaaactacgcctataatctttcttatcagttattagtgattatactcccaatcattacgactccctatgtaacacgggtcttttcttcggatgatttagggacgtatggatactttaattccattgtcacctactttatcctcttagcgacgctgggagttgctaactatgggactaaggtcatttcagggcatcgcgaggaaattcaaaaaaacttttggggaatctactctctacaattaggggcaacagttctttctctggccttatatgttcttctttgtctgatccttccctttatgcaaaatccggtagcctacattctaggcttgagtttagtttcaaaaggtttagacatttcctggctctttcaagggttggaagattttcgaaagattactgttcgaaatatcacggtcaagcttgttggagtcatttcaatcttcctctttgtcaaatctgcaaatgacctatatctctatgtttttttgctaaccatatttgaactcttggggcaactaagtatgtggttgcctgctcgtgagtttattgatagacctcattttgatttagaatatgctaagcctcatttgaagcccgtcatattattatttcttcctcaagtagcaatctctttgtatgttaccttagatcgtaccatgcttggagcattggcttctacaaaagatgtagggatttatgatcaggctctaaagttggtaaatatacttttgaccttggtaacttccttgggaagtgttatgttgcctcgagtagctaacttattagcaacaggaaatcatagagcagtcaataagatgcatgaaatatccttcctcatttataatttagtgattttcccaatgatggcaggaattttaatcgtcaatgatgattttgttcagtttttccttgggcaggattttcaggatgcacgttatgcaattgccattatgagcttccgtatgttctttatcggttggaccaatatcatgggaactcagatactgatacctcacaatcaaaataaagagttcatgatttcaacaacagctcccgcaattgtcagtgtaggtttgaacttactattccttcctaaacttggatatataggagcagccattgtttctgtttcgacagaggcgttagtatgggcgatacaattatactttacccgtagatacctaaaagaagttccaattatcggatctttgacaaaaatagtcctagcatcagctatcatgtatggcattctgctaggttcaaaaacacttatacaattttcgccaactataaatgttatagtatttgcagtgcttggtggaatcatttatctttttgcaattctatctctaaaagtggtagatatcaaagaattaaaacatattattaaaaaataa

SEQ ID NO: 376 - DNA, 1416 bp, Streptococcus pneumoniae
atgaaggtattaaaaaactacttatataatctctcttatcaactattagttattattcttcctatcattacgactccttatgtgacgcgcatattttcttcagatgatttagggacatacggttacttcaactccattgttacctacttcattttacttgctactctgggtattgctaactatggaacgaaggagatatctgggaataggaagagtattcagaaaaatttctggggaatatatactttacagtttggagcgactactttttccatttttctctatatactagcctgtcttttcatcccagggatgaaaaacctagttgcatatatcttgggattgggtttagtctctaaaggactagatatttcatggttgtttcaagggttagaagattttcgtaaaattacggtcagaaatatcacagtgaagcttgttggtgtaagtgccattttcttgtttgtaaaatcagcgagtgatctatacttgtatgtttttcttctaacagcatttgaattgttgggtcagttaagtatgtggtttccagcccgtgaatatattggaaaaccacattttgatatagaatatgctaggcatcatctgaagccagttatcttgctattcttaccacaaattgctatctcgctttatgtgactttagatcgtacaatgttaggggcgctagcttctacaaaagatgtcggcatttatgatcaggctttgaagctggttaatattttactgaccttggtaacttccttgggaagtgttatgctgccacgcgttgcgcatttgttagcgacaggtgatcataaggtagtcaataagatgcatgaggtatccttcttaatttataatttggttatttttccgattatggcagggatgttgattgtgaatgatgattttgttcaatttttccttgggcaagactttcaagatgcacgctatgcaatttccattatgatcttccgtatgttctttatcggttggactaatataatgggaattcagatactgatacctcataatcaaaataaagaattcatgatttcaacaacaactcccgcaattatcagtgtaggtttgaacttactattccttcctaaactgggatatatcggggcagccattgtctctgttttaacagaggcacttgtatgggcaatccaattattctatactcgcagatatttaaaagaagttcctataatcggatcaatgtcaaaaattatactagcatctgccattatgtatggccttttactaagttcaaaaacagttatacatttttcaccgaccttaaatgtgctagcatttgtagcgcttggtggaatcatttatctttttgcaattctatctctgaaagtggtagatgtgaaagaattaaaacaaattattaggaaaaactag

SEQ ID NO: 377 - DNA, 1416 bp, Streptococcus pneumoniae
atgaaagtacttaaaaactatgcatacaatctatcttatcaattattggtgattatacttccaatcattacgaccccctatgtgacacgggtcttttcttcgaatgatctagggacatatggttattttaactccatcgttacttattttatcctcttagcaacgctaggggttgccaactatgggaccaaggtcatttcagggcatcgcaaagaaattgaaaaaaacttttggggaatctactctctgcaattaggtgcaacagttttttctctaaccttgtattgtcttctttgtctaactcttccctttatgcaaaatccagtagcctacattctaggcttggctttggtttctaaaggattagatatatcctggctttttcaagggctagaggattttcgaaagattactgttcgaaatatcacagtcaagttcgttggtgtaatctctatcttcctctttgtcaaatctgcagatgatctatacctctatgtctttttgctaaccatatttgaactcttggggcagctaagtatgtgggtgcctgctcgtgagtttattggtagacctcattttgatttagaatatgctagacatcatttgaaaccagtcatattattgttccttccgcaagtggcgatttccttgtatgttacgctagatcgtaccatgcttggagcgttagcttctacaaaagatgtagggatttatgaccaggctttaaagttggtaaatatccttctgaccttggtaacttccttgggaagtgtcatgttgcctagagtttcaaatcttttatcctcaggggatcataaagcagttaacaaaatgcatgagatatcatttttaatttataatttggttattttccccattatggcaggaatgctaattgtaaatgatgactttgttacctttttcctcgggcaagattttcaagaggcacgttatgcaatagcaattatgatttttagaatgttctttattggttggaccaatattatggggattcaaattttgatacctcataatcaaaataaagaatttatgctgtcaacaacaattcctgctattgttagtgttggattaaatcttctcttgcttcccaagtttggatatataggagcagcaattgtttctgtcttaacagaggcgctagtatgggctattcaattgtactatattcgtgcatacctaaaagatgttccgattattagaactatgataaagattattctagcctcagctatcatgtatagcgttttgctagtttctaagacatacatacatttttccccaactataaatgttttggtatttgtggtgttaggtggaatcatttatcttttttcagttttatctctaaaagtggtagatgtgatagaattaaaacaagtaattaggaaaaattag

SEQ ID NO: 378 - DNA, 1416 bp, Streptococcus pneumoniae
atgaaggtattaaaaaactacttatataatctctcttatcaactattagttattattcttcctatcattacgactccttatgtgacgcgcatattttcttcagatgatttagggacatacggttacttcaattccattgttacctacttcattttacttgctactctgggtattgctaactatggaacgaaggagatatctgggaataggaagagtattcagaaaaatttctggggaatatatactttacagtttggagcgactactttttccatttttctctatatactagcctgtcttttcatcccagggatgaaaaacctagttgcatatatcttgggattgggtttagtctctaaaggactagatatttcatggttgtttcaagggttagaagattttcgtaaaattacggtcagaaatatcacagtgaagcttgttggtgtaagtgccattttcttgtttgtaaaatcagcgagtgatctatacttgtatgtttttcttctaacagcatttgaattgttgggtcagttaagtatgtggtttccagcccgtgaatatattggaaaaccacattttgatatagaatatgctaggcatcatctgaagccagttatcttgctattcttaccacaaattgctatctcgctttatgtgactttagatcgtacaatgttaggggcgctagcttctacaaaagatgtcggcatttatgatcaggctttgaagctggttaatattttactgaccttggtaacttccttgggaagtgttatgctgccacgcgttgcgcatttgttagcgacaggtgatcataaggtagtcaataagatgcatgaggtatccttcttaatttataatttggttatttttccgattatggcagggatgttgattgtgaatgatgattttgttcaatttttccttgggcaagactttcaagatgcacgctatgcaatttccattatgatcttccgtatgttctttatcggttggactaatataatgggaattcagatactgatacctcataatcaaaataaagaattcatgatttcaacaacaactcccgcaattatcagtgtaggtttgaacttactattccttcctaaactgggatatatcggggcagccattgtctctgttttaacagaggcacttgtatgggcaatccaattattctatactcgcagatatttaaaagaagttcctataatcggatcaatgtcaaaaattatactagcatctgccattatgtatggccttttactaagttcaaaaacagttatacatttttcaccgaccttaaatgttctagcatttgtagcgcttggtggaatcatttatctttttgcaattctatctctgaaagtggtagatgtgaaagaattaaaacaaattattaggaaaaactag

SEQ ID NO: 379 - DNA, 1416 bp, Streptococcus pneumoniae
atgaaagtacttaaaaactacgcttacaatctttcatatcagttactaatcataatcttaccgattattaccactccctacgttactaggatttttagttcggataatctagggacttatggttattttaactcaattgtaacctattttcttttgctggctactttaggaataggaaattatggaacaaaagagatttcaggtaataggaaagctatccatcagaatttttggggtatttatagtatacaactgggagcatctctattctccactttcctttatgttattgtctgtctaattttaccagctatgcaaaatactgtagcctatattttaggcttaagtttgttttctaaaggtttagatatttcttggttatttcaaggtttagaggattttcggaaaattacagtaagaaatataactgtgaaagtcattgggataacctcgattttcttatttgtaaaatcagctagcgacctctacctttatgtttttcttttaacgatatttgagttattagggcaacttagtatgtgggtaccagctcgggaatttattgggaaaccgcactttgactggttgtatgctaaacaacatttgaagccagttatcttattatttttaccacaggtcgctatatcgctttatgtgactttagatagtactatgcttggttctctagcctcaataagagatgttggaatttatgatcaatctctgaaacttgtcaaaatattattaacattagtaacttcattaggaagtgtcatgttgcctagagtttcaaatcttttatcctcaggggatcataaagcagtcaacaaaatgcatgagatatcatttttaatttataatttggttattttccccattatggcaggaatgctaattgtaaatgatgactttgtaacctttttcctcgggcaagattttcaagaggcacgttatgcgatagcaattatgatttttagaatgttctttattggttggaccaatattatggggattcaaattttgatacctcataataaaaataaagaatttatgctgtcaacaataattcctgctattgttagtgtaggcttaaatcttctcttgcttccaaagctgggctatataggagcagcaattgtttctgtcttaacagaggcgctagtatgggctattcaattgtactatactcgtacatacttaaaagatgttccgattattagaactatgataaaaattattctagcctcagctatcatgtatagcgttttgctagtttctaagacatacatacatttttccccaactataaatgttttggtatttgtggtgttaggtggaatcatttatcttttttcagttttatctctaaaagtgatagatgtgatagaattaaaacaagtaattaggaaaaattag

SEQ ID NO: 380 - DNA, 1416 bp, Streptococcus pneumoniae
atgaaggttttaaaaaattatgcatataatctctcttaccagctactagtgataattcttcctattataacaacaccttatgtaactagggtatttagttcaaatgatttaggaacttttggctactttaattcgattgttgcttattttatttttttagcaacattaggagtagctaattatgggacaaaagagatttcagctcatagaaataatatccctaataatttttggggtatttatactttgcagtttactgcgacttgtatatcgatatttctatatattggtctgtgcttttcattttcatttatgcaaaatccagtagcttatatattggtattatctctattatctaggggattagatattacttggctttttcaaggactagaagattttaggaagataactgttagaaatatcactgtcaaactcataggtgttatttcaatatttttgttcataaagtctcagaatgatttatatctatatgtctttttattaactatatttgaatttttaggacaattaagtatgtggctgccagctctagaatttataggtaagccacaatttgatttcaattatgccaagaaacatcttaaaccggtgatattactgtttttacctcaaatagcgatatctttatatgttacgttaaataggacaatgcttggcgctttagcatcaacaacagatgttggaatttatgatcaatctttaaagttggtaaatatcctacttactttggtaacttcactaggtagtgttatgctaccacgtgtttcgaatcttttatcaacaggggataagaagtctgtgaatcgcatgaatcaattatctttctttatatacaatcttgtgatttttccaactattgcaggtatgttaattatcaataatgactttgtgaaatttttcttaggaaatgactttcaagaagcaaaatatgctattgcaatcatgatttttaggatgttttttattggttggacgaacattatggggattcaaattttgatacctcataataagaacaaagaatttttgacttcaacaacggttccagcaattattagtgttggtttaaatttgcttttactgcctgaatttggatatattggggctgcgattgtttctgtattaacagaaatatttgtatgggttatccaattgtattttacaagaaaatatttaaaagatgtaccaatttttaaatcaactgttaaaatattttcagcatcaattcttatgtatgttgctctttcatttttgaagtcaattttgacttttacacctattgttaatgttgttgtatgtgctattgtgggggcgatactttatggtttactaattttaatatttaaagtagttagcttaaaggagataaaagagcagctgttaaataattag

SEQ ID NO: 381 - DNA, 1416 bp, Streptococcus pneumoniae
atggaaacaaaagaaaaacgtttagttcataatacgattatttttacaattggaagtttaggttcaaaatttattcagtttttgttagttcctttgtatacctatactttaacagcttctcagtttgggattaccgaaattttattaaccgctgttaatcttttaatacctgtattttctatttccattgctgatggattattaagatttggattagataagaccttaagaagagaaaatgtcttgaaatcagcttttatcatttcgatacttgggactattttatccattatttcgattccaattttttcgctctatcctactttgtcagagtggatggtttattttatcatcattttgaacttgagaatgtatcgggatgtttttgcgattcaattgaaagttgaagggaaaaatacattatttgcttgcgatagtatgatttatacatttgttttatctttagcgagtattgtctttctagttcctttttctttaggcatttcaggttattttttcgcttatattgtcagtaatggaatttccattttctttatcttatttttcggaggagtctggaaaagttttactagcggaagatttgaaaaacaattgatgatacaactattgaaatattcagcacccatgattttaaatggtattgcatggtggattacaaatgcctcagatcggtttatgttgcaatggtttatggatgatagagcggtaggtttgtatggtgttgttgcaaagttacccttattaatcggaacgtttactggagtatttaatcaggcatggattatttcagcagtagaagagtttgaagaagaaaatgaagaatggttttatcaaaaagtttttcaccaatattatgcagcactctttttaagtgtttcagtatttttgcttctacttcaaccatttatgaaagtatatgttagtccaagcttttatgaagcatggcaatatgctccgtttttattattgtcaagtgtagtttcaggaatcgccgcatttatgacaggtttttatgttgcgcaaaagaaaaatttgaatattatatacacgacgattgctggtgcttttgcgaatattttgttaaatgcgatgtttattcctatgcttggagtattaggcgcttccattgcgacctttctatcatggtttgttattgcaatctatcgtatgaaagatgttgaaaactttgcttgttttccattagataaaaaagttttttggtatttatttctattatgtattcagaccattacgatgacttttctgccaatccttggcattgttttttcagtcgtattgattccttatttcttttaccaagaacaggaatttctagcagttttatttgataaaggaaggaaaaaagtatgttcattcaaaaaatcaaaaagataa

SEQ ID NO: 382 - DNA, 1425 bp, Streptococcus pneumoniae
atgaaagtactaaaaaattacgcgtataacctctcttatcaattattactcattatacttcccatcattacaactccctatgtgacacgggtattttctgcaaaggatcttggaacatatggatactttaattccattgttagctattttattttgttagctactttgggggtcgctagctatgggactaaggagatttcgagtaatagaaaaaatatcccgaagaatttctgggggatatatactcttcaatttggtgcaacatcattatcaattctcttatacactttattatgtttaggaatttacataatgagaaatcctgttgcgtatatattaggaatgagtttgatttctaaaggattagatatatcctggctttttcaggggttggaagattttcgtaagattatagtcagaaacatattagtgaagctcgttggtgtaatttcaatatttttattgataaaatcagcgaatgatctttatctttatgtctttctcttaacgatatttgagttattgggacaattaagtatgtggttacctgctcgaaagtttattggaaaaccacattttgatatagattatactaaatatcatttaaagccaattcttttattatttctccctcagattgctatatcactatatgcaactttagaccgtacgatgttaggaatgatatcatctacaaatgatgtcgggatctatgatcaagcattaaaattgatcaatattttattaactgtagtaacttcgttaggaagtgtcatgttgccgagagtatctaatctcttggcgacagggaatcataaggcagttaataaaatgcatgagatgtcatttctgatctataatttgattatttttcctattatggcaggaatgctaattgtaaatgatgattttgttaactttttcctcgggaaagattttcaagacgcacgttatgcgatagcaattatgatttttagaatgttctttattggttggagcaatattatgggatttcaaatgctaatcgtacataataagaataaagaatatatgatctcaacaactgtgcctgcaattcttagtgttgccttgaatttgttatttcttcctaaactcggatatataggagcggcaattgtatctgttttaacagaggcggtagtttgggggatacagttattctatactcgttcttatttgagagaagttcctattctaggttcattgtttaaaattatagtttcatcgggatttatgtatgtcaatttattatttgtaaaacatcttctgaacgtatcacctatgataaatgtagggatttatgctgttctgggagcaatgatttatctcatattaattttgatttttagggttataaatccaaatgagttaaaacaacaattattaaaaaataaaggagcttaa

SEQ ID NO: 383 - DNA, 1458 bp, Streptococcus pneumoniae
atgagcatgaaagaaaaatcaataaataaaaatgctattttgaatattattttaacactaacaaatattgtgtttccattaattacttttccttatatttctagaattctgaatccgtcaggaataggtgcaatctcttttttttcatctattggttcttatggtgttttagttgcctcgcttggaatttcaacttatgggattcgggtgattgcaaaggatcgttatcataaagataagataactaaaatattccaagaattgatagttattaatagtgtaatgtctattatagtgacgttcttccttgttttaatgagtttccgattagagcagttaagttcagaaaaaggtttattaataatcacatgtattactattttgtcctctccttttaatttgaattggttttatagtggaatagaagagtattcatatataacaaaacgttcaatattttttaaattagtttcgttgatattaacctttttatttgttaaaagtaaagacgattatattctatatgctgtgataacattgttttcaacattggcttccaattttataaatattttggagagtagaaaatacatcaattttaatttgagaagggatttagaatttagatatcatctaaaaccaatgtggtatttatttgcttcgttacttgcagttaatatttatataaatttggattctgtgatgttaggaattattaatggaaatgatgcggtaggtatttactcaatcgcatcaaaagtaaaatggattttattatcagtcgttacttcagtgagttcagttttattacccagattatcattttatagcaataaatatgacgaaacgaagtttaataatattttgagaaagtcatctacaataatttttatgatttcaattcctctaacaattttttttatgataaaagcgaaagagagtatattattgttaggtggagagcagtatattcaggcagttttagcaatgcaaatattaatgccgatcttggtaatatcaggtttttctaatattacaggtaatcagatattgatccctactggtaatgagaaatattttatgagagcagtttctataggagcaatagtaaatttatgtttaaatttgttgcttatgcctattttgggaattatcggaggtgcgatcgcaactctttgtgcagaatcagtccaaatgattattcaattttatttttctcgtaataaattaatgggtaatatttcattaaattcaatcaaaaaaatagcctattcatcaatttttgcgggtatactgttaatagtaatacaaaatattatagagaattttaatagcttcttaaatttagctgcatctagttttctatattttggagtatattttttcttattggtattgtttaaagaaagtacaattaagaaatttctaaatcaaattttttataaggatatttcatga

SEQ ID NO: 384 - DNA, 1188 bp, Streptococcus pneumoniae
atgttacttgttagtatttgcacggcaatgatttcattctctttattaactgtttccttactcctacctgctctatcagaatatagaagtcttattctaattttttcaattaccattatatttaatgttgtggggatggagtggttttttcaattgatggaagactatcaatatataacacttcgtgcaattatatttcaaattctatctttgatatttttattcatatttgtcaaaaataggaatgatatttatgtgtacgctctaattacagttgcagcaaatgcaggttctcaaatactgaatttatttagacttagaaaagaagttgaaatatttagatataaagactataatattagaaattattttaaaccgatgtttctgatatttttaaccttattgtctatgaatatgtatagatatttagatgttacttttctgggcttctttaaaaatgatcgaagtgtgggctactatagcttagctacaaaaataacaagtgctataatttcaatgttatcttctgttacagttattttgacacctcgtctagcatatcattataagaaggaggagtttgataaattctataaaattgcttatagttcctttgatttgattttattacttgctataccagttgttataggtgtatttagttttagttctattttagtagattttttgggtgggagtacttttgtttcttctgtgttaacggtggagatattaagtttaacaattctattctctaatttgaatgctttattaattacacctattttaacggttatgaatcgtgagaaagcggttttaaaaatttttattatcgcattgattttcaatgttattacaaacatgttacttattccagtaatggattttaatggttcagctttggttacagttttgacagaaggaattatttgcatattatctttaattagtataaaaagtgtgtttaatgttagaaggctatttaagaatctttttcaatatctggtagctagtatctttataattgttatcaagattgttatttctcagtacgtttcatcaaattatattatttttatttcaactgctttactaagcgcaattttatatttccttactttgatattattgagaaatgagttagtactacaattaataatagaagttagaaagaaaatatatagatag

SEQ ID NO: 385 - DNA, 1449 bp, Streptococcus pneumoniae
gtgattgtgaaaaggaaatccattgcccgaaattatttacttaatttgataaaaacgaccttgtctattctatttcccctgatatcatacccatatgtgtcgcgtattttatctgtggatgggctgggtgcaatcaatttttcaacctcgtttgttaattatttcgtactaattgcaatctttggaattaatgtctacgctgttagagaaggtgctaaatatcgagatgataaaaagttacttgggaagtttgtaacggagatgctacttgttagtatttgcatggcaatgatttcattctctttattaactatttccttactattacctgctctatcagaatatagaagtcttattctaattttttcaattaccattctatttaatgttgtgggaatggagtggttttttcaattgatggaagactatcaatatataacacttcgtgcaattatatttcaaattttatctttgctatctttattcatatttgtcaaagacaggaatgacatctatgtgtacgctctaattacagttgcggctaatgcaggttctcaaatgctgaatttatttaggcttagaaaagaagttgaaatatttagatataaagactataatattagacattattttaaaccgatgtttctgatatttttaaccttattatctatgaatatatatagatatttagatgttacttttctgggcttctttaaaaatgatcaaagtgtgggctactatagtttagctacaaaaataacaaatgttataatttcaatggtatcttctgttacagttattttgacacctcgtctagcataccattataagcaaaaggagtttgataaattctataaaattgcttatagctcctttgattttattttattacttgctataccagttgttataggggtattgagttttagttctattttagtggattttttgggaggaagtgcttttatttcttctgtgctaacagtggagatattaagtttaacaattctattctctaatttgaatgctttattaattacacctattttaacagttatgaatcatgagaaagcggttttaaaaatttttattatagcattgattttcaatgttattgcaaacatgttccttattccagtaatggattttaatggttcagctttggttacagttttgacagaaggaatcatttgcatattatctttaattagtataaaaaatgtatttgatgttaaaagattatttaagaatctttttcaatatctagtagctagtatatttataattgttgtcaagattgttatttctcagtacgtttcatcaaattatattatttttatttcaactgctttactaagcgcaattttatatttctttactttgatattattgagaaatgagttagtatcacaattaataatagaagtcagaaagaaaatatatagataa

SEQ ID NO: 386 - DNA, 1416 bp, Streptococcus pneumoniae; identical to SEQ ID NO: 378

SEQ ID NO: 387 - DNA, 1470 bp, Streptococcus pneumoniae
atgagggataatatgacaagttataaagttaaatctatcaggtataattttataatgaattttattttgactgtttcaaatttcatctttccattgttaacttttccctatgtttctagagtacttcaagttgaggcgaatggtactgtagcatatgtatcatctattgtatcttattttatgatgattgcttcacttggtattccaacgtatggcatacgtgctgcagcaaaagtacgtgatgataaaagaaaattatctaccattgtccaagaactattgattattaatgtaattcttgtattcttagtactcattgcatattttataatgttgtttactttacagtcaatgtacgtctataaagaattattctatatcaatgcaataggtatactattaaatgtcataggagttggttggttttttcaagcaattgaacaatatgattatattactcttagatcgattttcttcagacttttatcattagctatgattttcttacttatccatagtcctgaggattatattatgtatgcaggaatttctgtttttgctagtgtgggatcaaatattttaaattttaaacgattatttgaatatatatcatttaaaaagacagaaacttatcattttaaaccacatataaagccgattttgattttatttgctcagacattggtagtgtcgatttatacgaatcttgataccgttatgctaggaagtatgaaaggaacatatgatgtgggattatatactgcagcaacaaaactaaaggggattctcttgagtgttgttacttcattaggaaatgttttgcttccaagaatgtcctactatgctaataatcaaatgaaagatcggtttttggaaatcataacaaaagctttgaactttacgttattcttatcgcttccattgtcagtattctttatttcagtatcaaaggaaagtattttgctacttgctggggaaggatatttaggtgctgttataggaatgcaatttttaatgtttgcagttattcctaatgcacttacgggtttattaggtattcaagtattaacacctttagaaaaagaaaagtacgtattactttcagtgacttcaggagcgttaatagatttactactaaacttcattttaattccagcttatggtgtaagtggagcagcatttgccactatgattgcagaattcgttgtattatgcgttcagatttactatacgagagatttgctattaaagaggctacatcaaatagttggcattagatatctatgctcggtaggggtgagtactattggaattgttcttgtaaagaaattacaattaagtgattttttgttattatgtgtggaaggaatcattttctttggaagttacggtgtgattctagtgttgttaaaggatgatttcattcttgatttttttaaaattttctataagaaattaaagaaaatttaa

SEQ ID NO: 388 - DNA, 1545 bp, Streptococcus pneumoniae
atgagtcgtgtaaaaaatagtttttttaatattttagctggaatagttggtactatcatatcgagtgttttagctttcatagttagaacagtgtttatccgagttttgggagagacttatctaggtttcaatggtctatacaccaatatcttaacagttttatccctaactgaattagggatcggatcatcaattgcttatttgatgtacaaacctttagcagaaaaagatggggacaaattagctcaagtagttcaattctataaaaaaatatatcgagtgattggtatcattattcttattttaggtttgtgtctcgttccttttttaccagtcattgtaaatttaaaaaatgcagaagatttgaactatacagcattgtatttgctttatttagcagatacagtttctacttatctattctttgcatataaaagaggggtattggctgctgatcaaaagatctatgttgcgaatatctttgatataattataacgacaattttatctattttccaaattatcactttgatgatttttaaagacttttatctatatattattttaaaaattgctaaaaatcttactttgaacctagtaattagtttaaaagtagataaaatgtatccaagtattcataaatttaaagggatagctccgcttccaaaaatggaaagacgactagtttggaaaaatgtatatgctacctctgttcgacaaatctttaacgaattaatgaattcaacagatagtatagttatttccattgtcttaggaatagttatggttggtaagtattcgaactatgcttatattttatcaatcgtatatattttctttggaggaatttttaatccgattcaggcttctattggaaacctatctctgttagcttctatagagaaaaaaaatgaaatcttcaatagattacgttttataaattttttctttttatcattttgctcaagttgtcttctagtattggtaaatccttttatcactatttggattggagaaaattacactttatcatttacaggtgtgattgcaatagttggtatgttattcgttagacaaactggaaattgtacaaccatttttcgtttgggagaaggatattttagagattatcatttttctcctttaatcgcgggcattttaaatttagttgtttcagtaattttagtgaattatataggtattgcaggtgtttttgttggaacaatgttaggatttggtttccaatttatactggtggatacaattgttacttataaaaaagtacttagtcgtccactatcagagttttatttaagatggttgcagacgatcttattaactgtaggtttatgtattgcgagttactatttatctcgattggttagagtgaatagtatatatgatttgatactgttgtttgtagtggttattggatttaatttttttgctttatgttttatttattggcgaaatgatgattttcaatattttattcaattggttaaaaattttatgaaaaatttggaggaaaaaaatcatgattaa

SEQ ID NO: 389 - DNA, 1395 bp, Streptococcus pneumoniae
gtggtagttttaatattgccactattgattactccatatgttgctagaatattaggcgcacaccagttaggaatttatgattatgtcaattcaatcgttactttgatttacactttaggtttattaggtattggtcactatggaaatcgcgaatgtgcctatactagagatgataaagaggaactatcagagacattttggagtattttagctattcagattattctaggatttttttcactgttattcttgtatattttttctaacataaattcaaattatcaagtttactttcagcttttctctatttggctttttgggtctattcttgattgtacttggctctatcgtggcttagaggaaatgcagtatgttgtgttaaaaaatattattgctaagttattatttacgatagcgaccttcttatttgtaaaaagtgaaacagatttaactctatatgtgctcatatatggtctctctgttttagtagccaatttatcaacttatagtcaactaaggatgtttattagtagaccaaagataaatattaatcgttttacggagattattttagggagtctcaaactatttttaccaagtatcatcacgcaaattatgttatctgctgataaaattgttctagggtctttaagttctggaatcagcaatgtttcttactatagtaatgcagagaagattatacagataccactctctttaatagtagtattaaattctgttatgatgccccgaatagcaaatgaatttcgtaatagacggttagaaaatatgaaaaagtatctgacaactgcagctgaattttctttgttcttagctgttccattgtcgattggcctttatgctattgcagatacttttgttccgtggtttttaggcgaaagttttatgccatctatggatgctcttaaatggcttgctcctattgctgttggcaatgctttgctaggagtctcaggtagtcagtattttgttgcagttaataaaacaaaagtcttatttttttctaatactttagctgcaattttaaatattctattagatttgttacttgctccaatgtttggagtagtaggggtttgtttagcaactatagtatcacttacctcttctgttatagtgcaatactatataatgtcaaaggatatacctattatacctattttgaagtcattatttaaatatctcatttattctatgataatgggaatgattattattgtttcattcagaggatatccagcaaattatagaacaactattaagcaaatcgttcttgggatagtagtttactttggtttgaacttgttgacacgagatgtatttgtttttcaaacattagattttgtaaagaaaggattcaaaaaaagtagatga

SEQ ID NO: 390 - DNA, 1545 bp, Streptococcus pneumoniae; identical to SEQ ID NO: 340

SEQ ID NO: 391 - DNA, 1434 bp, Streptococcus pneumoniae
atgaaagttttgaaaaattttttatataatttttcatataaaatcttaacgatgattcttccttttgtaacggttccctatgtaactagagtttttaatccaacattgatgggtgaatataattataccgcctctatagtcgcctatttttcaacatttggtatgttaggaattgttatttatggaagtaaccaaattgcaaaagtaagtcataagggaaaaaaagaaatatctcatgttttttcttcagtctattattttcagttattttcaacaactctaacgatactggtatatctagcctatatttttctttttccaagtcaataccaacaatattttatagtacaaatcttttctctcattgcaataatgtttgatattagttggctatttcaaggattagaagattttaagcgaattgttatgcgaaatacagtagtcaagctaattggtgtcgtttctatatttacattgataaaatcccaatcagatatctatttgtatgtatttattattagtttttctacgttaatttcgcaaggcattatgtggttaagtgtaaagaactatgtcaatttggttcgagttggagtaaaagagatactatcaaactttaaacctaacttatctttctttcttccccaaatttcaatttcgatttacaatacgctagataaaataattttgggctctttaggtagtgtatttgatgttggaatttatacccaagctgtgaatataaactctgttttagttaatttagtagtgactctcagtgctgtacttcaacctagaatgaccaatttgcatgctcaaggtaaaaaggaggaagttcgcagggtaatgtccgcatcaatgttatttaatagcatactaacctttccggtagtagttggaatattacttgttagccaagaatttgttcatatgtttttgggaaaagagtatgctgaaacatttattgctctcaacattgttgttttctctctaattccgattgctttttcagagattgtggggcgtcaaactctgattccgacggataatgttcgatactttacaatttcagtaatgtcaggtgctgtagttagcattttattaaattttattgtcatacctttcggaggatataaaggagcggcgcttgtacatgtactcgttgagattattgtatgcgcattaatgtcatattatgctcgtaaatatttagatattttagctttactcagaatagcaattaaaccactggttacgagtgtagtgactgttattatagtgcatttgatgttttcttatatttggcctatttataatagcttgcttagtttattattaaagataatagtttattcagtaatatatggtattctattattactgacaagaacaataactcagaaagagatttctatgataagaaaaaccccctag

SEQ ID NO: 392 - DNA, 1416 bp, Streptococcus pneumoniae
atgaaagtacttaaaaactacgcttacaatctttcatatcagttactaatcataatcttaccgattattaccactccctacgttactaggatttttagttcggataatctagggacttatggttattttaactcaattgtaacctattttcttttgctggctactttaggaataggaaattatggaacaaaagagatttcaggtaataggaaagctatccatcagaatttttggggtatttatagtatacaactgggagcatctctattctccactttcctttatgttattgtctgtctaattttaccagctatgcaaaatactgtagcctatattttaggcttaagtttgttttctaaaggtttagatatttcttggttatttcaaggtttagaggattttcggaaaattacagtaagaaatataactgtgaaagtcattgggggaacctcgattttcttatttgtaaaatcagctagcgacctctacctttatgtttttcttttaacgatatttgagttattagggcaacttagtatgtgggtaccagctcgggaatttattgggaaaccgcactttgactggttgtatgctaaacaacatttgaagccagttatcttattatttttaccacaggtcgctatatcgctttatgtgactttagatagtactatgcttggttctctagcctcaataagagatgttggaatttatgatcaatctctgaaacttgtcaaaatattattaacattagtaacttcattaggaagtgtcatgttgcctagagtttcaaatcttttatcctcaggggatcataaagcagtcaacaaaatgcatgagatatcatttttaatttataatttggttattttccccattatggcaggaatgctaattgtaaatgatgactttgtaacctttttcctcgggcaagattttcaagaggcacgttatgcgatagcaattatgatttttagaatgttctttattggttggaccaatattatggggattcaaattttgatacctcataataaaaataaagaatttatgctgtcaacaataattcctgctattgttagtgtaggcttaaatcttctcttgcttccaaagctgggctatataggagcagcaattgtttctgtcttaacagaggcgctagtatgggctattcaattgtactatactcgtacatacttaaaagatgttccgattattagaactatgataaaaattattctagcctcagctatcatgtatagcgttttgctagtttctaagacatacatacatttttccccaactataaatgttttggtatttgtggtgttaggtggaatcatttatcttttttcagttttatctctaaaagtgatagatgtgatagaattaaaacaagtaattaggaaaaattag

SEQ ID NO: 393 - DNA, 1416 bp, Streptococcus pneumoniae
atgaaagtactaaaaaattacgcctacaatctttcttaccaattgttggtgattatactccctatcattacgacgccctatgtaacaagggtcttttcttcggatgatttagggacgtatggttattttaattccatcgttacttattttatcctcttagcgacgctaggagttgctaactatgggaccaaggtcatttcagggcatcgaaagcaaattcaaaaaaactttttgggaatctattctctgcaattaggtgcaacagttctttctctgtccttgtatgctcttctttgtctaactcttccctttatgcaaaatccggtagcctatattctaggcttgagtttagtttctaaaggtttagacatctcctggctctttcaagggttagaagattttcgtaaaattacggtcagaaatatcacagtgaagcttgttggtgtaagtgccattttcttgtttgtaaaatcagcgagtgatctatacttgtatgtttttcttctaacagcatttgaattgttgggtcagttaagtatgtggtttccagcccgtgaatatattggaaaaccacattttgatatagaatatgctaggcatcatctgaagccagttatcttgctattcttaccacaaattgctatctcgctttatgtgactttagatcgtacaatgttaggggcgctagcttctacaaaagatgtcggcatttatgatcaggctttgaagctggttaatattttactgaccttggtaacttccttgggaagtgttatgctgccacgcgttgcgcatttgttagcgacaggtgatcataaggtagtcaataagatgcatgaggtatccttcttaatttataatttggttatttttccgattatggcagggatgttgattgtgaatgatgattttgttcaatttttccttgggcaagactttcaagatgcacgctatgcaatttccattatgatcttccgtatgttctttatcggttggaccaatatcatgggaattcagatgctgataccttataatcaaaataaagaattcatgatttcaacaacagctcccgcaattatcagtgtaggtttgaacttactattccttcctaaactgggatatatcggggcagccattgtctctgttttaacagaggcacttgtatgggcaatccaattattctatactcgcagatatttaaaagaagttcctataatcggatcaatgtcaaaaattatactagcatctgccattatgtatggccttttactaagttcaaaaacagttatacatttttcaccgaccttaaatgttctagcatttgcagcgcttggtggaatcatttatctttttgcaattctatctctgaaagtggtagatgtgaaagaattaaaacaaattattaggaaaaactaa

SEQ ID NO: 394 - DNA, 1152 bp, Streptococcus pneumoniae
atggctttgttttcattgtcactgttttttacctttacatttatttatagagagtatacaactttcttatatatacagtctattcaaatccttgcaacagcaatagatatttcttggttttttacaggaatggaatattttaaaagaacagtcactagaaatatattgacaaagatattatctgcaatttgtattattggatttgttaaaagtagggaagatttatggttatatatattaattttagttctctcaacttttttagggaatttaactttatggacatatgtttttgatgcgattgaagtaattaattttaaaagcttgcaacttaaagagcatattaaacctgtatttgtgttatttatacctcaattggcaacaactctttttctcacattaaacaaattgttgttaggaaacttatcaacattatctcaatctggctattttgatagtgcagacaaagtgatacgaattttactgacatttatcacagctattgggacagtaatttttccaagactggcgaatagctttaatagaggagaaaaaagaaatgtcgaagaattattaaaactatcttttggtgctattaatattattgctttcccaatgattgttggtattatggtaatcggaaggccattttcaatccttttttttggaagtgaatttgaaggaattgaaattgttttggggatattatcaatagaattggtttttatgggctggtcatcagtcttaggtaaccaatttttagtagctgttaataagacgaagggcttaactgtttcagtgtttattgcttcattaatcttgctcatatcctctttcatattgattcctatgttaggagcttctggagcttcaatttcatcagttataggagaggcgacaatagctttggtgcagttatattatgtttcaaaacatactagattatgtttattatttagtgatatttctaaaattttagtttctagtctcataatgggaacgacttgtttggtaattgggagttgtttttatgatggtattatgaaactctgtgttcaaggaacagttggtttaatggtatattgtataatgatttggtatagcaggactgaaattgtaaatactatacttaaaaaaataaagatacagtag

SEQ ID NO: 395 - DNA, 1434 bp, Streptococcus pneumoniae
atgggggatgttatgaaaacaataaaaaattatgcttataaatctttttatcaactatttttgataatagtaccttttataacaatcccatatgtttctagaattttaggagcagagttaatagggataaattcttatacaaacactataataagttattttgtgttaatagcaaatcttgggattttgatatatggtaatagaaccatagcatatcatagagaaagtattgaggagcgtagtaaaaaattttgggaaattgtctcaattaagttgctggtagcaattgttgcctatgtcatatttattatatttttattcttttattcaaaatatagttgggtttttgtgattcaatctgtacaaattatagctacggcatttgatatatcttggctttttgatggtgtagaagattttaaacgtacagtagtgagaaattttctggttaaaataatttccattattctaatttttacttttgtcaagtctacagaggactttgacaaatacatatggataacagtaggctctactttaatgggtaatttgacattatggagttatttacaccattatattattaaaattccgataaaaagccttaagttgagtgaacacttagttcccattttgactttattcataccacaaattgcttctattgtatttatgtcaatcaataaaattttactaggtaatatttcgacgatatcgcaagcaggctactttgagaatgcggataaggtgattagaattcttctggctttagtatcatctattggagtagttgtttttcctaaagttgctcatgcgtataggagtggtgatatgaagagagtattagggttgacttatatgacttttgatgcagttaatattataactattccaattgtagttggaatagtttctattagccctacattctcttctattttttttgggacggaattccaaggaatagataaggttctgtctgttctagtattggagttaattttcatgggatatacttcggttttaggtagtcaatatctcattgtaacagggcaaacttactttttaagtatctcggtgttcctaggaatattttctacagtgatatcttctttctttttcattccgatatacggagcgcttggaagtgcgatttcatctgtaattggagaagcatctattatgattggtgaaatttacttgttaaggaatcaagttgatttctattatctatatagggatgtaccgaaatacatgattgctagcgctgttatgtatattagtatctcgtcattgaattattttatttcttcgccatttgtatctttgctatcaagtattgctatgggtgcagtgacttatgttactgttgtcttattactatgcccaagaatagtaataaaattattaaataaaaacacaagatttttttga

SEQ ID NO: 396 - DNA, 1458 bp, Streptococcus pneumoniae
atgagcatgaaagaaaaatcaataaataaaaatgctattttgaatattattttaacactaacaaatattgtgtttccattaattacttttccttatatttctagaattctgaatccgtcaggaataggtgcaatctcttttttttcatctattggttcttatggtgttttagttgcttcgcttggaatttcaacttatgggattcgggtgattgcaaaggatcgttatcataaagataagataactaaaatattccaagaattgatagttattaatagtgtaatgtctattatagtgacgttcttccttgttttaatgagtttccgattagagcagttaagttcagaaaaaggtttattaataatcacatgtattactattttgtcctctccttttaatttgaattggttttatagtggaatagaagagtattcatatataacaaaacgttcaatattttttaaattagtttcattgatattaacctttttatttgttaaaagtaaagacgattatattctatatgctgtgataacattgttttcaacattggcttccaattttataaatattttggagagtagaaaatacatcaattttaatttgagaagggatttagaatttagatatcatctaaaaccaatgtggtatttatttgcttcgttacttgcagttaatatttatataaatttggattctgtgatgttaggaattattaatggaaatgatgcggtaggtatttactcaatcgcatcaaaagtaaaatggattttattatcagtcgttacttcagtgagttcagttttattacccagattatcattttatagcaataaatatgacgaaacgaagtttaataatattttgagaaagtcatctacaataatttttatgatttcaattcctctaacaattttttttatgataaaagcgaaagagagtatattattgttaggtggagagcagtatattcaggcagttttagcaatgcaaatattaatgccgatcttgataatatcaggtttttccaatattacaggtaatcagatattgatccctactggtaatgagaaatattttatgagagcagtttctataggggcaatagtaaatttatgtttaaatttgttgcttatgcctatttggggaattatcggaggtgcgatcgcaactctttgtgcagaattagtccaaatgattattcaattttatttttctcgtaataaattaatgggtaatatttcattaaattcaatcaaaaaagtagcctattcatcaatttttgcgggtatactgttaatagtaatacaaaatattatagagaattttaatagcttcttaaatttagctgcatctagttttctatattttggagtatattttttcttattggtattgtttaaagaaagtacaattaagaaatttctaaatcaaattttttataaggatatttcatga

SEQ ID NO: 397 - DNA, 1434 bp, Streptococcus pneumoniae; identical to SEQ ID NO: 395

SEQ ID NO: 398 - DNA, 1425 bp, Streptococcus pneumoniae
atggatataagtaataagtttaaggtaggtatcctatatactgctttaggtaagtattctaatgttataattcaattatttgtaacggcggtattaagtagaatattaacacctgaagagtatggcgttgtagcggttgtcaatgtgtttttagtattctttcagatattggcagattcgggaataggacctgcaattgtacaaaataagacattaactcaaaatgatttaaatgatatttttactttaacaatttattcaggtttaattctttcgtttgtttttgtattgataggctatccaattagtatagtctatgggaatgaagtctatatcaaactgtactcattattaggtatgtgtgtactattttatactatgacaattgttcctcagtcaattttaacaagaaacatgaactttaaacggatgaacttattgagtctgattgccaatatttttagtggagttattggtgttgtgctggccatacatgatttcggggtatattctctgattttttctaatattatgaaggctactatattattttttgtttttttttcaagtgttgagttgagttttaagaaaaagatagagaaaacttcgttagttaaaattctaaatttttctaagtttcaatttatgtttaattttctaaactactttgcaagaaatttagacaatctcttgattggtagattcattaatcccgctgcgttgggttattatgataaggcttaccagttatcattatatcctaatcagattttattgcaggtgatttcacctgtaattcatccgataatgtcgaattttcaaaatgatagacaaaaaatggaagaagtttttgcaaaaatttttaatattctacttttaataggaatgccaatctcagtgtatttgtttttcaatgcaagtgatgtgataacatttatgtttggaaataattggtttcagagcgttccagtatttcaaatactatctgctagcatctggattcaaatggctaatagtccaataggaattttttatcaagcttcgaatcgtgttgatctgttatttaaggtaggattattagcgtcaggatttaatattatagctattgtgatagggctattatcgaaaagtatagtgacaattgctgctatgttaattatttcatttataatagggctatcactgaatctctacgttttatcacatgtagtctttaaaaatgatataattaaatatttaaaaataattttagttcaccttgtaacagttatcccatatattatattcaatatgattggctttagatttgaacagggtgtagttatcaatcttgtgttgcaaggactggtattgggactaatttggtctataggaatatttgtaacaggtcaatacaagcagttaataatgatgtttagtaggtga

SEQ ID NO: 399 - DNA, 1425 bp, Streptococcus pneumoniae
atgaatgttgaaaaaaatttgaaacgtggaattttctatactgcaatagggaagtattcgaatgtagttattcaattacttgtaacagcaattcttagccggattctctcacctgtagaatatggtatcgtagcagtagtaaatgtatttcttttattttttcaaatgttagcagattcaggaataggacctgctattgttcaaaataaagaactgaataagtcagatttaatcagcattttctcattaacgatttatagtggaatcgtactttcttgtatttttgctttactaggatatcctatgggggtgttatacggcgaaactatttatgttagtttatttccgcttttaggtctttgtgttttattttataccattacaattgtaccgcaagctattctaatgaaaacgatgaactttaaaatagttaattttctaactattttttctaacattgctagtggcttggtgggcgttattttagcggtatcacattttggtgtctattctctaattttttctaatattgtaagagcaatggttttatttatcgctttatattctaaagcaaagataaatttctattggaaagtttcaaaaagttctattggtaaaatttttgagttttcaaaattccaatttttgtttaatttttggaattattttgctagaaatttagataatttattaattggacgattgattagcccgtcagatcttggttattatgataaggcttatcaattatctttgtaccctaatcaaattttatcacaggttgtgacaccagcattgcatcctattatgtccaattttcaagataatatcagtaagataggcgaagtttatcttcagatttctagaatattcgtaatagttggcatacctatttcggcttatttatactttaatgctcaatatgttgttacatttatgtttggagataattggtcccaaagcgttccggtgtttcaaatattagcagcaacaatttggttacaaatggctaatagtcctactggtgcattttaccaagcaactaatcagacaaggttattgtttagaattggcttattgacatcttttattaatattttagctattgttattggtgtaatgttgcaaagcatccaatgtgttgcgtacatgcttttgatttctttcagtcttagtttagtaatcaatactctttacttaacaaaaaaagtattaaatatttctgctcaaaaatatataaaaccgatattaattaatttgacaattatcagtccctatattgtgtttaatttgtttatctcggattttgtaaacgaccttattttacatttatcagttcagtttgttattttatttttaatatggggaattgggatgtatattagcggagagtatcgacgagtcttcgcggttataagaaaataa

SEQ ID NO: 400 - DNA, 1416 bp, Streptococcus pneumoniae; identical to SEQ ID NO: 393

SEQ ID NO: 401 - DNA, 1209 bp, Streptococcus pneumoniae
atgtcagttagaaaactatctttaatagatatactggactatatgtggataatattaattattgtacagtgtcattcaatatatacagtaaaatctgaacctcttaatttaattgtgccatttttagttacaacagcaatgttgttgttactcaatttattacgattcactttgtcaataaaatatatatatatattaggtctatattttatagcaatggctacattttttttaactaacataggtgctaatgttactcccttaaatattacgaagtattttataatgcttcctttattttttttaataaatagtatttatgtgaataaagaaattttttcagcgttactatcaaaatttgtaaacgtagtagtaattttagcaattttttccttattcttttggatatttggaacacttctgaatgttgtacaccctacctcaacagtaattaatcaatggtctggtggtcaattaataaatagctactataatctatattttgaaactcaacagatgattttttttgggttccgtataatacggaattcaggaatatttgctgaatctcctatttgggggctaatactaagtattgcttatgttattgattttcttattttgaaatttgataaaaatagcaagcgtaatataattattctaactatgttaagtacaatttcaacgacaggtattattattgtcgggttagctatattatataaaataatgactactagtcgttggatgacaaaactcttacttctccctgtcacattgagcttaggactctctttattgctcctattggctgagaaatcagagacagtttcagctaatcttagagttgatgattataatattggttttatagtatggaagacaagtttgtggataggacacggattgaataatggtatattagcaattcaatcacatatctcaacgtttacaagaaatttaggctatagtaacacattatttgttattttggctcagggaggtttattattattcttgatttatttttctccaatgatactattattgtttaagaaaaatatcaatcttgattttaagtttgccattatcttattctttatcttagtaacaacaattatttttgaagggacatttttatttctctggattttaacattatcgtatagctacttttcctttgtaacattagataaaactggaacgtaa

SEQ ID NO: 402 - DNA, 1251 bp, Streptococcus pneumoniae
atggttttgttattacttacttttgcattctttttagttttccctgtgctgtcaataagtttaagtgttatagggctagtaaatgacaaaaagcgttcaaaaatctatttacttctaattagttttgcaatatcaattgttgcgctgcgctatattcctcatcctatggatgatggcgcttttcattttcgtgcgactacggcccttattcgctatgatagtattttcgaaatgtttaaagcgttttctaatggatggagagtagggaactatgactatggttccataccaatttttacttcattaatgtatcttgttcgaaatactcatcactatagtttactgagttttatttcagcttttataacttactttagttttggttacgtagttgttgagctatttaaggacctgggcaaggtttctaaactatcgtatgctacagtattaattgctgtactatgtttaaataattatagatatacaactagtggaatgagattttgtatggcagttgccttgatgatgcttttactatatttggagtcaaaaaaaggctatactagtttaaaaacaaccatatggtatttgttgcctgtaggtatccattcagcggttatttattttattggtttaaggttcttatttccattaatcaagaaagtgacgcttgctaaaagtttatttgtattattaggttttcctgttttgttcaacctagttccatggttagcaaatctgattggttggacttacttacaatcatttattcggaaaattgaagtctactctgacaattcatcctattcccagttttttaataccacattaacaatgaggctatatgttggaatagtgctgatggttttatttgtattgttatatttgggaatagtaaattcattgaaaacaactgatgattggcgttttagttttgttacaatgacttattatgtaactctgttatctatgggttcaataccatttcgtaatatttatgatagaaatcttttcttgctccttcctatgattgtagtttcaacctacatattgtttacatatagacatcaattgaaaattcttactaatcgtaatattgtttatggattaacgatgggtattctttgtctatcctgtgcagttggtgcattttataataataatttcccttttgcttttattgattttagtaagacagacttactcctaaaaaatatttttcaattcttttcaaatttaccttttacataa

SEQ ID NO: 403 - DNA, 1251 bp, Streptococcus pneumoniae
atggttttgttattacttgcttttgcattctttttagttttccctgtgttgtcaataagtttaagtgttatagggctagtaaatgacaaaaagcgttcaaaaatctatttactactaattagttttgcaatatcaattattgcgctgcgttatattcctcatcctacggatgatggcgcttttcattttcgtgcgactactgcccttattcgctatgatagtattttcgaaatgtttaaagcattttctaatgggtggatagtagggatctatgactatggctccataccaatttttacctcattaatgtattttgttcgcaacacccatcactatagtttactgagttttatttcagctttcataacttactttagttttggttacgtagttgttgacttatttaaggatttgggcaagttttctaaactatcgtatgctacagtattcattgctgtactatgtttaaataattatagatatacaactagtggaatgagattttgtatggcagttgccttgatgatgcttttactatatttggagtcaaaaaaaggctatactaggttaaaaacaactatttggtatttgttaccattaggtatccattcagcggttatttattttattggtttaaggttcttatttccattaattaggaaagtgacactcgctaaaagtttatttgtattattaggttttcctgttttgttcaacctagttccatggttagcaaatctgattggttggacttacttacaattttttattcgaaaaattgaagtctattctgacaattcatcctattcccagttttttaataccacattaacgatgaggctatatgttggaatagtactgatggttttatttgtattgttatatttgggaatagtaaattcattgaaaataagtgatgattggcgttttagttttgttacaatgacctattatgtaactctgttatctatgagttcaataccatttcgtaatatttatgatagaaatcttttcttgctccttcctatgattgtagtttcaacctacatattatttacatatagacgtcagttgaaaattcttactaatcgtaatattgtttatggactgacgataggtattctttgtctatcctgtgcagttggtgtattttataataataatttcccttttggttttattgattttagtaagacagacttactcctaaaaaatatttttcaattcttttcaaatttaccttttacataa

SEQ ID NO: 404 - DNA, 1251 bp, Streptococcus pneumoniae
atggttttgttattacttgcttttgcattctttttagttttccctgtgttgtcaataagtttaagtgttatagggctagtaaatgacaaaaagcgttcaaaaatctatttactactaattagttttgcaatatcaattattgcgctgcgttatattcctcatcctacggatgatggcgcttttcattttcgtgcgactactgcccttattcgctatgatagtattttcgaaatgtttaaagcattttctaatgggtggagagtagggaactatgactatggctccataccaatttttacctcattaatgtattttgttcgcaacacccatcactatagtttactgagttttatttcagctttcataacttactttagttttggttacgtagttgttgacttatttaaggatttgggcaagttttctaaactatcgtatgctacagtatttattgctgtactatgtttaaataattacagatatacaactagtggaatgagattttgtatggcagttgccttgatgatgcttttactatatttggagtcaaaaaaaggctatactagtttaaaaacaactatttggtatttgttaccattaggtatccattcagcggttatttattttattggtttaaggttcttatttccattaattaggaaagtgacactcgctaaaagtttatttgtattattaggttttcctgttttgttcaacctagttccatggttagcaaatctgattggttggacttacttacaattttttattcgaaaaattgaagtctactctgacaattcatcctattcccagttttttaataccacattaacaatgaggctatatgttggaatagtactgatggttttatttgtattgttatatttgggaatagtaaattcattgaaaataactgatgattggcgttttagttttgttacaatgacatattatgtaactctgttatctatgagttcaataccatttcgtaatatttatgatagaaatcttttcttgctccttcctatgattgtagtttcaacctacatattatttacatatagacgtcaattgaaaattcttactaatcgtaatattgtttatggactgacgataggtattctttgtctatcctgtgcagttggtgtattttataataataatttcccttttggttttattgattttagtaaaacggatttactcataaaaaatatttttcaattcttttcaaatttaccttttacgtaa

SEQ ID NO: 405 - DNA, 1173 bp, Streptococcus pneumoniae
atgagaatatcaccttcgaagctgatatttgcattttttagcagcttttatattcttgtaatccttttcccaagaggaatttttctaggacattctactttagcttctcgattatggattatttacaatggaagtttatatttatcaatattacttggtttaatatatgttattttaaaaatattatctcgaaatatcgccattcatcagcatgcttttttcatcttacaatattattttattgtaattattttaactttcatggtaagtgggtcaatcggtgttgggctacaatcgattttctatccaatattcatctatttattttttaatgaagttcgagataagaaatcaatagtgttaatttttgatatctttctttatatacttactcctttattcgttatcaatacgattgatgtttttctgaatttcacaaatatttttcatattacttttttaggacatgttcaggtgatttcccaatatagtgttatcggatttctcgtatcagcatattacttattagaagaaaagagaaatatattagtggcacagttgctgttgattttaacgattatcaattgttttttttcagatgtctctctctctaaagttattgctttgtttatgattgtttatattatttcatacaagttaagaaaactattctggaaaagaggaaggaagatatctgttgtaacttttattgcatctgttgtgatgttagcattagtaatttttggatattttttaccatatcttagatattttgattttacttttaatggtcgataccaaatttggcgcattgtgtatgctaccattcttcaagtgaaatggtttggatatggtttgtttggctttcaatttaaacttccgtggcagaaattgggagaagttggaataaattacactcataatcaagttttacagttagcacttgatagtggtattgttgggattatctcattctttactatgattttttatatgatcttttcaactaaaaatattcaaaattccacaataagctctctatttatttttgcctatttttgtctatttattataatgtttatagagagtgtcacttattacccatactattttattataatagttcttcagactttgtacttaaaattggaaagagaaagaaatgttaaatag

SEQ ID NO: 406 - DNA, 1173 bp, Streptococcus pneumoniae; identical to SEQ ID NO: 405

SEQ ID NO: 407 - DNA, 1212 bp, Streptococcus pneumoniae
atgatgaaaagaatttattatcaccttttggctattttggcttggactcttccaaattcttatgcttttattgattcgttaaaggtgttttttccgaatattagtttacaaattgcaggttctcttttggctgttatctcgataggaatttttatcactagaatatattatacaggatacgaagtttttatatctctattggtttgtatttttattttaattttttattcaactcgctttttttattcgattaatgtagagttgtatcagtctttcttcaattcctttctgattcgtccagttcctgggatattagtagcgatgctgttagctaagaataatcatataaaaggtttcataaaatggtcagaacctatgatgcttttctatacattgacatcatttttagcagccctatctcctagaaatagcattattatttaccaatctttatcttattatgccatgacagcttatatgattaatgtgtttaatattatatacaaagagaaaatattggaggagcaactgacgtatttcagattctcagcgtggaattttattagatattttttacttttgattcaagcttttaatgcactctcaggaggtgggagaggtgcctttatcttattaattatttttacaggaatattattaataagaattgctatgaattttaagttccttttctcattcattggaggcttatctgctatagctctagttgttttttgtataaaagatttggatttcacatggctcttgaatatggacggtggagagcgattgttgaacttcttcggcagacctgaacgtatatctacagataatagactattgatttatgatactgtttggtctgcaatacaggaaaaaccctttttaggatggggagttggttcaacttttttaaaatttaatggatattcacataatattgtattggatttattgcatgatatgggaagttttggtttattaattattctttcggctttctttgcttcttcagtaatattgtacaaattacgtaaaatagattggaaagtgaatttatttattctgatgtttctagaagtatatgttcacatgagtttcagtggaagctatcttgcagatggtagactatggtttttggtgatctttacttattgttactacaggtggaaagtgcaggaaaaggatgaagttagcaattttattatctga

SEQ ID NO: 408 - DNA, 1212 bp, Streptococcus pneumoniae
atgatgaaaagaatttattatcaccttttggctatttgggcttggactcttccaaattcttatgcttttattgattcgttaaaggtgttttttccgaatattagcttacaaattgcaggttctcttttggctgttgtctcaataggaatttttatcactagaatatattatacaagatatgaagtttttatatctctattggtttgtatttctattttaattttttattcaactcgctttttttattcgactaacgtagagttgtatcagtctttcttcaattcctttctgattcgtccagttcctgcgatattagtagcgatgctgttagctaagaataatcatataaaaggtttcataaaatggtcagaacctatgatgcttttctatacattgacatcatttttagcagccctatctcctagaaacagcattattacttaccaatctttatcttattatgccatgacagcttatatgattaatgtgtttaatattatatacaaagagaaaatattggaggagcaactgacgtatttcagattctcagcgtggaattttattagatattttttacttttgattcaagcttttaatgcactctcaggaggtgggagaggtgcctttattttattaattatttttacaggaatattattaataagaactgctatgaaatttaagttccttttctcatttattggaggcctgtctgctatagctctagttgttttttttacaaaaggtttggatttgacatggctcttgaatatggacggtggagagcgattgttgaacttcttcggcagacctgaacatatatctacagataatagactattgatttatgatactgtttggtctgcaatacaggaaaaaccctttttaggatggggagttggttcaacttttttaaaatttaatggatattcacataatattgtattggatttattgcatgatatgggaagttttggtttattaattattctttcggctttctttgcttcttcagtaatattgtacaaattacgtaaaatagattggaaagtgaatttatttattctgatgtttctagaagtatatgttcacatgagtttcagtggaagctatcttgcagatggtagactatggtttttggtgatctttacttattgttactacaggtggaaagtgcaggaaaaggatgaagttagcaattttattatctga

SEQ ID NO: 409 - DNA, 1212 bp, Streptococcus pneumoniae; identical to SEQ ID NO: 408

SEQ ID NO: 410 - DNA, 1152 bp, Streptococcus pneumoniae
atgagtagtataaaagtaagtatatatcagttacttttttacgtatcttttattattatagttggttcaaaatatttaagggaaacggtttttgtaaatgcatatcctctgttaagtgaattgataaaagttttaataattatagcatgtatctttttattattaaaatatatattagaaccacatactaaaaagttcattattttttcgatagttattttaacgataggagtagtagtatcttataattcagatagtttttttattttaatgccagtagtagcactaatattaaatatgagtaatatagatataaccaaggttataaaagtttggcttatagagattatagcattaatgatttttctagcaatatgttatagactaaatatagtaggggaagttataaactcagcaattagatcagatgggaaaatacgatatgctctagggtataaatattcaacatttagctcaaattactttttccatgttacgattttctatttatatttaagaaaacatatgattaagtatgtagagataataactttatttcttataaatttgtatctatatgctttaactgatacgagggcagtattctactactctacagcagctattgttatttgtttgttgttaaagatgtttaagataaaaacatatagcattattttgaataaatacagtatgttattttcagcaataatagctggagttttgtcatggagttatcggtatagactaccattttttgatcagttagatttaatattaacaggtagattacgacttggaagtgctgcttttaataattttcacataacattttttggacaaaaaataagatggatttatgaacaaaatatgttttctgaattgatatataattatgtggactcttcatacctaaatattttatttggatttggaataataattttactattaatattagttggatattatattattggagaaaaaaaactaagtagagatacatattatacaatgatgattgtgttcttatcactacattctacatttgatcctcaattaatagacatagtgtataatccagcgattttgtttttgggatatgttatatataatgaagatgagataaaaaatctaaataaaatttactaa

SEQ ID NO: 411 - DNA, 1173 bp, Streptococcus pneumoniae
atgaaaattagaatagaacctcagtattttctttataaatacctttggtttattatcctacttccaaaacagtttatgcaattaatattattctttttaattgctttaactcttttaccaacctatataaaagaaaaacaagtttttaaaatagatacaccgtctttttgtatggtgctatggactattatatattctatatctataatatttaattctctgattgatggattggctgttcaagtgttattttcagatttgagtaaagcatttaattggctaatagcagtatttttttataattattatttgaaaatgccaatcaatattgacaggataaagagatatatgtattataattttactatcttagttgtttttgtcggtttattctatatacaaagaggctccaatgtaattttgtttggaagaagtttgttagactgggacggatttacattagctactagttatggtgtaagatatacaggttttttagaatacgcaactttaaatggtcagttaattctttttttattaccgttaattagattgtttagatttagattttttacacaaactatcatttttgcttttcttctagaggttttggtactaagcaaatctagaatagcgattgttgcaatgcttatatatatagcatttgcagtagtcaatgagattaattcaaacaataaatggcttattggaattttctgtccaattattccttttatgttattttacaattttgaaaaaattaaacagattttttttcaaatgtttagttctagatcgggtagcaatgcgacacgctttagagtgtatgaggaatccctaaaagctattaatggaatggaaatgttacttggcgcaggtgtcagaattccctctacagtagatatattattggggtcacattctatgtatataagttttatttataggacaggagttttaggaagtataataataacagtaatgttttattatctgttttctaaatttttaaaatgtgattcatctgagagactaagaagtattggctatattctagctttgtcagtattttggctttttgaagagttagatccacattattggtgtttaattttatttttttcaacaataagtattttcataaacaatagaaaagaggaaatagttggatga

SEQ ID NO: 412 - DNA, 1200 bp, Streptococcus pneumoniae
atgactaagtacataaatagctgtatcaagttgttattcatatattcgttatttagtgaattgctatactcatattattctgtatctctattatttacgatacctgacttattattgttggcagcagcagtaattgtttttgtagattcttattctgcagggaaaataagagttaaaaatcctcacattagtttaatgctcttttttatattgctgatatttctattaattagttttacttggggtactcttaatatatatggatttgttatgagaggaagatatatactgggagcatttctagtttattttatgacaaatagctatctagatgatagaaccttttcatcattgataaacattgcttattttatgcagatattgaacctattattagttttacatcaaaatatagtattacatttgcaccctgacttcactaatgggatttttggttttactaattatgcaaatggaatccaaggattttattgcttagccttaagtgttttatctactgtatattatctgtatggtaaatggggaacaatgaagtcattaatattgattgctatttcgtgtataatttgtgcattagcagaaataaagattttttttgtcatttttatattttctattattctaatttttatatttcaaaagtcagaaacagtaaaaaaaatacgaataattagtacagctgctggaatatctcttctttttttaattgcctataaactcattgagatagttttacctgataatttgtatactttttttaatgttacaaaggcattatcatatgagaataggacagagtttgcgggacgaacaaatactatttccttcctatgggacaacttattttatcatgattatattagtgctatatttgggaaaggactaggttcatactctgttaattatatttatgaacttggtaaaatgcttgcagatggtggttttatttcagtgattttgctttattcatttttgttgtcattattcatacggggaactattactagaggaaaaaataagcaaagtgaaagactaattgtatccatcatagcttttgtggtgatgattagcattattgtttggaatagtacgttcactagatcaacttatcttgtatttttctttctagcaataggtaatgcagcgtataagtctactaaattaataagaagggattga

SEQ ID NO: 413 - DNA, 1173 bp, Streptococcus pneumoniae
atgaaaattagaatagagcctcaatattttctttataagtacctttggcttatcatactgctcccaaaacagtttatgcaattaatattattttctttaattgtcttaattctattaccggtctatataaaagatagacaaatttccaaaatagatgcaccgtctttttatatagttctatgggtcatgatgtattctatatcaataatttggaattttttaattagtggcttacctattcaagtgttgttttcagatttgagtaaggcattcaattggatattagcagtatttttttataattattatttgaaaaatcccattaacgttgacaagataaagaaatatatgttttataatttcgctatattagttattattgttgctttattctatgttcaaagaggcgctaatgtagtattgtttggaagaagcttattaggttgggacggattcgtatcagctaccagttacggagtaagatatgcaggatttttagaatattcaacattaaatgggcagttgattctttttttgttaccgttaattaggttatttaaacttagtttttttacacaagtaactattcttgcttttttgctacaggttttagtattgagtaaatctagaatagctattattgctctgattatatacatagtatttgtagtaatggttcagattacttcaattaataagcggatgattgtagcgttttatccaactatacctcttatattgctttataattgggaaaaaattaaacatatcttttttcaaatgtttaattcaagaacaggtagtaatgtgacacgttttatagtgtacgaagaatctcttaaagctattaatgggctgggaattttacttggagcaggaatcagaacacaatctacagtaggtatattattaggatcacattcaatgtatattagttttatttataggacagggattttaggaagtataataatagtaatactgttctattatttgttttctaaatttttaaaaagtgccccatctggaaagttaatcagtataggctatattttagctttattggtgttttggctttttgaagaattagatccacattattggtgtttaattttatttttttcaacaataagtattttcataaacaatagaaaagaggaaatagttggatga

SEQ ID NO: 414 - DNA, 1173 bp, Streptococcus pneumoniae; identical to SEQ ID NO: 413

SEQ ID NO: 415 - DNA, 1200 bp, Streptococcus pneumoniae
atgactaagtacataaatagctgtatcaagttgttattcatatattcgttatttagtgaattgctatactcatattattctgtatctctattatttacgatacctgacttattattgttggcagcagcagtaattgcttttgtagattcttattctgcagggaaaataagagttaaaaatcctcacattagtttaatgctcttttttatattgctgatatttctattaattagttttacttggggtactcttaatatatatggatttgttatgagaggaagatatatactgggagcatttctagtttattttatgacaaatagctatctagatgatagaaccttttcatcattgataaacattgcttattttatgcagatattgaacctattattagttttacatcaaaatatagtattacatttgcaccctgacttcactaatgggatttttggttttactgattatgcaaatggaatccaaggattttattgcttagccttaagtgttttatctactgtatattatctgtatggtaaatggggaacaatgaagtcattaatattgattgctatttcgtgtataatttgtgcattagcagaaataaagattttttttgtcatttttatattttctattattctaatttttatatttcaaaagtcagaaacagtaaaaaaaatacgaataattagtacagctgctggaatatctcttatttttttaattgcctataaactcattgagatagttttacctgataatttgtatactttttttaatgttacaaaggcattatcatatgagaataggacagagtttgcgggacgaacaaatactatttccttcctatgggacaacttattttattatgattatattagtgctatatttgggaaaggactaggttcatactctgttaattatatttatgaacttggtaaaatgcttgcagatggtggttttatttcagtgattttgctttattcatttttgttgtcattattcatacggggaactattactagaggaaaaaataagcaaagtgaaagactaattgtatccatcatagcttttgtggtgatgattagcattattgtttggaatagtacgttctctagaccaacttatcttgtatttttctttctagcaataggtaatgcagcgtataagtctactaaattaataagaagggattga

SEQ ID NO: 416 - DNA, 996 bp, Streptococcus pneumoniae
atgttaggtaaatctaagataaaatcgataatcattactccaattatacttcttataggttttattgtaagttatgataatggtaacgcctcctttgtagcatatactttgactcttatatattcttcaagggatattaattttcgtaatttggtaaggaatacaatgttctgccagattggagttgtaggaactgttatagtaagctctttacttggtattattcctaacgagctagccttaacatattcagaaggtgtgatccgctcacggtatggactaggttttcactacacatcatttactccgaattatttcctgagtatattgctggaatatgtgtatttaaaaggtgaaaaatattggacaataaaagagctttttatttgtgtactattaaatatagttatttataaatacacagatacccgtcttacttttattatggtatttatccttcttttgatatcgttttcgagaagatttgtatcaatcaatatgaattttaaatttctaaaatatttacttactcttatttatccaattatggcgtatatgacttattggctgacagcaaaatttgatagtagaaatagtttattatctttaataaataacttattgagtcaaaggttgagattcggtcaagaaggtttaaggagataccctctaagattatttggaactcatattcaatgggatgcttcggcgaattcttatctttatgtagattcgtcgtacattaatatacttataagctatggaacaataatttttttccttacgttaataagctattcaattataatgaaaaaagttataattaatcaaaataaaactttattgattgtgttaattttttggtcagtaagagcttgtattgacccacaattatttttattgtggttcaatccatttttgtttttaattgcgagaacgtttttagatgaaagagaggaaattggagatgatatcacttatatgtgtatcgaataa

SEQ ID NO: 417 - DNA, 1038 bp, Streptococcus pneumoniae
atgggaggaattggtttagtttatactatctgttcagataggccatttacagctttaaatctttttagaatttatatgtttcctgctgtaatatattttataataataaattgtaattttcgcaaggagagattactgatattgaagcaagcgcatgtatatactgcttgcatattagctttatggggaatttttcaggcgtgggtgttaaaagatcagtttttgattaaaataggatatccttcccaaggaaattttctaaagagtacagctttttatattggtggttttttcggtcaacaacgagttactagcactttttctgctcctaatttagctggagtttattttggaatatctttgatcattttactctctatatttgatactataaagagtaatagattggtattgttttcaattgtagcagcttttgttcttacatttagccgtagtgctattataagtactcttgtaggaatcgtattctttcaacgtaagaaattgttttcaacgacgaagattaatgttatgaccttggtaatcttccccttaatttttttgattgttttagttatattttatctttatcctgagaatgttattataaatatgctctatagcagttattcttcaacacttaatttgacagattcttcagcggttaagcatttggaagacttatggcttcctcttctaaaagttattgattatcctttgggacttggatttggtaataatggcccaattgttctgtcactctatcactcagcaaatttagtagaatcttctatttatcttcttgcatatgattttggtattttaggaatgttcatatatttgttcccttatttttatacgatttttgtatataaaaaatatttgcttagcggtgctatatgctgcttagttcttataacttatctatttcttccaaatgtagaaaattttgaaatcatattttttatttaccttttcattgggatggatgaactagcactatatacaaaaggaatgaatgtacatgaaattgattaa

SEQ ID NO: 418 - DNA, 1158 bp, Streptococcus pneumoniae
atgattaaagtgtctaaacagaatgctatattattcttattactgctcccgttttttatgccaacttattttgaaattttaccatttggatacttgttgtatagattaggcatgatgtttagcataggatatatttctttatcttacctaatattaaagaaaaatttttcttccatcacctggttgattgttatctatgagatattagtagtttctttaacaattttacaaaaaggtgaaatctatactgcaatatggaatgcagaaaaaattattggactggttttattagtagattactaccaagaaaatattattagcttcttaaaagttgtactggtacattttgagtggttgatttatatcaattttattacaattttattattttcagagcgctttttttctagattcacttctgcttatggttttacccaagaatggtttctaggttccgtcaataactttattttctggttttttccagctcttgtagtagcgtggttgtattattttttgtcaggtaaaatttttagaacgtgtagtttaacagttgttattgctttaacagaattaattagaggctcagcaacggggaggattgcagtatttctatttatcttatttgttacctcgtcttggttacgtaagattatttcgccaaagttagtatttataggtgttactgtcctaggaatttttgttgtatttctccaaaatgtagacttcttagagcctattgtggtacagttacttggtaaaaaaatgacatttagcaatcgtacctatatctggtcgaatgctgtgacagtaattcaaaagaatttcgttggtctaggtttacaatcatctgattatgtcattcgattgcttggaaatattaatggttatttacaacctacggtaacccacgcccataatgagtttttacaagttgcttttcaaacaggaattttaggatttattctttattgttggatatatattaaagtcttgatcaatgcagtaaagttgagtggaccaatagcgcaaactgttatgctgttttttgctgtttataatgttattgctataacagaaacgttagaactgagtcaaatatatatgttatttcctttgatttattttgtaatgcaatatactgcagaaagaaagtaa

SEQ ID NO: 419 - DNA, 1194 bp, Streptococcus pneumoniae
atgtttcgttttcagaatgggacagtatcaataaacatgagaggatttctgatgttcattgcaatgctcatttactgtcaaccagaatatttgaaatcaattccagttacgggacgtatttatagagatttcatgcatgtaggaattatgtttgtgacgttttcgtacatttcaaaaaaacggtttccaaaattttttgtatggctagttcttctatataactcttggcttacttttttaactattcaaaataacggagatataaaaagggcgatatttcaactcttgctgtttacaggctttgtatctatgatagacatattctctgaaaatatgcaggatttaatagaatcattgttattcacttttgagatggttgtgtatctaaactttgtcactgtactatttttcccagatggttttttccatcgtgtaaatgatgcatacggaataacaaaagaatggtttttgggagctgataataattttctatttaccttgtttcctgcaacacttatagctttattgtataagaatttaggtggagtaaaaatcagagcatattcattgatttcaatcattttagctactcaattgattaaaggatcgggaacggcaaaggttgggattgttgcgttgatactatttgatttagtacctcttgtcaaatacatacttaccccttacagaggtttggttatatcgtctacgttgagttttctaattgtagttactggtaaagtggaatttttaagagggattgttgaaggtattctagaaaaagatttgacgttcacttcccgaacttttatttgggataacgctatgtcagcaattcgagagaagccatttggttacggaatgatgatgttcactgataacgtccatatattagggaattttcctggttggatttggaaaggagccacccatgctcataataactttcttcaagttttatttcagggaggatatttaggatttggtgtttattttttaataattctaaagggagcaatgctatgtcgcaaatattgggatgatagaaatgcgagactactcttttatggactttttatttttacaataatatctataacagaaaacttagagatgcagatgatgtatttggtgattatactaccgtttttattgcctaatatattggttaaagaaaggggaaaaaatgaatag

SEQ ID NO: 420 - DNA, 1170 bp, Streptococcus pneumoniae
atgacaaaatattatattggtattttaatcactgtgttgtttgttttgttattagtaggccgtgggaagcttatttttgttaataaaaaattattatatcttgctaagatattagctataccaacaattgttcttttcctgtactcagtcttactagacgtaatgaacccagttgaatttgatggatattttagtaggttatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatgctaacagatgatagtttcaatggttcggtactagaaatgcatgaagttgcacctattacagctctctttattctgtactatttgtacaaatattttataaaagaaaatagtttttcttcagtatttcgtaatatcttaatagctctcattattctttttttaagccttaaacgaatcgtttttttgagtgtattaattatcataccagtatttttgataatttattggtatgataaaaaagtaagcaaactagggaaagaacgaaaaattttaagtttattaaatagcttttccttaatatttataacaggaatattcctttatgtttatagtgtaaaatctggttttatatatacatttattcaagaacataatattaattcgatggctagaacagatttatggaagggaattgaatcaacctatagtttcgcccctatatttatggggagagggatagggtttgtaacaaaatggatggataataattggatgactttgaatatcaatggtcttacagagtcaacggggctccataatgatattttgaagtactacattgagataggatttgtaggattatttatttatttttacactcttctttatagaaatgctaaacgtatatttgtaaaaattggtcataaagaatcatttatatattttgtattgataatgtttcagatgctgatatggtttacagataatatttcaacttaccataattttttatggattctaaatctactgctcttttctttaactaatacggatgcagaattggaaactcagaaattataa

SEQ ID NO: 421 - DNA, 1179 bp, Streptococcus pneumoniae
atgacaaaatattatattggtattttaatcattgtgttgtttgttttattattagtaggccgtgggaagcttatttttgttaataaaaaattattatatcttgctaagatattagctataccaacaattgttcttttcctgtactcagtcttactagacgtaatgaacccagttgaatttaatggatattttagtagattatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatactaacagatgatagtttcaatggttcggtactagaaatgcatgaagttgcacctattacagctctctttattctgtactatttgtacaaatattttataaaagaaaatagtttttcttcagtattttataatatcttaatagctctcattattctttttttaagccttaaacgaatcgttcttttgagtgtattaattatcataccagtatttttggtaatttattggtatgataaaaaagtaagtaaactagggaaagaacgaaaaattttaagtttattaaatatcttttccttaatatttataacaggaatattcctttatgtttatagtgtaaaatctgattttatatatacatttattcaagaacataatattaattcgatggctagaacagatttatggaagggagttgaatcaacctataatttcgcccctatatttatggggagagggatagggtttgtaacaaaatggatggataataattggatgactttgaatatcaatggtcttacagggacaatggggatccataatgatattttgaagtactacattgagataggatttgtaggattatttatttatttttacactcttctttatagaaatgctaaacgtatatttgtaaaaattggtcataaagaatcattcatatattttgtattgataatgtttcagatgctgatatggtttacagataatatttcaatttaccataattttttatggatcctaaatctactactcttttctttaactaattcggatacagaattggaaaatttagattttaaaaatttttaa

SEQ ID NO: 422 - DNA, 1179 bp, Streptococcus pneumoniae
atgacaaaatattatattggtattttaattattgtgttgtttgttttattattagtaggccgtgggaagcttatttttgttaataaaaaattattatatcttgctaagatattagctataccaacaattgttcttttcctgtactcagtcttactagacgtaatgaacccagttgaatttaatggatattttagtaggttatcaagtacgactatttttggtttgttagctatctttcaagctatagttgtttttcaattttttggacaaaaagtagtagattacacttttacagctatctccctcagctacttaaccagtatcattgttgcctttaggcagggaggacttagtcaatttatcttgatactaacagatgatagtttcaatggttcggtactagaaatgcatgaagttgcacctattacagccctctttattctgtactatttgtacaaatattttataaaagaaaatagtttttcttcagtatttcataatatcttaatagctctcattattctttttttaagccttaaacgaatcgtttttttgagtgtattaattatcataccagtatttttggtaatttattggtatgataaaaaagtaagcaaactagggaaagaacgaaaaattttaagtttattaaatatcttttccttaatatttataacaggaatattcttttatgtttatagtgtaaaatctgattttatatatacatttattcaagaacataatattaattcgatggctagaacagatttatggaagggagttgaatcaacctataatttcgcccctatatttatggggagagggatagggtttgtaacaaaatggatggataataattggatgactttgaatatcaatggtcttacaggatcaatggggatccataatgatattttgaagtactacattgagataggatttgtaggattatttatttatttttacactcttctttatagaaatgctaaacgtatatttgtaaaaattggtcataaagaatcattcatatattttgtattgataatgtttcagatgctgatatggtttacagataatatttcaatttaccataattttttatggatcctaaatctactactcttttctttaactaattcggatacagaattggaaaatttagattttaaaaatttttaa

SEQ ID NO: 423 - DNA, 1245 bp, Streptococcus pneumoniae
atgaagagacagaagtttgaatttatagagattctatactattttacagtgatgttatcagtgggaatgtttcttatgtttaccctcaacttatattggcacagaaacttattaactattttatctattgctctctcatttttaatgcttcctatactgattgttaatgctaaaagaatttctaaatctgcttttatctatggtacttttttatctatatgtattatatatgagatattaagagctaaaacactgtataattacagtgtgagtaatatttttttggcctctagacaatatatatggatttttctattttttgtattgatttacctttttaaaaacaaacaagaaaatatgagaaaaattttagataatacactcaatatttttatgttttctcttggaattagagcatttacttggtttttatatacgttatttcaagttgaattatttccatctattttaagagaattcggagatttgtggtatcgaaatgaattttcagtacgaatagatggaacaccattaattataataggtttgttaatttccacttttttctattttaaatttggaaataggaaatacttttattatttgttcttgatattaatgtatataacatttgtaaatcagacaagagtgctactggtttctgttttgatttcaatttttcttatgtttgtattttctagaagaacttctagattgctcacctctttaagttttgtaactataattatagcattcgtttatggaggtggattggattatattaaagcatatttaaatatagatgctggaacatttgatctgggattaggatttagatactgggagttgaaatactatctgggtttattagctaatgatgtatggaaacttggggttggaattctaacatcaagtaatataaatagtaattttattttggctgggccaagtgctgttaagatgtatctagatgatttaggttttttagagttattcgttcaatttggtgtagccgcaatttttatgtatggttatatattttataaattaatcaatttaattctaagaatgtcaaatgataaatatagagttgaccgcgccttttttattgcactcttaactaatttaataattacttcaatttctttaaatatctttggagcgcagagaagtttctcattggcaattgttcttgcattaatattctactatgactataggctgaaaaatgacatagaaaattag

SEQ ID NO: 424 - DNA, 1050 bp, Streptococcus pneumoniae
atgcttctctcaatgttgataattacttttatcaagtattttagaatatcaaaaaaatggaaagaggcagtttatttttggggttttattcctcttatcttgataggagcacttagagcttatgtaggaatagattataccacatatagtttggatcagataccggctgtattagctggtagtcagacagtcaaatttgaattgttagataaattagtagtgtacataggttactatctagctaatcagcagcattatttctatatttttgcaatatttcacattatcctgatgtggtttctgtataaatatattgttcaacagtcgtctaatgtaatgttgagtgtgttttttcttctaaccactgttttttttactttttctttatcaggaatacgccaatcaatagctacagcgattgttttttatgcgttgaagtatattaaacaaaaaaaaagtttacattatataatttatttagggattgcttgtttatttcattctagtgcagtaatctatatactttttttagttttagggaaaattaatattaataggttcgttggctttgcattaccgatgattgcatcattattttctttttctgggtctgaatttgtatctcgaataatactacatttaaatttttattcagaatacgttggttctcgttattatacaggagcatatgataaacaacaccaactattcacaatcgttatgtgtttatcggtatttctattatattatattgttccgaaaaaagagtgggttaatttaaaattatatctgaatattaattttgtgctattactagttgcaataatcatgccaatacttcctactccatcaagaactatttatatgtttgttcttgtacatgttattctaattcctaagttgatttcagttattaaagattataggtctaaggtaatcataacaatgttctttgttttaggttattttatattcttttctataacggttctacagaggaatgcatatgaaacactaccgtaccacacaatctttgagtatttatggtaa

SEQ ID NO: 425 - DNA, 1269 bp, Streptococcus pneumoniae
atgttagccttgacaataggatcagtggtatttgttaaaaaaacgtataggacgtttctcaatcccataattttatttgcaattttgtggtttattataacaactttgtcaaatttaaaactattccaacttttcgagattagtactcatacacaacaaattattttgcttgggacttctctatttgttatagggggagggatagcaatttggtttcgggataaattttattttaaagttggcggtaactcatattttacaacagactttgaaattaattataatttgttctttttgctaggtatcatttgtcttttctattatttgccagatttcttttcctcactggtatcattaataagaggtgggaatttaaacttagttagacaatctgcgcaggatgcagtagataccagtggattaaagaatttcattggaacatttattgttattcctagtgccatggtattagaaatattaggaatattagatttctggagcaataaaaaaggcagaaaactattttatcttaatttagctgtcatttttgtaagagttgttgcagatgctggtagaacgcctctatttaatgttgtcatatatttattacttactgttttagcaaataggttctctgaaaaaactgaaaagaagagaaaagttagtaaaattaagatagtaaattatggtatgctaggttcaattatcttatggctatcaactttatctaggacaacaacctccgtttataggattctttatttttatttcgcaatgtccccaatactattagaaaagtggagtagtatacttgattctgaaaaattagtaacaaagggtttggtttcgttgaacggtttctttttttcaatctcatatgtacttaaaaatctgtttagaataggttattctcaaagagtattagaagcatacacaatgattgcgaatactgatgcaatctggtataatattgcacctggtttgaccaaggcaaatgcatatgtctctctgttttggtttttttatgcggatggacgtttattaggagttttaataggatcccttctatatggtgccttttgtggctatatcttttgcaggtatatccagcaacaaaataaaaagaatttggctatgttactatttatttatcaaggtgtttttttctcttttattagatttccgttttcaaaatcaaattatgcaattgcattcgtactcttattattttttgcttttaagaagaaaggaatagagaaaagtgtttaa

SEQ ID NO: 426 - DNA, 1269 bp, Streptococcus pneumoniae; identical to SEQ ID NO: 425

SEQ ID NO: 427 - DNA, 1392 bp, Streptococcus pneumoniae
atgagaattagttttagtaaaaaaactatgctgtgtggtttattgtatattggtttaattttatccgtagtaacaatacctacaattgtaacatttttatattctttgctattcatcgggattgtaacagtcttaaattataattctattctagcaagtgacgaagatgcgaactctttttttgttgcattacctattattttatcttcttttcaaaatgtatatttgggatttggagcggatcgattaaattctgtcacactacaagttctgctatctattagcattgctattattacaattacagtttttctaggtattatcttaaatagattcaaatcaaaagaatttagttggttagttttaagtattcttgtaattataattcaatcagtaattcttttaatattctttcccactaccttgccagcatatttatcatctatgagaaatatactagccccactactgattttttatttttctatctatgggtttaaaaatattaatttacaaaaattttataaatatatgttcataattatattagttgttctaatttttggatttattgagtatatatacggaaatagtttatggacaagattgaatattaaaaagctttgggctttgaaggggctggcgattgagaatagggttgttcctgggaattggcactcttctgaattaattggcggtaaacaattaaggcgtatggtttcaacatttgccgatcccgtaaatctaggttcgtatctctttgcggcatttatgttagcttggtacaaaaataagaaactacttcaagttttattgctcgcttcttttgtactcagtgtaagtaaagcagcgtttttaagcatgttggtatatataataatttatacctgggttgtggataaaaataaaattttatcgatttttggaataataatttcaactgtattggggttgtatttttataattttagtcaagttagcagttatggtagtataaatgcacatatcgatggtttttttagtgcgttaagtactcccctccattacccatttggtatgggagtagggagtgtgggagttctagcttcaaaattaggttcacaaacagcactttcttcagaggttttagaaacaggaattggaatgataattgctcaattaggattcgtgggtgtgataatatatttgattttctttgtaaaactctccgtaataggaaagaatattaataataaaagagataaaattttatggtttactttaatttatagctttttggctaatgccttttttaacgaggttgcactttctcctaactcatgcaccttatattttttaattttaggactgctttataataaaaataaaattagaagcacagagtttagttaa

SEQ ID NO: 428 - DNA, 1188 bp, Streptococcus pneumoniae
atgactattaaaattaattatatgttttttgtttgtctctcttttttcggtatagttttaagtagttcacaggtaatagtgaatctgggtttgtccagtatagttcagtatatagcttattttttattgttgctatgtatattttttaccttaattaaaaatagtccggatgtaatcgctaatagaattgcttatttttctattatttcatttttgtttatcatagggattaatttacaaaatcttcctttctcaacaaagatttatttatcgttttccatgcttattatttctagtctatcgactttaccgattaaattaataaataatataaatgattttagaagaatatcctattttttattaaatggcatcctattatcaacatttttaggttggctatttaatatttcattagtaactgttgctgttgaggggattggttttgcatatggatttaatggtgggttgacgcataagaatttttatgcaattacaattttagtttcctatatcctattgtttattagtagaaaacatggtacgaagtaccaagttgatagcttagtattatggtttgatctttttttacttttagtatctaacacacggacaatatatataatattagttgttttttggatcgttgttcatagcggatttattaaatatataaagaaaaatcacagaccagtaattataacgacatggttagtaatttctttattatctataatattttttttcaaacacattataaataatagtgaatcctatactcatcgtgttttgggtattgttaatttttttaaatactacgaatctagcaaatttcatttattttttggtgatgctgagttagcctttggtgatatgacgaaaggttatactcacaatataagaagtgttttaggttgggatggaactgtggaaatgccgttgctaagtgttatgataaaaaatggttatgtcggcttaatagggtatggagtagtcttatttaaatttatatcctctgtattatctatggaagatagaagagtaaaaaatattggcttatccattctaatacctctacttctatcagcaatggttgaaaattatatcgttaatattagctttgtattcatgccggtctgtttttgtattttatgctcaataaaaaatatagaatttaaaaataattga

SEQ ID NO: 429 - DNA, 1251 bp, Streptococcus pneumoniae
atgattgacggagattttatgaatagaagacttgtaataaaaaagaaaagtataatgtttttcatgcttatgttatgtgttacggcattttatgggacaaattattggtatacaaatatatatatatatgattttatgattataacatcatgcttattatcaataatagtatttgtatgtaatggtattagaattgataaaacatattataaagggacaataatactgggtattgtaatgtggttttcaggacttattgctgcacaaggaaaagggctccctgctattgtaatctttaaagagagtttatatactattactccaataatgatatttttagctttccgacctatgataaagtcaataaaagacacttcattattccttcgtactatttctgctgccggtgttatatgtaatttaattgcttgtattgaaatgttctttgccaaaagaggatttgattttctaaatataagtgtatttgaaaaacttagaaatggtactcctagatttacaattggagaaacaataatagttcttagtttttttatctcctgtagtgttgtttttagtaaagggggaaagagaaatagacgcattttccatgttctaaatatagctctcacagctataaatttagtgtatattattaaaacaaggacactgaatttatatattttatcaacacttatgatggtgcctgttttaaaaaaaaatgtaaagaaacaacttaagttttggattgtgtttttgatatctataattcttacttttgtttctgcagaatattttattccaatagtcaaaaatttaattcatagtgatcatgggattcaaattagattcttgacgatagaatactatatagagtattttaaaaatcattacttatttggagcgggatatatttcttctagtccatattttgaaacttattctattgttaccggtccattaggacgttattatccaagcgatgtaggtttgattggacttatgtttagaagtgggattatcgggctaatttggttaataagttggttttatacaagcttgaagataattaaagataatacgattagaattccagctcattatgacttattaatgaagctagttatagtttttttgatgttttcttgtattaatttgataatcactgatgcacctagattcccatatattgcattagctatgttactttttgaatcaagttataccttgagttatgaaaacagttctaattag

SEQ ID NO: 430 - DNA, 1182 bp, Streptococcus pneumoniae
atgaaaatagtaatacaaagaaggtctttgccagaaattttagctcttagtgcactaactatatttttagttatgtcaatttttgatgtgactttttatgttcagtatctgccgaaaattgttcacaggatactgatagctattttgcttattctgcttgttataaaagagtcgtacaaaagaaagcttgattatagaactatcataagtctattttctactgttttaatttatctccttatagggaagatgagtacttttagttctaatattgcaataggtttcatttttatttatgttttacgcgatatcccttttaaaagtgtggcaaaaatttctttagcggtaagtgtgttcatgttactctttgttatagctagcgcaaagttgggagttattataaattatttagagatttctgggccccgagtccgtagctatctaggtttcagatatgccttgtatccatcaatcctacttatgaatgtcattgcgattacactttatttaaagcaaaacaatataaggtattggcagtggttattactaaccttgtctgtttactgggtatatggacaaactgattctcgcttaacattttacaattcttgtatattattagtcttcaatatactaataaagtggttccctgatattttgtctaagttaggaaatgtgtttaaaatttttaggctgacctttattataaatgcaataattagcttttggatttcaataaattatctcggatcaaacaattcttttgtaaacagtttcttattcaagttaaatcacatgttaggtggtcgacttttcttagctaataaatctctagaattattcggttttggtttgtttggaaaacaggttgactggaatggaaatggtctgaccattgaaggagtcaggaattatcagacctatctttatgtcgataatttgtatattcaaattctacaaaaatttggtttactcgttctagttttaatgttagcgttgttaacgttaacactgtttaaagctataaagaaagatcagtgggtcattgcttttattttaatcgtaatgagttttcaatccatgatagacgatctaaacatgtaccttcattacaatatcttttggattttgataggaagtttaatatacactcagtatcactattctaatgaggggacttaa

SEQ ID NO: 431 - DNA, 1431 bp, Streptococcus pneumoniae
gtgagtgaaggatgtgaaaaagtgaaaatacaaattgataaaatatactatctatttttttggctggtagtttcctttggatatttttttatgatgccaacctttacagaagatttagaaggcgttgtcagtgctagtttagtacaattgttattatttattataggatatgtatatgtatcttatttaaatcgttcttttatctcactgtatagtatctttatgattgtgttttacctttttcaaaatggacaggtattgttatattcacttggagtagagtatgattacttttatgttttaagatatgatgaaaccatcgtactacaatcagttattttttcaacacaatgtcttattgcagcctttatggctggagtcttttctacgaagaaagaggtatcaaaacccctttattcttatatggatcaattagaacgagaaaaactgattactgctggaaagttattttggggtgcgtttgcgatttttgctttaccctttatgatgatgaagtttgttatcaccagtacatctgggtattttgcgatgatacgtttcgttggaagcctacctacaataacagtgttatttgaaaaaatgttcattgcttcaagtgtttttttggttgtttatttaaaatctgaggaaacttggagcaagtttttaaaagttgttatcttaggttggagtataatggctgctttaacaggagatagaaccgtaggtttagcagggattgttacgttagctttaattcaaacattaattggtaatcgtaaaaaaaagattaagttttctcagtatgccttgttagttggggcagctgttgtagtgatgtatttaatgtcctttgcttttcaatttaggatgcaacaagattctaaagtttcaggactacaaactgcggtagtagaaatgataggaactctagggtttagttttttcccactcgtattaacgattagaattgttccaacttctattaacttttttagaggaaaatcatatattgcagccatcattacaggactaattccatctaattttgattttttacatttaacaaagacattatcagaatggaatgcatatccaacggaattattagatacaatctatcattatggtttcggattagattattctttaattgcggaagcctatattaattttggttcttatggatggatagcgatttttttcctttgttctctaatagcttattttgtaagagatgtggattttaaaaggaaggacaacttattctcacaatatgctagcttgattttgctgtattcatggtttacccttccaagaagaaaatcttattttatttttaataattttttttggtatgtattattttttggactagcactcatacttgtctctcgttctattaaacaacgaaaggaatttaaaaaatga

SEQ ID NO: 432 - DNA, 1233 bp, Streptococcus pneumoniae
atggttattaactttagtaaatataattggattttaattttaattttatcgagctcagaactgttttctaaagaaacaaatctatttttattactatttattctactatactatgttttagttcgtggaggtagactaccaatttcaattccaggagcgaaatattattttactttattaatggtggggctatttataggtattgggaatgtaataattaacgaaaatacagtatttaattttgtaaagcattttatctattacttattacctctatttttttggcagtttggatgtgttctgtcaaataacgataatttatcttacaagcaaaacaatattaaggaatcaattgttataacagcatttctattatcttcttatgatttaataggttcaatcattagtttcttgtctagaactaaggaaattgttgatttatatagtttacggtcagagtttggtcgaggttcctatataccaattattgcactgtatttagttcttgtttattctagagaaataaagttgagcaaaaattttaagggatttttgttattactgtttacaatctcgattttgatacatttttctaggacacattttattattttattgattctagtatttttttcaggatttgaaaaaaatatgaaaagtattaaaaaaataatgtttgcaggaacattgatattagcaggtgtaggtgttttatatttatattttccaaatttagttatagatttttataataaaatatctcaatcaacgacggaaatcacgttttctaaatcgtcatggtcccataaagatatcatatggaattggcgtggatatgaaatgtactctgccttaaatcattttaaatcttcaactttattagagcaaattttaggaggaggatttggaacagttttatacataggcgaatatgcgtatttcgtaagcgatttaccgtatcttctttttttacataatggatatttcactactttacttgtttttggtgttagtggggtcatactattcgttttatgggtacttagtttatttagttatagcaagtatgttaatgatacccaagattcaaattttataaaaggtcttgctgtggttatattatttaccacatattttgtaaatggtcctcttttttctgtttcacaagccaccttcttgctttattttgcactttttagtaataatcatggagaattagatgaagatttttaa

SEQ ID NO: 433 - DNA, 1233 bp, Streptococcus pneumoniae; identical to SEQ ID NO: 432

SEQ ID NO: 434 - DNA, 996 bp, Streptococcus pneumoniae
atgttaggtaaatctaagataaaatcgataatcattactccaattatacttcttataggttttattgtaagttatgataatggtaacgcctcctttgtagcatatactttgactcttatatattcttcaagggatattaattttcgtaatttggtaaggaatacaatgttctgccagattggagttgtaggaactgttatagtaagctctttacttggtattattcctaacgagctagccttaacatattcaggaggtgtgatccgctcacggtatggactaggttttcactacacatcatttactccgaattatttcctgagtatattgctggaatatgtgtatttaaaaggtgaaaaatattggacaataaaagagctttttatttgtgtactattaaatatagttatttataaatacacagatacccgtcttacttttattatggtatttatccttcttttgatatcgttttcgagaagatttgtatcaatcaatatgaattttaaatttctaaaatatttacttactcttatttatccaattatggcgtatatgacttattggctgacagcaaaatttgatagtagaaatagtttattatctttaataaataacttattgagtcaaaggttgagattcggtcaagaaggtttaaggagataccctctaagattatttggaactcatattcaatgggatgcttcggcgaattcttatctttatgtagattcgtcgtacattaatatacttataagctatggaacaataatttttttccttacgttaataagctattcaattataatgaaaaaagttataattaatcaaaataaaactttattgattgtgttaattttttggtcagtaagagcttgtattgacccacaattatttttattgtggttcaatccatttttgtttttaattgcgagaacgtttttagatgaaagagaggaaattggagatgatatcacttatatgtgtatcgaataa

SEQ ID NO: 435 - DNA, 1182 bp, Streptococcus pneumoniae; identical to SEQ ID NO: 430

SEQ ID NO: 436 - DNA, 1263 bp, Streptococcus pneumoniae
atgattaattcgtctaagtatcttaaatttgtcttatattatcagataatatcattttattttttaggtattttttataggctatctacttttaatcaatatttatatgtaatagcagatatagtttttatatcagtatacttattgatatttgagcgtaaaatcgcttatactaatataaaactatttattatctttattttattctatctatttggaagttttcaaggatatgatagtggtggtgcatttattatatttttactaatttatttaaaatatattttattcaaatttataattataaatataccgcaatccaatttaataagagcatttacaaacataggtattttaaatacggtcattttattagcagaagttttaagtcacggacatattaatttatttgtgaatcattatactttggcgcagaaaatagaaaccttaaataaggttgggacaaaccttgcggttttacgtggggggtttgaaaatcccttagtgacatctgtaatgctatcttctacattattattttttatgactattgagaaggctctcttacgtaatatactaattatgtctaacttatttttgattatggcaacagaaaaaagaacaggaattctaattagtatcgcgttgttgttctgttactatttcagaaaaaatttaaaaacaaagagtgttagtaagtttataataaaattcttgggaggattgtttttcttgggatttgctttattagccataaatatgataacaatttctggtcgtagcatttctcagatgataatagagaggttttcatcattaagtagtggttcagacttttctgctatacataggtcaatggcttttaaaataggcatagaaataatttggagtaggaatatattgaatatattattcggtaatggtttctactttttgccaacttttatgtacaataataatattacgattacaaagttgggttttttagtaatagataattcttatttatcttttttagccgattttggcatgatgcctttaataagtataatgttttatcttatagggggtatctttaagaatcttgtttcgaatgagactgataatatttcagaagtagttatgtttagcttaattgcattgcttttatcggcgagtgtgttcgatattttaaactggtatcagagtactcttttaacatgcttttttgttgcttatttaagtgtatatcatgattctagcaaaaggaaagggaataaatga

SEQ ID NO: 437 - DNA, 1230 bp, Streptococcus pneumoniae
atgaacaaaattacaataagagaattgataatactatttgtgttaattctttttatatttcaaaatcttttagaaaagtattttgtatatgctcagtatattgatgaaatagttgcattattatttctcattcattactttttaagagtagtgttaaataagagtatttcaaaaaaattttcccgacttgttcttcttttattaactatcatattaattactttaatttcaaatttttggaataatgtgcaaactaatattttaattatctccattgatttatttagtatttttaaatttatttttatatttttgggggcacaatcttttctcaatgacttaacgcataaagaaataaaaagaataattttacgagtttactttattagtaatatatacttatctgttttgattgtcttagcatttttaaatatttttataggcttaggaatgcatcaagaatatcgctatggtttaccaactttttcttttatttttggtacgccaggtcaagtaattaatctctcgattatattcttacttctatatcagttaaataaactttataattctaaaaataatctcattcatttagtattaatttttttattactgctgtctacattaaaaacacgggcaattgtcttagcaattgtgtttgtctatattctctatctatttgaaatacgaaatatttcatcaatgaaaaagagagtacttcctgttctaggcttgggagcagtggttgggtttgaacaatttaaaacttattttttgacttctgatactccaaggctcacattatttaaatacggtatgcttacaatgagacgctattttcctctgggaagtggttttgctacatatggatccgatattgctgctaaaaactattctctgttatattatcaatatggatttcataacagatatggaatgaatccatatgatattagatttttaaatgataatttttatccaatgatttttgcacaatttggatttttaggaggaatactttatgtatttctactattagattattttagattattattacgtgttgcaagtataaatgataaggttattaaaacttcagtatttatttatatctttaatgttgtcttatcgtctattcaatcatcttatccgggaacgaattcaatggtaataacaacgtttctaatctgtttaatattgagatatagtgacaagtggaggattttatga

SEQ ID NO: 438 - DNA, 1230 bp, Streptococcus pneumoniae; identical to SEQ ID NO: 437

SEQ ID NO: 439 - DNA, 1224 bp, Streptococcus pneumoniae
atgcatgttagattagatggtttgctggactatatatttctatttagtgtgattattacttgtaatactatgtattcaactagtcaaggatttgatggactagggaaatgggcgactctgttacttgtggtatcagtttttctgaaattgcttatctctagaatatctatgaaggcaatcaatgtgattgtgtcgcgttctttaatatttatattaattattctactcatagtaatattaaatggttttaagatttctgagacaagtttcgtctattattttgtattatttccgatttttatgatgattttgcagatgtactatgatgttaatgaaatcgcaaatctgatacggaaatttgttcgtataatatttcttttagcaattggctctctcctattttggcttattggtagtgtatttcatattatatccccaacggtttatgtgttgaattattggaatggtgggggaatagtagaagggtactataatcttcattttgaagcacaaaaaatagagattttgggggcgatactgataagaaatacggggatttttgctgaagcacctatgtggagtttggtattgagccttgcattgatatttcaaacgcttcatataaaaaaatggaatttcacaacttggactcttattattacaattatgacaactacgtcaacaacaggggtttatataataggtttgattttcctatatgttttattttcaaaaacaagtggtgtgaagagatatgtttctagtttatttattttagcgattatatgttgtttttcaatattatgggataataaatctggaactggttcagcaactatacgctttgatgattataaggctggttttttggcatggcagaaaagtcctatttggggattaggtatttcagacggtcttagaaccattgaacaacatatggatagaactgttagatataatcttgggtatagcaatagcttttttgttgttttagctcaaggagggataatgttggcttcgtattacttttatccagtaattaaaattattttgaataaatttagttcgaatgatttgaagttttctgcgctattaataattttccttatgataactactatatttatagagacctatatgtttttatttgtcatttctttatactattctcttgattttggggacgatagagattgtcatgagaaacagtacattactaattaa

SEQ ID NO: 440 - DNA, 1251 bp, Streptococcus pneumoniae
atggttttgttattacttacttttgcattctttttagttttccctgtgctgtcaataagtttaagtgttatagggctagtaaatgacaaaaagcgttcaaaaatctatttacttctaattagttttgcaatatcaattgttgcgctgcgctatattcctcatcctatggatgatggcgcttttcattttcgtgcgactacgacccttattcgctatgatagtattttcgaaatgtttaaagcgttttctaatggatggagagtagggaactatgactatggttccataccaatttttacttcattaatgtatcttgttcgaaatactcatcactatagtttactgagttttatttcagcttttataacttactttagttttggttacgtagttgttgagctatttaaggacctgggcaaggtttctaaactatcgtatgctacagtattaattgctgtactatgtttaaataattatagatatacaactagtggaatgagattttgtatggcagttgccttgatgatgcttttactatatttggagtcaaaaaaaggctatactagtttaaaaacaaccatatggtatttgttgcctgtaggtatccattcagcggttatttattttattggtttaaggttcttatttccattaatcaagaaagtgacgcttgctaaaagtttatttgtattattaggttttcctgttttgttcaacctagttccatggttagcaaatttgattggttggacttacttacaatcatttattcggaaaattgaagtctactctgacaattcatcctattcccagttttttaataccacattaacaatgaggctatatgttggaatagtgctgatggttttatttgtattgttatatttgggaatagtaaattcattgaaaacaactgatgattggcgttttagttttgttacaatgacttattatgtaactctgttatctatgggttcaataccatttcgtaatatttatgatagaaatcttttcttgctccttcctatgattgtagtttcaacctacatattgtttacatatagatatcaattgaaaattcttactaatcgtaatattgtttatggattaacgatgggtattctttgtctatcctgtgcagttggtgcattttataataataatttcccttttacttttattgattttagtaagacagacttactcctaaaaaatatttttcaattcttttcaaatttaccttttacataa

SEQ ID NO: 441 - DNA, 1140 bp, Streptococcus pneumoniae
atgataaaaagaaaaatatatataatctgtgtattactgtcgtattttttacctagtttttttacagttggatcaatggcgactttatttgctttgctttctactttagttaagctatttgtaacttttgattatattcccaaagttcttttacagcataaagtaacactgattgatattctagttgttctatttttattttttcaagcatttgcagcttttcaaagccaaacgttgtaccttaactatgttggaggtcagtttttccttttaggactatattcatttttgaaacactttttattgttggattgtaaaacaacaataaaaagcctctttttaacctttatcttgtttttgtgtgttcaagtaataactcaattgctttttccggtcggtttcgattctttgcaccctacaggagataatagactttactttttaggaagaaagaacatagctaccccgtatatcattgttggattaggtggtttttacttattaaataaaaaaatgaatgagtttatatcactaaaggagattatttttttaggattgttcggaattttatcatttttaacacaatccagtacagcgattatatgttatgggctttttattttcatacgtctattgggattaaaggaaaatataggaaaactatattctttagtcagtatggctgtatatgtttgtttttctctttctataatcttttcacaaagtacaattttatcaacttttacagcgattttttccaaaaatgcaactttttcaggacgtatcaatatttggcaattagcgattcgtatttttgaagagaatttttggtttggacgaggcctcaacgttaattttaatgcttggacaaatgggattatcgttaattcagcacacaataccttattagatatacttgctcgaactggtatatttccaggtgttttatttatagtgcttcttatgaatctgtttcttggaaaatatagaatagaatcaaaaacactgttggctatgttagcctcatttatgatttatattacaatggagacttcatctatgagcatattgctgctaattattgttctttgtgtatattggccacgtggagaggagaagttgcatgagcaagttacttaa

SEQ ID NO: 442 - DNA, 1209 bp, Streptococcus pneumoniae
atgtcagttagaaaactatctttaatagatatactggactatatgtggataatattaattattgtacagtgtcattcaatatatacagtaaaatctgaacctcttaatttaattgtgccatttttagttacaacagcaatgttgttgttactcaatttattacgattcactttgtcaataaaatatatatatatgttagggctatattttatagcaatggctacattttttttaactaacataggtgctaatgttactcccttaaatattacgaagtattttataatgcttcctttattttttttaataaatagtatttatgtgaataaagaaattttttcagcgttactatcaaaatttgtaaacgtagtagtaattttagcaattttttccttattcttttggatatttggaacacttctgaatgttgtacaccctacctcaacagtaattaatcaatggtctggtggtcaattaataaatagctactataatctatattttgaaactcaacagatgattttttttgggttccgtataatacggaattcaggaatatttgctgaatctcctatttgggggctaatactaagtattgcttatgttattgattttcttattttgaaatttgataaaaatagcaagcgtaatataattattctaactatgttaagtacaatttcaacgacaggtattattattgtcgggttagctatattatataaaataatgactactagtcgttggatgacaaaactcttacttctccctgtcacattgagcttaggactctctttattgctcctattggctgagaaatcagagacagtttcagctaatcttagagttgatgattataatattggttttatagtatggaaggcaagtttgtggataggacacggattgaataatggtatattagcaattcaatcacatatctcaacgtttacaagaaatttaggctatagtaacacattatttgttattttggctcagggaggtttattattattcttgatttatttttctccaatgatactattattgtttaagaaaaatatcaatcttgattttaagtttgctattatcttattctttatcttagtaacaacaattatttttgaagggacatttttatttctctggattttaacattatcgtatagctacttttcctttgtaacattagataaaactggaacgtaa

SEQ ID NO: 443 - DNA, 1371 bp, Streptococcus pneumoniae
atgctttatatattattttatttattattattattttttattataaattatgcttttgtaagaggagattattttcacccaacaattatctttaactttacttttttgatagcaacactgttttgcattttaaatgctcaagagtatgctattgagttcaatggaggtacagtttttattgtgacgatttcaatgctcgtttttacaataataacagtattatcaaagaaaatatttaagactaatactttcacctttaagaataaattaaaatatctatatgtcagtaaaagtctaatttttacaataattattattcagattttaaatcttattttcttttatcgatacgaacaagctttattcagcgcttatgttggcggtcgtggaagtttctcgcaaataattaataactatgaccaattagtaaagttttttactgaagacttagtcggattgggggtcaggtctccgtttttttatagagtgtttaatagcttcatatctcctatttcttacgtgattatatacattatttgtcacaattatgtggcaacaagaagaatagataaatttcaactcctagttccttgtttatttgcaattcaaatttttatgactggtagtagagggtccttgtttaaaattttgacttttatattagttgttttttatattttactactgcgtaataaaggacttagtaaattgagttctcgctttgcttttaaggtccttattgttgtagtggcagttgctcctctatttgtactttatttaaatgcaacggggcgttccggtggagtaggtcaagatgggattattgaagctcttaatcgagaactattcaagtacattggcgctccgttattaaattttaacaattttattgctggatatgatccgtatagaagtgactcagtatattggggagaacagactttttatggcttttataaattcttgtatagtaaactacacttgatagattttaatattaaaagtattattggtagtgattcttttgtgaagagtaccaatggattgcctacagggaatgtctttacaactttttatacgtttttttatgacttcacttatttcggcattattccattagtatctgtaattgcattatattttgttccaacttatagtaggatattgggtacagaatattgtgcaaaaaaatatttatttgattataagttatttatctatgcttatttgtttaatgatgttataatgttgattttttcaaatcgattttttgaaagtatattaacagttggttttttacgttttttaataactagtgccattttagtctggataataaaacaaaatattgtttttaagtataaaaaatag

SEQ ID NO: 444 - DNA, 1194 bp, Streptococcus pneumoniae
atgaaaatcaccatcaaaaggtattcactgccagaaattttaggactagcagcattagctatattcctactattttcgttattaagtataactctttatgcccaatatatatctattaaagtatattttattgcactttttggcattattttattaatatttctgaaggaacttatatctgagagttataatcttaaatctgtgattggattattcgtgatttttactctctgcttcatcataggtaccgtcacaaattacgcatacttttttattttaggtctgtttttgatttatagtcttagaaatctccctttttcagacgtagccaaagtctcctttatcattagtattagtgtattgctcctagtagtattgagtagtaagcttggtattattcctgattatgttgagatttggcctaccagagttagacactatcttggtttccgatattcattacttccatcaacgcttatgatgaatatcatagctatatcattttttttaaagcaagaaaatgcttcatatccgcgtcttcttgtattgtttttactttcaggatggttatacgtagaaacagattcacgtttgacttttattaattcgtgtttgtttctactggttaatttaataatgaaattgtcaccttccatcattgaaaaggtagggagactattaaaacttttttcgttcacctactttattaatgcctgtttgagttactggattgcgaagacttacttaaacacctcaaatgttgttttgaatcaattcttttaccaagtggatcaatttcttggaggacgtatttattattctaatcgttcgttatctttatatggatataagttgttaggtcaaaagattgattggattggaaatggcttgagcatacaaggggagcgtagcacagaaacatatttgtatgtggataatttatatatccagattcttcaacattttggtttgatagtttcggtaattattttaagcctattaactataactttagtaaagcttttgaaaaaggggcagatggtattaggaattattttagttattttgagttttcatgctttaattgatgatttgatactaaatctatattataatatattttgggttttgataggaatgttgatatataaaaagtatcaattttatgacaaaaagcagttgacaatagaataa

SEQ ID NO: 445 - DNA, 1230 bp, Streptococcus pneumoniae
atgaaaatcactatcaaaaggtattcactgccagaaattttaggactagcagcattagctatattcctcctattttcgctattggatgtaagtttttacgttcagtatatatctcctatgctatataaagctgcaatctttttgataattatgctaataattttgaaggaatttgtatctgggagtaatagctttgagtctctattgggtttactaggggtttctacgctctattttatcataggtagtgtaacgagctatacttcttttatagtcttagggctatctctgatatatagcttgagggatattcccttttcaaaagtagtcaaagtagcctttatcattagtgttgctatgttgttccttgtgatattgagtagtaagataggttttattcctgattacattgagttttcatctactagagttcgacactatcttggattccgatattcattatttccatcaacgatcatgatgaatattatagctatatcattttttttgaagcaagaaacagtatcgtatcagcgcctttttatattatttttagtttcaatgtacctatatttagaaactgattcacgactgacttttatcagttcgagtttgttactattagctaatttggctatgaagctaatccctactgttattgaaaaatttagtcttttattaaaatttttcacgcttacctattttgttaatgcctatttaagttattggatttcaaaaaattacttaaggacatcaaatgccttgttgaatcatttttttcatcaagcagatcaattcttaggggggcgtatttatttatctaatcgttcgttaagtttatatggttatgggattcttgggcagaaaattgcttgggttggaaacgccttgaatgcgcaaggagaacgaagtacggatgcatatttatatgtagataatctatatattcagattcttcaacattttgggctgatagctttaattattattttaagtttactgactgcgactttagtgaagcttttgagaaagggacaaattgtcttaagtattattatagtttctctgagtttccatgctttgattgacgatttgatattgagtatccattataatattttttggatattattaggtagtttaatctactcaaattatcagttttctgaagaaaggtatggagaagttgagaatgattccttacgtagaactgtaagggaaggttattaa

SEQ ID NO: 446 - DNA, 1194 bp, Streptococcus pneumoniae
atgaaaatcactatcaaaaggtattcactgccagaaattttaggattagcagcattagctatattcctactattttcgttattaagtataactctttatgcccaatatatatctattaaagtatattttattgcactttttggcattattttattaatatttctgaaggaacttatatctgagagttataatcttaaatctgtgattggattattcgtgatttttactctctgcttcatcataggtaccgtcacgaattacgcatacttttttattttaggtctgtttttgatttatagtcttagaaatctccctttttcagacgtagccaaagtctcctttatcattagtattagtgtattgctcctagtagtattgagtagtaagcttggtattattcctgattatattgagatttcgcctaccagagttagacactatcttggtttccgatattcattacttccatcaacgcttatgatgaatatcatagctatatcattttttttaaagcaagaaaacgcttcatatccgcgtcttcttgtattgtttttactttcaggatggttatacgtagaaacagattcacgtttgacttttattaattcgtgtttgtttctactggttaatttaataatgaaattgtcaccttccatcattgaaaaggtagggagactgttaaaacttttttcgttcacctactttattaatgcctgtttgagttactggattgcgaagacttacttaaacacctcaaatgttgttttgaatcaattcttttaccaagcggatcaatttcttggaggacgtatttattattctaatcgttcgttatctttatatggatataagttgttaggtcaaaagattgattggattggaaatggcttgagcatacaaggggagcgtagcacagaaacatatttgtatgtggataatttatatatccagattcttcaacattttggtttgatagtttcggtaattattttaagtctattaactataactttagtaaagcttttgaaaaaggggcagatggtattaggaattattttagttattttgagttttcatgctttaattgatgatttgatactaaatctatattataatatcttttgggttttgataggaatgttgatatataaaaaatatcaattttatgataaaaagcagttgacaatagaataa

SEQ ID NO: 447 - DNA, 1218 bp, Streptococcus pneumoniae
atgaaagtagtaataaaaagaaagtctttgccagagattttagctcttagtgcactaactatatttttagttgtgtcaattttagatgtgactttttatgttcagtatcttccaaaagttgtttataagatactgatagctatttcgcttattctgctagttataaaagagttgtacaaaagaaagtttgattatagaactatcataagtctatttgctacagttttgatgtatctccttatagggagggtgagtactcttaactctaatattgcaataggaattattttcatttatgctttacgcgatattccttttaaaagcgtggcaaaaacttctttagcagtaagtgtactcatattactgtttgttataactagcgcaaaattgggagttattataaattatttagagatttctgggactcgagtccgtagttatttaggatttaggtacgcactattcccatctatcttgatgatgaacatagtcgctattgtattttatttaaagcaaaataaaatacaatattggcaatggttattgttatctttatctgcttattggctgtatgatgaaacggattctcgattaacattttatagttcctgtatattgttgatatgcagtttattaataagatggatcccagaactattctctaagttcggatatatatttaaagcttttaaactcacctttattataaatgcagttgttagtttttgggtctcctttacctatctcaatttgagtcattcttatatcaataatctttttttaaaattagactatatattaggtggtcgtatatacttgatgaataaatcgttgaatttgtatggttttggattattcggacgaccagttgaatggaatggaaatggtctgaccattgaaggagttagaaactatcagacctatctgtatgttgataatttgtatgttcaaattttacaaaaatttggtttactcgctctaggaataatgctcctagtcctaactttaaccttgtttaaagtgattaagagacgcgaatgggttctgtcttttattttgatattgatgagttttcattccatgattgatgatttgaatttgtaccttcattacaatattttttggatattattaggtagtttaatctaccccgattatcaattttctgatgaaagtgatgaagagttaggggaaaattcttttgaagaaattatatag

SEQ ID NO: 448 - DNA, 1197 bp, Streptococcus pneumoniae
atgttcattctatattattctttactaaataataatatgataaaaattaaatttgaaaatctactggttagtattgttgtatctgtagtgatattttttaatactatatctactacaatgctggatagaactttttttcaagctaaagcaaattatcttttattagtagttttgttattaggtcttcgttttatgtttccaacacgtatttcatttaaatatctaatattttcttctttgctattattatcaggaattttagtatattttcagactggaaaacttaattttcttgtctattcagcccttctggttttattagttaatgtagatatgaaagtagttttgaaaacgtatctcattgtagcgggcattcttgtactaagtgtatttcttttatctctcttaggtgcagttcccaatttacaatataatcgcgaaggagttattcggaattcatttggatttatttatcctacagattttgcatcccactgtttctatcttttcttggctttttcttatttattaaaagataaatttatatggattaggtctctaataggtgtacttttatctttctttattataaaatattgtgatgcacgtttaaatgccttatcaattattttagcaacaattatttttatatttttctactttaatgaagataagaaattccggatctattcaatttccccttattctgtagttctatgttcgtcaataatgatgtatttatcttatattttctcttggggatctccatttttagtatctatcaataaattaattacaggtagattagctttggggaaaaatgcatttaatacttttgatgtacatctatttggaactagagacgtattatttaaggggtcaggaggtaaaacggaggctgttattgattataactacgtagattcatcgtatgttcaaatgttatttacatatggtgttatacctcttgtactgcttatatgtatgtatgttgtaatttcgagagaacagtataaacagggtcaatatttatatgtagtaatattatctttagtggctctaaactgcatgattgaagctttttggtttgtccctacttacaatatattcatgtttttgttattcacaaaaaatacttttcttaataagtcgttgaataattttaatcctttggataaaacggaattgttgaactga

SEQ ID NO: 449 - DNA, 1431 bp, Streptococcus pneumoniae
gtgagtgaaggatgtgaaaaagtgaaaatacaaattgataaaatatactatctatttttttggctggtagtttcctttggatatttttttatgatgccaacctttacagaagatttagaaggcgttgtcagtgctagtttagtacaattgttattatttattataggatatgtatatgtatcttatttaaatcgttcttttatctcactgtatagtatctttatgattgtattttacctttttcaaaatggacaggtattgttatattcacttggagtagagtatgattacttttatgttttaagatatgatgaaaccatcgtactacaatcagttattttttcaacacaatgtcttattgcagcctttatggctggagtcttttctacgaagaaagaggtatcaaaacccctttattcttatatggatcaattagaacgagaaaaactgattactactggaaagttattttggggtgcgtttgcgatttttgctttaccctttatgatgatgaagcttgttatcaccagtacatctgggtattttgcgatgatacgtttcgttggaagcctacctacaataacagtgttatttgaaaaaatgttcattgcttcaagtgtttttttgattgtttatttaaaatctgaggaaacttggagcaagtttttaaaagttgttatcttaggttggagtataatggctgctttaacaggagatagaaccgtaggtttagcagggattgttacgttagctttaattcaaacattaattggtaatcgtaaaaaaaagattaagttttctcagtatgccttgttagttggggcagctgttgtagtgatgtatttaatgtcctttgcttttcaatttaggatgcaacaagattctaaagtttcaggactacaaactgcggtagtagaaatgataggaactctagggtttagttttttcccactcgtattaacgattagaattgttccaacttctattaacttttttagaggaaaatcatatattgcagccatcattacaggactaattccatctaattttgattttttacatttaacaaatacattatcagaatggaatgcatatccaacggaattattagatacaatctatcattatggtttcggattagattattctttaattgcggaagcctatattaattttggttcttatggatggatagcgatttttttcctttgttctctaatagcttattttgtaagagatgtggattttaaaagaaaggacaacttattctcacaatatgctagcttgattttgctgtattcatggtttacccttccaagaagaaaatcttattttatttttaataattttttttggtatgtattattttttggactagcactcatacttgtctctcgttctattaaacaacgaaaggaatttaaaaaatga

SEQ ID NO: 450 - DNA, 1278 bp, Streptococcus pneumoniae
atgtttttagtttttttactgttttttggtgtatttctattatttccaattataactacaccagtcttattactcccaatagtttatagatttagatatagcagatattatttaatgttatttgttataggtatttccttaattgcgctaaggtatattccgtattttactgatgatggagcctatcattataaagcagcttatttatttcagttttatgataatgtttttgattggtttgggaacttgatgtctaaaaatattccgacggaagagtatggttattataattatcccttatttgccttattgctatatatattcagtaagacaggtacctattcactagttagttttacagtcattctgattgtttatttcttatatactaaaataatatatgaaatctatcgggaatataatatctcaaaatttttatttttattagctttattaacaatggttgcaatcgtaaatgttcgttttacaactagtggtatgaggtatcatttagcaggggccattattgttttcttattctataaagaaataaagaatggatttgaactaaataaaactttattttattatcttattccaatattaatccattctagtgcagtaatatttgtagcgactagattaatatttccatggtttaaagatgcgagtttttttaagaaaataattattctattttctttaccaatttttacgctcttatctccattgttgcaaactctcaatgttgaatatttatcatttttattagagaagtttaatgcttatcaaaaaacagaaatatttataaaattatatagtacatctgatttaataaatgtatatttaggtgttcttatttctttattatatatttttttataccatacaacatttcgttttcagaagaatcttaatatgaaactttttttatcttttgttttgtacatttgtttattaactttatcagtactcccatttttaacaatattagatcgttttgtttggtttgtttatcctttagttgccatttcgatgattttacatattggctacaataaaacccatattgaaagagtacaatacattagaaataattatttaccatttttcattgtattaaccctatgctttattggtggagttatcggaaataggagatttttagattttttaagattggtagatttcaatacaatggagatactcactaaaaatgtatttgattatttttctgatttgcaccatttctctttgagtgaagttttaagacgataa

SEQ ID NO: 451 - DNA, 1233 bp, Streptococcus pneumoniae
gtgaaaaaaagtagtacgttacatatagggcttattctttttttgattgtactgagtaaaaacttctttaatttagtaccaaatatagcactgttttctgattttaatcttatgattattataatcatcatgacagttgttaatattaaatactataatcgctcaaccaaatatcagtatcgttggtatattattttcacattgatatttgtgttgtatagtgcaattaatgagaaactattatatggccaaccatttctattaggtttacttcctcaaagacaattttttttatgtttaatgacttatttccctttaagaaagtatttccaagaacaaaatattagtttaaaaaaactttatgttggtattatgaatttgggatctatttcatcgttaatttacattttacaaaagatagtaatattatatgggggaacacaatttgttaacgtaatgtatagttttgccggatattttagcgggtatagattgtatgtaggtagttctttaattattttagcaacattaatttctaccgcttattttttagaaacattaaaactcaagtatctgtgtcaccttatattaggttggtttactcaaatttggataacgcaaggtcgaatagagatgattgttttgctaatttcaacgatagtttgcataatagttcaaggaagattaactagaaagaaaataatttattggttgctgacaattttgggactgtggatgattactctaacacctatttttgaaaatattgttggagctattttaaaagtagatggagcaggtagaggaagtgattcattgactattcgagcaattggacgacagttatattgggatcaattgaatgagacaacttcaaaacttctttttggaacaggatatcctaattataattatgctttagcctttcaacgtacaggtttttcaagcaatatttttttaacagacaatggttttatgacacatatatatatatatggtattgtgggttcctcaattatgggattattatttttaaaatatcttaaaaattcaataaagtatgctagacaatctgcagatatgattccgttgatgtatattatttctttactaattgtagcctataatattattctttggtattggaatgcggacggtacttttatattagtaatcatgatttgcgcattggagcatggagaacaattattacatcagaggaatggagtgtga

SEQ ID NO: 452 - DNA, 1260 bp, Streptococcus pneumoniae
atgattaaatttaattattctaaattagcattaatttaccagtttataataattttttttctgggtgcttggtataagaatagcattcagtttaaaatttttttgatatctattgatactatcatttctttattttatttatataaaaataatttatctagaagtgacttaaaaaaagtttatatcttgataactacagtgttagtaagcttatttttaccatcgtttatgaatagagtatcttttatacttctcatattgcaaaaattatatatatttaaccatattattaatgatataaagtttgaatatttgctctcagtctttctacaatttattgtactactaaccttttgtatgggattagaattcatttcattaggacaatttaatccgtttaaaacattttatgcaccaagagttcttcttgatatgagttttaaagtcggaacacctttatattttttgagatcaagtttagagcatcctctaataacttcgattgtattagtagttactggccctttcttattccttttagaaaagaaatggttgagatatgtatgtgtgtttctaaatattagtttaatcttttttattcagaagagaactgcgtatatattggtatcgattggagttgtatgttttgctttgtattatctaaaatatattaatcgtagatttcaattgaataaaataacttcagtgatatttttaatgacactatttataattgctttatcctttataagggtgcaaggtgattatgtattaaatattatttttagtaaattttctgctttacaagatgcagatagtttctcgctaaataatcgtgtaattaatgctaaaactggattagatgtaattttcgatcaaaatgttataaatatattcattgggaatggatatgactttttacctaatttttttgaacaatacaatgtgtatgtaattcggaatggtttttatgtgattgacaatacatatatttcatttcttgccgactatggaatgattggtctgttcttagtattgttatatattataaatgcgatccgaaaaagtatgactaatatttacaaaataaagagtaaaaaagaacaattatttatgttgtatagtattataggtattatcaatttaatggtatcaatagctttttttgatatatatgcttggtacacgctattaacgttattgatttttttaatatcagtagtgacttcttattgtaaatttcattccaataagttttag

SEQ ID NO: 453 - DNA, 1260 bp, Streptococcus pneumoniae
atgattaaatttaattattctaaattagcattaatttaccagtttataataattttttttctgggtgcttggtataagaatagcattcagtttaaaatttttttgatatctattgatactatcatttctttattttatttatataaaaataatttatctagaagtgacttaaaaaaagtttatatcttgataactacagtgttagtaagcttatttttaccatcgtttatgaatagagtatcttttatacttctcatattgcaaaaattatatatatttaaccatattattaatgatataaagtttgaatatttgctctcagtctttctacaatttattgtactactaaccttttgtatgggattagaattcatttcattaggacaatttaatccgtttaaaacattttatgcaccaagagttcttcttgatatgagttttaaagtcggaacacctttatattttttgagatcaagtttagagcatcctctaataacttcgattatattagtagttactggccctttcttattccttttagaaaagaaatggttgagatatgtatgtgtgtttctaaatattagtttaatcttttttattcagaagagaactgcgtatatattggtatcgattggagttgtatgttttgctttgtattatctaaaatatattaatcgtagatttcaattgaataaaataacttcagtgatatttttaatgacactatttataattgctttatcctttataagggtgcaaggtgattatgtattaaatattatttttagtaaattttctgctttacaagatgcagatagtttctcgctaaataatcgtgtaattaatgctaaaactggattagatgtaattttcgatcaaaatgttataaatatattcattgggaatggatatgactttttacctaatttttttgaacaatacaatgtgtatgtaattcggaatggtttttatgtgattgacaatacatatatttcatttcttgccgactatggaatgattggtctgttcttagtattgttatatattataaatgcgatccgaaaaagtatgactaatatttacaaaataaagagtaaaaaagaacaattatttatgttgtatagtattataggtattatcaatttgatggtatcaatagctttttttgatatatatgcttggtacacgccattaacgttattgatttttttaatatcagtagtgacttcttattgtaaatttcattccaataagttttag

SEQ ID NO: 454 - DNA, 1194 bp, Streptococcus pneumoniae
atgaaaatcactatcaaaaggtattcactgccagaaattttaggattagcagcattagctatattcctactattttcgttattaagtataactctttatgcccaatatatatctattaaagtatattttattgcactttttggcattattttattaatatttctgaaggaacttatatctgagagttataatcttaaatctgtgattggattattcgtgatttttactctctgcttcatcataggtaccgtcacgaattacgcatacttttttattttaggtctgtttttgatttatagtcttagaaatctccctttttcagacgtagccaaagtctcctttatcattagtattagtgtattgctcctagtagtattgagtagtaagcttggtattattcctgattatattgagatttcgcctaccagagttagacactatcttggtttccgatattcattacttccatcaacgcttatgatgaatatcatagctatatcattttttttaaagcaagaaaacgcttcatatccgcgtcttcttgtattgtttttactttcaggatggttatacgtagaaacagattcacgtttgacttttattaattcgtgtttgtttctactggttaatttaataatgaaattgtcaccttccatcattgaaaaggtagggagactgttaaaacttttttcgttcacctactttattaatgcctgtttgagttactggattgcgaagacttacttaaacacctcaaatgttgttttgaatcaattcttttaccaagcggatcaatttcttggaggacgtatttattattctaatcgttcgttatctttatatggatataagttgttaggtcaaaagattgattggattggaaatggcttgagcatacaaggggagcgtagcacagaaacatatttgtatgtggataatttatatatccagattcttcaacattttggtttgatagtttcggtaattattttaagtctattaactataactttagtaaagcttttgaaaaaggggcagatggtattaggaattattttagttattttgagttttcatgctttaattgatgatttgatactaaatctatattataatatcttttgggttttgataggaatgttgatatataaaaagtatcaattttatgataaaaagcagttgacaatagaataa

SEQ ID NO: 455 - DNA, 954 bp, Streptococcus pneumoniae
atgcatagtcttaaatatggaataaatagattaaaactaacttccttaatattgttttttttaacatttattttgaatctttttaatggttctacatttctagaaataacgtacttcttcttattatatacaattatattaaaagataataaaaaatttttcaaaatacattttattacacttacagtattcgtgttaacaattatattattgtcccatattggctatattagtaattttgtgatatatagatcaaatggtggtattaggaatagtttaggatttgtacacccgaattcattagggttaatgatctattccttaaccgtttgtagtttaaatctttttgaaataaaaagattcaaattcttcttctatttatttttattattgatgaatttatggatattacagttagcagactctagaacatcaggatatataagtattttagtaattgtcttttgttacttttttgataaaataggtaagttaaatagaccgttgagtagtttttttattaatttggctattacaatgtctgttttgtttataactttaatgtcaattaattttgaagccagtcatattttccaattcttaaataaattgttcactaatcgaatttttagtagttatgtattttttagagaatatggggttactctatttggcaaaagaattgatggaattgtctcattagttcaaggaacagtcataattgacagtggttatataggattgctattacaaaaaggaattattttttttgtgttatttttgctatttattttttctagaataaaagaatactcatttacactaagagaaggtattctactgattagtgtatttatttctcttatgtttgagagctatggttacaccgtatttctattcccaatattattttttgattatttaggaaaaaggaaggaaagtattaatgaataa

SEQ ID NO: 456 - DNA, 1212 bp, Streptococcus pneumoniae; identical to SEQ ID NO: 408

SEQ ID NO: 457 - DNA, 1089 bp, Streptococcus pneumoniae
atgatttcagctttattttatataagttttttaacaaatctattagtcgttgtcaataggatgcgtataaattccctagcatttttatcttggttaattttgttttttatctatgcaggaaatatccgagaaggattttcagatttaattcattatcgtgcaagatatgctgtagggaaaagtagtctgtatttttcagaccatgcgtataatttttttgcagattggttttcacatcactccatctcttttcaagtttttttagcatgtatttttctattctcctcagcatgtttctattttgttgctaaaaaattgcattgtaattataatcttctgattttattattatctcttttttatttcttttatactttagaagttctaaggttctttttagcaactagtgtagcacttatagcacactattatctttcacaaaatcatcgattcatattcataggattgatgttcatagcgtttctgtttcacggatctatcatttatttcttaccatttattattttttatagacaaaaaaatactagtaagatgctatgggcattggtatttatatctatggtgttaattgtgacaaatctattggtaggtaataattctagctacttgactaaaatatttgaactgctagggagcgaggtcactaaaagtcgtgttatgtattatacagcgaaatcgactcgtcttggttttatattgtatgatagttactatattttcaacctactaattagtattaaccttaaaaagattgcaaaaacagtttcaaatgcacctcaggaagtgaccaattttataaatctggtgtatcaacatagtttttttagtacaatttttttaccactaattatgtttagtacagcatttactcgctatttaatttttactgttgttttaaattttatagctattgcagctttacaaccatttatgcaacaatcatttataaaattaaagagtgggctttatattattttcggagttttaggtgctacttgtttttggtggtatctaagagaaaatgtcctttatttttatgaggcacttttgccaaatttatttaactga

SEQ ID NO: 458 - DNA, 1212 bp, Streptococcus pneumoniae
atgatgaaaagaatttattatcaccttttggctattttggcttggactcttccaaattcttatgcttttattgattcgttaaaggtgttttttccgaatattagtttacaaattgcaggttctcttttggctgttatctcgataggaatttttatcactagaatatattatacaggatacgaagtttttatatctctattggtttgtatttttattttaattttttattcaactcgctttttttattcgactaatgtagagttgtatcattctttcttcaattcctttctgattcgtccagttcctgggatattagtagcgatgctgttagctaagaataatcatataaaaggtttcataaaatggtcagaacctatgatgcttttctatacattgacatcatttttagcagccctatctcctagaaatagcattattatttaccaatctttatcttattatgccatgacagcttatatgattaatgtgtttaatattatatacaaagagaaaatattggaggagcaactgacgtatttcagattctcagcgtggaattttattagatattttttacttttgattcaagcttttaatgcactctcaggaggtgggagaggtgcctttatcttattaattatttttacaggaatattattaataagaattgctatgaattttaagttccttttctcattcattggaggcttatctgctatagctctagttgttttttgtataaaagatttggatttcacatggctcttgaatatggacggtggagagcgattgttgaacttcttcggcagacctgaacgtatatctacagataatagactattgatttatgatactgtttggtctgcaatacaggaaaaaccctttttaggatggggagttggttcaacttttttaaaatttaatggatattcacataatattgtattggatttattgcatgatatgggaagttttggtttattaattattctttcggctttctttgcttcttcagtaatattgtacaaattacgtaaaatagattggaaagtgaatttatttattctgatgtttctagaagtatatgttcacatgagtttcagtggaagctatcttgcagatggtagactatggtttttggtgatctttacttattgttactacaggtggaaagtgcaggaaaaggatgaagttagcaattttattatctga

SEQ ID NO: 459 - DNA, 1023 bp, Streptococcus pneumoniae
atgctgaaatttgtaaatattgcgaaaagtggtagtttaaatatcttaattttagctagttcagtatttttattgctacttcacacagtgatttacggctttaataaattgaagctattttgtctatccttcgtattatttttatgttttctaatttattttcaaggattagtttatctggaattgcttaatttttttctgatttactcaactctaagagtgagtaatattcaatttaacaagactcattggtcagtgttttttattttttttatattagtagttttatttgctaatgcaggaattattaaaagtgtattcatttttcgaactggagggttagtaagaggatcattagggtttgtccatccgaattcactaggtctcatggcttatgcaattacattaaatacattgtacgtctttaaccccaatagatacaaaattattttctactgcggtcttcttatatttaattattttatatttgccataacggactctagaacttctttctcaataagtttattgattattttaagttgttttatttttgattttagaggtattctatacaaggttgttaaaaattcatttatttatatagtaattgtagtaagttcgctatttattattttagcaactacctattttgaagtgagggaacctttcatttttttgaataaactttttaccaatagaatttacagtggtaatgtttttgtagctgaatatggatataatctttttgggaattttatagagggaacactacctactttagcaggagatgtaattattgatagtggttatgtgggtttaattcttcgtttaggtgtattattttttataggctacttgttgtttatgttgttaagaataaaacaaaatatttttttattgaaggaagcaatcttgattagcagtgcctttatttcattaatgtttgaatcctatgggttttctggttttattttcccagttttatttgtggattttgtgggtagaagaaaggaattataa

SEQ ID NO: 460 - DNA, 1218 bp, Streptococcus pneumoniae
atgaaagtagtaataaaaagaaagtctttgccagagattttagctcttagtgcactaactatatttttagttgtgtcaattttaaatgtgactttttatgttcagtatcttccaaaagttgtttataagatactgatagctatttcgcttattctgctagttataaaagagttgtacaaaagaaagtttgattatagaactatcataagtctatttgctacagttttgatgtatctccttatagggagggtgagtactcttaactctaatattgcaataggaattattttcatttatgctttacgcgatattccttttaaaagcgtggcaaaaacttctttagcagtaagtgtactcatattactgtttgttataactggcgcaaaattgggagttattataaattatttagagatttctgggactcgagtccgtagttatttaggatttaggtacgcactattcccatctatcttgatgatgaacatagtcgctattgtattttatttaaagcaaaataaaatacaatattggcaatggttattgttatctttatctgcttattggctgtatgatgaaacggattctcgattaacattttatagttcctgtatattgttgatatgcagtttattaataagatggatcccagaactattctctaagttcggatatatatttaaagcttttaaactcacctttattataaatgcagttgttagtttttgggtctcctttacctatctcaatttgagtcattcttatatcaataatctttttttaaaattagactatatattaggtggtcgtatatacttggtgaataaatcgttgaatttgtatggttttggattattcggacgaccagttgaatggaatggaaatggtctgaccattgaaggagttagaaactatcagacctatctgtatgttgataatttgtatgttcaaattttacaaaaatttggtttactcgctctaggaataatgctcctagtcctaactttaaccttgtttaaagtgattaagagacgcgaatgggttctgtcttttattttgatattgatgagttttcattccatgattgatgatttgaatttgtaccttcattacaatattttttggatattattaggtagtttaatctaccccgattatcaattttctgatgaaagtgatgaagagttaggggaaaattcttttgaagaaattatatag

SEQ ID NO: 461 - DNA, 1209 bp, Streptococcus pneumoniae; identical to SEQ ID NO: 442

SEQ ID NO: 462 - DNA, 1182 bp, Streptococcus pneumoniae
atgaaaatttcaattacaaatttatatatatggatgttgacagtaatgtttatattaatgtggattccaagtgttcctatatttaattatgtagatgaactattaacgttgatgatacttccgtatgtgattatgaattttagagctcttctactaaaaagtagattagtaattataggtgcaattggttttttattaataggattaatatcaacatacctttcagatcttagccgccctttgatagctatagtatcggattttttcacatttattaagtttcctattacctatttatattttagatatttatttacaaaacaaaattcaacttattttttgaatattttatatattcttagtaagtttgttgtggttattgcttttatttgctgtttattaaattatagttttgaactaggcatgacctatgatatccgctttggaataggtagttttcagtttatatataataacccaggattttttctagccttgatggttgtttgttattcacttataaattactctaataattcaggtaattttaaatataaaattttagctattttaaatatggttgcaactctacgatcagtggcatttggagttattgccctactgctagttttgagtgtgtttataggaagaaatttaagattatatcacttatttatattgattccgtcgattatttttgtaggtaatggtgctataagtacatatttcggtgatttggaaacacctagaaaacttctattccagggaggtataaaggtttttcagaaatttttcccattaggggcaggttttgcttcatatgggagcaacgctgcttatgtgtattactctcccctgtattttcaatttggttttcatcatatttggggtcttaatttagaatatgggaatatagctaatgataacttttggccgatgatttttgcacagtttggttttattggttctatattttatatactgatgttcatggctattatgaaggatttattcattaaatctatgtataaaaatcaagtggtatctttatctttaattataggtcatttgttaatttcctcagttggtgcaaatataattactggtgtgttaggtgtaagtatgatatgtgcatatgcctcaatggctcatgttttagatgatgtagaggtaaattattga

SEQ ID NO: 463 - DNA, 1416 bp, Streptococcus pneumoniae
atgattataaaactagataaacaaaaatgttattttattatgttacttttattgatgtgcgtagtacagttgactgcaagtgtttcaatgggagtaataagtatagctctaacagtattaccaatattgtggaatttaaataatccatccaaattattattttcacagattctttatttgtttcagttccaattctatgtaagttatttatcatttccgttaggaatttcctttatctgtgatattattaatttatttttgttattcaatatttttaaagattccaaactattacagtgggaattacggaatattaatttaaaaacaattatttttgtttgggtcttgttttttttgataggtattttttcaaattttttgtatgatttaaatattgcgcttattgtatggtccctccgtaattatataagatttatcatttttttcatttcttgttgtttgtatattgataaatacagtataaatctaggtgaatacttaataaagctattctattggtttaatatattttttacctcgttccaatattttgtgcttttaaaatcaggagattttcttggaggaatttttggaaacgaattgggaatttctaatacctacttgcatattcttttaattctcatcttgatattatctgttgttaattatgtatcagataattcgagtttagttattttaacaagttatatcgtttcaactctatacgtagctgctttgtcagagttaaagattatttttgtggaattacctattattattattttgactttactatttaaaagattaggaataaaaatactgcttaaaataatatctatcacttgcatagtggtggtcgctttggcaatctcaattcctattttatatgaattattccctatatttgacaatttttttaaattagaaaaattatttggctattcaacaggaaactattcaactagttctgattttggtcggttaagttcaattgttcaaataatctcaactattttttatggagatgtttggcagactttatttggtattggtttaggagaggcggaaatttcaaaaattccgatatttagtggttccttttattataattacgaatacacccattactatctgttcactttatcatatgtttttattgagaatggatttataggattgttcctattcatgtttatgccaatttaccttgctttaaagatgatttttgagcgagggacatctaaatatgatcttcttctgatagtattatctatttctttatttatgctcttgttgtataataatacaatgaggtcagagatatattatctttatttatttgttctttcatggggagttgcaatcaatagaggaaatgggggatgttatgaaaacaataaaaaattatgcttataa

SEQ ID NO: 464 - DNA, 1122 bp, Streptococcus pneumoniae
atgattattataatcatcatgacagttgttaatattaaatactataatcgctcaaccaaatatcagtatcgttggtatattattttcacattgatatttgtgttgtatagtgcaattaatgagaaactattatatgggcaaccatttctattaggtttacttcctcaaagacaattttttttatgtttaatgacttatttccctttaagaaagtatttccaagaacaaaatattagtttaaaaaaactttatgttggtattatgaatttgggatctatttcatcgttaatttacattttacaaaagatagtaatattatatgggggaacacaatttgttaacgtaatatatagttttgccggatattttagcgggtatagattgtatgtaggtagttctttaattattttagcaacattaatttctaccgcttattttttagaaacattaaaactcaagtatctgtgccaccttatattaggttggtttactcaaatttggataacgcaaggtcgaatagagatgattgttttgctaatttcaacgatagtttgcataatagtccaaggaagattaactagaaagaaaataatttattggttgctgacaattttgggactgtggatgattactctaacacctatttttgaaaatattgttggagctattttaaaagtagatggagcaggtagaggaagtgattcattgactattcgagcaattggacgacagttatattgggaacaattgaatgagacaacttcaaaacttctttttggaacaggataccctaattataattatgctttagcctttcaacgtacaggtttttcaagcaatatttttttaacagacaatggttttatgacatatatatatatatttggtattgtgggttcctcaattatgggattattatttttaaaatatcttaaaaattcaataaagtatgctagacaatctgcagatatgattccgttgatgtatattatttctttactaattgtagcctataatattattctttggtattggaatgcggacggtacttttatattagtaatcatgatttgcgcattggagcatggagaacaattattacatcagaggaatggagtgtga

SEQ ID NO: 465 - DNA, 1416 bp, Streptococcus pneumoniae; identical to SEQ ID NO: 463

SEQ ID NO: 466 - DNA, 1140 bp, Streptococcus pneumoniae
atgataaaaagaaaaatatatataatctgtgtattactgtcgtattttttacctagtttttttacagttggatcaatggcaactatatttaccctactctccacagtaattaggctattcgtaactatatatcatgttcctaaagttttttcacagtataaggtaacgctgattgacattttggttgttctgtttttgttttttcaagtgtttgtgggggttcaaagtcagacagtgaatcttaactatattggaggacagtttttctttttaggattatattcatttttgaaatactttttattgtttgattgtaaaacaacaattgaaagcctctttttaacctttatcttgtttttgtgtgttcaagtaataactcaattgctttttccggttggtttcgactctttgtaccctacaggagataatagactttactttttaggaagaaaaaacatagccaccccatatatcattgttggattaggtagtttttacttattaaataaaaaaatgaatgagtttatatcactaaaggagattatttttttaggattgttcggaattttatcatttttaacacaatccagtacagcgattatatgttatgtgctttttatttttatgcgtatgttaggtttaaaggaaaatataggaaaaccatattctttagtcagtataactgtgtatatttgttttagtttttctataattttttcacaaagtacaattttatcaacttttacagcaattttttccaaaaatgcaaccttttcaggacgtatcaatatttggcagttagcgattcgtatttttgaagaaaatttttggtttggaaaaggacttaatattgatttcaatgcttggacaaatgggattatcgttaactccgcacacaataccttactagatatacttgctcgaacgggtatatttccgggtattttatttgtagtggttcttctaagtctatttcttggaaaatatagagtaaaatcaaaaacgctactgacaatgttgatttcattcatggtttatattacgatggaggctacatctgtaagtattcttctattaataattgctatttgtgtgtattggtcttttggagaggagaagttatatgagcaagttacttaa

SEQ ID NO: 467 - DNA, 1140 bp, Streptococcus pneumoniae
atgataaaaagaaaaatatatataatctgtgtattactgtcgtattttttacctagtttttttacagttggctcaatggcgactttatttgctttgctttctactttagttaagctatttgtgacttttgattatattcccaaagttcttttacaacataaagtaacactgattgatattctagttgttctatttttattttttcaagcatttgcagcttttcaaagccaaacgttgtaccttaactacgttggaggtcagtttttccttttaggactatattcatttttgaaacactttttattgttggattgtaaaacaacaataaaaagcctctttttaacctttatcttgtttttgtgtgttcaggtaataactcaattgctttttccggtcggtttcgattctttgcaccctacaggagataatagactttactttttaggaagaaagaacatagctaccccgtatatcattgttggattaggtggtttttacttattaaataaaaaaatgaatgagttcatatcactaaaggagattatttttttaggattgttcggaattttatcatttttaacacaatccagtacagcgattatatgttatgggctttttattttcatacgtctattgggattaaaggaaaatataggaaaactatattctttagtcagtatggctgtatatgtttgtttttctctttctataatcttttcacaaagtacaattttatcaacttttacagcgattttttccaaaaatgcaactttttcaggacgtattaatatttggcaattagcgattcgtatttttgaagagaatttttggtttggacgaggcctcgacgttaattttaatgcttggacaaatgggattatcgttaattcagcacacaataccttattagatatacttgctcgaactggcatatttccaggtgttttatttatagtgcttcttatgaatctgtttcttggaaaatatagaatagaatcaaaaacactgttggctatgttagcctcatttatgatttatattacaatggagacttcatctatgagcatattgctgctaattattgttctttgtgtatattggccacgtggagaggagaagttgcatgagcaagttacttaa

SEQ ID NO: 468 - DNA, 1233 bp, Streptococcus pneumoniae
atggcagttagaaaactatctttagtaaatatactagattatgtatggttaacattaattattttacagtgttattctatatacaccgtaaagtctgaacctcttactttaatcgtgccattattaattacaaccgtaacgttgttgttattcaatttattacgttttactttatcattaaattttatacataaatttatctatatgttagggctgtattttacagcaatagttacattttttttaactaacattggtgctaatgttactatcttaaatattacgaagtactttatattgttccctttattttttttaataaatagcatgtatatgaatagaaagattcttccagcattactttctaaatttgtaaatgtggtagtaattttagcaatgttttccttatttttttggatatttgggacacttctgaatgttgtacatcctacctcgacagtaattaatcagtggtctggtggtcgattaataaatagctactataacctatattttgaaacccaacagatgactttttttgggatccatataatacgaaattcaggaatatttgctgaatctcctatttgggggttaatactaagtatcgcttatgttattgatttacttattttaaaatctgataaaaataataagcgtaatataattattctaacaatgttaagcacgatttcaacggcaggtataagtattatcgggttagctatattatataaaatagctacgaatcgtcataaggcaaaacttttactattccctgtcacagtaggtttaggcttctctttgttactcctattgactgagaaatcagagacagtttcagcaaatcttagagttgatgattataatattggttttatagtatggaagacaagtttgtggataggacacggattgaataatggtatattagcaattcaatcacatattccaacgtttataagaggtgtaggctatagtaacacattatttgttattttggctcagggaggattatttctatttctaatctatttttctcctatgatcctattattatttaagaaaaaaattaattttgattttaaatttgcgattatcctatttttaattttagtaacaacaattatttttgagggaacgtacttatttttctggattttagcattatcttatagctatttgttctttatgattttagataaaaacagtattcaaaatagacaattataa

SEQ ID NO: 469 - DNA, 1425 bp, Streptococcus pneumoniae; identical to SEQ ID NO: 398

SEQ ID NO: 470 - DNA, 1446 bp, Streptococcus pneumoniae
atggggaataaatccataaagttgaatgcattattaaatattgtcctgacgctatcaaatatcattttcccattaatcacttttccttatatatctagaatattgaatccaaatggtataggtttaacttcattttttagttcaatagggaattatggtattttacttgcttctctgggaatttcaacttatggtatcaaagcagtagcaagtgttagagatgatagagataagttgtcaaaagtagtacaggagttaatgattataaacgttgctatgtctataataacaactgcaatactattatttatgactatacttataacacaattgaatagagaattttcactcctattgatcacatgtgggactattttatcttctcctttcgccttaaattggttgtatagtggaatggaagaatatacgtatattactactaggtcagtagtgtttaaaattctatcattaatattgatttttctacttgtgaaaaggccagaggattatattgtttttgctagtatttcattgttttcttctctaagttcaaatatcttaaatctatggcatagccgacatttcattaatattaaattatataaaaatttacaatttaaatatcattttaaaccaatgtggtatttatttgcctcattacttgcagtaaatatttatactaatttagatacagtgatgctcggttttattaatggtaatgaggctgtgggatactattctgtggcatcaaaggttaagtggattttgctttctcttattacatctattagtgcagttttgctaccgagactttcattttatattagtaaaaatgacacctcgaattttataaaaatgttaaaggagtcatctgcggttatattttttattgcgattccattgatggtattctttattgtagaggcgaaagatagtatcttattactaggaggaagtcagtatcttcctgcgactttagcgatgcaaatacttatgccaattttacttatttctggtttctcgaatattacaggaaatcaaatattgattccaatgaatagagaaaaatattttatggttgcagtaacgattggtgctgtgattaatcttattttgaatctactgttaatgcctaagtttggaattattggtgcttctgtcgcaactctttttgcggaattgtcgcagatgacggtacaattacatttttcaaaagaatatttagtatcaaatatatcgataaagagtttggttaatgtgataattgcaacagttgtttctataataccactaatcattttgaatcagctgataacgataactataccattttattctctaatgctagcaggttttgctttcttttcattatatttagtaattctgcttttattaaaggaggaagtgacgattcaattattttctcttcttgcaaagaagaagtaa

SEQ ID NO: 471 - DNA, 1446 bp, Streptococcus pneumoniae
atggggaataaatccataaagttgaatgcattattaaatattgtcctgacgctatcaaatatcattttcccattaatcacttttccttatatatctagaatattgaatccaaatggtataggtttaacttcattttttagttcaatagggaattatggtattttacttgcttctctgggaatttcaacttatggtatcaaagcagtagcaagtgttagagatgatagagataagttgtcaaaagtagtacaggagttaatgattataaacgttgctatgtctataataacaactgcaatactattatttatgactatatttataacacaattgaatagagaattttcactcctattgatcacatgtgggactattttatcttctcctttcgccttaaattggttgtatagtggaatggaagaatatacgtatattactactaggtcagtagtgtttaaaattctatcattaatattgatttttctacttgtgaaaaggccagaggattatattgtttttgctagtatttcattgttttcttctctaagttcaaatatcttaaatctatggcatagccgacatttcattaatattaaattatataaaaatttacaatttaaatatcattttaaaccaatgtggtatttatttgcctcattacttgcagtaaatatttatactaatttagatacagtgatgctcggttttattaatggtaatgaggctgtgggatactattctgtggcatcaaaggttaagtggattttgctttctcttattacatctattagtgcagttttgctaccgagactttcattttatattagtaaaaatgacacctcgaattttataaaaatgttaaaggagtcatctgcggttatattttttattgcgattccattgatggtattctttattgtagaggcgaaagatagtatcttattactaggaggaagtcagtatcttcctgcgactttagcgatgcaaatacttatgccaattttacttatttctggtttctcgaatattacaggaaatcaaatattgattccaatgaatagagaaaaatattttatggttgcagtaacgattggtgctgtgattaatcttattttgaatctactgttaatgcctaagtttggaattattggtgcttctgtcgcaactctttttgcggaattgtcgcagatgacggtacaattacatttttcaaaagaatatttagtatcaaatatatcgataaagagtttggttaatgtgataattgcaacagttgtttctacaataccactaatcattttgaatcagctgataacgataactataccattttattctctaatgctagcaggttttgctttcttttcattatatttagtaattctgcttttattaaaggaggaagtgacgattcaattattttctcttcttgcaaagaagaagtaa

SEQ ID NO: 472 - DNA, 1422 bp, Streptococcus pneumoniae
atgaatactaaaattaaaaatataataactagtttttcttatgttatttcttcaaatctgctcatagttttaacctcatcactagttgttttgatttttcctaaaataatgggggtaactgagtacagttactggcaactttatattttttatctgacctatatcggttttttccacttgggttggattgatgggatttatctcaaatatggtggcttagaatatacaaatttagatagaaaacagttttattctcagatgattctattttctagtttcttaatgctaatctcgctggtattatttactttgaacctaataactgtaagggatgaaaacgcaagatatatttataatatggctatcattagcatgatagtcacaaacttaagaacactctatgtttatatcttgcagatgacaaatcgcttgaaggatagttcagtcattctaattagtgatcgcgttttatatgtactccttttattcatgtttattgtatttggatggcatgagtacaaggtcatgatttgggctgatattctaggtcgaacattttctctcatgctttccttctggatttgtaaagatattgtgtttcagcctttgtcaaaatttatcttagatttcaaggagtcccttgataatatccgtgttggaatcaatttaatgctatctaacattgcgagtagcatgattataggcattgttcgtatgggaattcaatggaattggaatattgaaacattcgggaaagtgtcattaactttgagtatatctaatttattaatgacttttattaatgccattggattagttatctttcctttgataaagcgaacaaagactgagaatttacctaaaatttattctaatttaagaaatgctttgatgttggttatgttcgcaatcttgctcttctactatcctttaaaatttattcttgatatttggcttcctgcttataaggatgccttagttttcatggccctaatttttcctatgtcagtttatgaagggaaaatggctttggtgataaatacatatttaaaagcaatgagaatggaaaaagacattctcaaaattaatgctttggttatgttaactagtatagtagtgacattagtgactactatactactaaataatttggggctgacagttgtatctatagttattttacttgctttaagaagtataatagctgaattaattttatccaaaaaactgaagatatcagtcaagcaagacattgctttagagttacttatgacgattatatttatttcttcaagttggtatctctctatttggattgcagtaataatttatttattggcgtatactttatatttgtatttaaagcacaaagatatcagaatgtatatagaatactttaaaaatcataaaaaaatatcataa

SEQ ID NO: 473 - DNA, 1326 bp, Streptococcus pneumoniae
atggttccgttatatacaagcgtactatctacatctgattatggaacatatgatttatttaatacgacaatctcattgctaattccaattatttcaattaatatttctgaaggggttcttcgatttgcattagatgaaaaaaatgacagtagtatagtatattcaattggctggaatattataataaaaggtttcttagttgtagtattaggaattattttcaataatatctttaatatattcccattacttaaagaaaattccataacttttttattactttatttatcaactattgtttatcaatttctttcttcatttattagaggtatagataaagtttctatattatcaattgctgctattctcaatacaatttctatattggggtttaacattctatttcttataattatacctctaggtttggttggatatttttggtctaatatattgggtttggtgctacctagtttatacttgatatataagatatctcaatataacattaagtacactagccttcagaataaaaagttacagcaaagacttgttagttacagtattcccttaatcttaaattcattaggttggtggattaataatgcaatagatagatacgtagttattgcattttgtggtgtagcggtgaatggcatctattcagttggttataaaatcccatcaattttaaatatttttgcaaatatatttaaccaagcgtggatattatcatctgtaaagtcttatcgtgatgaagatagtgaatatttcttttctcaagtttataataagtataacatgattatggtattaatttccgggctactaatttcttgtagtaaaatacttgctaagtttctatatatgaatgaattttatgatgcttggaagtttgttccgtttttactaattgcaaatgtttttggagctatttcaggatttgctggtggaattttttccgcggttaaagattcaaaaatatatagtcagtcaaccttagtaggagcgatagtgaatattattttcacatttgtttttgtatattattatggagctataggtgctgctattgccactatgatatcttactttgttgtttggataatacgtgttcatacaatgagaaaatatataaagttaaaaatttttattagaagagatgtattttcctatgtattgcttatttttcagtcaatagtcttatggttggaaaatagttatattctttatcctatacaagttgtgttattcttattattagttatgttattttataaagaaataaagagtattattggtgaattaaaaaagtttctaacataa

SEQ ID NO: 474 - DNA, 1386 bp, Streptococcus pneumoniae
atgagtaaatataaggaattagcaaaaaatacaggtatttttgctttggctaacttttcatcaaagattttaatttttttgttagtacctatatatacacgggtacttaccactacggaatatggtttttatgacttagtctatacaactattcagctttttgtaccaatcttgacattaaatatatctgaagccgttatgaggttcctaatgaaagatggtgtttctaaaaaatcagtcttttcaattgctgttttagatatatttattggatcaattgcttttgctttattgttgttagtaaataacctgttttctttatcagatttaatttctcaatacagtatttacatatttgtaatctttgttttctataccctaaataattttttgatacaattttctaagggaattgataaaattggtgttacagctatctctggggtcataagtacagcagttatgcttgccatgaatgtcattcttctagtagtatttgattggggattgttaggtttctttattgctaatgtttgtggatatgttataccttgtatttatatagtttctagattaagattgtgggaattatttgaaattaagattgataaaaaattacaatgggaaatggtttactatgctttgccattagttttaaatatattaagctggtgggtaaataatacttcagatagatatattgtgactgcaatagtaggtatacaagctagtgcaattatttcagtcgcttataaaattccacagattttgtctacgattagtgcaatttttattcaatcatggcaaatctctgcaattaaaatacaagaagataagagcgatactacatttgtatcaaacatgctcctatactataatgcacttctattgattatagcgagtggtatcatattatttgtcaaacctatttctaatatattgtttggtatttccttctattctgcgtgggagctggtaccatttttaataattagcagtctattcaatgcaatttcaggttgtataggagcaattatgggagctaaaatggatactcataacattgcaaagtcggctttagttgggatgattgcaaatattattttaaatatagtgttaacattcttaatgggtccacagggaattacaatttctaccctgatagctagttttcttattttttatatgcgaaaagatagtgtaaaagaaattaattcagaaacatatagagcaatttatttatcgtggatactattagttgtagaggcctgtctgctaatttatatggattttattattggtgctttaatagcaatggtaataaacttatttttactaaaagatgttatcaaacctttatatttaaaaattttcaaaagaaattag

SEQ ID NO: 475 - DNA, 1416 bp, Streptococcus pneumoniae; identical to SEQ ID NO: 371

SEQ ID NO: 476 - DNA, 1416 bp, Streptococcus pneumoniae
atgaaaatactaaaaaactatgcctataatctttcttatcaattgttggtgatcatactccctatcattacgactccctatgtaacgagggtttttagttctgacgatttaggaacgtatggctactttagctccattgttacctattttaccttgcttgcaactcttggtgttgccaactacggtaccaaagagatttcagcacatcgtaaggaaattgggaagaatttctggggaatttattctctccagtttggtgcaacttggctatccattttgctttatcttgccctttgtttcttatttacttcaatgcaaaatccggtagcttatatattgggattaagtttagtgtcaaaaggtttggatatttcttggttatttcaaggtttggaggattttagaaagattacagttcggaacatcactgttaagttagttggtgcaatctctattttcctatttgtaaaatcagctaatgacttatacttatatgtatttcttcttaccattttcgaattgttagggcaattaagcatgtggttacccgcaagacaatttattggtaaatcatattttgattggaaatacgctaagaagcatttgaaaccagttatcttgttattcttgccacagattgccatctcgctttatgtgactttggaccgtacgatgctgggggtgttggcttcgacaaaggatgttggaatttatgatcaagctttgaagcttgttaatattttactgactttggttacctcactggggagtgttatgttgccacgagtatctagccttttatcgtctggggattacaaagccgttaataagatgcacgagatgtcttttttaatctataacttggtgattttcccaatcatagcaggaatgttgattgtcaacgatgattttgttcgtttcttccttgggcaaaattttcaagatgctcgctatgcgattgctattatgaccttccgtatgttctttattggttggaccaatataatgggaattcaaatcttgattccgcacaataaaaataaggaatttatggtttcaacgacagttcccgctattctgagtgtgggattgaatttgttgcttcttccccaatttggttatataggtgctgcgattgtttcagttttgactgaggcgtttgtatgggggattcagttattctatacacgtttttatttaaaagaagttcctattatcggttcaatgacaaaaactgcacttgcatctgctgttatgtatggtctcttgctaggttcaaaaacatctatacatttttcgccaaccataaatgttttaatattttcggtgctaggcggaattatctatctttttgcaattctatctctgaaagtggtagatgtgaaagaattaaaacaaataatcaggaaaaattag

SEQ ID NO: 477 - DNA, 1413 bp, Streptococcus pneumoniae
atgaaattgaagtttcttataacaaatttgtttcatgtctttttgtctaatctgattacaattgtcacatcggttatagttgtactaatcttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagatggccttggtgattaatacatatttaaaggcgctaaggatggaaagagatattctcaaaataaatactttgattatgttgttcagtatgttagttaccctaataactactctattattaaataatttagagctaacggttatatctatagttgttttgctagctttacgtagtataatagcagaactaattctatctaaaaaacttgatatatcagttgagcaagacattgtgttagaattacttatgacaattatatttatttcttcaagttggtacttaccgatttggctcgcagtaatagtttatttgttagcgtatactttatacttgtatctaaagcgtaaagatacaaaaatgtatatagaatattttagaaagaaaatatttgaataa

SEQ ID NO: 478 - DNA, 1413 bp, Streptococcus pneumoniae
atgaaattgaagtttcttataacaaatttgtttcatgtctttttgtctaatctgattacaattgtcacatcggttatagttgtactaattttaccaaaaattatgggagtaactgagtatagttattggcaactatatattttttacctaacatatattggtttttttcatctgggttggattgatggaatttatcttaaatatggtggattagagtaccagaatttagataagaaacagttttattctcaaatacttcaatttttcagttttttaattttaatttcttttctattatttggttttaacttattgattgtgacagatccaaatgcaaaatatatttataacatgactattattagtatgatagttacaaatttaagaatgttattcgtttatattttgcagatgacaaatcgattaaaggatagctctataattctgataagtgatcgcgttatatatatttttcttttatttctgtttattatatttaaatggcatgaatacaaggtaatgatttgggcggatgttttaggaaggacattttctctcctactttctttttggatttgtaaagatattgtttttcaatccttatccgagttcatattggatctgagagagtcttttgacaatatccgtgttggaatcaatttaatgttatccaatattgcaagtagtatgattattggtattgttcgaatgggaattcaatggaattggaatatcgaaacattcgggaaagtatcactgacgctaagcatctctaatttattaatgacttttattaatgcgattggtttagttgtctttcctttgttaaaacggacaaaaacggaaaatttatctaaaatttattccaacttaagaaatgttttgatgctgatcatgtttgcaatattgctcttttattatcctttaaaaattattctagatctttggttgccagcttatcgggatgcgttgatttttatggctcttatttttcctatgtcaatttatgaagggaagatggccttggtgattaatacatatttaaaggcgctaaggatggaaagagatattctcaaaataaatactttgattatgttgttcagtatgttagttaccctaataactactctattattaaataatttagagctaacggttatatctatagttgttttgctagctttacgtagtataatagcagaactaattctatctaaaaaacttgatatatcagttgagcaagacattgtgttagaattacttatgacaattatatttatttcttcaagttggtacttaccgatttggctcgcagtaatagtttatttgttagcgtatactttatacttgtatctaaagcgtaaagatacaaaaatgtatatagaatattttagaaagaaaatatttgaataa

SEQ ID NO: 479 - DNA, 1461 bp, Streptococcus pneumoniae
atgactaaaaaatctattactagaaattttattttcaatacaattctaactatgtccagttttatttttccactcatcagtttcccatatgtttcaagggttctgatggcagatgggttgggacgagtaacgttcgcttcttcaattattgattatgccctaacatttgcaatgttagggatgccattatatggtgttaaagtctgtgcaggatatattaaaaataaggagaagctaagtcagactgtcagagaattgttagtcattaatttattagtaggatttcttgtgatgcttcttttagcaactatggtaataataattcctcgttttagacaggaatggcagcttattattatcacgtcatctacaattcctctcaatattataggaatagagtggttatacaaagcattggaagactattcgtatatttcaattagaacattgacttttaaaatattaggtttccttttgatgtttttagtcgtccgaacaaaagatgactatatggtatatgcggcaattacagttttagctagtcatggttcttttattttgaatttttttagatctagaactttcctactagaaaatatctctcaaagattgaatttaaagcaacatataaagccgttattaattttattttttttatctgtatcatggactatttataagaatacagatgtcgttatgatgggatttctaactagtgatacagaaattggttactattcaacagcattaaaaattcgttctatagtgctcagtgtagtaacttctttagggacagttgtactacctcgtttagtcaaatattataaagaaggaaagtataatgaagcaaagaaaatactaaacaaaagttcttcatttattatgctaagttctttatattttattggctatattgtaattaatgccagagaaattattctttttattgcgggtaggaattacttaggggctatcccgacacttcaagtatctatttttagtgcaatatttgttggctactctattatgtatggaaccaatatcctagtatctataggtaaagaaaatgtaaccattcaggcaagtattattggtgttgttttaaatatttgtttgaattttattatgatacccaaatttgctgcactaggggcaggtattgcaacttctataggagaagctgttatggtattatatgaaataatatacttgggaaaggatggttggagctattttgaaagattaaatattttaaagattattgtagtatttatatttagtacgtttatgttatatattatgaaagatttttttgttggctatcctttatttatttatattgtaatttcaggggttatttatagtattatatatatatttggtcttatgatactaagagaaaatttgctttcaagttggaaaatatcaatactaaatcggtttaatattattaaagaatga

SEQ ID NO: 480 - DNA, 1305 bp, Streptococcus pneumoniae; identical to SEQ ID NO: 354

SEQ ID NO: 481 - DNA, 1140 bp, Streptococcus pneumoniae; identical to SEQ ID NO: 466

SEQ ID NO: 482 - DNA, 1245 bp, Streptococcus pneumoniae
atgaagagacagaagtttgaatttatagagattctatactattttacagtgatgttatcagtgggaatgtttcttatgtttaccctcaacttatattggcacagaaacttattaactattttatctattgctctctcatttttaatgcttcctatactgattgttaatgctaaaagaatttctaaatctgcttttatctatggtacttttttatctatatgtattatatatgagatattaagagctaaaacactgtataattacagtgtgagtaatatttttttggcctctagacaatatatatggatttttctattttttgtattgatttacctttttaaaaacaaacaagaaaatatgagaaaaattttagataatacactcaatatttttatgttttctctcggaattagagcatttacttggtttttatatacgttatttcaagttgaattatttccatctattttaagagaattcggagatttgtggtatcgaaatgaattttcagtacgaatagatggaacaccattaattataataggtttgttaatttccacttttttctattttaaatttggaaataggaaatacttttattatttgttcttgatattaatgtatataacatttgtaaatcagacaagagtgctactggtttctgttttgatttcaatttttcttatgtttgtattttctagaagaacttctagattgctcacctctttaagttttgtaactataattatagcattcgtttatggaggtggattggattatattaaagcatatttaaatatagatgctggaacatttgatctgggattaggatttagatactgggagttgaaatactatctgggtttattagctaatgatgtatggaaacttggggttggaattctaacatcaagtaatataaatagtaattttattttggctgggccaagtgctgttaagatgtatctagatgatttaggttttttagagttattcgttcaatttggtgtagccgcaatttttatgtatggttatatattttataaattaatcaatttaattctaagaatgtcaaatgataaatatagagttgaccgcgccttttttattgcactcttaactaatttaataattacttcaatttctttaaatatctttggagcgcagagaagtttctcattggcaattgttcttgcattaatattctactatgactataggctgaaaaatgacatagaaaattag

SEQ ID NO: 483 - DNA, 1338 bp, Streptococcus pneumoniae
atgagttatttatttttactttgccttacattattcttattgactatattctatttctttgcttttattcaagatttaattgctcctccagtagttatgtctgtaatgtttctaattagttcagtatttgcactggttaattcaaaaaactggaatattgaatatagtggaatagcctatattctcataattagtggtattattatattttcgattcctttaatggcattaaaatcacctaattttaatactgaggttaagattgctgatcgattaattgatatccaattttggaaaattgctctaactattataattgatctctttattttgtatctttacaggaaggaaatatacaaccttgttcttagtaatggatatacggggtcaaatattcagtggttttttagaaatgcaacgagttatgaaggtgaattgacagtgcgaacttttattcgagttctcattcgtgttattgacgtatctgcttatatttttggatatacttttattaataattttcttatctatcgccataaacgccctaaagacatattacttttagtacctttattaatatttatttcaaaaactttaatatcaggaggccggcaagatattattaaaattctgattgcctatgtaatcatgatgtatatccaacaaaaacggaaagttggatggaatagagtcatatctcataaatatattcaccttggatttgttggtttaatagcaggtattccagcattttactactctttgtttttagccggtcgttcaacgactaggacgctatttgagagtgtttcgacctatctaggaggctcaattcagcattttaatcagtatattgaaaatccattagatcctggtgaagtttttggcagtgaaacattggtgcctatattaaatatattaggggaaatgggcctagttaattatcgtagtacaattcatttagaatttcggacactaggagttactgtaggaaatgtttatactttttttagaagacccttgcatgattttggtctagttggtatgtatgtatttgtctttgctgtaggtgctttttttgctatttattatttagttctgagaaagaaacaggttggttttaatttggatattcataccattatttattcttatgtcttttattggatttttttatcatcaatcgagcaatactcgttcacaatgattagtctatatacacttgtatttattgtgttggtttactttatggctatcttttactggtgtacagattttaaaagaggaaaactgatttttaaaatttctgactcaagtatcaaattaaaagaagaataa

SEQ ID NO: 484 - DNA, 1164 bp, Streptococcus pneumoniae
atgctctctctatatacaggaaatggtgttactcctgttattgttattttattttttttgtcggcattgtattctttatatcgaaaaagcaaattaagtataaatttaaattttttgtttatatatatttttttagcctactatttgttattaacagttatattcacacaagatttattggtgatgacttctaaatttatattatttccaatatttgtttattatataatacctaaaaatataaatggcgttataagaatactatctatttttaaatctttcattggttttactgctatttttggattatatgaatatatgcaacatttcaatcttatggtgaattttgtaaaaatagatgcagttaaatggatacaaacgatgaatttgaatagtgtgtattatcccagttcaatatttctccactacacctattttgcatatgttttgttactagcttttattttggtaattgttatcccatataagaaccgagtgttaaatttagtttataaaacacttattgcaatttcaatttttttgccccaatctcgtatagtttggatagcttttggggtaattttaattctttcgtttatactaaatagacaggggatattaacctatagaaagttgagtgttatagtacttattttaattatagtagtctctctctgcctatattttgatgtgtttgcgtttatttctaattatataagtttaagattttcttcactttatcgttatggactggctgatggttctctaggtcaacgtattggaactcttagaaattgggaaaattatttagactctgatactataaaggctattattgggtcaggatttggtggtgtcaatagttacttgagtacttattcattttttaatggctatactacagctgattcaacagtgacaagtttccttattgatactggggttattggcttatttatctttttattatctatgtttcagatattagtaatagtggtaaaaggaaaaggaatttttagagaattagcgttatatactattgtagcttcttctatagttagtttaacaattgatttttgggcaaattatgttattcttcatatattttacgtggttattatctgtgcatttataggaatgagattgactaagcaagaaaaataa

SEQ ID NO: 485 - DNA, 1071 bp, Streptococcus pneumoniae
atgttgatgttgtgtgtatttttaacattaattaagaatactctcaacgtgtttgcaaatagaatcatatattttttgattatttcatttttgtttattattgggattaatttacaaaatcttccattatcaagaaagatttatttatcattctctatgttaattatttctagcttatccaccttaccgataaagctaataaataatctcagtgatttaagaaggatatcatattacttattgcacagcatatttttatctgtatttttaggtttggtttttaaaatatctttagtaacagttgctgtagagggaattggcttttcatatggttttaatggaggtttgactcataaaaatttttatgcaattacaattttagtttcctatattctactatatgtcagcagaaaatatgacgctaaacatcagattgatagttttgtattatggttagatctttttttacttttaatatctaatacgcgaacagtttatataatactagttgttttttggattattattaatagaaattttataaataatattaaaaaagagcatagactggtagtgacagcaacgacaatagtcatctctttactggcgttaacattttttttcaaacatataattaataatagtgaatcatatagccatcgagtgttaggtgttgtgaatttttttaaatattatgaatcagataggtttcatttgttttttggggatgctgaattagcctttggaaatacgacgaagggttatggacacaatattagaagtgtgttaggttgggatggaacggtagagatgcctttactcagtgtaatgattaaaaatggttatgttgggttagttggttacattatagtgctgtttaagtttatatcttcaataatctcagttaaaaatagcacaaaaaaaaatattggattatcaatctttattccgttacttctatctgcaacagttgaaaattatatcgtcaacattagctttgtattcatgcctgtctgcttttgtattttatgttcaattaaaaatataaaattagtaaataacagaaagtaa

SEQ ID NO: 486 - DNA, 1224 bp, Streptococcus pneumoniae; identical to SEQ ID NO: 439

SEQ ID NO: 487 - DNA, 1224 bp, Streptococcus pneumoniae
atgcatgttagattagatggtttgctggactatatatttctatttagtgtgattattacttgtaatactatgtattcaactagtcaaggatttgatggactagggaaatgggcgactctgttacttgtggtatcagtttttctgaaattgcttatctctagaatatctatgaaggcaatcaatgtgattgtgtcgcgttctttaatatttatattaattattctactcatagtaatattaaatggttttaagatttctgagacaagtttcgtctattattttgtattatttccgatttttatgatgattttgcagatgtactatgatgttaatgaaatcgcaaatctgatacggaaatttgttcgtataatatttcttttagcaattggctctctcctattttggcttattggtagtgtatttcatattatatccccaacggtttatgtgttgaattattggaatggtgggggaatagtagaagggtactataatcttcattttgaagcacaaaaaatagagattttgggggcgatactgataagaaatacggggatttttgctgaagcacctatgtggagtttggtattgagccttgcattgatatttcaaacgcttcatataaaaaaatggaatttcacaacttggattcttattattacaattatgacaactacgtcaacaacaggggtttatataataggtttgattttcctatatgttttattttcaaaaacaagtggtgtgaagagatatgtttctagtttatttattttagcgactatatgttgtttttcaatattatgggataataaatctggaactggttcagcaactatacgctttgatgattataaggctggttttttggcatggcagaaaagtcctatttggggattaggtatttcagacggtcttagagccattgaacaacatatggatagaactgttagatataatcttgggtatagcaatagcttttttgttgttttagctcaaggagggataatgttggcttcgtattacttttatccagtaattaaaattattttgaataaatttagttcgaatgatttgaagttttctgcgctattaataattttccttatgataactactatatttatagagacctatatgtttttatttgtcatttctttatactattctcttgattttggggacgatagagattgtcatgagaaacagtacattactaattaa

SEQ ID NO: 488 - DNA, 1344 bp, Streptococcus pneumoniae
atgcttttaaatttcttattcatatctatttttctattaattatcattacatttatattatttgagggggatttttttcaacctgcagtaattttaacaatcgcttattttatttcgattgcaagtgctctagttaatagaaatgtttggggaacagaactccatttcaaaaccttttatttgatattgttaggggttgctacatttgttatagtttccttgttgacaaaattgtcgtacaggcctaaagtggagggaatttcgcatgaagaattgaaagaaataaatccttcaaagataatctatgtcattcttctgactctaaatcttgttatgttatttctttatatccgtgaaattcagaaagtagtattgttttcaggtagaagtttttctaatattacagatttgataagtaactataggtacctatcttattattcaaatgaagtagaaaatcgtgtaagtggaatgattaatcaactatctaaaattattccagcgactacacttatttctttatatatatttatgaataattattttataactaaacaaataaagaaaaatttcatttatttgattccaatagctatattctttgtctatgcaatcattagtggtggtagattgccccttataaggttagttgttggatctctgttgatattgtatatatactctgtgtacgggagtcctaaatctcaacttaccaaaagttttaaaatgatcactcgctctctgtttacatttcttattttgatagttttattctttcttttaaaatttgtattagggcgctcctctcaggaagattttatcagttacatcactcgttatatgggaggttcaattcaactatttgatttatttgttatagatccgatacgacgtaacaaagaactaggtgcagaaactttttcgggaatttatgagatgcttgcaaaattaggatttgacaataatattataaaaggcttagaatggagagtgtctcctaattattattctttagggaatgtgtatactgcaattagacgttattattcagactttggtgtaattggtattgtaatttgtcagagttttacagcgtggttatatactttaggttatgaaaaagttagacattattctttagttacaaatgttcaaagatttaggttgatcctattagcagcttcattttatccaatatttttaaatggtatcgaggatgtgttttatatttcaatggttaccattggatatggaatacaaattgttatcttttatctggtcttttgggttcttctgaaagttcaggttgactttaacaaaggtaaattaacgataaatagatga

SEQ ID NO: 489 - DNA, 1344 bp, Streptococcus pneumoniae
atgcttttaaatttcttattcatatctatttttctattaattatcattacatttatattatttgagggggatttttttcaacctgcagtaattttaacaatcgcttattttatttcgattgcaagtgctctagttaatagaaatgtttggggaacagaactccatttcaaaaccttttatttgatattgttaggggttgctacatttgttatagtttccttgttgacaaaattgtcgtacaggcctaaagtggagggaatttcgcatgaagaattgaaagaaataaatccttcaaagataatctatgtcattcttctgactctaaatcttgttatgctatttctttatatccgtgaaattcagaaagtagtattgttttcaggtagaagtttttctaatattacagatttgataagtaactataggtacctatcttattattcaaatgaagtagaaaatcgtgtaagtggaatgattaatcaactatctaaaattattccagcgactacacttatttctttatatatatttatgaataattattttataactaaacaaataaagaaaaatttcatttatttgattccaatagctatattctttgtctatgcaatcattagtggtggtagattgccccttataaggttagttgttggagctctgttgatattgtatatatactctgtgtacgggagtcctaaatctcaacttaccaaaagttttaaaatgatcactcgctctctgtttacatttcttattttgatagttttattctttcttttaaaatttgtattagggcgctcctctcaggaagattttatcagttacatcactcgttatatgggaggttcaattcaactatttgatttatttgttatagatccgatacgacgtaacaaagaactaggtgcagaaactttttcgggaatttatgagatgcttgcaaaattaggatttgacaataatattataaaaggcttagaatggagagtgtctcctaattattattctttagggaatgtgtatactgcaattagacgttattattcagactttggtgtaattggtattgtaatttgtcagagttttacagcgtggttatatactttaggttatgaaaaagttagacattattctttagttacaaatgttcaaagatttaggttgatcctattagcagcttcattttatccaatatttttaaatggtatcgaggatgtgttttatatttcaatggttaccattggatatggaatacaaattgttatcttttatctggtcttttgggttcttctgaaagttcaggttgactttaacaaaggtaaattaacgataaatagatga

SEQ ID NO: 490 - DNA, 1164 bp, Streptococcus pneumoniae
atgccgtatctaaataatgtaccgcaaaataaattttctataagattatttgatattgctatagttttattgactttaaagatgttagtttcttcgattccagtttttgactttgtgtttcctcaaaaatttcaaaatatccttgttattttaggttatatattgatttttttacatatttttgagaaaagaaagtacacattacaatttataattagtataattctaattactacattactgctttatactagtatacaaatgcaaaattatgtttactttacgagttggtttatgttgattggtacgattcattatgatttaagaagagtaataaaaattatatttattgtatcattaagtattatgttcatatcaatttttatatcacttcttatgtatataattgattataaacgagaaattcttattaatatccgaagaaacgaaactgtcagagcatttacatttggttttatacatcccaataaatttactattgtactatctaacttatgtcttatgtttatttggttaatcaaagatcgtttgaaatattatcatgtaactttttgtctttttattcagttattcttttattttttcacgcagactagaacagctctactagtatctatagtaatttttgctcttttatatatttatatgtttgttgaaaatcttgaacttagatggataggatactcattcttttgtatttctacttttttaggagttttggcatttcaattttatccttctaataataagttttcaatttttattgataatatcctgactggacgtattaaattagctgcatacgcaagaaccttttttggttatacattctggggtcaatatgttgataaagaaattgtttgggatcctatttggggattaactagtttcacttttgattcgttttattccttcttgatgagtaatgctggcatcatatggctacttatcttatcagtcttatttgtaaaactacaaaaatatttagacaataaaagtttgattttattattagcttggtctatgtatgcggtaacagaaactgatttaatatttccaagttatggattccagttcttatttttaagtatactttttactaatactagtacatgttctacaataatgttaaagaataattaa

SEQ ID NO: 491 - DNA, 1179 bp, Streptococcus pneumoniae; identical to SEQ ID NO: 421

SEQ ID NO: 492 - DNA, 800 bp, Artificial Sequence
  Concensus sequence of the 3′ end of the cpsA gene and the 5′ end of the cpsB gene of Streptococcus pneumoniae
tttcttgaaaatgattgacttattgggaggggtagatgttcataatgatcaagagttttcagctctacatgggaagttccatttcccagtagggaatgtccatctagactctgagcaggctctaggttttgtacgtgaacgctactcactagccgatggagaccgtgaccgtggtcgcaaccaacaaaaggtgattgtggctatccttcaaaaattaacgtcaaccgaagcactgaaaaattatagtacgatcattaatagcttgcaagattctatccaaacaaatatgccacttgagactatgataaatttggtcaatgctcagttagaaagtggagggaattataaagtaaattctcaagatttaaaaggtacaggtcggatggatcttccttcttatgcaatgccagacagtaacctctatgtgatggaaatagatgatagtagtttagctgtagttaaagcagctatacaggatgtgatggagggtagatgaaatgatagacatccattcgcatatcgtttttgatgtagatgacggtcccaagtcaagagaggaaagcaaggctctcttggcagaatcctacaggcagggggtgcgaaccattgtctctacctctcaccgtcgcaagggcatgtttgaaactccggaagagaagatagcagaaaactttcttcaggttcgggaaatagctaaggaagtggcgagtgacttagtcattgcttacggggctgaaatttactacacaccagatgttctggataagctggaaaaaaagcgagattccgaccctcaatga